SEQUENCE LISTING (4 sequences)

SEQ ID NO: 1 - protein, 192 aa, synthetic construct
MKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVR

SEQ ID NO: 2 - protein, 198 aa, synthetic construct
MKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRHHHHHH

SEQ ID NO: 3 - protein, 192 aa, synthetic construct
MKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVSGRLVQYRGEVQAMLGQSTEELRVRLASHLRKLRKRLLRDADDLQKRLAVYQAGAREGAERGLSAIRERLGPLVEQGRVR

SEQ ID NO: 4 - protein, 206 aa, synthetic construct
MKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVSGRLVQYRGEVQAMLGQSTEELRVRLASHLRKLRKRLLRDADDLQKRLAVYQAGAREGAERGLSAIRERLGPLVEQGRVRHHHHHHGGSSGSGC

(19) United States
(12) Patent Application Publication
(10) Pub. No.: US 2026/0041785 A1
(43) Pub. Date: Feb. 12, 2026

(54) POLYGLYCEROL-CONJUGATED LIPIDS AND LIPID NANOPARTICLE COMPOSITIONS COMPRISING THE SAME

(71) Applicant: Generation Bio Co., Cambridge, MA (US)

(72) Inventors: Nolan Gallagher, Cambridge, MA (US); Andrew Milstead, Cambridge, MA (US); Douglas A. Rose, Cambridge, MA (US); Sandy Zhang, Cambridge, MA (US); Daniele Vinciguerra, Cambridge, MA (US); Matthew G. Stanton, Cambridge, MA (US)

(21) Appl. No.: 19/238,030

(22) Filed: Jun. 13, 2025

Related U.S. Application Data
(63) Continuation of application No. 19/134,283.
(60) Provisional application No. 63/429,267, filed on Dec. 1, 2022.
(60) Provisional application No. 63/429,226, filed on Dec. 1, 2022.
(60) Provisional application No. 63/449,617, filed on Mar. 3, 2023.
(60) Provisional application No. 63/449,610, filed on Mar. 3, 2023.
(60) Provisional application No. 63/452,077, filed on Mar. 14, 2023.
(60) Provisional application No. 63/467,116, filed on May 17, 2023.
(60) Provisional application No. 63/467,045, filed on May 17, 2023.
(60) Provisional application No. 63/592,852, filed on Oct. 24, 2023.

(51) Int. Cl. A61K 47/69

(52) U.S. Cl. CPC A61K 47/6911

(57) ABSTRACT

The present disclosure provides novel polymer-conjugated lipids, e.g., comprising DODA conjugated to a polyglycerol or a polyglycerol derivative. The present disclosure also provides lipid nanoparticles (LNPs) formulation using the polymer-conjugated lipids and methods of treating a disease by administering the LNP formulations.

### RELATED APPLICATIONS

The instant application is a continuation of U.S. application Ser. No. 19/134,283, filed on May 30, 2025, which is a 35 U.S.C. § 371 national stage filing of International Application No. PCT/US2023/082037, filed on Dec. 1, 2023; which claims priority to U.S. Provisional Application No. 63/429,267, filed on Dec. 1, 2022; U.S. Provisional Application No. 63/429,226, filed on Dec. 1, 2022; U.S. Provisional Application No. 63/449,610, filed on Mar. 3, 2023; U.S. Provisional Application No. 63/449,617, filed on Mar. 3, 2023; U.S. Provisional Application No. 63/452,077, filed on Mar. 14, 2023; U.S. Provisional Application No. 63/467,045, filed on May 17, 2023; U.S. Provisional Application No. 63/467,116, filed on May 17, 2023; and U.S. Provisional Application No. 63/592,852, filed on Oct. 24, 2023. The entire contents of the foregoing applications are expressly incorporated herein by reference in their entireties.

### STATEMENT REGARDING SEQUENCE LISTING

The instant application contains a Sequence Listing which has been submitted electronically in .XML format and is hereby incorporated by reference in its entirety. Said .XML copy, created on Jan. 18, 2024, is named “131698-10520_Sequence_Listing.xml” and is 5,303 bytes in size. The sequence listing contained in this .XML file is part of the specification and is hereby incorporated by reference herein in its entirety.

### BACKGROUND

Lipid-based nanoparticles have played a pivotal role in the successes of COVID-19 vaccines and many other nanomedicines, such as Doxil® and Onpattro®, and have therefore been considered as a frontrunner among nanoscale drug delivery systems. However, effective targeted delivery of biologically active substances, such as therapeutic nucleic acids, represents a continuing medical challenge. This has severely limited broad applications of nucleic acids such as mRNA and DNA in protein replacement therapy, gene therapy, gene editing, and vaccination.

Lack of effective methods and vehicles for intracellular delivery represents a major barrier to a broad use of nucleic acid therapeutics. Generally, intracellular delivery of mRNA or DNA is more challenging than intracellular delivery of small oligonucleotides, in part due to the fact that mRNA and DNA molecules (which typically range from 300 kDa to 5,000 kDa, or ˜1-15 kb) are significantly larger than other types of RNAs, such as small interfering RNAs (siRNA, which are typically ˜14 kDa) or antisense oligonucleotides (ASOs, which typically range from 4 kDa to 10 kDa).

Furthermore, intracellular delivery of nucleic acid therapeutics to targeted cells is hindered by the activation of the innate and/or adaptive immune responses. Whereas it is possible to avoid RNA sensing by myeloid dendritic cells (MDCs) by chemically modifying RNA cargo (e.g., with lmΨ, 2′OMe, etc.), there are no known chemical modifications to a DNA cargo that can limit pattern recognition receptor (PRR) sensing and still maintain transcriptional activity.

An alternative approach to gene therapy is the recombinant adeno-associated virus (rAAV) vector platform that packages heterologous DNA in a viral capsid. However, there are several major disadvantages to using rAAV vectors as a gene delivery vector. One major drawback associated with rAAV is its limited viral packaging capacity of about 4.5 kb of heterologous DNA. Another major drawback is capsid immunogenicity that prevents re-administration to patients.

Thus, there remains a need for effective delivery vehicles that enable safe and effective delivery of nucleic acid therapeutics to desired cell populations.

### SUMMARY OF THE INVENTION

Accordingly, in some aspects, the present disclosure provides a polymer-conjugated lipid, comprising:
(i) a polyglycerol (PG) or a PG derivative;
(ii) a lipid moiety represented by Formula (I)

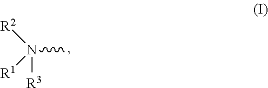

or a pharmaceutically acceptable salt thereof, wherein:
R¹ is absent, hydrogen, C₁-C₆ alkyl, or a hydrophobic tail comprising 10-30 carbon atoms;
R² is absent, hydrogen, C₁-C₆ alkyl, or a hydrophobic tail comprising 10-30 carbon atoms;
wherein, when R¹ and R² are each hydrogen, C₁-C₆ alkyl, or a hydrophobic tail comprising 10-30 carbon atoms, N is positively charged; and
R³ is a hydrophobic tail comprising 10-30 carbon atoms; and
(iii) a linker conjugating the PG or the PG derivative to the lipid moiety,
wherein [symbol] in Formula (I) is a bond conjugating the lipid moiety and the linker.

In some embodiments, the PG derivative is a carboxylated PG. In some embodiments, the carboxylated PG is a glutarylated PG. In one embodiment, the glutarylated PG is 3-methyl glutarylated PG. In one embodiment, the carboxylated PG is 2-carboxycyclohexane-1-carboxylated PG.

In some embodiments, the PG or the PG derivative is linear or branched. In some embodiments, in the lipid moiety represented by Formula (I) R¹ is absent, and wherein R² and R³ are each independently a hydrophobic tail comprising 14, 15, 16, 17, 18, 19, 20, 21, or 22 carbon atoms.

In some embodiments, R² and R³ are each independently a hydrophobic tail comprising 16, 17, 18, 19, 20, 21, or 22 carbon atoms. In some embodiments, R² and R³ are each independently a hydrophobic tail comprising 18 carbon atoms, and wherein the lipid moiety is dioctadecylamine (DODA).

In some embodiments, the lipid moiety conjugated to a linker is represented by the following structure:

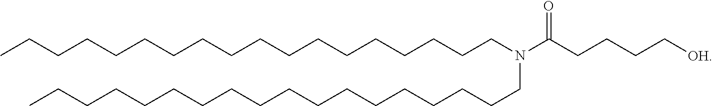 [SMILES: CCCCCCCCCCCCCCCCCCN(CCCCCCCCCCCCCCCCCC)C(=O)CCCCO]

In some embodiments, the PG or the PG derivative comprises about 5 to 100 monomeric units, or an average of 5 to 100 monomeric units.

In some embodiments, the PG or the PG derivative comprises about 5, 6, 7, 8, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 monomeric units, or an average of 5, 6, 7, 8, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 monomeric units.

In some embodiments, the PG or the PG derivative comprises about 8, 34, 45, 46, or 58 monomeric units, or an average of 8, 34, 45, 46, or 58 monomeric units. In some embodiments, the PG or the PG derivative comprises about 8 monomer units, or an average of 8 monomeric units. In some embodiments, the PG or the PG derivative comprises about 34 monomeric units, or an average of 34 monomeric units. In some embodiments, the PG or the PG derivative comprises about 45 monomeric units, or an average of 45 monomeric units. In some embodiments, the PG or the PG derivative comprises about 46 monomeric units, or an average of 46 monomeric units. In some embodiments, the PG or the PG derivative comprises about 58 monomeric units, or an average of 58 monomeric units.

In some embodiments, the linker is an alkyl linker, a glycerol linker, a phosphate linker, a phosphate ester linker, an ether linker, an ester linker, a diester linker, an amide linker, a diamide linker, an amine linker, a peptide linker, a phosphoethanolamine linker, a phosphocholine linker, a carbamate linker, a diamide alkyl linker, a cleavable linker, or any combination thereof. In some embodiments, the linker is selected from the group consisting of —(CH₂)ₙ—, —C(O)(CH₂)ₙ—, —C(O)O(CH₂)ₙ, —OC(O)(CH₂)ₙC(O)O—, and —NH(CH₂)ₙC(O)O—, wherein n is an integer ranging from 1 to 20. In some embodiments, the linker is a glutaryl linker or a succinyl linker. In some embodiments, the linker is —C(O)(CH₂)ₙ—, and wherein n is 2, 3, 4, 5, or 6. In some embodiments, n is 4.

In some aspects, the present disclosure provides a polymer-conjugated lipid represented by the following structure:

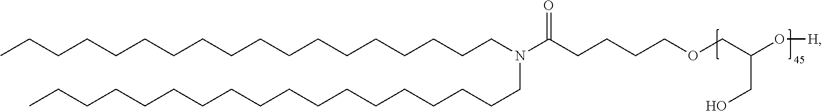

or a pharmaceutically acceptable salt or ester thereof, or a deuterated analogue thereof.

In some aspects, the present disclosure provides a polymer-conjugated lipid represented by the following structure:

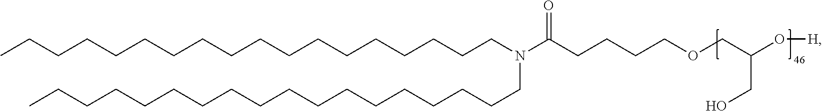

or a pharmaceutically acceptable salt or ester thereof, or a deuterated analogue thereof.

In some aspects, the present disclosure provides a polymer-conjugated lipid represented by the following structure:

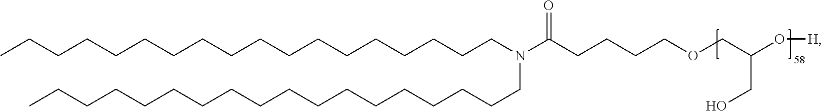

or a pharmaceutically acceptable salt or ester thereof, or a deuterated analogue thereof.

In some aspects, the present disclosure provides a polymer-conjugated lipid represented by the following structure:

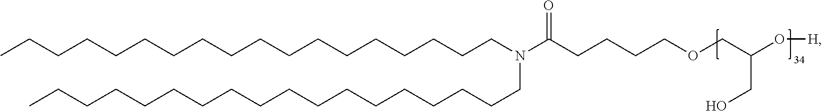

or a pharmaceutically acceptable salt or ester thereof, or a deuterated analogue thereof.

In some aspects, the present disclosure provides a polymer-conjugated lipid represented by the following structure:

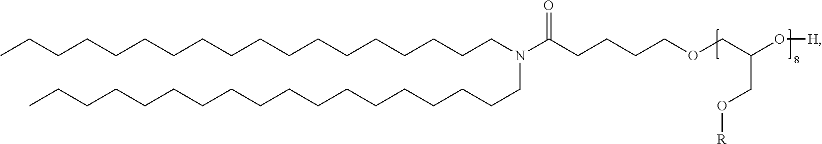

wherein R is

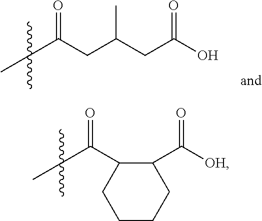

or a pharmaceutically acceptable salt or ester thereof, or a deuterated analogue thereof.

In some embodiments, the polymer-conjugated lipid of the disclosure further comprises a reactive species conjugated to the PG or the PG derivative, wherein the reactive species is functionalized to be conjugated to a targeting moiety. In some embodiments, the reactive species is a click chemistry reagent or maleimide. In some embodiments, the click chemistry reagent is selected from the group consisting of a dibenzocyclooctyne (DBCO) reagent, a transcylooctene (TCO) reagent, a tetrazine (Tz) reagent, an alkyne reagent, and an azide reagent.

In some embodiments, the polymer-conjugated lipid of the disclosure further comprises a targeting moiety conjugated to the PG or the PG derivative via the reactive species. In some embodiments, the targeting moiety is conjugated to the PG or the PG derivative via a dibenzocyclooctyne (DBCO)-azide conjugation, an azide-alkyne conjugation, a TCO-Tz conjugation, or a thiol-maleimide conjugation. In some embodiments, the targeting moiety is capable of binding to a liver cell. In some embodiments, the liver cell is a hepatocyte. In some embodiments, the targeting moiety is N-acetyl galactosamine (GalNAc) or a GalNAc derivative. In some embodiments, the targeting moiety is a tri-antennary GalNAc conjugate or a tetra-antennary GalNAc conjugate.

In some embodiments, the targeting moiety is selected from the group consisting of an ApoE protein, an ApoE polypeptide, an ApoB protein, an ApoB polypeptide, and a fragment or a variant thereof. In some embodiments, the targeting moiety is an antibody or an antibody fragment, wherein the antibody or the antibody fragment is capable of specifically binding an antigen present on the surface of a cell. In some embodiments, the antibody or the antibody fragment is a monoclonal antibody (mAb), a single chain variable fragment (scFᵥ), a heavy chain antibody (hcAb), a nanobody (Nb), a heavy-chain-only immunoglobulin (HCIg), an immunoglobulin new antigen receptor (IgNAR), variable domain of immunoglobulin new antigen receptor (V_{NAR}), a single-domain antibody, or a variable heavy chain-only antibody (V_{HH}).

In some aspects, the present disclosure also provides a lipid nanoparticle (LNP) comprising:
(i) a therapeutic nucleic acid (TNA);
(ii) an ionizable lipid;
(iii) a sterol; and
(iv) a first lipid-anchored polymer, wherein the first lipid-anchored polymer comprises the polymer-conjugated lipid of the disclosure.

In some embodiments, the LNP further comprises a helper lipid. In some embodiments, the helper lipid comprises a phospholipid or a phosphatidylcholine (PC). In some embodiments, the helper lipid is selected from the group consisting of 1,2-distearoyl-sn-glycero-3-phosphocholine (DSPC), hydrogenated soybean PC (HSPC), phosphatidylserine (PS), 1,2-dioleoyl-sn-glycero-3-phosphocholine (DOPC), 1,2-dipalmitoyl-sn-glycero-3-phosphocholine (DPPC), 1-palmitoyl-2-oleoyl-sn-glycero-3-phosphocholine (POPC), 1,2-dilauroyl-sn-glycero-3-phosphocholine (DLPC), 1-margaroyl-2-oleoyl-sn-glycero-3-phosphocholine (MOPC), 1-palmitoyl-2-linoleoyl-sn-glycero-3-phosphocholine (PLPC), 1-stearoyl-2-myristoyl-sn-glycero-3-phosphocholine (SMPC), 1,2-dimyristoyl-sn-glycero-3-phosphocholine (DMPC), 1,2-dihexanoyl-sn-glycero-3-phosphocholine (DHPC), 1,2-dierucoyl-sn-glycero-3-phosphocholine (DEPC), 1-palmitoyl-2-oleoyl-glycero-3-phosphocholine (POPC), and 1,2-dioleoyl-sn-glycero-3-phosphoethanolamine (DOPE). In one embodiment, the helper lipid is DSPC.

In some embodiments, the helper lipid is represented by Formula (II):

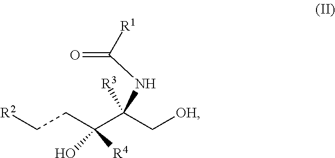

or a pharmaceutically acceptable salt or an ester thereof, wherein:
[symbol] is a single bond or a double bond;
R¹ is C₁-C₁₇ alkyl or C₂-C₁₇ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments, [symbol] is a double bond.

In some embodiments, R¹ is C₁₀-C₂₀ alkenyl, R² is C₁₀-C₂₀ alkyl and R³ is hydrogen.

In some embodiments, the helper lipid represented by Formula (II) is:

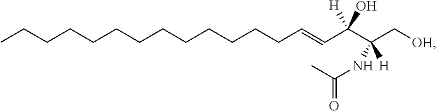 [SMILES: CCCCCCCCCCCCC/C=C/[C@@H](O)[C@H](CO)NC(C)=O]

or a pharmaceutically acceptable salt or ester thereof, or a deuterated analogue thereof.

In some embodiments, the helper lipid represented by Formula (II) is:

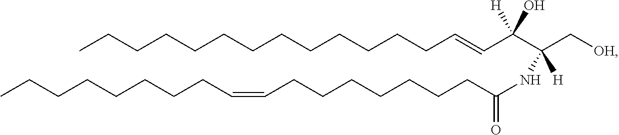 [SMILES: CCCCCCCC/C=C\CCCCCCCC(=O)N[C@@H](CO)[C@H](O)/C=C/CCCCCCCCCCCCC]

or a pharmaceutically acceptable salt or ester thereof, or a deuterated analogue thereof.

In some embodiments, the helper lipid represented by Formula (II) is:

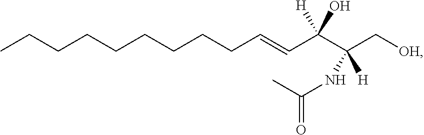 [SMILES: CCCCCCCCC/C=C/[C@@H](O)[C@H](CO)NC(C)=O]

or a pharmaceutically acceptable salt or ester thereof, or a deuterated analogue thereof.

In some embodiments, the helper lipid represented by Formula (II) is:

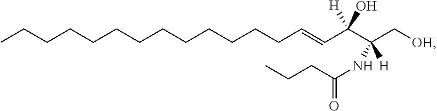 [SMILES: CCCCCCCCCCCCC/C=C/[C@@H](O)[C@H](CO)NC(=O)CCC]

or a pharmaceutically acceptable salt or ester thereof, or a deuterated analogue thereof.

In some embodiments, the helper lipid represented by Formula (II) is:

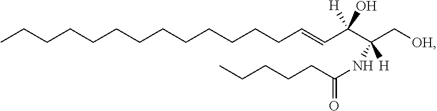 [SMILES: CCCCCCCCCCCCC/C=C/[C@@H](O)[C@H](CO)NC(=O)CCCCC]

or a pharmaceutically acceptable salt or ester thereof, or a deuterated analogue thereof.

In some embodiments, the helper lipid represented by Formula (II) is:

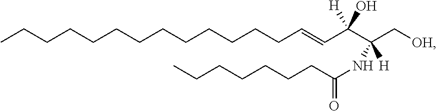 [SMILES: CCCCCCCCCCCCC/C=C/[C@@H](O)[C@H](CO)NC(=O)CCCCCCC]

or a pharmaceutically acceptable salt or ester thereof, or a deuterated analogue thereof.

In some embodiments, the sterol is selected from the group consisting of cholesterol, beta-sitosterol, stigmasterol, beta-sitostanol, campesterol, brassicasterol, and a derivative thereof. In one embodiment, the sterol is cholesterol.

In some embodiments, the ionizable lipid is represented by:
a) Formula (A):

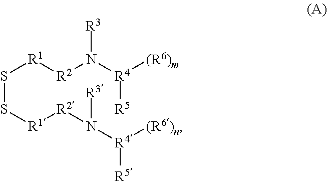

or a pharmaceutically acceptable salt thereof, wherein:
R¹ and R^{1′} are each independently optionally substituted linear or branched C₁₋₃ alkylene;
R² and R^{2′} are each independently optionally substituted linear or branched C₁₋₆ alkylene;
R³ and R^{3′} are each independently optionally substituted linear or branched C₁₋₆ alkyl;
or alternatively, when R² is optionally substituted branched C₁₋₆ alkylene, R² and R³, taken together with their intervening N atom, form a 4- to 8-membered heterocyclyl;
or alternatively, when R^{2′} is optionally substituted branched C₁₋₆ alkylene, R^{2′} and R^{3′}, taken together with their intervening N atom, form a 4- to 8-membered heterocyclyl;
R⁴ and R^{4′} are each independently —CR^{a}, —C(R^{a})₂CR^{a}, or —[C(R^{a})₂]₂CR^{a};
R^{a}, for each occurrence, is independently H or C₁₋₃ alkyl;
or alternatively, when R⁴ is —C(R^{a})₂CR^{a}, or —[C(R^{a})₂]₂CR^{a} and when R^{a} is C₁₋₃ alkyl, R³ and R⁴, taken together with their intervening N atom, form a 4- to 8-membered heterocyclyl;
or alternatively, when R^{4′} is —C(R^{a})₂CR^{a}, or —[C(R^{a})₂]₂CR^{a} and when R^{a} is C₁₋₃ alkyl, R^{3′} and R^{4′}, taken together with their intervening N atom, form a 4- to 8-membered heterocyclyl;
R⁵ and R^{5′} are each independently hydrogen, C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5; or
b) Formula (B):

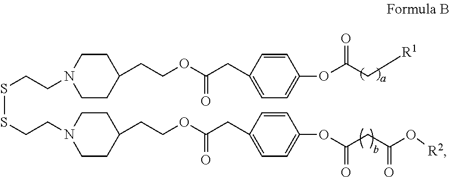

or a pharmaceutically acceptable salt thereof, wherein:
a is an integer ranging from 1 to 20;
b is an integer ranging from 2 to 10;
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl; or
c) Formula (C):

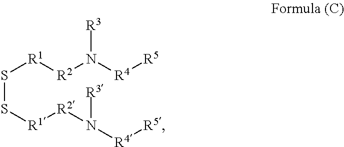

or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R;
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆)cycloalkyl; and
R^{a} and R are each halo or cyano; or
d) Formula (D):

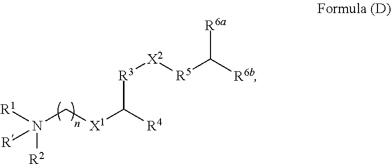

or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all positively charged;
R¹ and R² are each independently hydrogen, C₁-C₆ alkyl, or C₂-C₆ alkenyl;
R³ is C₁-C₁₂ alkylene or C₂-C₁₂ alkenylene;
R⁴ is C₁-C₁₈ unbranched alkyl, C₂-C₁₈ unbranched alkenyl, or

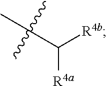

wherein:
R^{4a} and R^{4b} are each independently C₁-C₁₆ unbranched alkyl or C₂-C₁₆ unbranched alkenyl;
R⁵ is absent, C₁-C₈ alkylene, or C₂-C₈ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6;
wherein, in some embodiments, R⁴ is C₁-C₁₆ unbranched alkyl, C₂-C₁₆ unbranched alkenyl, or

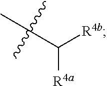

wherein R^{4a} and R^{4b} are as defined above; or
e) Formula (E):

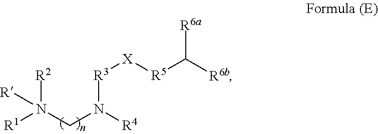

or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is positively charged;
R¹ and R² are each independently hydrogen or C₁-C₃ alkyl;
R³ is C₃-C₁₀ alkylene or C₃-C₁₀ alkenylene;
R⁴ is C₁-C₁₆ unbranched alkyl, C₂-C₁₆ unbranched alkenyl, or

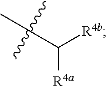

wherein:
R^{4a} and R^{4b} are each independently C₁-C₁₆ unbranched alkyl or C₂-C₁₆ unbranched alkenyl;
R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}, —OC(═O)O—, —OSi(R^{a})₂O—, —C(═O)(CR^{a})₂C(═O)O—, or OC(═O)(CR^{a}₂)C(═O)—; wherein:
R^{a}, for each occurrence, is independently hydrogen or C₁-C₆ alkyl; and
n is an integer selected from 1, 2, 3, 4, 5, and 6; or
f) an ionizable lipid selected from the group consisting of: any of the ionizable lipids in Table 1, 4, 5, 6, or 7.

In some embodiments, the ionizable lipid is Lipid 87, represented by the following structure:

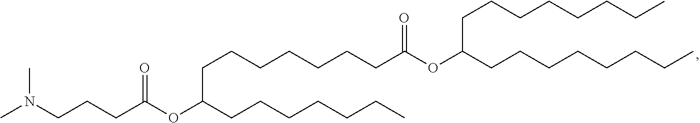 [SMILES: CCCCCCCCC(CCCCCCCC)OC(=O)CCCCCCCC(CCCCCCC)OC(=O)CCCN(C)C]

or a pharmaceutically acceptable salt or ester thereof, or a deuterated analogue thereof.

In some embodiments, the ionizable lipid is represented by the following structure:

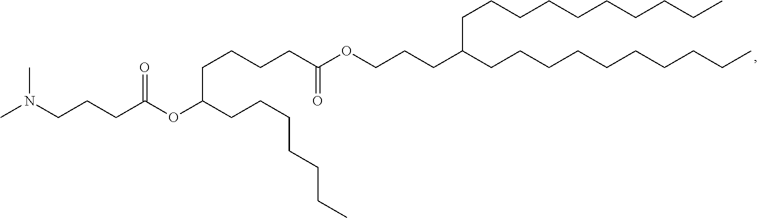 [SMILES: CCCCCCCCCCC(CCCCCCCCCC)CCCOC(=O)CCCCC(CCCCCCC)OC(=O)CCCN(C)C]

or a pharmaceutically acceptable salt or ester thereof, or a deuterated analogue thereof.

In some embodiments, the ionizable lipid is represented by the following structure:

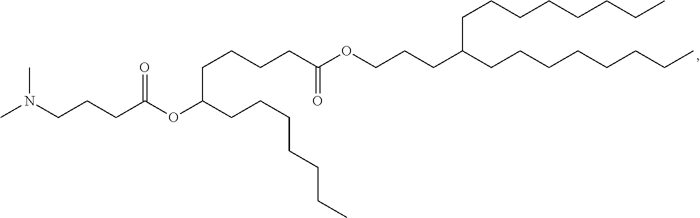 [SMILES: CCCCCCCCC(CCCCCCCC)CCCOC(=O)CCCCC(CCCCCCC)OC(=O)CCCN(C)C]

or a pharmaceutically acceptable salt or ester thereof, or a deuterated analogue thereof.

In some embodiments, the ionizable lipid is represented by the following structure:

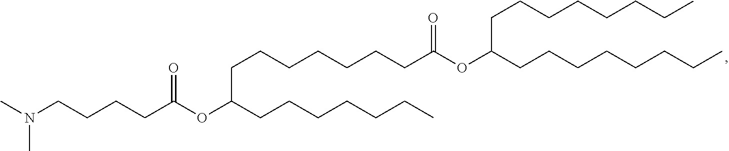 [SMILES: CCCCCCCCC(CCCCCCCC)OC(=O)CCCCCCCC(CCCCCCC)OC(=O)CCCCN(C)C]

or a pharmaceutically acceptable salt or ester thereof, or a deuterated analogue thereof.

In some embodiments, the ionizable lipid is represented by the following structure:

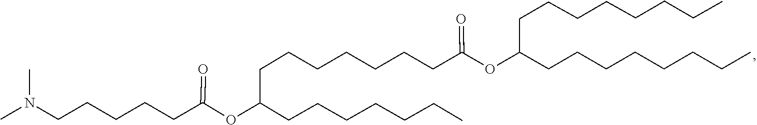 [SMILES: CCCCCCCCC(CCCCCCCC)OC(=O)CCCCCCCC(CCCCCCC)OC(=O)CCCCCN(C)C]

or a pharmaceutically acceptable salt or ester thereof, or a deuterated analogue thereof.

In some embodiments, the ionizable lipid is represented by the following structure:

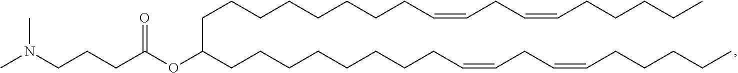 [SMILES: CCCCC/C=C\C/C=C\CCCCCCCCC(CCCCCCCC/C=C\C/C=C\CCCCC)OC(=O)CCCN(C)C]

or a pharmaceutically acceptable salt or ester thereof, or a deuterated analogue thereof.

In some embodiments, the ionizable lipid is represented by the following structure:

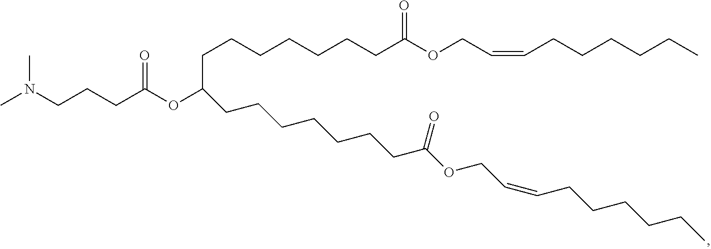 [SMILES: CCCCCC/C=C\COC(=O)CCCCCCCC(CCCCCCCC(=O)OC/C=C\CCCCCC)OC(=O)CCCN(C)C]

or a pharmaceutically acceptable salt or ester thereof, or a deuterated analogue thereof.

In some embodiments, the ionizable lipid is represented by the following structure:

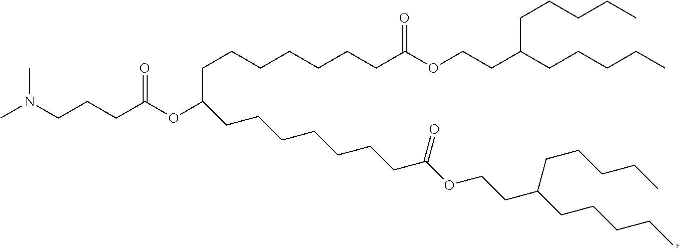 [SMILES: CCCCCC(CCCCC)CCOC(=O)CCCCCCCC(CCCCCCCC(=O)OCCC(CCCCC)CCCCC)OC(=O)CCCN(C)C]

or a pharmaceutically acceptable salt or ester thereof, or a deuterated analogue thereof.

In some embodiments, the ionizable lipid is represented by the following structure:

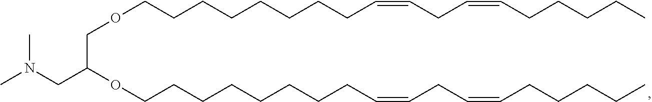 [SMILES: CCCCC/C=C\C/C=C\CCCCCCCCOCC(CN(C)C)OCCCCCCCC/C=C\C/C=C\CCCCC]

or a pharmaceutically acceptable salt or ester thereof, or a deuterated analogue thereof.

In some embodiments, the ionizable lipid is represented by the following structure:

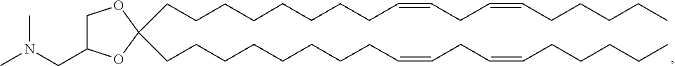 [SMILES: CCCCC/C=C\C/C=C\CCCCCCCCC1(CCCCCCCC/C=C\C/C=C\CCCCC)OCC(CN(C)C)O1]

or a pharmaceutically acceptable salt or ester thereof, or a deuterated analogue thereof.

In some embodiments, the ionizable lipid is represented by the following structure:

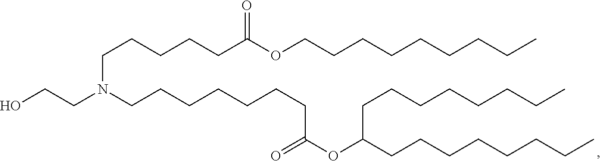 [SMILES: CCCCCCCCCOC(=O)CCCCCN(CCO)CCCCCCCC(=O)OC(CCCCCCCC)CCCCCCCC]

or a pharmaceutically acceptable salt or ester thereof, or a deuterated analogue thereof.

In some embodiments, the ionizable lipid is represented by the following structure:

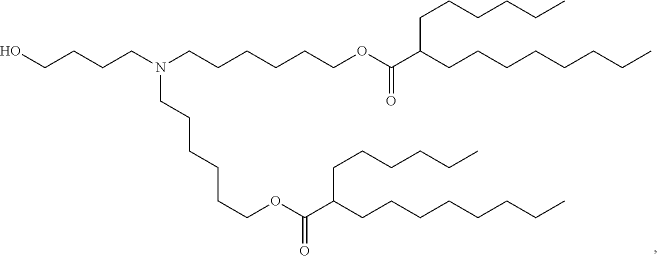 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt or ester thereof, or a deuterated analogue thereof.

In some embodiments, the ionizable lipid is Lipid Z, represented by the following structure:

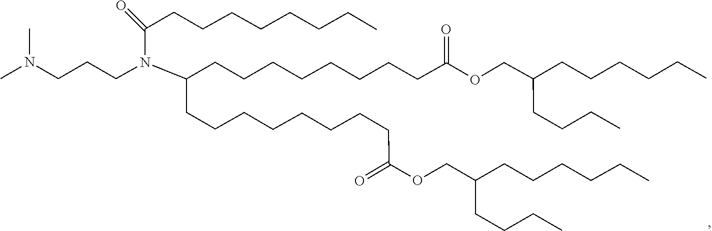 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt or ester thereof, or a deuterated analogue thereof.

In some embodiments, the ionizable lipid is represented by the following structure:

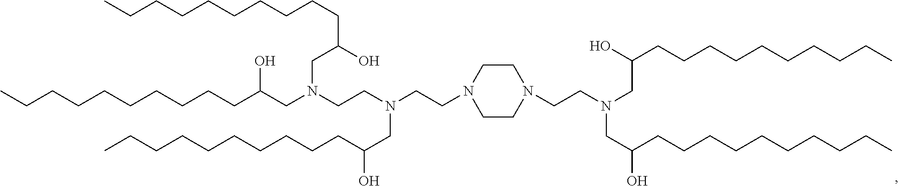 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

or a pharmaceutically acceptable salt or ester thereof, or a deuterated analogue thereof.

In some embodiments, the ionizable lipid is Lipid A, represented by the following structure:

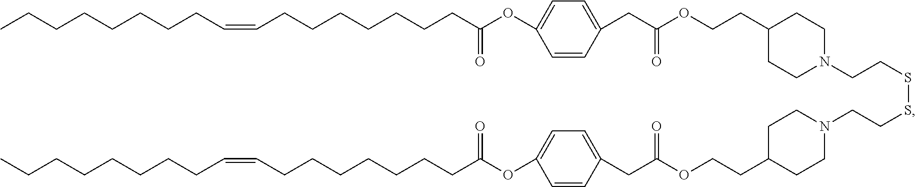 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

or a pharmaceutically acceptable salt or ester thereof, or a deuterated analogue thereof.

In some embodiments, the ionizable lipid is represented by the following structure:

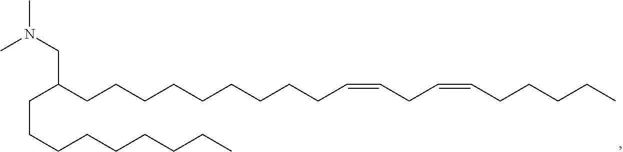 [SMILES: CCCCC/C=C\C/C=C\CCCCCCCCCC(CCCCCCCCC)CN(C)C]

or a pharmaceutically acceptable salt or ester thereof, or a deuterated analogue thereof.

In some embodiments, the LNP of the disclosure further comprises a second lipid-anchored polymer, wherein the second lipid-anchored polymer comprises:
(i) a lipid moiety comprising at least one hydrophobic tail;
(ii) a polymer;
(iii) a linker, wherein the polymer is conjugated to the lipid moiety via the linker; and
(iv) a reactive species conjugated to the polymer, wherein the reactive species is functionalized to be conjugated to a targeting moiety.

In some embodiments, the polymer of the second lipid-anchored polymer is selected from the group consisting of polyethylene (PE), polypropylene (PP), polyethylene glycol (PEG), polyvinyl alcohol (PVOH), polysarcosine (pSar), poly(2-methacryloyloxyethyl phosphorylcholine) (PMPC), polyglycerol (PG), and a derivative of any of the foregoing. In some embodiments, the PG derivative is a carboxylated PG. In one embodiment, the carboxylated PG is a glutarylated PG or 2-carboxycyclohexane-1-carboxylated PG. In one embodiment, the glutarylated PG is 3-methyl glutarylated PG.

In some embodiments, the PG or the PG derivative is linear or branched.

In some embodiments, the reactive species is a click chemistry reagent or maleimide. In some embodiments, the click chemistry reagent is selected from the group consisting of a dibenzocyclooctyne (DBCO) reagent, a transcylooctene (TCO) reagent, a tetrazine (Tz) reagent, an alkyne reagent, and an azide reagent.

In some embodiments, the LNP of the disclosure further comprises a targeting moiety conjugated to the polymer via the reactive species. In some embodiments, the targeting moiety is conjugated to the polymer via a dibenzocyclooctyne (DBCO)-azide conjugation, an azide-alkyne conjugation, a TCO-Tz conjugation, or a thiol-maleimide conjugation. In some embodiments, the targeting moiety is capable of binding to a liver cell. In some embodiments, the liver cell is a hepatocyte. In some embodiments, the targeting moiety is N-acetyl galactosamine (GalNAc) or a GalNAc derivative. In some embodiments, the targeting moiety is a tri-antennary GalNAc conjugate or a tetra-antennary GalNAc conjugate.

In some embodiments, the targeting moiety is selected from the group consisting of an ApoE protein, an ApoE polypeptide, an ApoB protein, an ApoB polypeptide, and a fragment or a variant thereof.

In some embodiments, the targeting moiety is an antibody or an antibody fragment, wherein the antibody or the antibody fragment is capable of specifically binding an antigen present on the surface of a cell. In some embodiments, the antibody or the antibody fragment is a monoclonal antibody (mAb), a single chain variable fragment (scFᵥ), a heavy chain antibody (hcAb), a nanobody (Nb), a heavy-chain-only immunoglobulin (HCIg), an immunoglobulin new antigen receptor (IgNAR), variable domain of immunoglobulin new antigen receptor (V_{NAR}), a single-domain antibody, or a variable heavy chain-only antibody (V_{HH}).

In some embodiments, the linker is selected from the group consisting of an alkyl linker, a glycerol linker, a phosphate linker, a phosphate ester linker, an ether linker, an ester linker, a diester linker, an amide linker, a diamide linker, an amine linker, a peptide linker, a phosphoethanolamine linker, a phosphocholine linker, a carbamate linker, a diamide alkyl linker, a cleavable linker, and any combination thereof. In some embodiments, the linker is selected from the group consisting of —(CH₂)ₙ—, —C(O)(CH₂)ₙ—, —C(O)O(CH₂)ₙ—, —OC(O)(CH₂)ₙC(O)O—, and —NH(CH₂)ₙC(O)O—, wherein n is an integer ranging from 1 to 20. In some embodiments, the linker is a glutaryl linker or a succinyl linker. In some embodiments, the linker is —C(O)(CH₂)ₙ—, and wherein n is 2, 3, 4, 5, or 6. In one embodiment, n is 4.

In some embodiments, in the LNP of the disclosure, the lipid moiety of the second lipid-anchored polymer is represented by Formula (I)

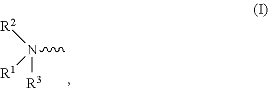

or a pharmaceutically acceptable salt thereof, wherein:
R¹ is absent, hydrogen, C₁-C₆ alkyl, or a hydrophobic tail comprising 10-30 carbon atoms;
R² is absent, hydrogen, C₁-C₆ alkyl, or a hydrophobic tail comprising 10-30 carbon atoms;
wherein, when R¹ and R² are each hydrogen, C₁-C₆ alkyl, or a hydrophobic tail comprising 10-30 carbon atoms, N is positively charged; and
R³ is a hydrophobic tail comprising 10-30 carbon atoms.

In some embodiments, R¹ is absent, and wherein R² and R³ are each independently a hydrophobic tail comprising 14, 15, 16, 17, 18, 19, 20, 21, or 22 carbon atoms. In some embodiments, R² and R³ are each independently a hydrophobic tail comprising 16, 17, 18, 19, 20, 21, or 22 carbon atoms. In some embodiments, R² and R³ are each independently a hydrophobic tail comprising 18 carbon atoms, and wherein the lipid moiety is dioctadecylamine (DODA).

In some embodiments, the lipid moiety of the second lipid-anchored polymer comprises a moiety selected from the group consisting of 1,2-dipalmitoyl-sn-glycero-3-phosphocholine (DPPC), 1-palmitoyl-2-oleoyl-glycero-3-phosphocholine (POPC), 1-palmitoyl-2-oleoyl-sn-glycero-3-phosphoethanolamine (POPE), 1-palmitoyl-2-oleoyl-sn-glycero-3-phospho-(1′-rac-glycerol) (POPG), 1,2-dipalmitoyl-sn-glycero-3-phosphoethanolamine (DPPE), 1,2-distearoyl-sn-glycero-3-phosphoethanolamine (DSPE), 1,2-dielaidoyl-sn-phosphatidylethanolamine (DEPE), 1-stearoyl-2-oleoyl-sn-glycero-3-phosphoethanolamine (SOPE), 1,2-dioleoyl-sn-glycero-3-phosphoglycerol (DOPG), 1,2-dipalmitoyl-sn-glycero-3-phosphoglycerol (DPPG), 18-1-trans PE, 1,2-dioleoyl-sn-glycero-3-phospho-L-serine (DOPS), 1,2-diphytanoyl-sn-glycero-3-phosphoethanolamine (DPHyPE), dioctadecylamine (DODA), dihexadecylamine, distearoyl-rac-glycerol (DSG), 1,2-dipalmitoyl-rac-glycerol (DPG), 1,2-dioleoyl-sn-glycero-3-phosphoethanolamine (DOPE), and a derivative thereof.

In some embodiments, the lipid moiety of the second lipid-anchored polymer comprises a moiety selected from the group consisting of DOPE, DSPE, DSG, DODA, DPG, and a derivative of any of the foregoing. In some embodiments, the lipid moiety of the second lipid-anchored polymer comprises DSPE.

In some embodiments, the polymer of the second lipid-anchored polymer has an average molecular weight of between about 500 Da and about 5000 Da. In some embodiments, the polymer has an average molecular weight of between about 1500 Da and about 5000 Da. In some embodiments, the polymer has an average molecular weight of about 2000 Da.

In some embodiments, the second lipid-anchored polymer is represented by the following structure:

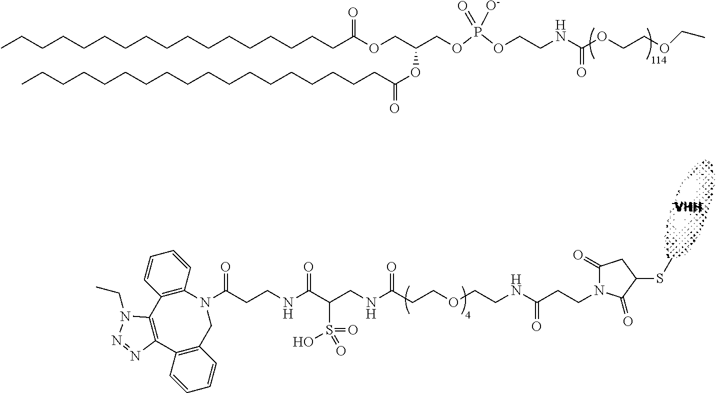

or a pharmaceutically acceptable salt or ester thereof, or a deuterated analogue thereof.

In some embodiments, the second lipid-anchored polymer is represented by the following structure:

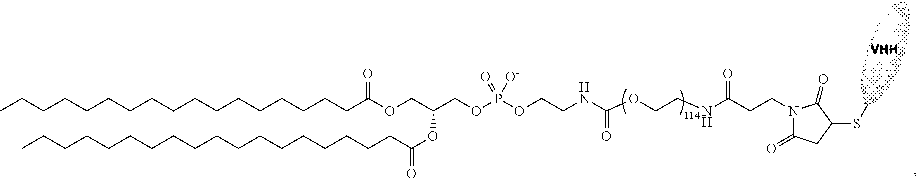

or a pharmaceutically acceptable salt or ester thereof, or a deuterated analogue thereof.

In some embodiments, the ionizable lipid is present in the LNP in an amount of about 20 mol % to about 60 mol % of the total lipid present in the LNP. In some embodiments, the ionizable lipid is present in the LNP in an amount of about 35 mol % to about 50 mol % of the total lipid present in the LNP.

In some embodiments, the sterol is present in the LNP in an amount of about 20 mol % to about 50 mol % of the total lipid present in the LNP. In some embodiments, the sterol is present in the LNP in an amount of about 30 mol % to about 45 mol % of the total lipid present in the LNP.

In some embodiments, the helper lipid is present in the LNP in an amount of about 1 mol % to about 40 mol % of the total lipid present in the LNP. In some embodiments, the helper lipid is present in the LNP in an amount of about 5 mol % to about 15 mol % of the total lipid present in the LNP.

In some embodiments, the first lipid-anchored polymer is present in the LNP in an amount of about 0.5 mol % to about 5 mol % of the total lipid present in the LNP. In some embodiments, the first lipid-anchored polymer is present in the LNP in an amount of about 1.5 mol % to about 3 mol % of the total lipid present in the LNP.

In some embodiments, the second lipid-anchored polymer is present in the LNP in an amount of about 0.05 mol % to about 5 mol % of the total lipid present in the LNP. In some embodiments, the second lipid-anchored polymer is present in the LNP in an amount of about 0.1 mol % to about 1 mol % of the total lipid present in the LNP.

In some aspects, the present disclosure also provides a lipid nanoparticle (LNP) comprising:
(i) a therapeutic nucleic acid (TNA);
(ii) an ionizable lipid, wherein the ionizable lipid is heptadecan-9-yl 9-((4-(dimethylamino)butanoyl)oxy)hexadecanoate, having the following structure:

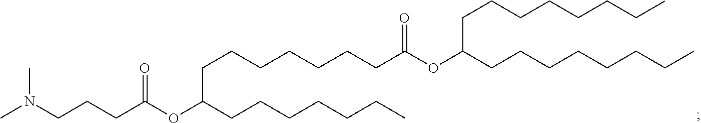 [SMILES: CCCCCCCCC(CCCCCCCC)OC(=O)CCCCCCCC(CCCCCCC)OC(=O)CCCN(C)C]

(iii) a sterol, wherein the sterol is cholesterol;
(iv) a helper lipid, wherein the helper lipid is DSPC;
(v) a first lipid-anchored polymer, wherein the first lipid-anchored polymer comprises DODA conjugated to a linear PG via a linker; and
(vi) a second lipid-anchored polymer, wherein the second lipid-anchored polymer comprises DSPE conjugated to PEG.

In some embodiments, the liner PG comprises about 30-60 monomeric units, or an average of 30-60 monomeric units.

In some embodiments, the linear PG comprises about 34 monomeric units or about 45 monomeric units, or an average of 34 monomeric units, or an average of 45 monomeric units.

In some embodiments, the PEG has an average molecular weight of between about 1000 Da and about 5000 Da. In some embodiments, the PEG has an average molecular weight of 2000 Da.

In some embodiments, in the LNP of the disclosure:
the ionizable lipid is present in the LNP in an amount of about 35 mol % to about 50 mol % of the total lipid present in the LNP;
the sterol is present in the LNP in an amount of about 30 mol % to about 45 mol % of the total lipid present in the LNP;
the helper lipid is present in the LNP in an amount of about 5 mol % to about 15 mol % of the total lipid present in the LNP;
the first lipid-anchored polymer is present in the LNP in an amount of about 1.5 mol % to about 3 mol % of the total lipid present in the LNP; and
the second lipid-anchored polymer is present in the LNP in an amount of about 0.1 mol % to about 1 mol % of the total lipid present in the LNP.

In some embodiments, the TNA comprised in the LNP of the disclosure is selected from the group consisting of a minigene, a plasmid, a minicircle, a small interfering RNA (siRNA), a microRNA (miRNA), a guide RNA (gRNA), an antisense oligonucleotide (ASO), a ribozyme, a deoxyribozyme, a closed-ended DNA (ceDNA), a ssDNA, a ministring, a Doggybone™, a protelomere closed ended DNA, a dumbbell linear DNA, a dicer-substrate dsRNA, a small hairpin RNA (shRNA), an asymmetrical interfering RNA (aiRNA), mRNA, tRNA, rRNA, gRNA, a DNA viral vector, a viral RNA vector, a non-viral vector, and a combination thereof.

In some embodiments, the TNA is ceDNA.

In some embodiments, the TNA is a single-stranded nucleic acid or a double-stranded nucleic acid. In some embodiments, the single-stranded nucleic is mRNA. In some embodiments, the single-stranded nucleic acid is a DNA molecule (ssDNA).

In some embodiments, the ssDNA is a linear ssDNA comprising at least one nucleic acid sequence of interest flanked by at least one stem-loop structure at the 3′ end. In some embodiments, the at least one stem-loop structure at the 3′ end is sufficient to prime replication and/or transcription.

In some embodiments, the stem structure at the 3′ end comprises a partial DNA duplex of between 4-500 nucleotides. In some embodiments, the stem structure at the 3′ end comprises a partial DNA duplex of between 4-50 nucleotides.

In some embodiments, the loop structure at the 3′ end comprises between 3-500 unbound nucleotides. In some embodiments, the loop structure at the 3′ end comprises a minimum of 3 unbound nucleotides.

In some embodiments, the ssDNA comprises at least two stem-loop structures at the 3′ end. In some embodiments, the ssDNA comprises at least three stem-loop structures at the 3′ end. In some embodiments, the ssDNA comprises at least four or more stem-loop structures at the 3′ end. In some embodiments, the at least one stem-loop structure at the 3′ end comprises a hairpin DNA structure. In some embodiments, the at least one stem-loop structure at the 3′ end comprises a DNA structure selected from the group consisting of: a cruciform DNA structure, a hammerhead DNA structure, a quadraplex DNA structure, a bulged DNA structure, and a multibranched loop structure. In some embodiments, the at least one stem-loop structure at the 3′ end does not comprise the A, A′, D, and D′ regions that would be present in a wild-type AAV ITR. In some embodiments, the at least one stem-loop structure at the 3′ end does not comprise the A, A′, B, B′, C, C′, D, and D′ regions that would be present in a wild-type AAV ITR. In some embodiments, the at least one stem-loop structure at the 3′ end does not comprise a rep binding element (RBE) that would be present in a wild-type ITR. In some embodiments, the at least one stem-loop structure at the 3′ end does not comprise a terminal resolution site (trs) that would be present in a wild-type ITR.

In some embodiments, the stem structure at the 3′ end comprises four or more nucleotides that are modified to be exonuclease resistant. In some embodiments, the nucleotides are phosphorothioate-modified nucleotides.

In some embodiments, at least one stem-loop structure at the 3′ end further comprises a functional moiety.

In some embodiments, the ssDNA molecule further comprises a 5′ end, comprising at least one stem-loop structure. In some embodiments, the ssDNA comprises at least two stem-loop structures at the 5′ end. In some embodiments, the ssDNA comprises at least three stem-loop structures at the 5′ end. In some embodiments, the ssDNA comprises at least four or more stem-loop structures at the 5′ end. In some embodiments, the at least one stem-loop structure at the 5′ end comprises a hairpin DNA structure. In some embodiments,
the at least one stem-loop structure at the 5′ end comprises a DNA structure selected from the group consisting of: a cruciform DNA structure, a hammerhead DNA structure, a quadraplex DNA structure, a bulged DNA structure, and a multibranched loop structure.

In some embodiments, the at least one stem-loop structure at the 5′ end does not comprise the A, A′, D, and D′ regions that would be present in a wild-type AAV ITR. In some embodiments, the at least one stem-loop structure at the 5′ end does not comprise the A, A′, B, B′, C, C′, D, and D′ regions that would be present in a wild-type AAV ITR. In some embodiments, the at least one stem-loop structure at the 5′ end does not comprise a rep binding element (RBE) that would be present in a wild-type ITR. In some embodiments, the at least one stem-loop structure at the 5′ end does not comprise a terminal resolution site (trs) that would be present in a wild-type ITR.

In some embodiments, the stem structure at the 5′ end comprises four or more nucleotides that are modified to be exonuclease resistant. In some embodiments, the nucleotides are phosphorothioate-modified nucleotides.

In some embodiments, the loop structure at the 5′ end further comprises one or more nucleic acids to stabilize the ends. In some embodiments, the loop structure at the 5′ end further comprises one or more nucleic acids that are chemically modified. In some embodiments, the loop structure at the 5′ end further comprises one or more aptamers. In some embodiments, the loop structure at the 5′ end further comprises one or more synthetic ribozymes.

In some embodiments, the loop structure at the 5′ end further comprises one or more antisense oligonucleotides (ASOs). In some embodiments, the loop structure at the 5′ end further comprises one or more short-interfering RNAs (siRNAs). In some embodiments, the loop structure at the 5′ end further comprises one or more antiviral nucleoside analogues (ANAs).

In some embodiments, the loop structure at the 5′ end further comprises one or more triplex forming oligonucleotides. In some embodiments, the loop structure at the 5′ end further comprises one or more gRNAs or gDNAs. In some embodiments, the loop structure at the 5′ end further comprises one or more molecular probes.

In some embodiments, the ssDNA molecule is devoid of any viral capsid protein coding sequences. In some embodiments, the ssDNA molecule is synthetically produced in vitro. In some embodiments, the ssDNA molecule is synthetically produced in vitro in a cell-free environment.

In some embodiments, the ssDNA molecule does not activate or minimally activates an immune pathway. In some embodiments, the immune pathway is an innate immune pathway. In some embodiments, the innate immune pathway is selected from the group consisting of the cGAS/STING pathway, the TLR9 pathway, an inflammasome-mediated pathway, and a combination thereof.

In some embodiments, the ssDNA molecule is capable of expressing at least one therapeutic protein or a therapeutic fragment thereof. In some embodiments, the at least one therapeutic protein is selected from the group consisting of an antibody, an enzyme, a coagulation factor, a transcription factor, a replication factor, a growth factor, a hormone, and a fusion protein. In some embodiments, the at least one therapeutic protein is useful for treating a genetic disorder selected from the group consisting of melanoma, hemophilia A (clotting factor VIII (FVIII) deficiency) and hemophilia B (clotting factor IX (FIX) deficiency), cystic fibrosis (CFTR), familial hypercholesterolemia (LDL receptor defect), hepatoblastoma, Wilson disease, phenylketonuria (PKU), congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis, amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis, Stargardt macular dystrophy (ABCA4), ornithine transcarbamylase (OTC) deficiency, Usher syndrome, alpha-1 antitrypsin deficiency, progressive familial intrahepatic cholestasis (PFIC) type I (ATP8B1 deficiency), type II (ABCB11), type III (ABCB4), or type IV (TJP2) and Cathepsin A deficiency.

In some aspects, the present disclosure also provides a pharmaceutical composition comprising the LNP of the disclosure and a pharmaceutically acceptable carrier.

In some aspects, the present disclosure also provides a method of treating a genetic disorder in a subject in need thereof, the method comprising administering to the subject an effective amount of the LNP of the disclosure or the pharmaceutical composition of the disclosure.

In some embodiments, the subject is a human.

In some embodiments, the genetic disorder is selected from the group consisting of sickle cell anemia; melanoma; hemophilia A (clotting factor VIII (FVIII) deficiency); hemophilia B (clotting factor IX (FIX) deficiency); cystic fibrosis (CFTR); familial hypercholesterolemia (LDL receptor defect); hepatoblastoma; Wilson's disease; phenylketonuria (PKU); congenital hepatic porphyria; an inherited disorder of hepatic metabolism; Lesch Nyhan syndrome; a thalassaemia; xeroderma pigmentosum; Fanconi's anemia; retinitis pigmentosa; ataxia telangiectasia; Bloom's syndrome; retinoblastoma; a mucopolysaccharide storage disease; a Niemann-Pick Disease; Fabry disease; Schindler disease; GM2-gangliosidosis Type II (Sandhoff Disease); Tay-Sachs disease; Metachromatic Leukodystrophy; Krabbe disease; a mucolipidosis (ML); Sialidosis Type II, a glycogen storage disease (GSD); Gaucher disease; cystinosis; Batten disease; Aspartylglucosaminuria; Salla disease; Danon disease (LAMP-2 deficiency); Lysosomal Acid Lipase (LAL) deficiency; a neuronal ceroid lipofuscinoses (NCL); a sphingolipidoses, galactosialidosis; amyotrophic lateral sclerosis (ALS); Parkinson's disease; Alzheimer's disease; Huntington's disease; spinocerebellar ataxia; spinal muscular atrophy (SMA); Friedreich's ataxia; Duchenne muscular dystrophy (DMD); a Becker muscular dystrophy (BMD), dystrophic epidermolysis bullosa (DEB); ectonucleotide pyrophosphatase 1 deficiency; generalized arterial calcification of infancy (GACI); Leber Congenital Amaurosis; Stargardt disease; wet macular degeneration (wet AMD); ornithine transcarbamylase (OTC) deficiency; Usher syndrome; alpha-1 antitrypsin deficiency; a progressive familial intrahepatic cholestasis (PFIC); and Cathepsin A deficiency.

In some aspects, the present disclosure also provides a method of providing anti-tumor immunity to a subject in need thereof, the method comprising administering to the subject an effective amount of the LNP of the disclosure or the pharmaceutical composition of the disclosure.

In some aspects, the present disclosure also provides a method of treating a subject having a disease, disorder or condition associated with an elevated expression of a tumor antigen, the method comprising administering to the subject an effective amount of the LNP of the disclosure or the pharmaceutical composition of the disclosure.

In some aspects, the present disclosure also provides a method of treating a blood disease, disorder or condition in a subject in need thereof, the method comprising administering to the subject an effective amount of the LNP of the disclosure or the pharmaceutical composition of the disclosure.

In some aspects, the present disclosure also provides a method of synthesizing a polymer-conjugated lipid of the disclosure, comprising:
a) reacting a lipid moiety which is conjugated to a linker with 2,3-epoxy-1-(1-ethoxyethoxy)propane (EEGE) in the presence of a base under argon atmosphere, or in the presence of an organocatalyst, to produce a lipid moiety conjugated to a linker and polymerized EEGE; and
b) subjecting the lipid moiety conjugated to a linker and polymerized EEGE to acidic conditions to produce the polymer-conjugated lipid.

In some embodiments, the base is a phosphazene base. In some embodiments, the phosphazene base is P4-t-Bu.

In some embodiments, the organocatalyst is an N-heterocyclic carbene (NHC) or an N-heterocyclic olefin (NHO). In some embodiments, the acidic conditions comprise HCl, Br, HI, HClO₄, HClO₃, H₂SO₄, or HNO₃.

In some embodiments, the lipid moiety comprises DODA.

In some embodiments, the lipid moiety conjugated to a linker is represented by the following structure:

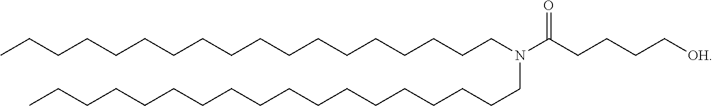 [SMILES: CCCCCCCCCCCCCCCCCCN(CCCCCCCCCCCCCCCCCC)C(=O)CCCCO]

In some embodiments, the polymer-conjugated lipid is represented by the following structure:

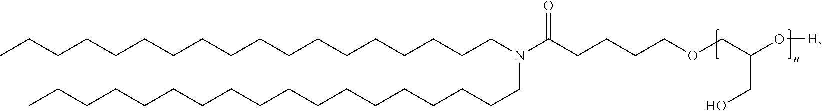

wherein n is a number ranging from 10 to 100.

In some embodiments, n is about 34, 45, 46, or 58.

### BRIEF DESCRIPTION OF THE DRAWINGS

FIG. 1A depicts a MALDI-TOF spectrum of DODA-PG34.

FIG. 1B depicts a MALDI-TOF spectrum of DODA-PG45.

FIG. 1C depicts a MALDI-TOF spectrum of DODA-PG58.

FIG. 1D shows “Scheme 1” showing synthesis of DODA-PG41 and DODA-PG46.

FIG. 1E shows “Scheme 2” showing synthesis of DODA-PG45 and DODA-PG58.

FIG. 2A shows the total flux measured by the total photon counts per the region of interest, i.e., the liver, measured in mice by In Vivo Imaging System (IVIS) at Day 4 post-dosing for LNP formulations of the disclosure and a negative control (PBS).

FIG. 2B shows the total flux measured by the total photon counts per the region of interest, i.e., the liver, measured in mice by IVIS at Day 7 post-dosing for LNP formulations of the disclosure and a negative control (PBS).

FIG. 2C shows the total flux measured by the total photon counts per the region of interest, i.e., the liver, measured in mice by IVIS across two collection days (Day 4 and Day 7) post-dosing for LNP formulations of the disclosure and a negative control (PBS).

FIG. 2D shows percent change in body weight (BW) of mice at Day 1 post-dosing with LNP formulations of the disclosure.

FIG. 3 shows luciferase activity for LNP formulations of the disclosure containing different lipid-anchored polymers.

FIG. 4A is a schematic depicting the proposed mechanism of opsonization-driven cell uptake of LNPs.

FIG. 4B is a schematic depicting the assay used for evaluating opsonization-driven cell uptake of LNPs.

FIG. 4C shows DiD fluorescence area normalized to area of live nuclei measured for LNP formulations of the disclosure containing different lipid-anchored polymers.

FIG. 5 shows DiD fluorescence area normalized to area of live nuclei for LNP formulations of the disclosure containing different amounts of polyglycerol-conjugated lipids and a control.

FIG. 6 shows the amount of endosomal escape measured as the amount of luciferase expression normalized to DiD uptake in mouse hepatocytes treated with LNP formulations of the disclosure containing different amounts of polyglycerol-conjugated lipids and a control.

FIG. 7 shows the whole blood clearance of the Control LNP, and the different Lipid Z carrying LNPs of the disclosure.

FIG. 8A shows the total flux quantified by total photon counts per the region of interest, i.e., the liver, measured in mice by IVIS at Day 7 post-dosing with LNP formulations of the disclosure and a negative control (DPBS).

FIG. 8B shows the percentage change in body weight at Day 1 of mice injected with LNP formulations of the disclosure.

FIG. 9A shows the levels of IFN-alpha measured in mice following administration of LNP formulations of the disclosure.

FIG. 9B shows the levels of IFN-gamma measured in mice following administration of LNP formulations of the disclosure.

FIG. 9C shows the levels of IL-6 measured in mice following administration of LNP formulations of the disclosure.

FIG. 9D shows the levels of IL-18 measured in mice following administration of LNP formulations of the disclosure.

FIG. 10 shows DiD fluorescence area normalized to area of live nuclei for LNP formulations of the disclosure containing different amounts of polyglycerol-conjugated lipids and a control, and formulated with DSPE-PEG5K-N₃ using a mole percentage of 0.5%.

### DETAILED DESCRIPTION

The present disclosure provides polymer-conjugated lipids, comprising, e.g., a polyglycerol (PG) conjugated to dioctadecylamine (DODA), such as DODA-PG34, DODA-PG45 and DODA-PG58, and methods of their synthesis. The present disclosure also provides lipid nanoparticles (LNPs) comprising, inter alia, polymer-conjugated lipids of the disclosure, and methods of treatment of various disorders comprising administering to a subject in need thereof LNPs of the disclosure. It has been surprisingly discovered that LNPs comprising polymer-conjugated lipids of the present disclosure are characterized by low levels of undesirable opsonization-driven uptake of LNPs into non-target cells, and balanced with desirable levels of endosomal escape, thereby achieving advantageous stealth/endosomal escape tradeoff, as described herein.

### I. Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6th Edition, published by Lippincott Williams & Wilkins, Philadelphia, PA, USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN—1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al. Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used in this specification and the appended claims, the singular forms “a”, “an” and “the” include plural references unless the content clearly dictates otherwise.

As used herein, the term “about,” when referring to a measurable value such as an amount, a temporal duration, and the like, is meant to encompass variations of 20% or +10%, more preferably +5%, even more preferably +1%, and still more preferably +0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The term “consisting of” refers to compositions, methods, processes, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the disclosure.

As used herein the terms, “administration,” “administering” and variants thereof refers to introducing a composition or agent (e.g., ceDNA, ssDNA, mRNA, etc.) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

The term “immunogenicity of an LNP” or “immunogenicity of a composition comprising an LNP”, as used herein, refers to the ability of a composition comprising LNPs of the present disclosure to stimulate an undesired immune response in a subject after the LNPs of the disclosure or a composition comprising the LNPs of the disclosure are administered to the subject. In some embodiments, the immune response, e.g., before and after administration of a composition comprising LNPs of the present disclosure, may be measured by measuring levels of one or more pro-inflammatory cytokines. Exemplary pro-inflammatory cytokines that may be used to determine immunogenicity of LNPs of the present disclosure or a composition comprising LNPs of the present disclosure include, but are not limited to, granulocyte colony stimulating factor (G-CSF), interleukin 1 alpha (IL-1α), interleukin 1 beta (IL-1β), interleukin 6 (IL-6), interleukin 8 (IL-8 or CXCL8), interleukin 11 (IL-11), interleukin 17 (IL-17), interleukin 18 (IL-18), interferon α (IFN-α), interferon β (IFN-β), interferon γ (IFN-γ), C-X-C motif chemokine ligand 10 (CXCL10 or IP-10), monocyte chemoattractant protein 1 (MCP-1), CD40L, CCL2, CCL3, CCL4, CCL5, CCL11, tumor necrosis factor α (TNF-α), and combinations thereof.

The term “off-target delivery”, as used herein, refers to delivery of LNPs of the disclosure to non-target cells. For example, an LNP of the disclosure comprising GalNAc targets delivery of the LNP to hepatocytes, and off-target delivery of the LNP refers to the delivery of the LNP to random, non-target cells that are not, for example, hepatocytes. In some embodiments, the non-target cell may be a blood cell, e.g., a leukocyte, a neutrophil, an eosinophil, a basophil, a macrophage, or a monocyte. In some embodiments, the non-target cell may be an immune cell, such as a T-cell, B-cell or a macrophage. In some embodiments, the non-target cell may be a liver sinusoidal endothelial cell (LSEC cell), a spleen cell or a Kupffer cell.

After administration to a subject, an LNP may be delivered to a non-target cell, e.g., one or more of blood cells listed above, and may result in expression of a therapeutic nucleic acid (TNA) in the non-target cell, or may be degraded once engulfed by, e.g., a macrophage. In some embodiments, a reference LNP that does not contain a polyglycerol-conjugated lipid, may be characterized by a higher rate of delivery to a non-target cell, e.g., one or more of blood cells listed above, as compared to an LNP of the present disclosure. In some embodiments, an LNP of the present disclosure results in an uptake level of TNA (e.g., ceDNA or mRNA) in a non-target cell, e.g., a blood cell, that is lower than that of a reference LNP. In some embodiments, the reference LNP is an LNP that does not comprise a polymer-conjugated lipid. In some embodiments, the blood cell is a cell selected from the group consisting of a red blood cell, a leukocyte, a neutrophil, a macrophage, a monocyte, a T-cell, a B-cell, a macrophage and a peripheral blood mononuclear cell.

As used herein, the term “aqueous solution” refers to a composition comprising in whole, or in part, water.

As used herein, the term “bases” includes purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

As used herein, the terms “carrier” and “excipient” are meant to include any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, the term “ceDNA” refers to capsid-free closed-ended linear double stranded (ds) duplex DNA for non-viral gene transfer, synthetic or otherwise. According to some embodiments, the ceDNA is a closed-ended linear duplex (CELiD) CELiD DNA. According to some embodiments, the ceDNA is a DNA-based minicircle. According to some embodiments, the ceDNA is a minimalistic immunological-defined gene expression (MIDGE)-vector. According to some embodiments, the ceDNA is a ministering DNA. According to some embodiments, the ceDNA is a dumbbell shaped linear duplex closed-ended DNA comprising two hairpin structures of ITRs in the 5′ and 3′ ends of an expression cassette. According to some embodiments, the ceDNA is a Doggybone™ DNA. Detailed description of ceDNA is described in International Patent Application No. PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International Patent Application Nos. PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International Application PCT/US2019/14122, filed on Jan. 18, 2019, the entire content of which is incorporated herein by reference.

As used herein, the term “closed-ended DNA vector” refers to a capsid-free DNA vector with at least one covalently closed end and where at least part of the vector has an intramolecular duplex structure.

As used herein, the terms “ceDNA vector” and “ceDNA” are used interchangeably and refer to a closed-ended DNA vector comprising at least one terminal palindrome. In some embodiments, the ceDNA comprises two covalently-closed ends.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the term “ceDNA genome” refers to an expression cassette that further incorporates at least one inverted terminal repeat (ITR) region. A ceDNA genome may further comprise one or more spacer regions. In some embodiments the ceDNA genome is incorporated as an intermolecular duplex polynucleotide of DNA into a plasmid or viral genome.

As used herein, the terms “DNA regulatory sequences,” “control elements,” and “regulatory elements,” are used interchangeably herein, and refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

The “ITR” can be artificially synthesized using a set of oligonucleotides comprising one or more desirable functional sequences (e.g., palindromic sequence, RBS). The ITR sequence can be an AAV ITR, an artificial non-AAV ITR, or an ITR physically derived from a viral AAV ITR (e.g., ITR fragments removed from a viral genome). For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. Typically, ITR sequences can be derived not only from AAV, but also from Parvovirus, lentivirus, goose virus, B19, in the configurations of wildtype, “doggy bone” and “dumbbell shape”, symmetrical or even asymmetrical ITR orientation. Although the ITRs are typically present in both 5′ and 3′ ends of an AAV vector, ITR can be present in only one of end of the linear vector. For example, the ITR can be present on the 5′ end only. Some other cases, the ITR can be present on the 3′ end only in synthetic AAV vector. For convenience herein, an ITR located 5′ to (“upstream of”) an expression cassette in a synthetic AAV vector is referred to as a “5′ ITR” or a “left ITR”, and an ITR located 3′ to (“downstream of”) an expression cassette in a vector or synthetic AAV is referred to as a “3′ ITR” or a “right ITR”.

As used herein, a “wild-type ITR” or “WT-ITR” refers to the sequence of a naturally occurring ITR sequence in an AAV genome or other dependovirus that remains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompasses for use herein include WT-ITR sequences as result of naturally occurring changes (e.g., a replication error).

As used herein, the term “substantially symmetrical WT-ITRs” or a “substantially symmetrical WT-ITR pair” refers to a pair of WT-ITRs within a synthetic AAV vector that are both wild type ITRs that have an inverse complement sequence across their entire length. For example, an ITR can be considered to be a wild-type sequence, even if it has one or more nucleotides that deviate from the canonical naturally occurring canonical sequence, so long as the changes do not affect the physical and functional properties and overall three-dimensional structure of the sequence (secondary and tertiary structures). In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR” are used interchangeably and refer to an ITR with a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e., its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single synthetic AAV genome that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single stranded AAV genome that are wild-type or mutated (e.g., modified relative to wild-type) dependoviral ITR sequences and are inverse complements across their full length. In one non-limiting example, both ITRs are wild type ITRs sequences from AAV2. In another example, neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a synthetic AAV vector is referred to as a “5′ ITR” or a “left ITR”, and an ITR located 3′ to (downstream of) an expression cassette in a synthetic AAV vector is referred to as a “3′ ITR” or a “right ITR”.

As used herein, the terms “substantially symmetrical modified-ITRs” or a “substantially symmetrical mod-ITR pair” refers to a pair of modified-ITRs within a synthetic AAV that are both that have an inverse complement sequence across their entire length. For example, the modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

As used herein, the phrase an “effective amount” or “therapeutically effective amount” of an active agent or therapeutic agent, such as a therapeutic nucleic acid, is an amount sufficient to produce the desired effect, e.g., inhibition of expression of a target sequence in comparison to the expression level detected in the absence of a therapeutic nucleic acid. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art.

As used herein, the term “expression” refers to the cellular processes involved in producing RNA and proteins and as appropriate, secreting proteins, including where applicable, but not limited to, for example, transcription, transcript processing, translation and protein folding, modification and processing. As used herein, the phrase “expression products” include RNA transcribed from a gene (e.g., transgene), and polypeptides obtained by translation of mRNA transcribed from a gene.

As used herein, the term “expression vector” refers to a vector that directs expression of an RNA or polypeptide from sequences linked to transcriptional regulatory sequences on the vector. The sequences expressed will often, but not necessarily, be heterologous to the host cell. An expression vector may comprise additional elements, for example, the expression vector may have two replication systems, thus allowing it to be maintained in two organisms, for example in human cells for expression and in a prokaryotic host for cloning and amplification. The expression vector may be a recombinant vector.

As used herein, the term “flanking” refers to a relative position of one nucleic acid sequence with respect to another nucleic acid sequence. Generally, in the sequence ABC, B is flanked by A and C. The same is true for the arrangement AxBxC. Thus, a flanking sequence precedes or follows a flanked sequence but need not be contiguous with, or immediately adjacent to the flanked sequence.

As used herein, the term “spacer region” refers to an intervening sequence that separates functional elements in a vector or genome. In some embodiments, spacer regions keep two functional elements at a desired distance for optimal functionality. In some embodiments, the spacer regions provide or add to the genetic stability of the vector or genome. In some embodiments, spacer regions facilitate ready genetic manipulation of the genome by providing a convenient location for cloning sites and a gap of design number of nucleotide.

As used herein, the terms “expression cassette” and “expression unit” are used interchangeably, and refer to a heterologous DNA sequence that is operably linked to a promoter or other DNA regulatory sequence sufficient to direct transcription of a transgene of a DNA vector, e.g., synthetic AAV vector. Suitable promoters include, for example, tissue specific promoters. Promoters can also be of AAV origin.

As used herein, a “vector” or “expression vector” is a replicon, which can be a nucleic acid construct designed for delivery to a host cell or for transfer between different host cells. As used herein, a vector can be viral or non-viral in origin in the final form. For the purpose of the present disclosure, a “vector” generally refers to synthetic, capsid-free AAV, for example a single-stranded (ss) synthetic vector or a nicked ceDNA vector. Accordingly, the term “vector” encompasses any genetic element that is capable of replication or expression when associated with the proper control elements and that can transfer gene sequences to cells. In some embodiments, a vector can be a recombinant vector or an expression vector. It is to be understood that the term “single-stranded (ss) synthetic vector” as used herein includes a single-stranded AAV-like vector that may not have any viral sequence(s).

As used herein, the term “genetic disease” refers to a disease, partially or completely, directly or indirectly, caused by one or more abnormalities in the genome, especially a condition that is present from birth and can be treated by a single-stranded (ssDNA) molecule as described herein. The abnormality may be a mutation, an insertion or a deletion. The abnormality may affect the coding sequence of the gene or its regulatory sequence. The genetic disease may be, but not limited to phenylketonuria (PKU), melanoma, hemophilia A (clotting factor VIII (FVIII) deficiency) and hemophilia B (clotting factor IX (FIX) deficiency), cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and mucopolysaccharide storage diseases (e.g., Hurler syndrome (MPS Type I), Scheie syndrome (MPS Type I S), Hurler-Scheie syndrome (MPS Type I H-S), Hunter syndrome (MPS Type II), Sanfilippo Types A, B, C, and D (MPS Types III A, B, C, and D), Morquio Types A and B (MPS IVA and MPS IVB), Maroteaux-Lamy syndrome (MPS Type VI), Sly syndrome (MPS Type VII), hyaluronidase deficiency (MPS Type IX)), Niemann-Pick Disease Types A/B, C1 and C2, Fabry disease, Schindler disease, GM2-gangliosidosis Type II (Sandhoff Disease), Tay-Sachs disease, Metachromatic Leukodystrophy, Krabbe disease, Mucolipidosis Type I, II/III and IV, Sialidosis Types I and II, Glycogen Storage disease Types I and II (Pompe disease), Gaucher disease Types I, II and III, Fabry disease, cystinosis, Batten disease, Aspartylglucosaminuria, Salla disease, Danon disease (LAMP-2 deficiency), Lysosomal Acid Lipase (LAL) deficiency, neuronal ceroid lipofuscinoses (CLN1-8, INCL, and LINCL), sphingolipidoses, galactosialidosis. Also included in genetic disorders are amyotrophic lateral sclerosis (ALS), Parkinson's disease, Alzheimer's disease, Huntington's disease, spinocerebellar ataxia, spinal muscular atrophy, Friedreich's ataxia, Duchenne muscular dystrophy (DMD), Becker muscular dystrophies (BMD), dystrophic epidermolysis bullosa (DEB), ectonucleotide pyrophosphatase 1 deficiency, generalized arterial calcification of infancy (GACI), Leber Congenital Amaurosis (LCA, e.g., LCA10 [CEP290]), Stargardt macular dystrophy (ABCA4), or Cathepsin A deficiency.

The terms “peptide,” “polypeptide,” and “protein” are used interchangeably herein, and refer to a polymeric form of amino acids of any length, which can include chemically or biochemically modified or derivatized amino acids, and polypeptides having modified peptide backbones.

According to some embodiments, a polypeptide of the disclosure is an ApoE or an ApoB polypeptide. According to some embodiments, the ApoE polypeptide is a functional fragment (or a functional portion) of the full length ApoE polypeptide. According to some embodiments, the ApoE polypeptide is a functional fragment (or a functional portion) of the full length ApoB polypeptide. According to some embodiments, the ApoE polypeptide is 30 amino acids in length or less. According to some embodiments, the ApoB polypeptide is 30 amino acids in length or less.

As used herein, the term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and D-acyloxyacids, are also within the group designated as amphipathic lipids. Additionally, the amphipathic lipids described above can be mixed with other lipids including triglycerides and sterols.

As used herein, the term “polyglycerol” refers to an organic compound that is a polymeric condensation product of glycerol. Polyglycerols obtained from the dehydration of glycerol can have linear, branched, or cyclic structures. In some embodiments, the polyglycerol of the disclosure is linear or branched. In one embodiment, the polyglycerol is linear. In one embodiment, the polyglycerol is branched. In some embodiments, the term “polyglycerol” encompasses a population of polyglycerol molecules. A population of polyglycerol molecules may comprise a distribution of polyglycerol molecules of different lengths, i.e., a distribution of polyglycerol molecules comprising different numbers of monomeric units. Thus, the term “average molecular weight”, when used herein in reference to polyglycerol, refers to an average molecular weight of a population of polyglycerol molecules. The average molecular weight of a polyglycerol may be determined by any method known in the art, e.g., MALDI-MS or NMR. The term “average”, when used herein in reference to the number of monomeric units present in a polyglycerol, refers to an average number of monomeric units per polyglycerol molecule in a population of polyglycerol molecules. Thus, the language, e.g., “an average of 45 monomeric units”, when used herein in reference to the number of monomeric units present in a polyglycerol, refers to an average of 45 monomeric units per polyglycerol molecule in a population of polyglycerol molecules. The average number of monomeric units per polyglycerol molecule may be calculated based on an average molecular weight of a polyglycerol.

In some embodiments, a polyglycerol may comprise an average of 8 to 100 monomeric units, e.g., an average of 8 to 40 monomeric units, an average of 15-75 monomeric units, an average of 20 to 50 monomeric units, an average of 30 to 70 monomeric units, an average of 40 to 90 monomeric units or an average of 50 to 100 monomeric units. In some embodiments, the polyglycerol of the disclosure comprises an average of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 monomeric units.

As used herein, the term “linear”, as it refers to a polyglycerol or an aliphatic hydrocarbon chain, means that the chain is unbranched.

As used herein, the term “polyglycerol derivative” or a “PG derivative” refers to polyglycerol in which free alcohol groups have been modified. In some embodiments, the polyglycerol derivative of the disclosure is linear or branched. In one embodiment, a polyglycerol derivative is linear. In one embodiment, the polyglycerol derivative is branched.

In some embodiments, the term “polyglycerol derivative” encompasses a population of polyglycerol derivative molecules. A population of polyglycerol derivative molecules may comprise a distribution of polyglycerol derivative molecules of different lengths, i.e., distribution of polyglycerol derivative molecules comprising different numbers of monomeric units. Thus, the term “average molecular weight”, when used herein in reference to a polyglycerol derivative, refers to an average molecular weight of a population of polyglycerol derivative molecules. An average molecular weight of a polyglycerol derivative may be determined by any method known in the art, e.g., MALDI-MS or NMR. The term “average”, when used herein in reference to the number of monomeric units present in a polyglycerol derivative, refers to an average number of monomeric units per polyglycerol derivative molecule in a population of polyglycerol derivative molecules. Thus, the language, e.g., “an average of 8 monomeric units”, when used herein in reference to the number of monomeric units present in a polyglycerol derivative, refers to an average of 8 monomeric units per polyglycerol derivative molecule in a population of polyglycerol derivative molecules. An average number of monomeric units per polyglycerol derivative molecule may be calculated based on an average molecular weight of a polyglycerol derivative.

In some embodiments, a polyglycerol derivative of the disclosure may comprise an average of 8 to 100 monomeric units, e.g., an average of 8 to 40 monomeric units, an average of 15-75 monomeric units, an average of 20 to 50 monomeric units, an average of 30 to 70 monomeric units, an average of 40 to 90 monomeric units or an average of 50 to 100 monomeric units. In some embodiments, the polyglycerol of the disclosure comprises an average of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 monomeric units.

In some embodiments, a polyglycerol derivative may be a carboxylated polyglycerol, i.e., a polyglycerol in which the free alcohol groups have been modified by converting them into a moiety comprising one or more carboxylate groups, e.g., 2-carboxycyclohexane-1-carboxylated polyglycerol. In some embodiments, a polyglycerol derivative may be a glutarylated polyglycerol, i.e., a polyglycerol in which free alcohol groups have been modified by converting them into a glutarate or a glutarate derivative, e.g., 3-methyl glutarylated polyglycerol. In some embodiments, a polyglycerol derivative may be conjugated to a lipid moiety, e.g., a lipid moiety represented by Formula (I) as described herein. In some embodiments, a polyglycerol derivative that is conjugated to a lipid moiety is represented by the following structural formula:

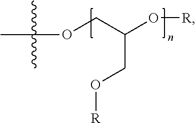

wherein:
n is an integer ranging from 8 to 100; and
R is selected from the group consisting of OH and

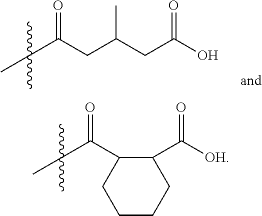

As used herein, the term “hydrophobic tail” refers to a hydrocarbon chain, i.e., a chain containing carbon and hydrogen atoms, that can be saturated or unsaturated. In one embodiment, the hydrophobic tail may comprise 10-30 carbon atoms, e.g., 14, 15, 16, 17, 18, 19, 20, 21, or 22 carbon atoms. In some embodiments, the hydrocarbon chain of a hydrophobic tail is unsaturated, i.e., does not comprise double or triple bonds. In other embodiments, the hydrocarbon chain of a hydrophobic tail is unsaturated, comprising one or more double bonds and/or one or more triple bonds. In some embodiments, the hydrocarbon chain may be a linear chain. In other embodiments, the hydrocarbon chain may be a branched chain. Non-limiting examples of backbone hydrophobic tail of the present disclosure include the hydrophobic tails present in lauric acid, myristic acid, myristoleic acid, octadecylamine, palmitic acid, stearic acid, arachidic acid, palmitoleic acid, sapienic acid, oleic acid, elaidic acid, vaccenic acid, linoleic acid, linoelaidic acid, ax-linolenic acid, arachidonic acid, eicosapentaenoic acid, and a derivative thereof.

As used herein, the term “click chemistry reaction product” refers to a moiety formed by two click chemistry reagents of a “click pair”. In some embodiments, the click chemistry reaction product is a product of a reaction between: a) a tetrazine reagent (i.e., a reagent comprising a tetrazine moiety) and a transcyclooctene reagent (i.e., a reagent comprising a transcyclooctene moiety); b) a tetrazine reagent and a norbornene reagent (i.e., a reagent comprising a norbornene moiety); or c) a an azide reagent (i.e., a reagent comprising an azide moiety) and an alkyne reagent, e.g., a dibenzocyclooctyne (DBCO) reagent.

As used herein, the term “lipid-anchored polymer”, which may be used herein interchangeably with the term “lipid conjugate”, refers to a molecule comprising a lipid moiety covalently attached to a polymer via a linker. Without wishing to be bound by a specific theory, it is believed that a lipid-anchored polymer can inhibit aggregation of LNPs and provide steric stabilization. Exemplary lipid-anchored polymers include, but are not limited to polymer-conjugated lipids as described herein, PEGylated lipids such as, e.g., PEG coupled to dialkyloxypropyls (e.g., PEG-DAA conjugates), PEG coupled to diacylglycerols (e.g., PEG-DAG conjugates), PEG coupled to dimyristolglycerol (e.g., PEG-DMG), PEG coupled to distearoyl glycerol (e.g., PEG-DSG), PEG coupled to poly(2-methacryloyloxyethyl phosphorylcholine) (e.g., PEG-PMPC), PEG coupled to 1,2-distearoyl-sn-glycero-3-phosphoethanolamine (e.g., PEG-DSPE), polyglycerol coupled to dioctadecylamine (e.g., DODA-PG), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in International Patent Application Publication No. WO 2010/006282. PEG, PG or POZ can be conjugated directly to the lipid or may be linked to the lipid via a linker moiety. Any linker moiety suitable for coupling the PEG, PG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

Exemplary linkers that may be used to conjugate a lipid moiety to the polymer in a lipid-anchored polymer of the present disclosure may be selected from the group consisting of an alkyl linker, a glycerol linker, a phosphate linker, a phosphate ester linker, an ether linker, an ester linker, a diester linker (e.g., a glutaryl linker, a succinyl linker), an amide linker, a diamide linker, an amine linker, a peptide linker, a phosphoethanolamine linker, a phosphocholine linker, a carbamate linker, a diamide alkyl linker, a cleavable linker, a click reaction product, and any combination thereof. In some embodiments, the linker may be selected from the group consisting of —(CH₂)ₙ—, —C(O)(CH₂)ₙ—, —C(O)O(CH₂)ₙ—, —OC(O)(CH₂)ₙC(O)O—, and —NH(CH₂)ₙC(O)O—, wherein n is an integer ranging from 1 to 20. In some embodiments, the linker is —C(O)(CH₂)ₙ—, and wherein n is 2, 3, 4, 5, or 6. In one specific embodiment, n is 4.

As used herein, the term “lipid encapsulated” refers to a lipid particle that provides an active agent or therapeutic agent, such as a nucleic acid (e.g., a ceDNA), with full encapsulation, partial encapsulation, or both. In a preferred embodiment, the nucleic acid is fully encapsulated in the lipid particle (e.g., to form a nucleic acid containing lipid particle).

As used herein, the terms “lipid particle” or “lipid nanoparticle” or “LNP” refers to a lipid formulation that can be used to deliver a therapeutic agent such as nucleic acid therapeutics to a target site of interest (e.g., cell, tissue, organ, and the like). In one embodiment, the lipid particle of the disclosure is a nucleic acid containing lipid particle, which is typically formed from a cationic lipid, a non-cationic lipid, and optionally a conjugated lipid that prevents aggregation of the particle. In other preferred embodiments, a therapeutic agent such as a therapeutic nucleic acid may be encapsulated in the lipid portion of the particle, thereby protecting it from enzymatic degradation. In one embodiment, the lipid particle comprises a nucleic acid (e.g., ceDNA, ssDNA, mRNA, etc.) and a lipid comprising one or more tertiary amino groups, one or more phenyl ester bonds and a disulfide bond.

According to some embodiments, lipid particles of the disclosure typically have a mean diameter of from about 20 nm to about 75 nm, about 20 nm to about 70 nm, about 25 nm to about 75 nm, about 25 nm to about 70 nm, from about 30 nm to about 75 nm, from about 30 nm to about 70 nm, from about 35 nm to about 75 nm, from about 35 nm to about 70 nm, from about 40 nm to about 75 nm, from about 40 nm to about 70 nm, from about 45 nm to about 75 nm, from about 50 nm to about 75 nm, from about 50 nm to about 70 nm, from about 60 nm to about 75 nm, from about 60 nm to about 70 nm, from about 65 nm to about 75 nm, from about 65 nm to about 70 nm, or about 20 nm, about 25 nm, about 30 nm, about 35 nm, about 40 nm, about 45 nm, about 50 nm, about 51 nm, about 52 nm, about 53 nm, about 54 nm, about 55 nm, about 56 nm, about 57 nm, about 58 nm, about 59 nm about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, or about 75 nm (+3 nm) in size.

Generally, the LNPs of the disclosure have a mean diameter selected to provide an intended therapeutic effect. For example, the LNPs of the disclosure have a mean diameter that is compatible with a target organ, such that the LNPs of the disclosure are able to diffuse through the fenestrations of a target organ (e.g., liver) or a target cell subpopulation (e.g., hepatocytes).

According to some embodiments, the lipid particles of the disclosure typically have a mean diameter of less than about 75 nm, less than about 70 nm, less than about 65 nm, less than about 60 nm, less than about 55 nm, less than about 50 nm, less than about 45 nm, less than about 40 nm, less than about 35 nm, less than about 30 nm, less than about 25 nm, less than about 20 nm in size.

As used herein, the term “cationic lipid” refers to any lipid that is positively charged at physiological pH. The cationic lipid in the lipid particles may comprise, e.g., one or more cationic lipids such as 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), “SS-cleavable lipid”, or a mixture thereof. In some embodiments, a cationic lipid can also be an ionizable lipid, i.e., an ionizable cationic lipid. i.e. The term “cationic lipids” also encompasses lipids that are positively charged at any pH, e.g., lipids comprising quaternary amine groups, i.e., quaternary lipids. Any cationic lipid described herein comprising a primary, secondary or tertiary amine group may be converted to a corresponding quaternary lipid, for example, by treatment with chloromethane (CH₃Cl) in acetonitrile (CH₃CN) and chloroform (CHCl₃).

As used herein, the term “ionizable lipid” refers to a lipid, e.g., cationic lipid, having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipids be present in the charged or neutral form. Generally, ionizable lipids have a pKa of the protonatable group in the range of about 4 to about 7. In some embodiments, ionizable lipid may include “cleavable lipid” or “SS-cleavable lipid”.

As used herein, the term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

As used herein, the term “non-cationic lipid” refers to any amphipathic helper lipid as well as any other neutral lipid or anionic lipid.

As used herein, the term “cleavable lipid” or “SS-cleavable lipid” refers to an ionizable lipid comprising a disulfide bond cleavable unit. Cleavable lipids may include cleavable disulfide bond (“ss”) containing lipid-like materials that comprise a pH-sensitive amine, e.g., a tertiary amine, and self-degradable phenyl ester. For example, a SS-cleavable lipid can be an ss-OP lipid (COATSOME® SS-OP), an ss-M lipid (COATSOME® SS-M), an ss-E lipid (COATSOME® SS-E), an ss-EC lipid (COATSOME® SS-EC), an ss-LC lipid (COATSOME® SS-LC), an ss-OC lipid (COATSOME® SS-OC), and an ss-PalmE lipid (see, for example, Formulae I-IV), or a lipid described by Togashi et al., (2018) Journal of Controlled Release “A hepatic pDNA delivery system based on an intracellular environment sensitive vitamin E-scaffold lipid-like material with the aid of an anti-inflammatory drug” 279:262-270. Additional examples of cleavable lipids are described in U.S. Pat. Nos. 9,708,628, and 10,385,030, the entire contents of which are incorporated herein by reference. In one embodiment, cleavable lipids comprise a tertiary amine, which responds to an acidic compartment, e.g., an endosome or lysosome for membrane destabilization and a disulfide bond that can be cleaved in a reducing environment, such as the cytoplasm. In one embodiment, a cleavable lipid is a cationic lipid. In one embodiment, a cleavable lipid is an ionizable cationic lipid. Cleavable lipids are described in more detail herein.

As used herein, the term “organic lipid solution” refers to a composition comprising in whole, or in part, an organic solvent having a lipid.

As used herein, the term “liposome” refers to lipid molecules assembled in a spherical configuration encapsulating an interior aqueous volume that is segregated from an aqueous exterior. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient. Liposome compositions for such delivery are typically composed of phospholipids, especially compounds having a phosphatidylcholine group, however these compositions may also include other lipids.

As used herein, the term “local delivery” refers to delivery of an active agent such as an interfering RNA (e.g., siRNA) directly to a target site within an organism. For example, an agent can be locally delivered by direct injection into a disease site such as a tumor or other target site such as a site of inflammation or a target organ such as the liver, heart, pancreas, kidney, and the like.

As used herein, the term “nucleic acid” refers to a polymer containing at least two nucleotides (i.e., deoxyribonucleotides or ribonucleotides) in either single- or double-stranded form and includes DNA, RNA, and hybrids thereof. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), Doggybone™ DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), messenger RNA (mRNA), rRNA, tRNA, gRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA) or guide RNA (gRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), single stranded DNA (ssDNA) molecules, plasmids, bacmids, DOGGYBONE™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

As used herein, “nucleotides” contain a sugar deoxyribose (DNA) or ribose (RNA), a base, and a phosphate group. Nucleotides are linked together through the phosphate groups.

As used herein, the terms “single-stranded DNA molecule”, “ssDNA molecule”, or “SSD molecule” refer to a deoxyribonucleic acid (DNA) molecule comprising at least one single-stranded nucleic acid sequence flanked by at least one stem-loop structure at the 3′ end. In some embodiments, the single-stranded DNA molecule further comprises at least one stem-loop structure at the 5′ end. As used herein, a single-stranded DNA molecule may comprise regions of double-stranded DNA (or partial duplexes), e.g., a stem-loop structure, e.g., an inverted terminal repeat or portion thereof, at the terminal end(s), e.g., the 3′ end and/or the 5′ end. In some embodiments, a ssDNA molecule is a synthetic ssDNA molecule. In some embodiments, a ssDNA molecule comprises at least one stem-loop structure at the 5′ end and at least one stem-loop structure at the 3′ end.

As used herein, the term “single-stranded (ss) synthetic DNA molecules”, “single-stranded (ss) synthetic vectors”, “synthetic production of ssDNA molecules” and “synthetic production of ss vectors” refers to a single-stranded (ss) synthetic DNA molecule (ssDNA), a single-stranded vector and synthetic production methods thereof in an entirely cell-free environment. The production may involve one or more molecules in a manner that does not involve replication or other multiplication of the molecule by or inside of a cell or using a cellular extract. Synthetic production avoids contamination of the produced molecule with cellular contaminants, e.g., cellular proteins or cellular nucleic acid, viral protein or DNA, insect protein or DNA and further minimizes unwanted cellular-specific modification of the molecule during the production process, e.g., methylation or glycosylation or other post-translational modification.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans, as well as any carrier or diluent that does not cause significant irritation to a subject and does not abrogate the biological activity and properties of the administered compound.

As used herein, the term “gap” refers to a discontinued portion of synthetic DNA vector of the present disclosure, creating a stretch of single stranded DNA portion in otherwise double stranded ceDNA. The gap can be 1 nucleotide to 100 nucleotides in length in one strand of a duplex DNA. Typical gaps, designed and created by the methods described herein and synthetic vectors generated by the methods can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 nt long in length. Exemplified gaps in the present disclosure can be 1 nt to 10 nt long, 1 to 20 nt long, 1 to 30 nt long in length.

As used herein, the term “nick” refers to a discontinuity in a double stranded DNA molecule where there is no phosphodiester bond between adjacent nucleotides of one strand typically through damage or enzyme action. It is understood that one or more nicks allow for the release of torsion in the strand during DNA replication and that nicks are also thought to play a role in facilitating binding of transcriptional machinery.

As used herein, the term “ceDNA” refers to capsid-free closed-ended linear double-stranded (ds) duplex DNA for non-viral gene transfer, synthetic or otherwise. Detailed description of ceDNA is described in International Patent Application No. PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International applications PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International application PCT/US2019/14122, filed Jan. 18, 2019, the entire content of which is incorporated herein by reference. As used herein, the terms “ceDNA vector” and “ceDNA” are used interchangeably. According to some embodiments, the ceDNA is a closed-ended linear duplex (CELiD) CELiD DNA. According to some embodiments, the ceDNA is a DNA-based minicircle. According to some embodiments, the ceDNA is a minimalistic immunological-defined gene expression (MIDGE)-vector. According to some embodiments, the ceDNA is a ministring DNA. According to some embodiments, the ceDNA is a Doggybone™ DNA. According to some embodiments, the ceDNA comprises one or more phosphorothioate-modified nucleotides. According to some embodiments, the ceDNA comprises no phosphorothioate-modified nucleotides.

As used herein, the term “neDNA” or “nicked ceDNA” refers to a closed-ended DNA having a nick or a gap of 1-100 nucleotides in a stem region or spacer region upstream of an open reading frame (e.g., a promoter and transgene to be expressed).

As used herein, the terms “inverted terminal repeat” or “ITR” refer to a nucleic acid sequence located at the 5′ and/or 3′ terminus of the ssDNA molecules disclosed herein, which comprises at least one stem-loop structure comprising a partial duplex and at least one loop.

As used herein, the term “stem-loop structure” refers to a nucleic acid structure comprising at least one double-stranded region (referred to herein as a “stem”) and at least one single-stranded region (referred to herein as a “loop”). In some embodiments, a stem-lop structure is a hairpin structure. In some embodiments, a stem-loop structure comprises more than one stem and more than one loop. In some embodiments, a loop is located at the end of a stem (such that a single loop connects the two strands of a duplex stem, e.g., as in a hairpin structure). In some embodiments, a loop may be located between two stems (which may be referred to herein as a “bulge” or a “bubble”), such that the loop connects two strands of different stems. In some embodiments, as described in more detail herein, a stem-loop structure may comprise more complex secondary structures comprising multiple stems and multiple loops.

According to some embodiments, the 5′ and/or 3′ terminus of certain ssDNA molecules comprise inverted terminal repeats (ITRs) of about 145 nucleotides at both ends, or fragments thereof. The terminal 125 nucleotides in each ITR form a palindromic double-stranded T-shaped hairpin structure, in which the A-A′ palindrome forms the stem, and the two smaller palindromes, B-B′ and the C-C′, form the cross-arms of the T. The other 20 nucleotides in ITR remain single-stranded, and are called the D sequence. The D(−) sequence (also referred to herein as “the ssD(−) sequence”) is at the 3′ end, and the complementary D(+) sequence (also referred to herein as “the ssD(+) sequence”) is at the 5′ end. Second-strand DNA synthesis turns both ssD(−) and ssD(+) sequences into a double-stranded (ds) D(±) sequence, each of which comprises a D region and a D′ region. Ling et al. J Virol. 2015 Jan. 15; 89(2):952-61, WO2016081927A2, incorporated by reference in its entirety herein, described ssD(+)-sequence-substituted ssAAV genomes. ssD(−) and ssD(+) have been reported to contain one or more transcription factor binding sites and to be required for packaging and replication (Ling et al. J Virol. 2015 Jan. 15; 89(2):952-61; WO2016081927A2, incorporated by reference in its entirety herein).

According to some embodiments, the ITR may be a viral ITR (e.g., AAV or other dependovirus), a sequence derived or modified from a viral ITR (e.g., truncation, deletion, substitution, insertion and/or addition), or an entirely artificial sequence (e.g., the ITRs contain no sequences derived from a virus). The ITR may further comprise one stem-loop structure (e.g., a “hairpin”), or more than one stem-loop structure. For example, the ITR may comprise two stem-loop structures (e.g., a “hammerhead”, “doggy-bone”, or “dumbbell”), three stem-loop structures (e.g., “cruciform”), or more complex structures (e.g., quadruplex stem-loop structure). The ITR may comprise an aptamer sequence or one or more chemical modifications. The ITR can be made entirely out of an aptamer sequence having at least one stem region and at least one loop region.

As used herein, the term “substantially symmetrical WT-ITRs” or a “substantially symmetrical WT-ITR pair” refers to a pair of WT-ITRs within a single-stranded DNA (ssDNA) molecule that are both wild type ITRs that have an inverse complement sequence across their entire length. For example, an ITR can be considered to be a wild-type sequence, even if it has one or more nucleotides that deviate from the canonical naturally occurring canonical sequence, so long as the changes do not affect the physical and functional properties and overall three-dimensional structure of the sequence (secondary and tertiary structures). In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same ssD(−)/ssD(+), A-A′, C-C′ and B-B′ loops in 3D space. A substantially symmetrical WT-ITR can be functionally confirmed as WT by determining that it has an operable Rep binding site (RBE or RBE′) and terminal resolution site (TRS) that pairs with the appropriate Rep protein. One can optionally test other functions, including transgene expression under permissive conditions.

As used herein, the phrases “modified ITR” or “mod-ITR” or “mutant ITR” are used interchangeably and refer to an ITR with a mutation in at least one or more nucleotides as compared to the WT-ITR from the same serotype. The mutation can result in a change in one or more of ssD(−) or ssD(+), A, A′, C, C′, B, B′ regions in the ITR, and can result in a change in the three-dimensional spatial organization (i.e., its 3D structure in geometric space) as compared to the 3D spatial organization of a WT-ITR of the same serotype.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides. According to some embodiments, the nucleic acid is a single-stranded DNA (ssDNA) molecule described by the present disclosure. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone (dbDNA™) DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

An “inhibitory polynucleotide” as used herein refers to a DNA or RNA molecule that reduces or prevents expression (transcription or translation) of a second (target) polynucleotide. Inhibitory polynucleotides include antisense polynucleotides, ribozymes, and external guide sequences. The term “inhibitory polynucleotide” further includes DNA and RNA molecules, e.g., RNAi that encode the actual inhibitory species, such as DNA molecules that encode ribozymes.

“Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

By “receptor” is meant a polypeptide, or portion thereof, present on a cell membrane that selectively binds one or more ligands. The term “receptor” as used herein is intended to encompass the entire receptor or ligand-binding portions thereof. These portions of the receptor particularly include those regions sufficient for specific binding of the ligand to occur.

As used herein, the term “subject” refers to a human or animal, to whom treatment, including prophylactic treatment, with the therapeutic nucleic acid according to the present disclosure, is provided. Usually, the animal is a vertebrate such as, but not limited to a primate, rodent, domestic animal or game animal. Primates include but are not limited to, chimpanzees, cynomolgus monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the phrase “subject in need” refers to a subject that (i) will be administered a ceDNA lipid particle (or pharmaceutical composition comprising a ceDNA lipid particle) according to the described disclosure, (ii) is receiving a ceDNA lipid particle (or pharmaceutical composition comprising aceDNA lipid particle) according to the described disclosure; or (iii) has received a ceDNA lipid particle (or pharmaceutical composition comprising a ceDNA lipid particle) according to the described disclosure, unless the context and usage of the phrase indicates otherwise.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the term “systemic delivery” refers to delivery of lipid particles that leads to a broad biodistribution of an active agent such as an interfering RNA (e.g., siRNA) within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of LNPs can be by any means known in the art including, for example, intravenous, subcutaneous, and intraperitoneal. In a preferred embodiment, systemic delivery of LNPs is by intravenous delivery.

As used herein, the terms “effective amount”, which may be used interchangeably with the terms “therapeutic amount”, “therapeutically effective amount”, an “amount effective”, or “pharmaceutically effective amount” of an active agent (e.g., a ceDNA as described herein), refers to an amount that is sufficient to provide the intended benefit of treatment or effect, e.g., expression or inhibition of expression of a target sequence in comparison to the expression level detected in the absence of a therapeutic nucleic acid. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art. Dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods. Additionally, the terms “effective amount”, “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein. In one aspect of any of the aspects or embodiments herein, “therapeutic amount”, “therapeutically effective amounts” and “pharmaceutically effective amounts” refer to non-prophylactic or non-preventative applications.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, the terms “treat,” “treating,” and/or “treatment” include abrogating, inhibiting, slowing or reversing the progression of a condition, ameliorating clinical symptoms of a condition, or preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s). In one aspect of any of the aspects or embodiments herein, the terms “treat,” “treating,” and/or “treatment” include abrogating, inhibiting, slowing or reversing the progression of a condition, or ameliorating clinical symptoms of a condition.

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “combination therapy” refers to treatment regimens for a clinical indication that comprise two or more therapeutic agents. Thus, the term refers to a therapeutic regimen in which a first therapy comprising a first composition (e.g., active ingredient) is administered in conjunction with a second therapy comprising a second composition (active ingredient) to a patient, intended to treat the same or overlapping disease or clinical condition. The first and second compositions may both act on the same cellular target, or discrete cellular targets. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies.

As used herein, the term “alkyl” refers to a saturated monovalent hydrocarbon radical of 1 to 20 carbon atoms (i.e., C₁₋₂₀ alkyl). “Monovalent” means that alkyl has one point of attachment to the remainder of the molecule. In one embodiment, the alkyl has 1 to 12 carbon atoms (i.e., C₁₋₁₂ alkyl) or 1 to 10 carbon atoms (i.e., C1-10 alkyl). In one embodiment, the alkyl has 1 to 8 carbon atoms (i.e., C₁₋₈ alkyl), 1 to 7 carbon atoms (i.e., C₁₋₇ alkyl), 1 to 6 carbon atoms (i.e., C₁₋₆ alkyl), 1 to 4 carbon atoms (i.e., C₁₋₄ alkyl), or 1 to 3 carbon atoms (i.e., C₁₋₃ alkyl). Examples include, but are not limited to, methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like. A linear or branched alkyl, such as a “linear or branched C₁₋₆ alkyl,” “linear or branched C₁₋₄ alkyl,” or “linear or branched C₁₋₃ alkyl” means that the saturated monovalent hydrocarbon radical is a linear or branched chain. As used herein, the term “linear” as referring to aliphatic hydrocarbon chains means that the chain is unbranched.

The term “alkylene” as used herein refers to a saturated divalent hydrocarbon radical of 1 to 20 carbon atoms (i.e., C₁₋₂₀ alkylene), examples of which include, but are not limited to, those having the same core structures of the alkyl groups as exemplified above. “Divalent” means that the alkylene has two points of attachment to the remainder of the molecule. In one embodiment, the alkylene has 1 to 12 carbon atoms (i.e., C₁₋₁₂ alkylene) or 1 to 10 carbon atoms (i.e., C₁₋₁₀ alkylene). In one embodiment, the alkylene has 1 to 8 carbon atoms (i.e., C₁₋₈ alkylene), 1 to 7 carbon atoms (i.e., C₁₋₇ alkylene), 1 to 6 carbon atoms (i.e., C₁₋₆ alkylene), 1 to 4 carbon atoms (i.e., C₁₋₄ alkylene), 1 to 3 carbon atoms (i.e., C₁₋₃ alkylene), ethylene, or methylene. A linear or branched alkylene, such as a “linear or branched C₁₋₆ alkylene,” “linear or branched C₁₋₄ alkylene,” or “linear or branched C₁₋₃ alkylene” means that the saturated divalent hydrocarbon radical is a linear or branched chain.

The term “alkenyl” refers to straight or branched aliphatic hydrocarbon radical with one or more (e.g., one or two) carbon-carbon double bonds, wherein the alkenyl radical includes radicals having “cis” and “trans” orientations, or by an alternative nomenclature, “E” and “Z” orientations.

The term “alkenylene” refers to aliphatic divalent hydrocarbon radical of 2 to 20 carbon atoms (i.e., C₂₋₂₀ alkenylene) with one or two carbon-carbon double bonds, wherein the alkenylene radical includes radicals having “cis” and “trans” orientations, or by an alternative nomenclature, “E” and “Z” orientations. “Divalent” means that alkenylene has two points of attachment to the remainder of the molecule. In one embodiment, the alkenylene has 2 to 12 carbon atoms (i.e., C₂₋₁₂ alkenylene), 2 to 10 carbon atoms (i.e., C₂₋₁₀ alkenylene). In one embodiment, the alkenylene has 2 to four carbon atoms (C₂-4). Examples include, but are not limited to, ethylenylene or vinylene (—CH═CH—), allyl (—CH₂CH═CH—), and the like. A linear or branched alkenylene, such as a “linear or branched C₂₋₆ alkenylene,” “linear or branched C₂₋₄ alkenylene,” or “linear or branched C₂₋₃ alkenylene” means that the unsaturated divalent hydrocarbon radical is a linear or branched chain.

The term “cycloalkylene”, as used herein refers to a divalent saturated carbocyclic ring radical having 3 to 12 carbon atoms as a monocyclic ring, or 7 to 12 carbon atoms as a bicyclic ring. “Divalent” means that the cycloalkylene has two points of attachment to the remainder of the molecule. In one embodiment, the cycloalkylene is a 3- to 7-membered monocyclic or 3- to 6-membered monocyclic. Examples of monocyclic cycloalkyl groups include, but are not limited to, cyclopropylene, cyclobutylene, cyclopentylene, cyclohexylene, cycloheptylene, cyclooctylene, cyclononylene, cyclodecylene, cycloundecylene, cyclododecylene, and the like. In one embodiment, the cycloalkylene is cyclopropylene.

The terms “heterocycle,” “heterocyclyl,” heterocyclic and “heterocyclic ring” are used interchangeably herein and refer to a cyclic group which contains at least one N atom has a heteroatom and optionally 1-3 additional heteroatoms selected from N and S, and are non-aromatic (i.e., partially or fully saturated). It can be monocyclic or bicyclic (bridged or fused). Examples of heterocyclic rings include, but are not limited to, aziridinyl, diaziridinyl, thiaziridinyl, azetidinyl, diazetidinyl, triazetidinyl, thiadiazetidinyl, thiazetidinyl, pyrrolidinyl, pyrazolidinyl, imidazolinyl, isothiazolidinyl, thiazolidinyl, piperidinyl, piperazinyl, hexahydropyrimidinyl, azepanyl, azocanyl, and the like. The heterocycle contains 1 to 4 heteroatoms, which may be the same or different, selected from N and S. In one embodiment, the heterocycle contains 1 to 3 N atoms. In another embodiment, the heterocycle contains 1 or 2 N atoms. In another embodiment, the heterocycle contains 1 N atom. A “4- to 8-membered heterocyclyl” means a radical having from 4 to 8 atoms (including 1 to 4 heteroatoms selected from N and S, or 1 to 3 N atoms, or 1 or 2 N atoms, or 1 N atom) arranged in a monocyclic ring. A “5- or 6-membered heterocyclyl” means a radical having from 5 or 6 atoms (including 1 to 4 heteroatoms selected from N and S, or 1 to 3 N atoms, or 1 or 2 N atoms, or 1 N atom) arranged in a monocyclic ring. The term “heterocycle” is intended to include all the possible isomeric forms. Heterocycles are described in Paquette, Leo A., Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs (John Wiley & Sons, New York, 1950 to present), in particular Volumes 13, 14, 16, 19, and 28; and J. Am. Chem. Soc. (1960) 82:5566. The heterocyclyl groups may be carbon (carbon-linked) or nitrogen (nitrogen-linked) attached to the rest of the molecule where such is possible.

If a group is described as being “optionally substituted,” the group may be either (1) not substituted, or (2) substituted. If a carbon of a group is described as being optionally substituted with one or more of a list of substituents, one or more of the hydrogen atoms on the carbon (to the extent there are any) may separately and/or together be replaced with an independently selected optional substituent.

Suitable substituents for an alkyl, alkylene, alkenylene, cycloalkylene, and heterocyclyl, are those which do not significantly adversely affect the biological activity of the molecule. Unless otherwise specified, exemplary substituents for these groups include linear, branched or cyclic alkyl, alkenyl or alkynyl having from 1 to 10 carbon atoms, aryl, heteroaryl, heterocyclyl, halogen, guanidinium [—NH(C═NH)NH₂], —OR₁₀₀, NR₁₀₁R₁₀₂, —NO₂, —NR₁₀₁COR₁₀₂, —SR₁₀₀, a sulfoxide represented by —SOR₁₀₁, a sulfone represented by —SO₂R₁₀₁, a sulfonate —SO₃M, a sulfate —OSO₃M, a sulfonamide represented by —SO₂NR₁₀₁R₁₀₂, cyano, an azido, —COR₁₀₁, —OCOR₁₀₁, —OCONR₁₀₁R₁₀₂ and a polyethylene glycol unit (—OCH₂CH₂)ₙR₁₀₁ wherein M is H or a cation (such as Na⁺ or K⁺); R₁₀₁, R₁₀₂ and R₁₀₃ are each independently selected from H, linear, branched or cyclic alkyl, alkenyl or alkynyl having from 1 to 10 carbon atoms, a polyethylene glycol unit (—OCH₂CH₂)ₙ—R₁₀₄, wherein n is an integer from 1 to 24, an aryl having from 6 to 10 carbon atoms, a heterocyclic ring having from 3 to 10 carbon atoms and a heteroaryl having 5 to 10 carbon atoms; and R₁₀₄ is H or a linear or branched alkyl having 1 to 4 carbon atoms, wherein the alkyl, alkenyl, alkynyl, aryl, heteroaryl and heterocyclyl in the groups represented by R₁₀₀, R₁₀₁, R₁₀₂, R₁₀₃ and R₁₀₄ are optionally substituted with one or more (e.g., 2, 3, 4, 5, 6 or more) substituents independently selected from halogen, —OH, —CN, —NO₂, and unsubstituted linear or branched alkyl having 1 to 4 carbon atoms. Preferably, the substituent for the optionally substituted alkyl, alkylene, alkenylene, cycloalkylene, and heterocyclyl described above is selected from the group consisting of halogen, —CN, —NR₁₀₁R₁₀₂, —CF₃, —OR₁₀₀, aryl, heteroaryl, heterocyclyl, —SR₁₀₁, —SOR₁₀₁, —SO₂R₁₀₁, and —SO₃M. Alternatively, the suitable substituent is selected from the group consisting of halogen, —OH, —NO₂, —CN, C₁₋₄ alkyl, —OR₁₀₀, NR₁₀₁R₁₀₂, —NR₁₀₁COR₁₀₂, —SR₁₀₀, —SO₂R₁₀₁, —SO₂NR₁₀₁R₁₀₂, —COR₁₀₁, —OCOR₁₀₁, and —OCONR₁₀₁R₁₀₂, wherein R₁₀₀, R₁₀₁, and R₁₀₂ are each independently —H or C₁₋₄ alkyl.

The term “halogen”, as used herein, refers to F, Cl, Br or I. “Cyano” is —CN.

The terms “amine” or “amino” are used herein interchangeably and refer to a functional group that contains a basic nitrogen atom with a lone pair.

The term “pharmaceutically acceptable salt”, as used herein, refers to pharmaceutically acceptable organic or inorganic salts of an ionizable lipid of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the disclosure.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited in any manner to the particular methodology, protocols, and reagents, etc., described herein and as such can vary. The terminology used herein is for the purpose of describing particular embodiments only and is not intended to limit the scope of the present disclosure, which is defined solely by the claims.

### II. Polymer-Conjugated Lipids

In some embodiments, the present disclosure provides a polymer-conjugated lipid that comprises: (i) a polyglycerol (PG) or a PG derivative; (ii) a lipid moiety; and (iii) a linker conjugating the PG or the PG derivative to the lipid moiety.

### Polyglycerol and Polyglycerol Derivative

The PG or the PG derivative comprised in the polymer-conjugated lipid of the disclosure may be linear or branched. In one specific embodiment, the PG or the PG derivative is linear. In another embodiment, the PG or the PG derivative is branched.

The polymer-conjugated lipid of the disclosure may comprise PG or a PG derivative comprising an average of 5 to 100 monomeric units, e.g., an average of 10 to 100 monomeric units, e.g., an average of 10 to 40 monomeric units, an average of 15-75 monomeric units, an average of 20 to 50 monomeric units, an average of 30 to 70 monomeric units, an average of 40 to 90 monomeric units or an average of 50 to 100 monomeric units. In some embodiments, the polyglycerol of the disclosure comprises an average of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 monomeric units.

In one embodiment, PG or the PG derivative of the disclosure comprises an average of 8 monomeric units.

In one embodiment, the PG or the PG derivative of the disclosure comprises an average of 34 monomeric units.

In one embodiment, the PG or the PG derivative of the disclosure comprises an average of 45 monomeric units.

In one embodiment, the PG or the PG derivative of the disclosure comprises an average of 46 monomeric units.

In one embodiment, the PG or the PG derivative of the disclosure comprises an average of 58 monomeric units.

In one embodiment, the polymer-conjugated lipid of the disclosure comprises PG.

In one embodiment, the polymer-conjugated lipid of the disclosure comprises PG derivative.

The PG derivative comprised in the polymer-conjugated lipid of the disclosure may be a carboxylated PG, e.g., 2-carboxycyclohexane-1-carboxylated polyglycerol.

The PG derivative comprised in the polymer-conjugated lipid of the disclosure may also be a glutarylated PG, e.g., 3-methyl glutarylated PG.

In some embodiments, the PG derivative comprised in the polymer-conjugated lipid of the disclosure is represented by the following structural formula:

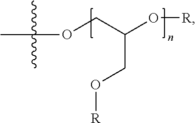

wherein:
n is an integer ranging from 8 to 100; and
R is selected from the group consisting of

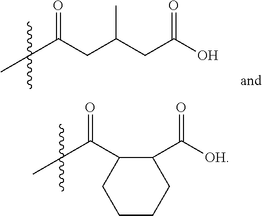

### Lipid Moiety

In some embodiments, the lipid moiety comprised in the polymer-conjugated lipid of the disclosure is represented by Formula (I)

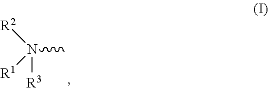

or a pharmaceutically acceptable salt thereof, wherein:
R¹ is absent, hydrogen, C₁-C₆ alkyl, or a hydrophobic tail comprising 10-30 carbon atoms;
R² is absent, hydrogen, C₁-C₆ alkyl, or a hydrophobic tail comprising 10-30 carbon atoms;
wherein, when R¹ and R² are each hydrogen, C₁-C₆ alkyl, or a hydrophobic tail comprising 10-30 carbon atoms, N is positively charged; and
R³ is a hydrophobic tail comprising 10-30 carbon atoms;
wherein [symbol] in Formula (I) is a bond conjugating the lipid moiety and the linker.

In some embodiments, R¹ is absent, and R² and R³ are each independently a hydrophobic tail comprising 14, 15, 16, 17, 18, 19, 20, 21, or 22 carbon atoms. In some embodiments, R² and R³ are each independently a hydrophobic tail comprising 16, 17, 18, 19, 20, 21, or 22 carbon atoms. In one specific embodiment, R² and R³ are each independently a hydrophobic tail comprising 18 carbon atoms, and the lipid moiety is dioctadecylamine (DODA).

### Linker

The linker conjugating the polyglycerol or the polyglycerol derivative to the lipid moiety in the polymer-conjugated lipid of the present disclosure may be an alkyl linker, a glycerol linker, a phosphate linker, a phosphate ester linker, an ether linker, an ester linker, a diester linker (e.g., a glutaryl linker, a succinyl linker, etc.), an amide linker, a diamide linker, an amine linker, a peptide linker, a phosphoethanolamine linker, a phosphocholine linker, a carbamate linker, a diamide alkyl linker, a cleavable linker, or any combination thereof.

In some embodiments, the linker may be selected from the group consisting of —(CH₂)ₙ—, —C(O)(CH₂)ₙ—, —C(O)O(CH₂)ₙ, —OC(O)(CH₂)ₙC(O)O—, and —NH(CH₂)ₙC(O)O—, wherein n is an integer ranging from 1 to 20. In some embodiments, the linker is —C(O)(CH₂)ₙ—, and n is 2, 3, 4, 5, or 6. In one embodiment, n is 4.

In some embodiments, the polymer-conjugated lipid of the disclosure is DODA-PG34 represented by the following structure:

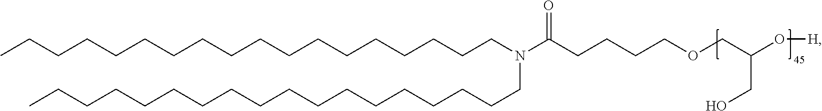

or a pharmaceutically acceptable salt or ester thereof, or a deuterated analogue thereof.

In some embodiments, the polymer-conjugated lipid of the disclosure is DODA-PG45 represented by the following structure:

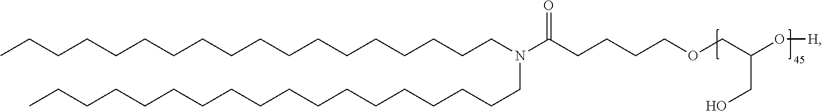

or a pharmaceutically acceptable salt or ester thereof, or a deuterated analogue thereof.

In some embodiments, the polymer-conjugated lipid of the disclosure is DODA-PG46 represented by the following structure:

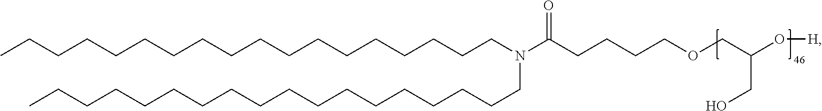

or a pharmaceutically acceptable salt or ester thereof, or a deuterated analogue thereof.

In some embodiments, the polymer-conjugated lipid of the disclosure is DODA-PG58 represented by the following structure:

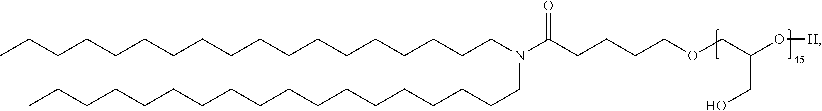

or a pharmaceutically acceptable salt or ester thereof, or a deuterated analogue thereof.

In some embodiments, the polymer-conjugated lipid of the disclosure may be represented by the following structure

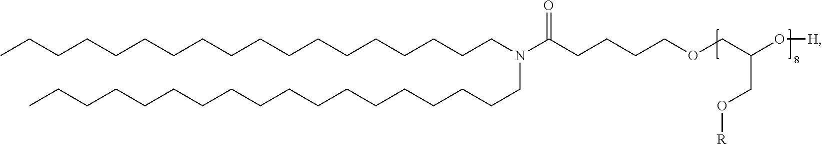

wherein R is

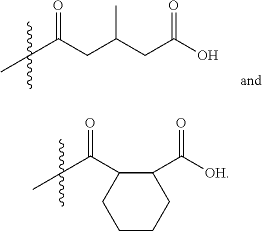

or a pharmaceutically acceptable salt or ester thereof, or a deuterated analogue thereof.

The polymer-conjugated lipid of the present disclosure may also comprise a targeting moiety. The various targeting moieties that may be comprised in the polymer-conjugated lipid of the disclosure are described herein in the section “Targeting Moiety”.

The polymer-conjugated lipid of the present disclosure may also comprise a reactive species conjugated to the PG or the PG derivative. The reactive species present in the polymer-conjugated lipid of the present disclosure may be used for conjugation, e.g., to a targeting moiety which has been functionalized with a complementary reactive species, i.e., a reactive species capable of reacting with the reactive species comprised in the polymer-conjugated lipid of the present disclosure. In some embodiments, the reactive species conjugated to the polymer-conjugated lipid of the present disclosure may be a thiol reagent, a maleimide reagent, or click chemistry reagent, e.g., a reagent selected from the group consisting of an alkyne reagent, such as a dibenzocyclooctyne (DBCO) reagent, a transcyclooctene (TCO) reagent, a tetrazine (TZ) reagent and an azide (AZ) reagent.

In some embodiments, the polymer-conjugated lipid of the present disclosure may comprise a targeting moiety that has been conjugated to the polymer-conjugated lipid via the reactive species. For example, the polymer-conjugated lipid of the present disclosure comprising an azide reagent as the reactive species may be reacted with a targeting moiety functionalized with a DBCO reagent as a complementary reactive species to produce a polymer-conjugated lipid comprising a targeting moiety.

In another example, the polymer-conjugated lipid of the present disclosure comprising a thiol reagent may be reacted with a targeting moiety functionalized with a maleimide reagent to produce a polymer-conjugated lipid comprising a targeting moiety. Any targeting moiety described herein may be conjugated to a polymer-conjugated lipid of the present disclosure.

### Synthesis of Polymer-Conjugated Lipid

A polymer-conjugated lipid of the present disclosure, wherein the polymer is PG, may be synthesized by a method comprising:
(a) reacting a lipid moiety which is conjugated to a linker with 2,3-epoxy-1-(1-ethoxyethoxy)propane (EEGE) in the presence of a base, or in the presence of an organocatalyst, under argon atmosphere to produce a lipid moiety conjugated to a linker and polymerized EEGE; and
(b) subjecting the lipid moiety conjugated to a linker and polymerized EEGE to acidic conditions to produce the polymer-conjugated lipid.

In some embodiments, molar ratio of the lipid moiety conjugated to a linker to EEGE may be vaned from about 1:20 to about 1:100. For example, in step (a), the molar ratio of the lipid moiety conjugated to a linker to EEGE may be about 1:20 to about 1:40, about 1:25 to about 1:50, about 1:30 to about 1:60, about 1:50 to about 1:75 or about 1:60 to about 1:100, e.g., about 1:20, about 1:25, about 1:30, about 1:35, about 1:40, about 1:45, about 1:50, about 1:55, about 1:60, about 1:65, about 1:70, about 1:75, about 1:80, about 1:85, about 1:90 or about 1:100. In one embodiment, the ratio of a lipid moiety conjugated to a linker to EEGE may be about 1:50. In another embodiment, the ratio of a lipid moiety conjugated to a linker to EEGE may be about 1:60.

In some embodiments, the ratio of the lipid moiety conjugated to a linker to EEGE in step (a) determines the average number of monomeric units present in the PG portion of the polymer-conjugated lipid in the final product.

The base useful for carrying out step (a) of the method may be a phosphazene base, such as P4-t-Bu.

In some embodiments, the organocatalyst useful for carrying out step (a) of the method may be an N-heterocyclic carbene (NHC) or an N-heterocyclic olefin (NHO).

In some embodiments, step (a) may be carried out overnight.

The acidic conditions useful for carrying out step (b) of the method comprise a strong acid, such as hydrochloric acid (HCl). Other strong acids that may be used in this step comprise hydrobromic acid (HBr), hydroiodic acid (HI), perchloric acid (HClO₄), chloric acid (HClO₃), sulfuric acid (H₂SO₄), and nitric acid (HNO₃).

In some embodiments, the lipid moiety is DODA and the lipid moiety conjugated to a linker is represented by the following structure:

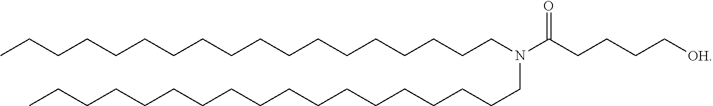 [SMILES: CCCCCCCCCCCCCCCCCCN(CCCCCCCCCCCCCCCCCC)C(=O)CCCCO]

In some embodiments, the polymer-conjugated lipid is DODA-PG, wherein the PG comprises an average of 5-100 monomeric units. In some embodiments, the polymer-conjugated lipid is DODA-PG34, DODA-PG45, DODA-PG46 or DODA-PG58.

### III. Lipid Nanoparticles (LNPs)

The present disclosure also provides lipid nanoparticles (LNPs) comprising: (i) a therapeutic nucleic acid (TNA); (ii) an ionizable lipid; (iii) a sterol; and (iv) a first lipid-anchored polymer, and optionally further comprising a helper lipid, wherein the first lipid-anchored polymer comprises the polymer-conjugated lipid of the present disclosure. Also provided herein are LNPs consisting essentially of: (i) a therapeutic nucleic acid (TNA); (ii) an ionizable lipid; (iii) a sterol; and (iv) a first lipid-anchored polymer, and optionally further consisting essentially of a helper lipid, wherein the first lipid-anchored polymer comprises the polymer-conjugated lipid of the present disclosure. Also provided herein are LNPs consisting of: (i) a therapeutic nucleic acid (TNA); (ii) an ionizable lipid; (iii) a sterol; and (iv) a first lipid-anchored polymer, and optionally further consisting of a helper lipid, wherein the first lipid-anchored polymer comprises the polymer-conjugated lipid of the present disclosure.

### A. Ionizable Lipids

In some embodiments, the ionizable lipid is present in the LNP provided by the present disclosure in an amount of about 20 mol % to about 60 mol %, about 35 mol % to about 50 mol %, of the total lipid present in the LNP.

In some embodiments, the LNPs provided by the present disclosure comprise an ionizable lipid. Exemplary ionizable lipids in the LNPs of the present disclosure are described in International Patent Application Publication Nos. WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, WO2013/086354, and WO2021/102411, and US Patent Application Publication Nos. US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

In some embodiments, the ionizable lipid in the LNPs of the present disclosure is MC3 (6Z,9Z,28Z,31Z)-heptatriaconta-6,9,28,31-tetraen-19-yl-4-(dimethylamino) butanoate (DLin-MC3-DMA or MC3) represented by the following structural formula:

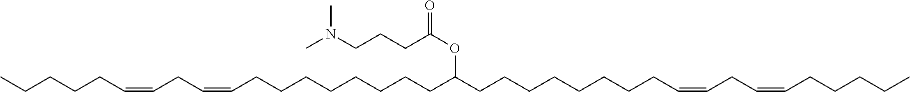 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety.

In some embodiments, the ionizable lipid is selected from the group consisting of N-[1-(2,3-dioleyloxy)propyll-N,N,N-trimethylammonium chloride (DOTMA); N-[1-(2,3-dioleoyloxy)propyll-N,N,N-trimethylammonium chloride (DOTAP); 1,2-dioleoyl-sn-glycero-3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl-sn-glycero-3-ethylphosphocholine (14:1), N1-[2-((1S)-1-[(3-aminopropyl)amino]-4-[di(3-amino-propyl) aminolbutylc arboxamidoiethy11-3,4-di[oleyloxy]-benzamide (MVL5); Dioctadecylamido-glycylspermine (DOGS); 3b-[N—(N′,N′-dimethylaminoethyl)carbamoyl] cholesterol (DC-Chol); Dioctadecyldimethylammonium Bromide (DDAB); a Saint lipid (e.g., SAINT-2, N-methyl-4-(dioleyl)methylpyridinium); 1,2-dimyristyloxypropyl-3-dimethylhydroxyethylammonium bromide (DMRIE); 1,2-dioleoyl-3-dimethyl-hydroxyethyl ammonium bromide (DORIE); 1,2-dioleoyloxypropyl-3-dimethylhydroxyethyl ammonium chloride (DORI); Di-alkylated Amino Acid (DILA2) (e.g., C18:1-norArg-C16); Dioleyldimethylammonium chloride (DODAC); 1-palmitoyl-2-oleoyl-sn-glycero-3-ethylpho sphocholine (POEPC); and 1,2-dimyristoleoyl-sn-glycero-3-ethylphosphocholine (MOEPC). In some variations, the condensing agent, e.g. a cationic lipid, is a lipid such as, e.g., Dioctadecyldimethylammonium bromide (DDAB), 1,2-dilinoleyloxy-3-dimethylaminopropane (DLinDMA), 2,2-dilinoleyl-4-(2dimethylaminoethyl)-[1,31-dioxolane (DLin-KC2-DMA), heptatriaconta-6,9,28,31-tetraen-19-yl-4-(dimethylamino)butanoate (DLin-MC3-DMA), 1,2-Dioleoyloxy-3-dimethylaminopropane (DODAP), 1,2-Dioleyloxy-3-dimethylaminopropane (DODMA), Morpholinocholesterol (Mo-CHOL), (R)-5-(dimethylamino)pentane-1,2-diy1 dioleate hydrochloride (DODAPen-C1), (R)-5-guanidinopentane-1,2-diy1 dioleate hydrochloride (DOPen-G), and (R)—N,N,N-trimethyl-4,5-bis(oleoyloxy)pentan-1-aminium chloride (DOTAPen).

Formula (A) In some embodiments, the ionizable lipid in the LNPs of the present disclosure is represented by Formula (A):

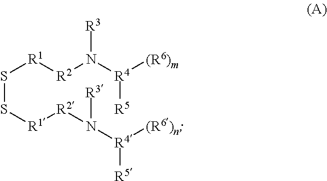

or a pharmaceutically acceptable salt thereof, wherein:
R¹ and R^{1′} are each independently C₁₋₃ alkylene;
R² and R^{2′} are each independently linear or branched C₁₋₆ alkylene, or C₃₋₆ cycloalkylene;
R³ and R^{3′} are each independently optionally substituted C₁₋₆ alkyl or optionally substituted C₃₋₆ cycloalkyl;
or alternatively, when R² is branched C₁₋₆ alkylene and when R³ is C₁₋₆ alkyl, R² and R³, taken together with their intervening N atom, form a 4- to 8-membered heterocyclyl;
or alternatively, when R^{2′} is branched C₁₋₆ alkylene and when R^{3′} is C₁₋₆ alkyl, R^{2′} and R^{3′}, taken together with their intervening N atom, form a 4- to 8-membered heterocyclyl;
R⁴ and R^{4′} are each independently —CH, —CH₂CH, or —(CH₂)₂CH;
R⁵ and R^{5′} are each independently hydrogen, C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5.

In some embodiments, R² and R^{2′} are each independently C₁₋₃ alkylene.

In some embodiments, the linear or branched C₁₋₃ alkylene represented by R¹ or R^{1′}, the linear or branched C₁₋₆ alkylene represented by R² or R^{2′}, and the optionally substituted linear or branched C₁₋₆ alkyl are each optionally substituted with one or more halo and cyano groups.

In some embodiments, R¹ and R² taken together are C₁₋₃ alkylene and R^{1′} and R^{2′} taken together are C₁₋₃ alkylene, e.g., ethylene.

In some embodiments, R³ and R^{3′} are each independently optionally substituted C₁₋₃ alkyl, e.g., methyl.

In some embodiments, R⁴ and R^{4′} are each —CH.

In some embodiments, R² is optionally substituted branched C₁₋₆ alkylene; and R² and R³, taken together with their intervening N atom, form a 5- or 6-membered heterocyclyl. In some embodiments, R^{2′} is optionally substituted branched C₁₋₆ alkylene; and R^{2′} and R^{3′}, taken together with their intervening N atom, form a 5- or 6-membered heterocyclyl, such as pyrrolidinyl or piperidinyl.

In some embodiments, R⁴ is —C(R^{a})₂CR^{a}, or —[C(R^{a})₂]₂CR^{a} and R^{a} is C₁₋₃ alkyl; and R³ and R⁴, taken together with their intervening N atom, form a 5- or 6-membered heterocyclyl. In some embodiments, R^{4′} is —C(R^{a})₂CR^{a}, or —[C(R^{a})₂]₂CR^{a} and R^{a} is C₁₋₃ alkyl; and R^{3′} and R^{4′}, taken together with their intervening N atom, form a 5- or 6-membered heterocyclyl, such as pyrrolidinyl or piperidinyl.

In some embodiments, R and R′ are each independently C₁₋₁₀ alkylene or C₂₋₁₀ alkenylene. In one embodiment, R and R′ are each independently C₁₋₈ alkylene or C₁₋₆ alkylene.

In some embodiments, R⁶ and R^{6′}, for each occurrence, are independently C₁₋₁₀ alkylene, C₃₋₁₀ cycloalkylene, or C₂₋₁₀ alkenylene. In one embodiment, C₁₋₆ alkylene, C₃₋₆ cycloalkylene, or C₂₋₆ alkenylene. In one embodiment the C₃₋₁₀ cycloalkylene or the C₃₋₆ cycloalkylene is cyclopropylene. In some embodiments, m and n are each 3.

In some embodiments, the ionizable lipid in the LNPs of the present disclosure may be selected from any one of the lipids listed in Table 1 below, or a pharmaceutically acceptable salt thereof.

TABLE 1

| Lipid No. | Lipid Structure |
|---|---|
| 1 | 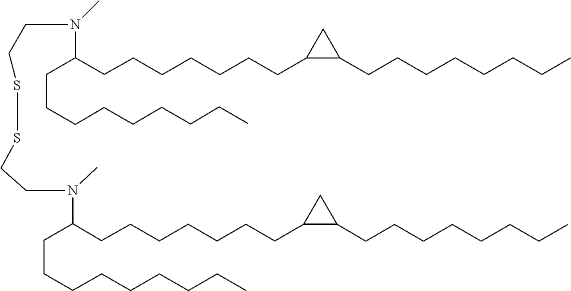 [SMILES: CCCCCCCCCC(CCCCCCCC1CC1CCCCCCCC)N(C)CCSSCCN(C)C(CCCCCCCCC)CCCCCCCC1CC1CCCCCCCC] |
| 2 | 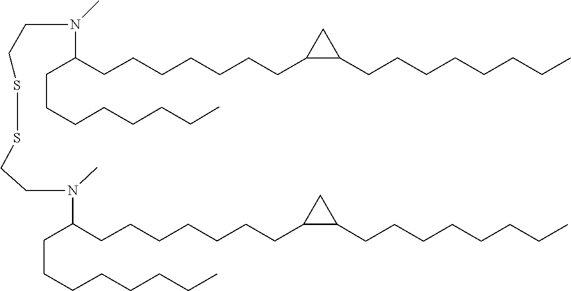 [SMILES: CCCCCCCCC1CC1CCCCCCCC(CCCCCCCC)N(C)CCSSCCN(C)C(CCCCCCCC)CCCCCCCC1CC1CCCCCCCC] |
| 3 | 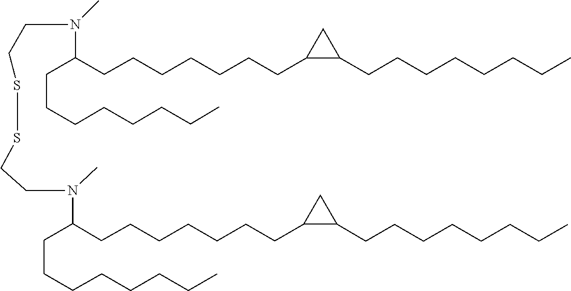 [SMILES: CCCCCCCCC1CC1CCCCCCCC(CCCCCCCC)N(C)CCSSCCN(C)C(CCCCCCCC)CCCCCCCC1CC1CCCCCCCC] |
| 4 | 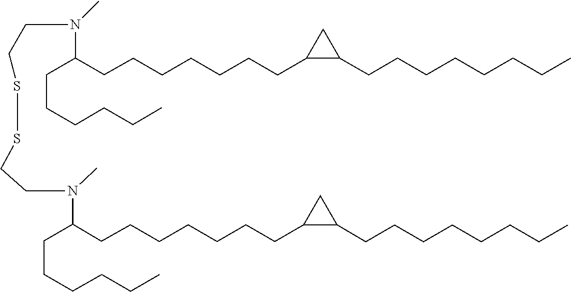 [SMILES: CCCCCCCCC1CC1CCCCCCCC(CCCCCC)N(C)CCSSCCN(C)C(CCCCCC)CCCCCCCC1CC1CCCCCCCC] |
| 5 | 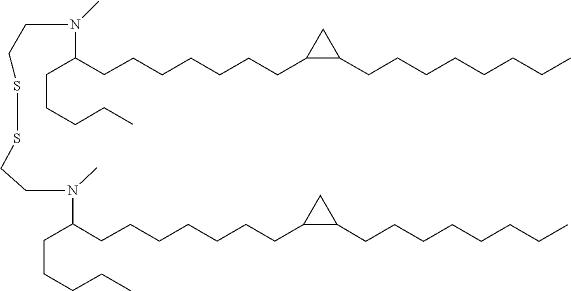 [SMILES: CCCCCCCCC1CC1CCCCCCCC(CCCCC)N(C)CCSSCCN(C)C(CCCCC)CCCCCCCC1CC1CCCCCCCC] |
| 6 | 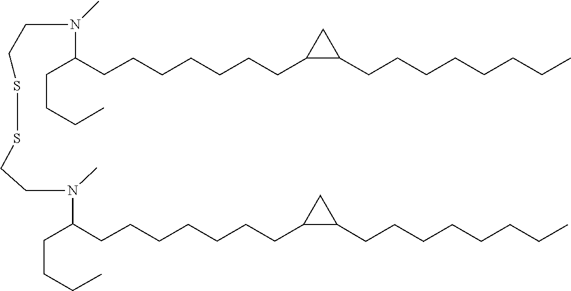 [SMILES: CCCCCCCCC1CC1CCCCCCCC(CCCC)N(C)CCSSCCN(C)C(CCCC)CCCCCCCC1CC1CCCCCCCC] |
| 7 | 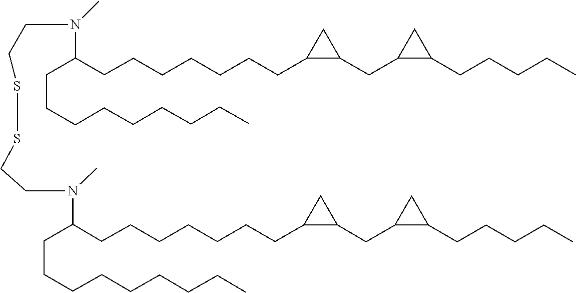 [SMILES: CCCCCCCCCC(CCCCCCCC1CC1CC1CC1CCCCC)N(C)CCSSCCN(C)C(CCCCCCCCC)CCCCCCCC1CC1CC1CC1CCCCC] |
| 8 | 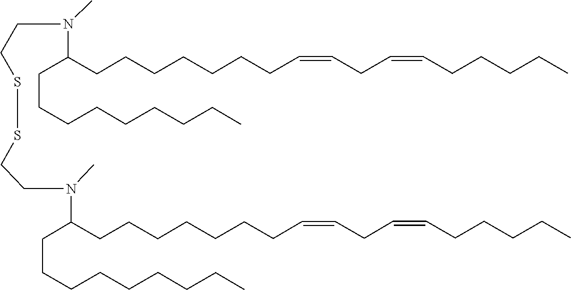 [SMILES: CCCCC/C=C\C/C=C\CCCCCCCC(CCCCCCCCC)N(C)CCSSCCN(C)C(CCCCCCC/C=C\C/C=C\CCCCC)CCCCCCCCC] |
| 9 | 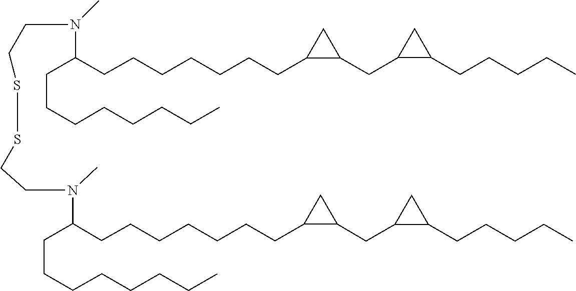 [SMILES: CCCCCCCCC(CCCCCCCC1CC1CC1CC1CCCCC)N(C)CCSSCCN(C)C(CCCCCCCC)CCCCCCCC1CC1CC1CC1CCCCC] |
| 10 | 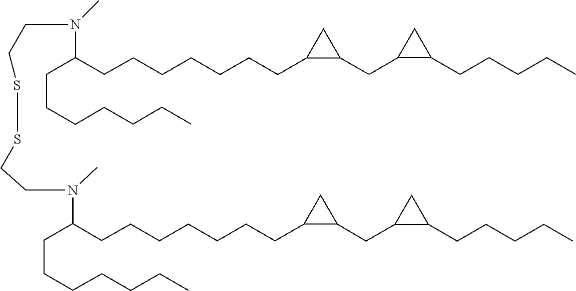 [SMILES: CCCCCCCC(CCCCCCCC1CC1CC1CC1CCCCC)N(C)CCSSCCN(C)C(CCCCCCC)CCCCCCCC1CC1CC1CC1CCCCC] |
| 11 | 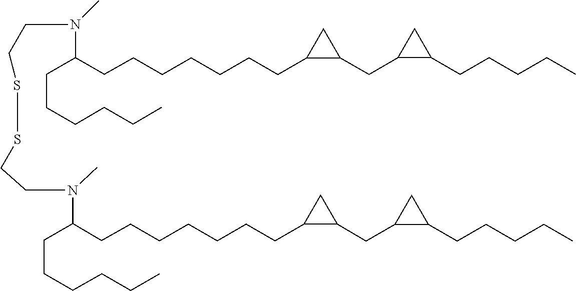 [SMILES: CCCCCCC(CCCCCCCC1CC1CC1CC1CCCCC)N(C)CCSSCCN(C)C(CCCCCC)CCCCCCCC1CC1CC1CC1CCCCC] |
| 12 | 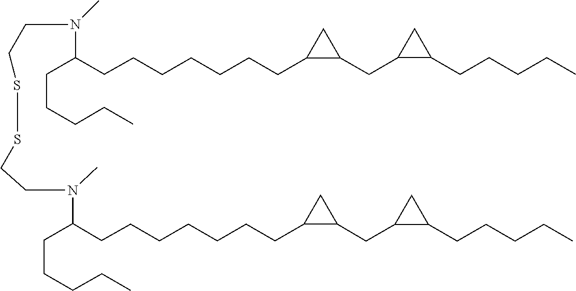 [SMILES: CCCCCC1CC1CC1CC1CCCCCCCC(CCCCC)N(C)CCSSCCN(C)C(CCCCC)CCCCCCCC1CC1CC1CC1CCCCC] |
| 13 | 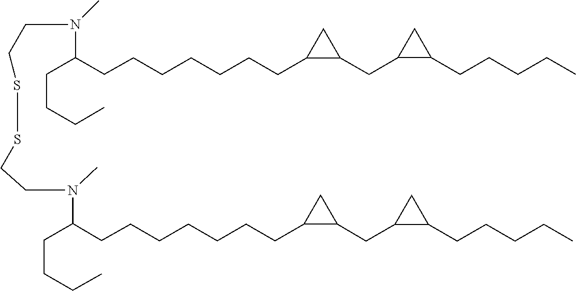 [SMILES: CCCCCC1CC1CC1CC1CCCCCCCC(CCCC)N(C)CCSSCCN(C)C(CCCC)CCCCCCCC1CC1CC1CC1CCCCC] |
| 14 | 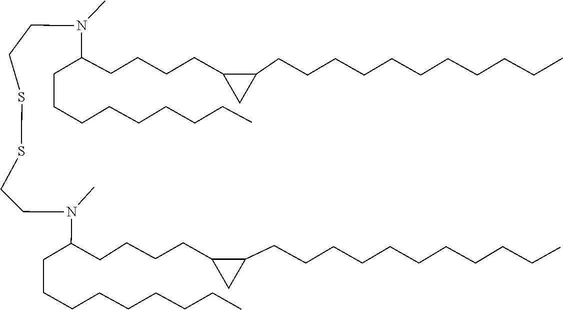 [SMILES: CCCCCCCCCCCC1CC1CCCCC(CCCCCCCCC)N(C)CCSSCCN(C)C(CCCCCCCCC)CCCCC1CC1CCCCCCCCCCC] |
| 15 | 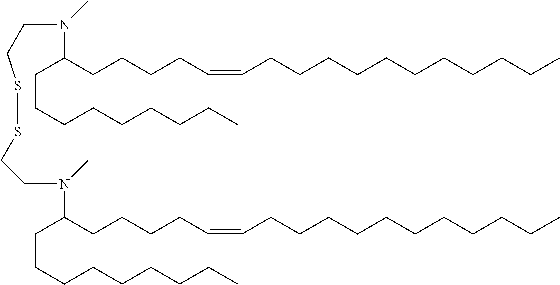 [SMILES: CCCCCCCCCCC/C=C\CCCCC(CCCCCCCCC)N(C)CCSSCCN(C)C(CCCC/C=C\CCCCCCCCCCC)CCCCCCCCC] |
| 16 | 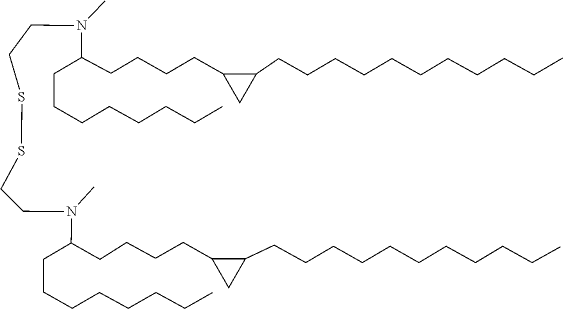 [SMILES: CCCCCCCCCCCC1CC1CCCCC(CCCCCCCC)N(C)CCSSCCN(C)C(CCCCCCCC)CCCCC1CC1CCCCCCCCCCC] |
| 17 | 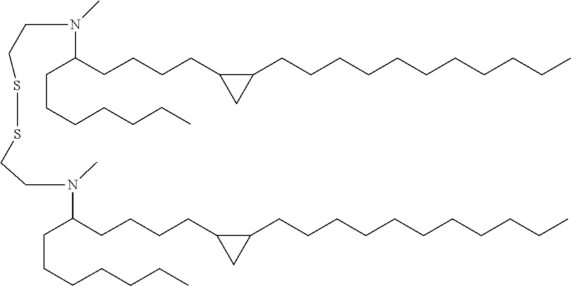 [SMILES: CCCCCCCCCCCC1CC1CCCCC(CCCCCCC)N(C)CCSSCCN(C)C(CCCCCCC)CCCCC1CC1CCCCCCCCCCC] |
| 18 | 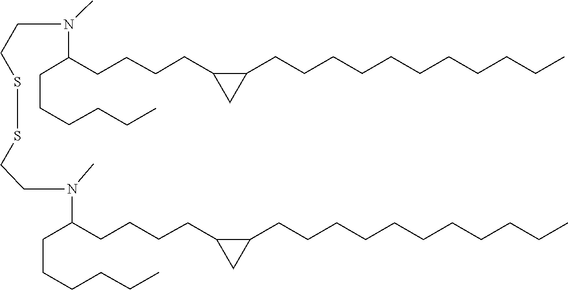 [SMILES: CCCCCCCCCCCC1CC1CCCCC(CCCCCC)N(C)CCSSCCN(C)C(CCCCCC)CCCCC1CC1CCCCCCCCCCC] |
| 19 | 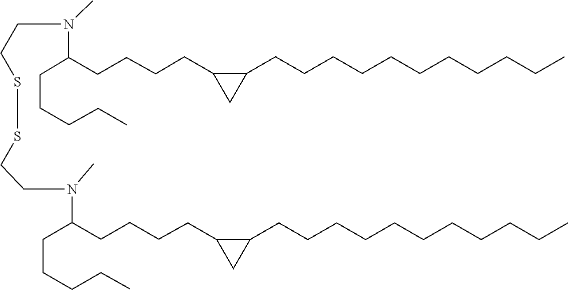 [SMILES: CCCCCCCCCCCC1CC1CCCCC(CCCCC)N(C)CCSSCCN(C)C(CCCCC)CCCCC1CC1CCCCCCCCCCC] |
| 20 | 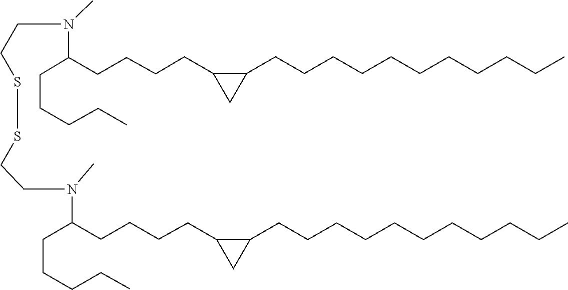 [SMILES: CCCCCCCCCCCC1CC1CCCCC(CCCCC)N(C)CCSSCCN(C)C(CCCCC)CCCCC1CC1CCCCCCCCCCC] |
| 21 | 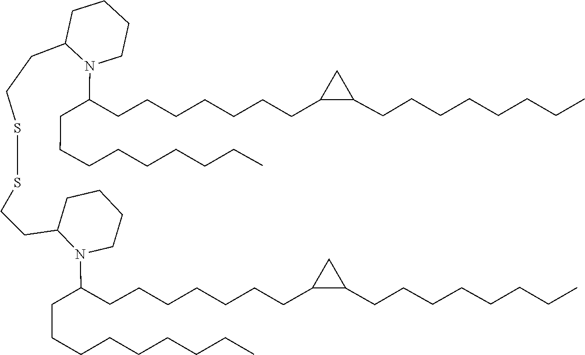 [SMILES: CCCCCCCCCC(CCCCCCCC1CC1CCCCCCCC)N1CCCCC1CCSSCCC1CCCCN1C(CCCCCCCCC)CCCCCCCC1CC1CCCCCCCC] |
| 22 | 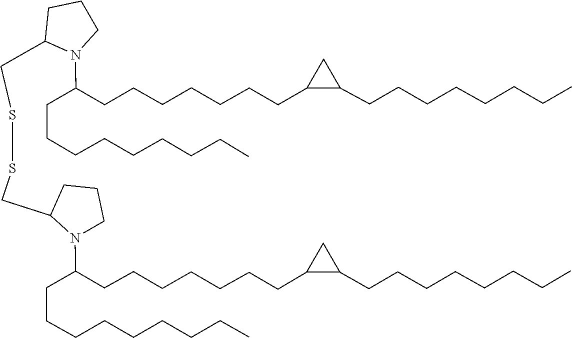 [SMILES: CCCCCCCCCC(CCCCCCCC1CC1CCCCCCCC)N1CCCC1CSSCC1CCCN1C(CCCCCCCCC)CCCCCCCC1CC1CCCCCCCC] |
| 23 | 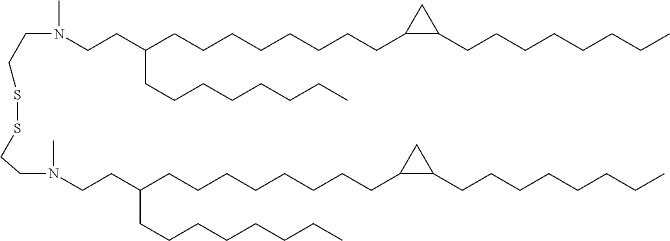 [SMILES: CCCCCCCCC(CCCCCCCCC1CC1CCCCCCCC)CCN(C)CCSSCCN(C)CCC(CCCCCCCC)CCCCCCCCC1CC1CCCCCCCC] |
| 24 | 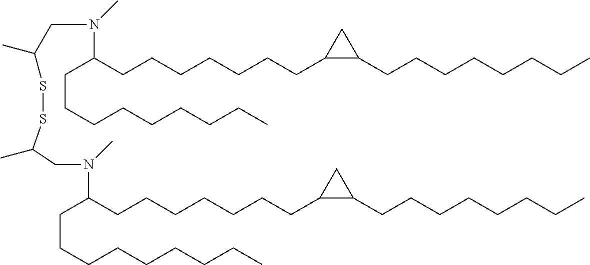 [SMILES: CCCCCCCCCC(CCCCCCCC1CC1CCCCCCCC)N(C)CC(C)SSC(C)CN(C)C(CCCCCCCCC)CCCCCCCC1CC1CCCCCCCC] |
| 25 | 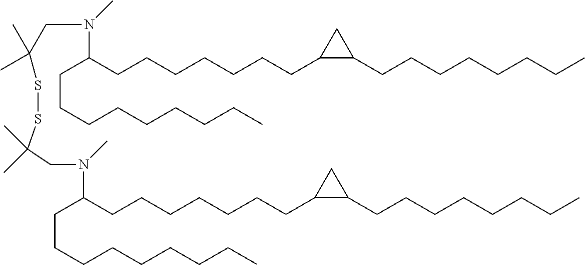 [SMILES: CCCCCCCCCC(CCCCCCCC1CC1CCCCCCCC)N(C)CC(C)(C)SSC(C)(C)CN(C)C(CCCCCCCCC)CCCCCCCC1CC1CCCCCCCC] |
| 26 | 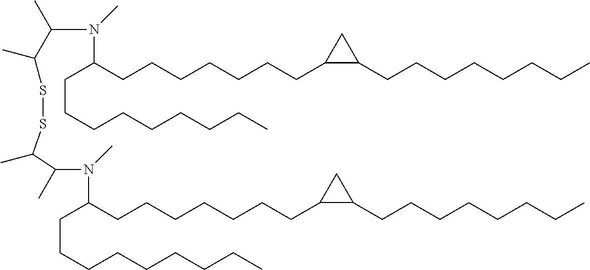 [SMILES: CCCCCCCCCC(CCCCCCCC1CC1CCCCCCCC)N(C)C(C)C(C)SSC(C)C(C)N(C)C(CCCCCCCCC)CCCCCCCC1CC1CCCCCCCC] |
| 27 | 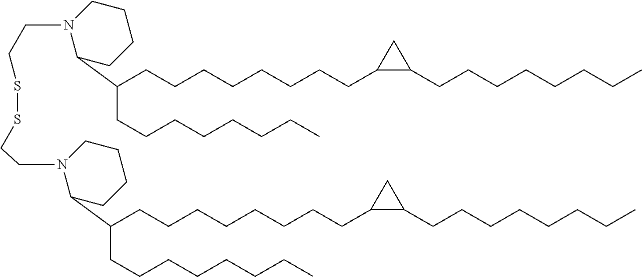 [SMILES: CCCCCCCCC1CC1CCCCCCCCC(CCCCCCCC)C1CCCCN1CCSSCCN1CCCCC1C(CCCCCCCC)CCCCCCCCC1CC1CCCCCCCC] |
| 28 | 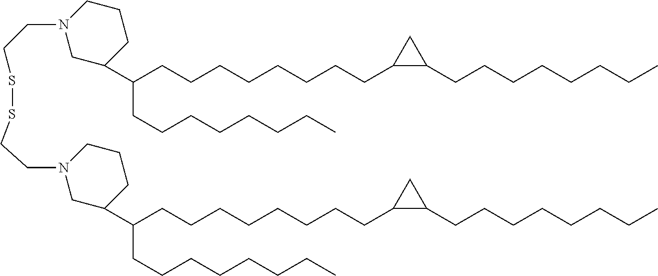 [SMILES: CCCCCCCCC(CCCCCCCCC1CC1CCCCCCCC)C1CCCN(CCSSCCN2CCCC(C(CCCCCCCC)CCCCCCCCC3CC3CCCCCCCC)C2)C1] |
| 29 | 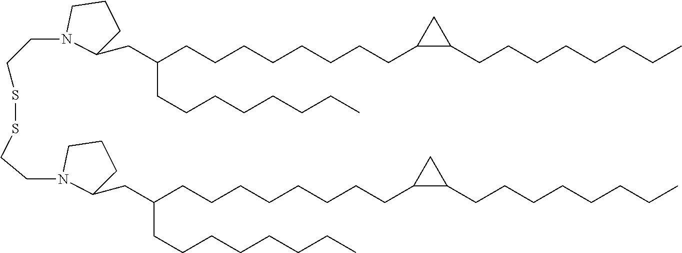 [SMILES: CCCCCCCCC(CCCCCCCCC1CC1CCCCCCCC)CC1CCCN1CCSSCCN1CCCC1CC(CCCCCCCC)CCCCCCCCC1CC1CCCCCCCC] |
| 30 | 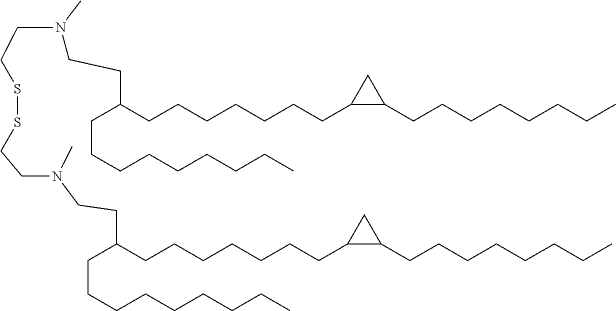 [SMILES: CCCCCCCCCC(CCCCCCCC1CC1CCCCCCCC)CCN(C)CCSSCCN(C)CCC(CCCCCCCCC)CCCCCCCC1CC1CCCCCCCC] |
| 31 | 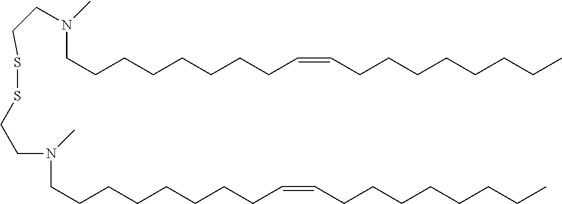 [SMILES: CCCCCCCC/C=C\CCCCCCCCN(C)CCSSCCN(C)CCCCCCCC/C=C\CCCCCCCC] |
| 32 | 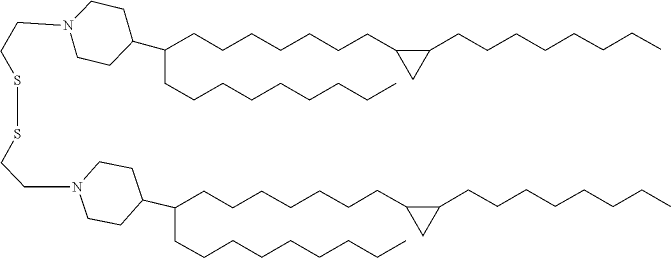 [SMILES: CCCCCCCCCC(CCCCCCCC1CC1CCCCCCCC)C1CCN(CCSSCCN2CCC(C(CCCCCCCCC)CCCCCCCC3CC3CCCCCCCC)CC2)CC1] |
| 33 | 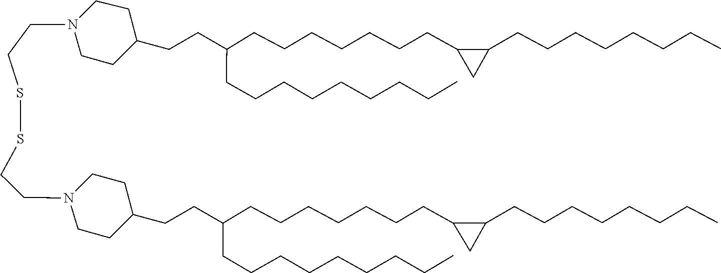 [SMILES: CCCCCCCCCC(CCCCCCCC1CC1CCCCCCCC)CCC1CCN(CCSSCCN2CCC(CCC(CCCCCCCCC)CCCCCCCC3CC3CCCCCCCC)CC2)CC1] |
| 34 | 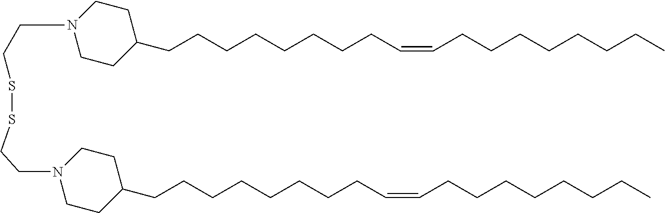 [SMILES: CCCCCCCC/C=C\CCCCCCCCC1CCN(CCSSCCN2CCC(CCCCCCCC/C=C\CCCCCCCC)CC2)CC1] |
| 35 | 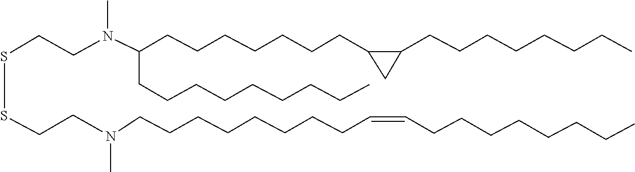 [SMILES: CCCCCCCC/C=C\CCCCCCCCN(C)CCSSCCN(C)C(CCCCCCCCC)CCCCCCCC1CC1CCCCCCCC] |
| 36 | 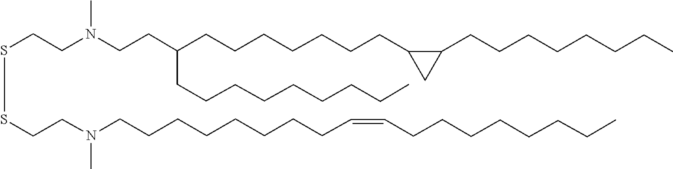 [SMILES: CCCCCCCC/C=C\CCCCCCCCN(C)CCSSCCN(C)CCC(CCCCCCCCC)CCCCCCCC1CC1CCCCCCCC] |
| 37 | 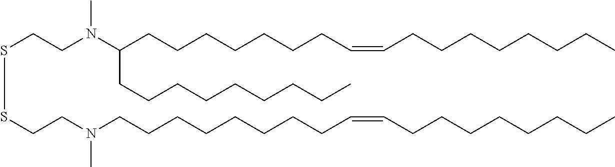 [SMILES: CCCCCCCC/C=C\CCCCCCCCN(C)CCSSCCN(C)C(CCCCCCC/C=C\CCCCCCCC)CCCCCCCCC] |
| 38 | 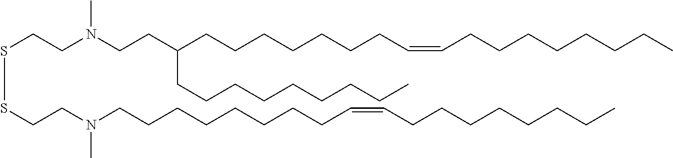 [SMILES: CCCCCCCC/C=C\CCCCCCCCN(C)CCSSCCN(C)CCC(CCCCCCC/C=C\CCCCCCCC)CCCCCCCCC] |
| 39 | 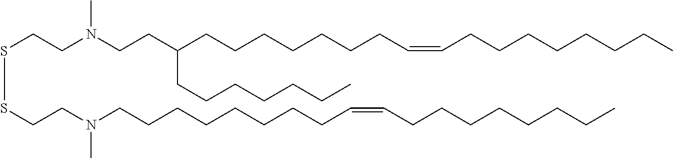 [SMILES: CCCCCCCC/C=C\CCCCCCCCN(C)CCSSCCN(C)CCC(CCCCCCC)CCCCCCC/C=C\CCCCCCCC] |
| 40 | 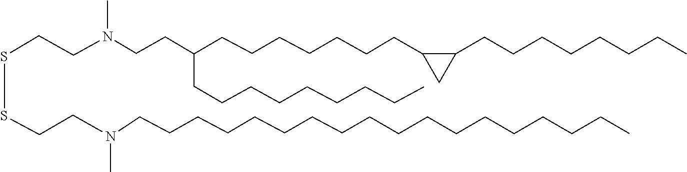 [SMILES: CCCCCCCCCCCCCCCCCCN(C)CCSSCCN(C)CCC(CCCCCCCCC)CCCCCCCC1CC1CCCCCCCC] |
| 41 | 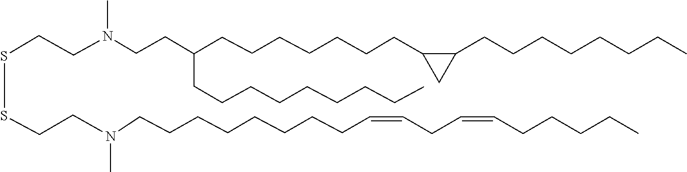 [SMILES: CCCCC/C=C\C/C=C\CCCCCCCCN(C)CCSSCCN(C)CCC(CCCCCCCCC)CCCCCCCC1CC1CCCCCCCC] |
| 42 | 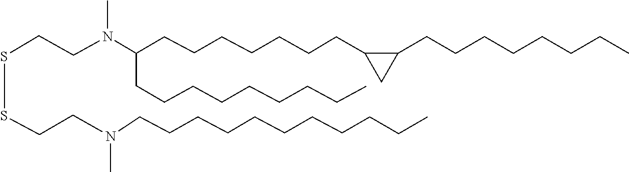 [SMILES: CCCCCCCCCCCN(C)CCSSCCN(C)C(CCCCCCCCC)CCCCCCCC1CC1CCCCCCCC] |
| 43 | 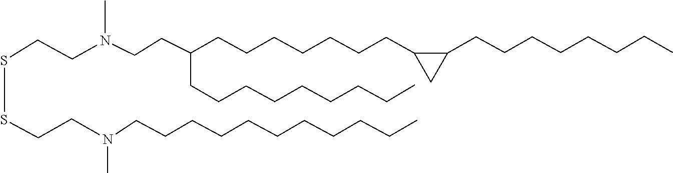 [SMILES: CCCCCCCCCCCN(C)CCSSCCN(C)CCC(CCCCCCCCC)CCCCCCCC1CC1CCCCCCCC] |
| 44 | 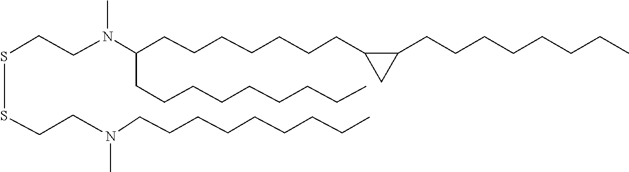 [SMILES: CCCCCCCCCC(CCCCCCCC1CC1CCCCCCCC)N(C)CCSSCCN(C)CCCCCCCCC] |
| 45 | 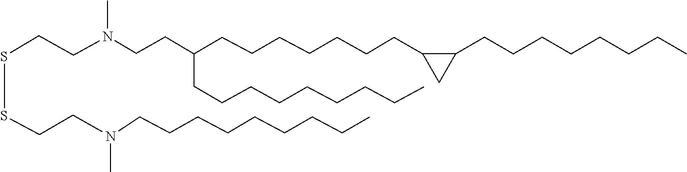 [SMILES: CCCCCCCCCC(CCCCCCCC1CC1CCCCCCCC)CCN(C)CCSSCCN(C)CCCCCCCCC] |
| 46 | 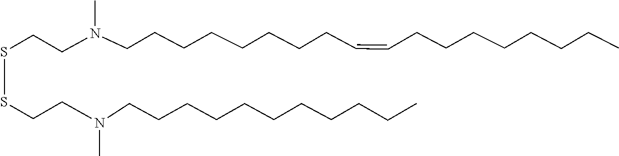 [SMILES: CCCCCCCC/C=C\CCCCCCCCN(C)CCSSCCN(C)CCCCCCCCCCC] |
| 47 | 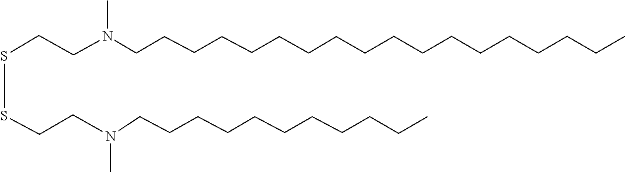 [SMILES: CCCCCCCCCCCCCCCCCCN(C)CCSSCCN(C)CCCCCCCCCCC] |
| 48 | 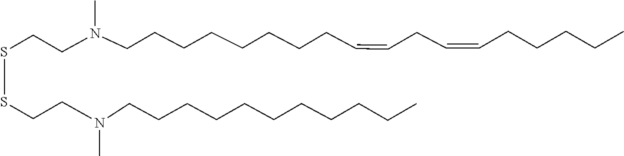 [SMILES: CCCCC/C=C\C/C=C\CCCCCCCCN(C)CCSSCCN(C)CCCCCCCCCCC] |
| 49 | 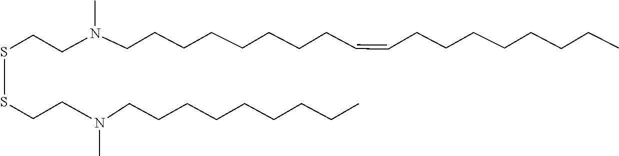 [SMILES: CCCCCCCC/C=C\CCCCCCCCN(C)CCSSCCN(C)CCCCCCCCC] |
| 50 | 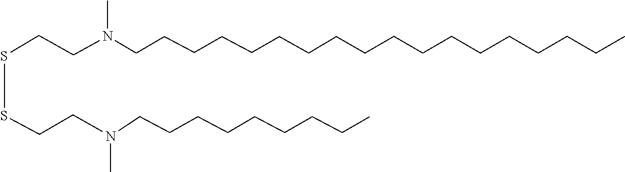 [SMILES: CCCCCCCCCCCCCCCCCCN(C)CCSSCCN(C)CCCCCCCCC] |
| 51 | 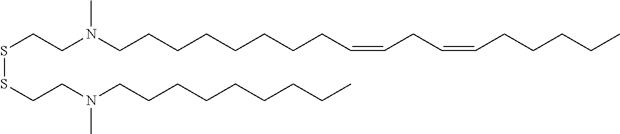 [SMILES: CCCCC/C=C\C/C=C\CCCCCCCCN(C)CCSSCCN(C)CCCCCCCCC] |

### Formula (B)

In some embodiments, the ionizable lipid in the LNPs of the present disclosure is represented by Formula (B):

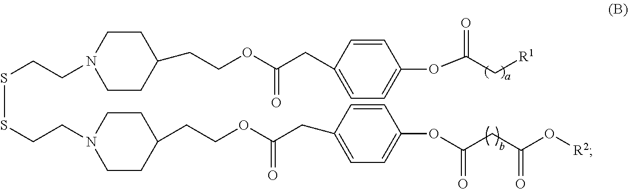

or a pharmaceutically acceptable salt thereof, wherein:
a is an integer ranging from 1 to 20 (e.g., a is 1, 2, 3, 4, 5, 6, 7, 8, 9, 10, 11, 12, 13, 14, 15, 16, 17, 18, 19, or 20);
b is an integer ranging from 2 to 10 (e.g., b is 2, 3, 4, 5, 6, 7, 8, 9, or 10);
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl.

In a second embodiment of Formula (B), the ionizable lipid of Formula (B) is represented by Formula (B-1):

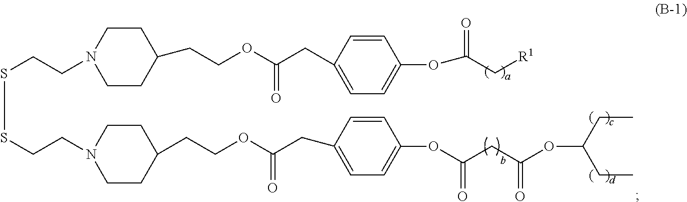

or a pharmaceutically acceptable salt thereof, wherein c and d are each independently integers ranging from 1 to 8 (e.g., 1, 2, 3, 4, 5, 6, 7, or 8), and wherein the remaining variables are as described for Formula (B).

In a third embodiment of Formula (B), c and d in Formula (B-1) are each independently integers ranging from 2 to 8, 3 to 8, 3 to 7, 3 to 6, 3 to 5, 4 to 8, 4 to 7, 4 to 6, 5 to 8, 5 to 7, or 6 to 8, wherein the remaining variables are as described for Formula (B-1).

In a fourth embodiment of Formula (B), c in Formula (B-1) is 2, 3, 4, 5, 6, 7, or 8, wherein the remaining variables are as described for Formula (B), or the second or third embodiment of Formula (B). Alternatively, c and d in Formula (B-1) are each independently 1, 3, 5, or 7, wherein the remaining variables are as described for Formula (B), or the second or third embodiment of Formula (B).

In a fifth embodiment of Formula (B), d in the cationic lipid of Formula (B-1) is 2, 3, 4, 5, 6, 7, or 8, wherein the remaining variables are as described for Formula (B), or the second, third or fourth embodiments of Formula (B). Alternatively, at least one of c and d in Formula (B-1) is 7, wherein the remaining variables are as described for Formula (B), or the second, third or fourth embodiments of Formula (B).

In a sixth embodiment of Formula (B), the ionizable lipid of Formula (B) or Formula (B-1) is represented by Formula (B-2):

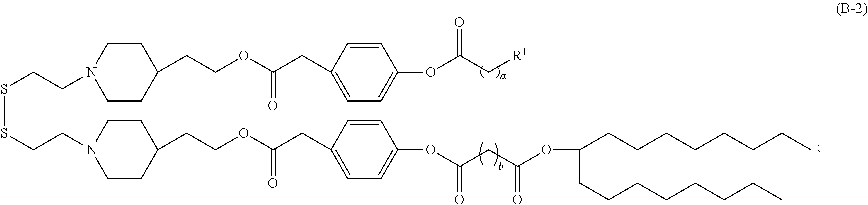

or a pharmaceutically acceptable salt thereof, wherein the remaining variables are as described for Formula (B) or Formula (B-1).

In a seventh embodiment of Formula (B), b in Formula (B), (B-1), or (B-2) is an integer ranging from 3 to 9, wherein the remaining variables are as described for Formula (B), or the second, third, fourth, fifth or sixth embodiments of Formula (B). Alternatively, b in Formula (B), (B-1), or (B-2) is an integer ranging from 3 to 8, 3 to 7, 3 to 6, 3 to 5, 4 to 9, 4 to 8, 4 to 7, 4 to 6, 5 to 9, 5 to 8, 5 to 7, 6 to 9, 6 to 8, or 7 to 9, wherein the remaining variables are as described for Formula (B), or the second, third, fourth, fifth or sixth embodiments of Formula (B). Alternatively, b in Formula (B), (B-1), or (B-2) is 3, 4, 5, 6, 7, 8, or 9, wherein the remaining variables are as described for Formula (B), or the second, third, fourth, fifth or sixth embodiments of Formula (B).

In an eighth embodiment of Formula (B), a in Formula (B), (B-1), or (B-2) is an integer ranging from 2 to 18, wherein the remaining variables are as described for Formula (B), or the second, third, fourth, fifth, sixth or seventh embodiment of Formula (B). Alternatively, a in Formula (B), (B-1), or (B-2) is an integer ranging from 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, 5 to 18, 5 to 17, 5 to 16, 5 to 15, 5 to 14, 5 to 13, 5 to 12, 5 to 11, 5 to 10, 5 to 9, 25 to 8, 5 to 7, 6 to 18, 6 to 17, 6 to 16, 6 to 15, 6 to 14, 6 to 13, 6 to 12, 6 to 11, 6 to 10, 6 to 9, 6 to 8, 7 to 18, 7 to 17, 7 to 16, 7 to 15, 7 to 14, 7 to 13, 7 to 12, 7 to 11, 7 to 10, 7 to 9, 8 to 18, 8 to 17, 8 to 16, 8 to 15, 8 to 14, 8 to 13, 8 to 12, 8 to 11, 8 to 10, 9 to 18, 9 to 17, 9 to 16, 9 to 15, 9 to 14, 9 to 13, 9 to 12, 9 to 11, 10 to 18, 10 to 17, 10 to 16, 10 to 15, 10 to 14, 10 to 13, 11 to 18, 11 to 17, 11 to 16, 11 to 15, 11 to 14, 11 to 13, 12 to 18, 12 to 17, 12 to 16, 12 to 15, 12 to 14, 13 to 18, 13 to 17, 13 to 16, 13 to 15, 14 to 18, 14 to 17, 14 to 16, 15 to 18, 15 to 17, or 16 to 18, wherein the remaining variables are as described for Formula (B), or the second, third, fourth, fifth, sixth or seventh embodiment of Formula (B). Alternatively, a in Formula (B), (B-1), or (B-2) is 2, 3, 4, 5, 6, 7, 8, 9, 10, 11, 12, 13, 14, 15, 16, 17, or 18, wherein the remaining variables are as described for Formula (B), or the second, third, fourth, fifth, sixth or seventh embodiment of Formula (B).

In a ninth embodiment of Formula (B), R¹ in Formula (B), Formula (B-1), or Formula (B-2) is absent or is selected from (C₅-C₁₅)alkenyl, —C(O)O(C₄-C₁₈)alkyl, and cyclopropyl substituted with (C₄-C₁₆)alkyl, wherein the remaining variables are as described for Formula (B), or the second, third, fourth, fifth, sixth, seventh or eighth embodiments of Formula (B). Alternatively, R¹ in Formula (B), Formula (B-1), or Formula (B-2) is absent or is selected from (C₅-C₁₅)alkenyl, —C(O)O(C₄-C₁₆)alkyl, and cyclopropyl substituted with (C₄-C₁₆)alkyl, wherein the remaining variables are as described for Formula (B), or the second, third, fourth, fifth, sixth, seventh or eighth embodiments of Formula (B). Alternatively, R¹ in Formula (B), Formula (B-1), or Formula (B-2) is absent or is selected from (C₅-C₁₂)alkenyl, —C(O)O(C₄-C₁₂)alkyl, and cyclopropyl substituted with (C₄-C₁₂)alkyl, wherein the remaining variables are as described for Formula (B), or the second, third, fourth, fifth, sixth, seventh or eighth embodiments of Formula (B). In another alternative, R¹ in the cationic lipid of Formula (B), Formula (B-1), or Formula (B-2) is absent or is selected from (C₅-C₁₀)alkenyl, —C(O)O(C₄-C₁₀)alkyl, and cyclopropyl substituted with (C₄-C₁₀)alkyl, wherein the remaining variables are as described for Formula (B), or the second, third, fourth, fifth, sixth, seventh or eighth embodiments of Formula (B).

In a tenth embodiment of Formula (B), R¹ is C₁ alkenyl, wherein the remaining variables are as described for Formula (B), or the second, third, fourth, fifth, sixth, seventh or eighth embodiments of Formula (B).

In an eleventh embodiment of Formula (B), the alkyl in C(O)O(C₂-C₂₀)alkyl, —C(O)O(C₄-C₁₈)alkyl, —C(O)O(C₄-C₁₂)alkyl, or —C(O)O(C₄-C₁₀)alkyl of R¹ in Formula (B), Formula (B-1), or Formula (B-2) is an unbranched alkyl, wherein the remaining variables are as described for Formula (B), or the second, third, fourth, fifth, sixth, seventh, eighth or ninth embodiments of Formula (B). In one embodiment, R¹ is —C(O)O(C₉ alkyl). Alternatively, the alkyl in —C(O)O(C₄-C₁₈)alkyl, —C(O)O(C₄-C₁₂)alkyl, or —C(O)O(C₄-C₁₀)alkyl of R¹ in Formula (B), Formula (B-1), or Formula (B-2) is a branched alkyl, wherein the remaining variables are as described for Formula (B), Formula (B-1), or Formula (B-2), or the second, third, fourth, fifth, sixth, seventh, eighth or ninth embodiments of Formula (B). In one embodiment, R¹ is —C(O)O(C₁₇ alkyl), wherein the remaining variables are as described for Formula (B), Formula (B-1), or Formula (B-2), or the second, third, fourth, fifth, sixth, seventh, eighth or ninth embodiments of Formula (B).

In a twelfth embodiment of Formula (B), R¹ in Formula (B), Formula (B-1), or Formula (B-2) is selected from any group listed in Table 2 below, wherein the wavy bond in each of the groups indicates the point of attachment of the group to the rest of the ionizable lipid molecule, and wherein the remaining variables are as described for Formula (B), Formula (B-1), or Formula (B-2), or the second, third, fourth, fifth, sixth, seventh or eighth embodiments of Formula (B). The present disclosure further contemplates the combination of any one of the R¹ groups in Table 2 with any one of the R² groups in Table 3 in Formula (B), wherein the remaining variables are as described for Formula (B), Formula (B-1), or Formula (B-2), or the second, third, fourth, fifth, sixth, seventh or eighth embodiments of Formula (B).

TABLE 2

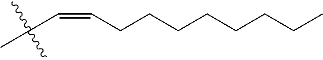 [SMILES: */C=C\CCCCCCCC]

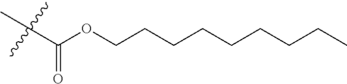 [SMILES: *C(=O)OCCCCCCCCC]

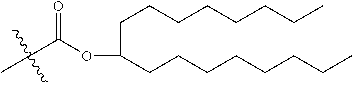 [SMILES: *C(=O)OC(CCCCCCCC)CCCCCCCC]

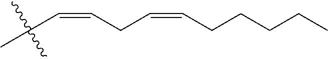 [SMILES: */C=C\C/C=C\CCCCC]

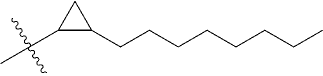 [SMILES: *C1CC1CCCCCCCC]

In a thirteenth embodiment, R² in Formula (B) or a pharmaceutically acceptable salt thereof is selected from any group listed in Table 3 below, wherein the wavy bond in each of the groups indicates the point of attachment of the group to the rest of the ionizable lipid molecule, and wherein the remaining variables are as described for Formula (B), Formula (B-1), or Formula (B-2), or the second, third, fourth, fifth, sixth, seventh or eighth, ninth, tenth, eleventh or twelfth embodiments of Formula (B).

TABLE 3

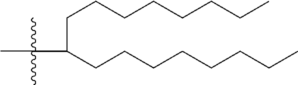 [SMILES: *C(CCCCCCCC)CCCCCCCC]

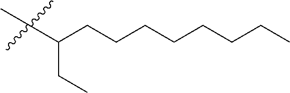 [SMILES: *C(CC)CCCCCCCC]

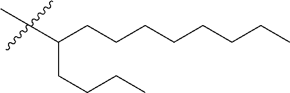 [SMILES: *C(CCCC)CCCCCCCC]

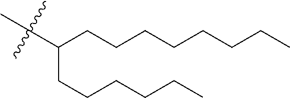 [SMILES: *C(CCCCCC)CCCCCCCC]

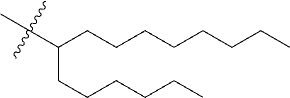 [SMILES: *C(CCCCCC)CCCCCCCC]

Table 4 below provides specific examples of ionizable lipids of Formula (B).

Pharmaceutically acceptable salts as well as ionized and neutral forms are also included.

TABLE 4 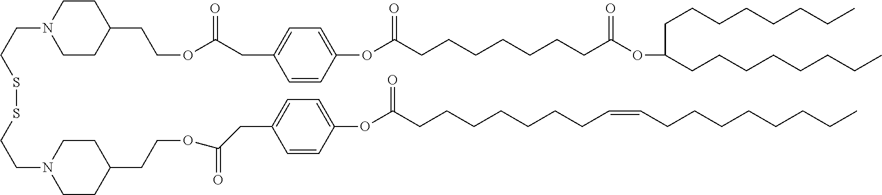 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]
Lipid 52 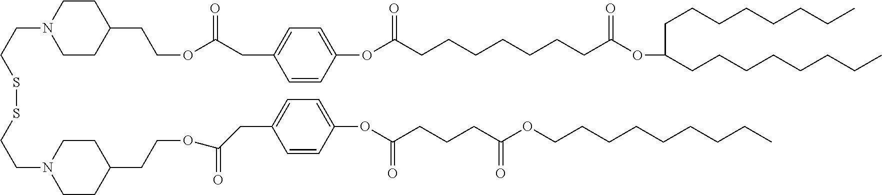 [SMILES: CCCCCCCCCOC(=O)CCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]
Lipid 53 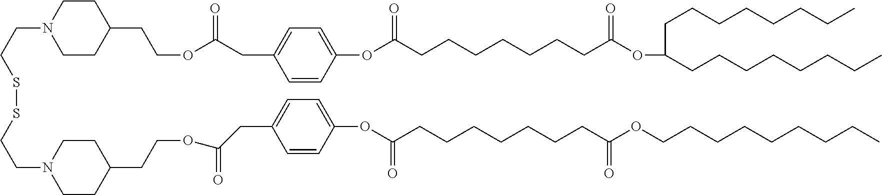 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]
Lipid 54 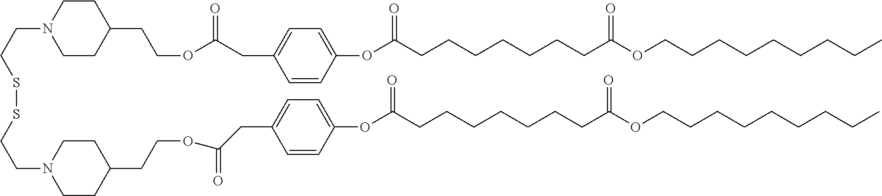 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OCCCCCCCCC)cc4)CC3)CC2)cc1]
Lipid 55 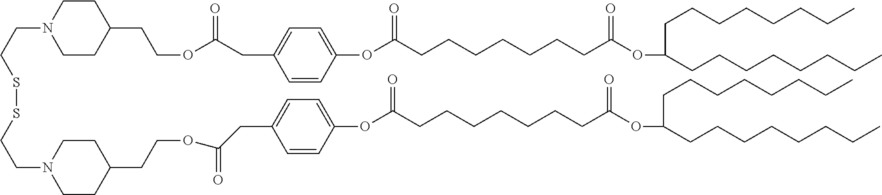 [SMILES: CCCCCCCCC(CCCCCCCC)OC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]
Lipid 56 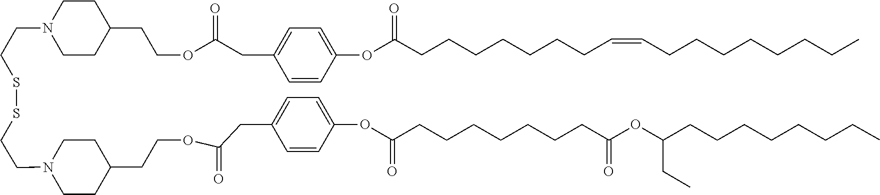 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CC)CCCCCCCC)cc4)CC3)CC2)cc1]
Lipid 57 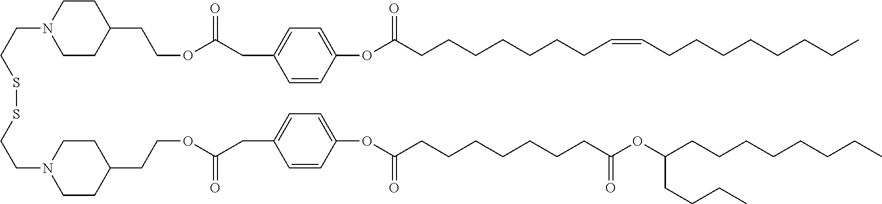 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]
Lipid 58 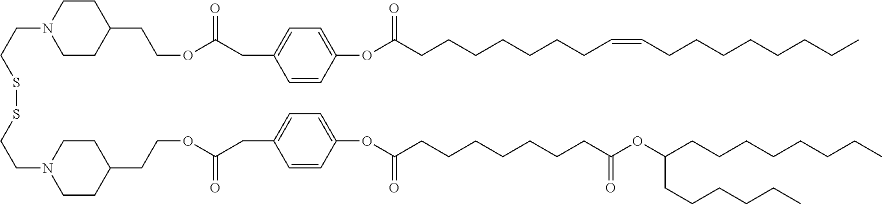 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCC)CCCCCCCC)cc4)CC3)CC2)cc1]
Lipid 59 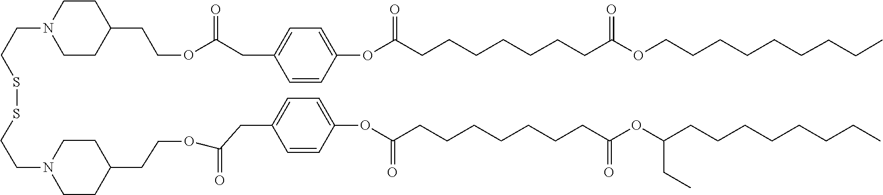 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CC)CCCCCCCC)cc4)CC3)CC2)cc1]
Lipid 60 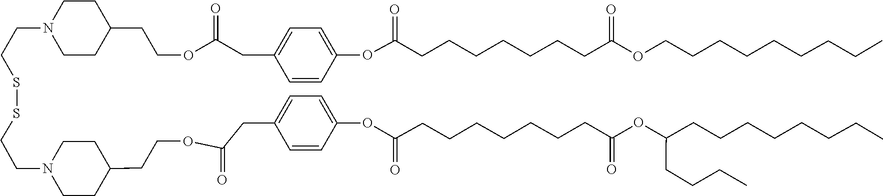 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]
Lipid 61 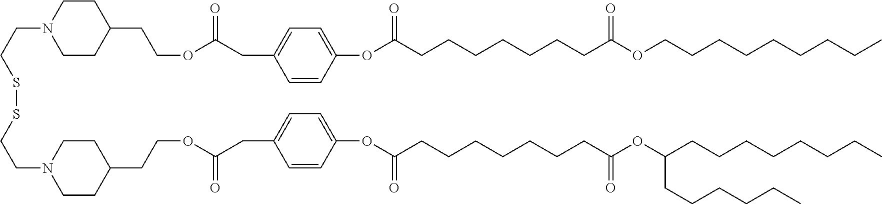 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCC)CCCCCCCC)cc4)CC3)CC2)cc1]
Lipid 62 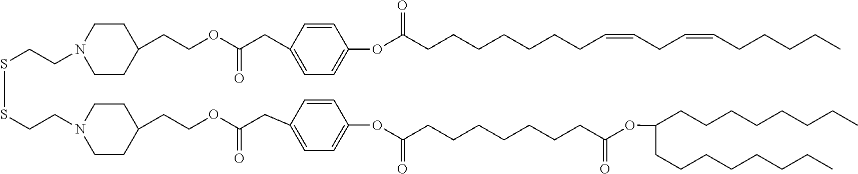 [SMILES: CCCCC/C=C\C/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]
Lipid 63 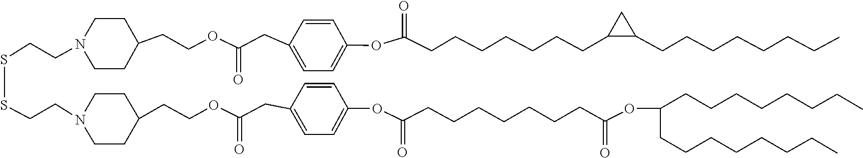 [SMILES: CCCCCCCCC(CCCCCCCC)OC(=O)CCCCCCCC(=O)Oc1ccc(CC(=O)OCCC2CCN(CCSSCCN3CCC(CCOC(=O)Cc4ccc(OC(=O)CCCCCCCC5CC5CCCCCCCC)cc4)CC3)CC2)cc1]
Lipid 64 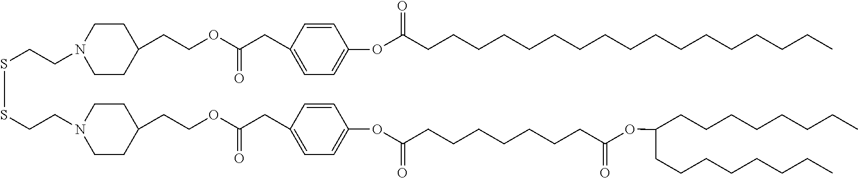 [SMILES: CCCCCCCCCCCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]
Lipid 65 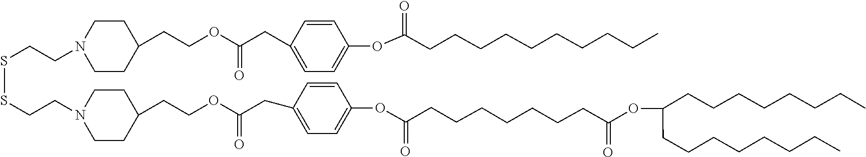 [SMILES: CCCCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]
Lipid 66 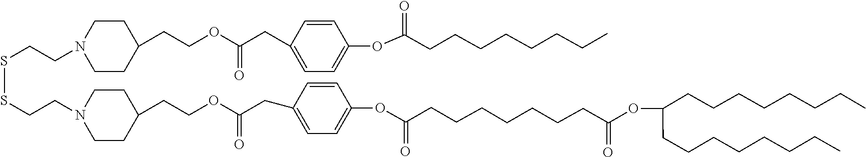 [SMILES: CCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]
Lipid 67 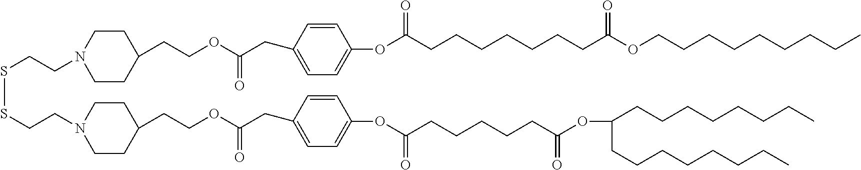 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OC(CCCCCCCC)CCCCCCCC)cc4)CC3)CC2)cc1]
Lipid 68 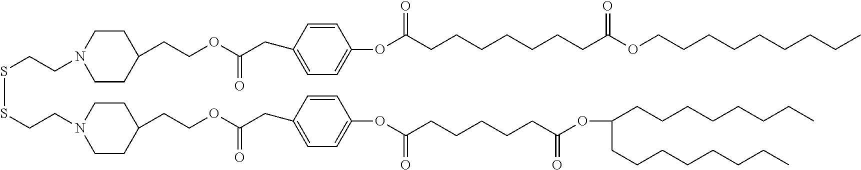 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OC(CCCCCCCC)CCCCCCCC)cc4)CC3)CC2)cc1]
Lipid 69 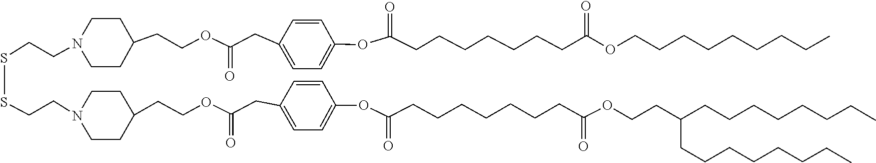 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OCCC(CCCCCCCC)CCCCCCCC)cc4)CC3)CC2)cc1]
Lipid 70 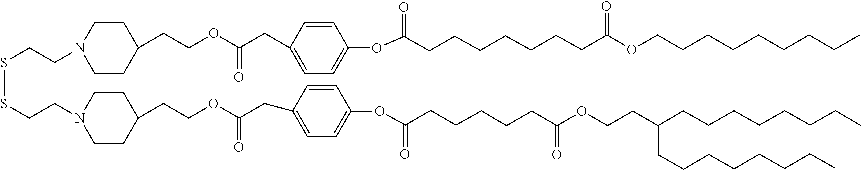 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OCCC(CCCCCCCC)CCCCCCCC)cc4)CC3)CC2)cc1]
Lipid 71

### Formula (C)

In some embodiments, the ionizable lipid in the LNPs of the present disclosure are represented by Formula (C):

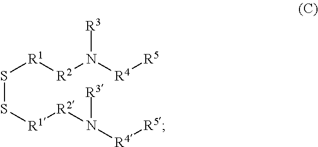

or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R;
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆)cycloalkyl; and
R^{a} and R are each halo or cyano.

In a second embodiment of Formula (C), R¹ and R¹ are each independently (C₁-C₆)alkylene, wherein the remaining variables are as described above for Formula (C). Alternatively, R¹ and R^{1′} are each independently (C₁-C₃)alkylene, wherein the remaining variables are as described above for Formula (C).

In a third embodiment of Formula (C), the ionizable lipid of the Formula (C) is represented by Formula (C-1):

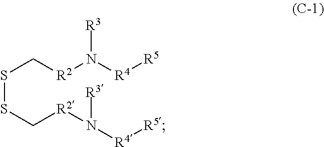

or a pharmaceutically acceptable salt thereof, wherein R² and R^{2′}, R³ and R^{3′}, R⁴ and R^{4′} and R⁵ and R^{5′} are as described above for Formula (C) or the second embodiment of Formula (C).

In a fourth embodiment, the ionizable lipid of Formula (C) is represented by Formula (C-2) or Formula (C-3):

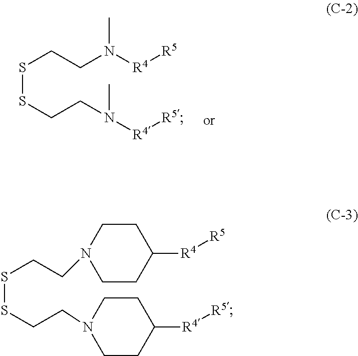

or a pharmaceutically acceptable salt thereof, wherein R⁴ and R^{4′} and R⁵ and R^{5′} are as described above for Formula (C).

In a fifth embodiment of Formula (C), the ionizable lipid of Formula (C) is represented by Formula (C-4) or (C-5):

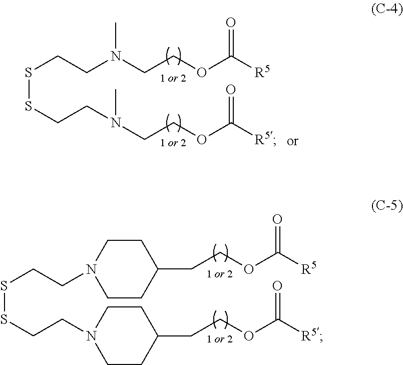

or a pharmaceutically acceptable salt thereof, wherein R⁵ and R′ are as described above for Formula (C).

In a sixth embodiment of Formula (C), the ionizable lipid of Formula (C) is represented by Formula (C-6), (C-7), (C-8), or (C-9):

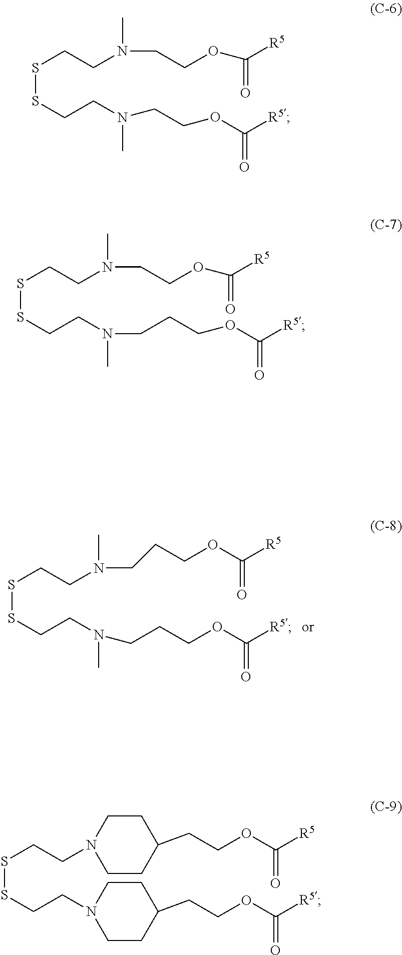

or a pharmaceutically acceptable salt thereof, wherein R⁵ and R^{5′} are as described above for Formula (XV).

In a seventh embodiment of Formula (C), at least one of R⁵ and R^{5′} in Formula (C), (C-1), (C-2), (C-3), (C-4), (C-5), (C-6), (C-7), (C-8), or (C-9) is a branched alkyl or branched alkenyl, and the remaining variables are as described above for Formula (C) or the second embodiment of Formula (C). Alternatively, one of R⁵ and R^{5′} in Formula (C), (C-1), (C-2), (C-3), (C-4), (C-5), (C-6), (C-7), (C-8), or (C-9) is a branched alkyl or branched alkenyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a branched alkyl or branched alkenyl, and the remaining variables are as described above for Formula (C) or the second embodiment of Formula (C). Alternatively, R^{5′} in Formula (C), (C-1), (C-2), (C-3), (C-4), (C-5), (C-6), (C-7), (C-8), or (C-9) is a branched alkyl or branched alkenyl, and the remaining variables are as described above for Formula (C) or the second embodiment of Formula (C).

In an eighth embodiment of Formula (C), R⁵ in Formula (C), (C-1), (C-2), (C-3), (C-4), (C-5), (C-6), (C-7), (C-8), or (C-9) is a (C₆-C₂₆)alkyl or (C₆-C₂₆)alkenyl, each of which are optionally interrupted with —C(O)O— or (C₃-C₆)cycloalkyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₆-C₂₆)alkyl or (C₆-C₂₆)alkenyl, each of which are optionally interrupted with —C(O)O— or (C₃-C₅)cycloalkyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₇-C₂₆)alkyl or (C₇-C₂₆)alkenyl, each of which are optionally interrupted with —C(O)O— or (C₃-C₅)cycloalkyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₈-C₂₆)alkyl or (C₈-C₂₆)alkenyl, each of which are optionally interrupted with —C(O)O— or (C₃-C₅)cycloalkyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₆-C₂₄)alkyl or (C₆-C₂₄)alkenyl, each of which are optionally interrupted with —C(O)O— or cyclopropyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₈-C₂₄)alkyl or (C₈-C₂₄)alkenyl, wherein said (C₈-C₂₄)alkyl is optionally interrupted with —C(O)O— or cyclopropyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₅-C₁₀)alkyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₁₄-C₁₆)alkyl interrupted with cyclopropyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₁₀-C₂₄)alkyl interrupted with —C(O)O—, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₁₆-C₁₈)alkenyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is —(CH₂)₃C(O)O(CH₂)₈CH₃, —(CH₂)₅C(O)O(CH₂)₈CH₃, —(CH₂)₇C(O)O(CH₂)₈CH₃, —(CH₂)₇C(O)OCH[(CH₂)₇CH₃]₂, —(CH₂)₇-C₃H₆—(CH₂)₇CH₃, —(CH₂)₇CH₃, —(CH₂)₉CH₃, —(CH₂)₁₆CH₃, —(CH₂)₇CH═CH(CH₂)₇CH₃, or —(CH₂)₇CH═CHCH₂CH═CH(CH₂)₄CH₃, and the remaining variables are as described above for Formula (C) or the second embodiment of Formula (C).

In a ninth embodiment, R^{5′} in Formula (C), (C-1), (C-2), (C-3), (C-4), (C-5), (C-6), (C-7), (C-8), or (C-9) is a (C₁₅-C₂₈)alkyl interrupted with —C(O)O—, and the remaining variables are as described above for Formula (C) or the second or eighth embodiments of Formula (C). Alternatively, R^{5′} in Formula (C), (C-1), (C-2), (C-3), (C-4), (C-5), (C-6), (C-7), (C-8), or (C-9) is a (C₁₇-C₂₈)alkyl interrupted with —C(O)O—, and the remaining variables are as described above for Formula (C) or the second or eighth embodiments of Formula (C). Alternatively, R^{5′} in Formula (C), (C-1), (C-2), (C-3), (C-4), (C-5), (C-6), (C-7), (C-8), or (C-9) is a (C₁₉-C₂₈)alkyl interrupted with —C(O)O—, and the remaining variables are as described above for Formula (C) or the second or eighth embodiments of Formula (C). Alternatively, R^{5′} in Formula (C), (C-1), (C-2), (C-3), (C-4), (C-5), (C-6), (C-7), (C-8), or (C-9) is a (C₁₇-C₂₆)alkyl interrupted with —C(O)O—, and the remaining variables are as described above for Formula (C) or the second or eighth embodiments of Formula (C). Alternatively, R^{5′} in Formula (C), (C-1), (C-2), (C-3), (C-4), (C-5), (C-6), (C-7), (C-8), or (C-9) is a (C₁₉-C₂₆)alkyl interrupted with —C(O)O—, and the remaining variables are as described above for Formula (C) or the second or eighth embodiments of Formula (C). Alternatively, R^{5′} in Formula (C), (C-1), (C-2), (C-3), (C-4), (C-5), (C-6), (C-7), (C-8), or (C-9) is a (C₂₀-C₂₆)alkyl interrupted with —C(O)O—, and the remaining variables are as described above for Formula (C) or the second or eighth embodiments of Formula (C). Alternatively, R^{5′} is a (C₂₂-C₂₄)alkyl interrupted with —C(O)O—, and the remaining variables are as described above for Formula (C) or the second or eighth embodiments of Formula (C). Alternatively, R^{5′} is —(CH₂)₅C(O)OCH[(CH₂)₇CH₃]₂, —(CH₂)₇C(O)OCH[(CH₂)₇CH₃]₂, —(CH₂)₅C(O)OCH(CH₂)₂[(CH₂)₇CH₃]₂, or —(CH₂)₇C(O)OCH(CH₂)₂[(CH₂)₇CH₃]₂, and the remaining variables are as described above for Formula (C) or the second or eighth embodiments of Formula (C).

In some embodiments, the ionizable lipid of Formula (C), (C-1), (C-3), (C-3), (C-4), (C-5), (C-7), (C-8), or (C-9) may be selected from any of the lipids listed in Table 5 below, or pharmaceutically acceptable salts thereof.

TABLE 5

| Lipid No. | Lipid Structure |
|---|---|
| 72 | 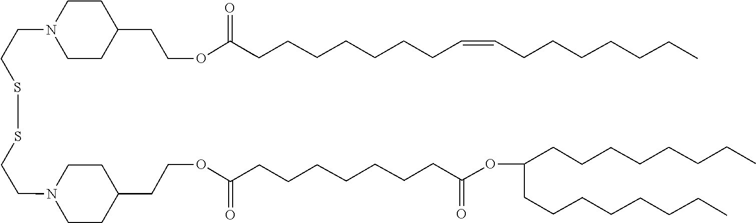 |
| 73 | 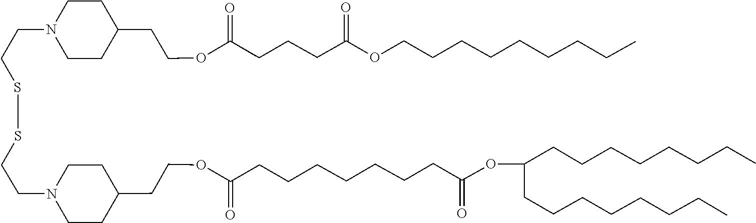 |
| 74 | 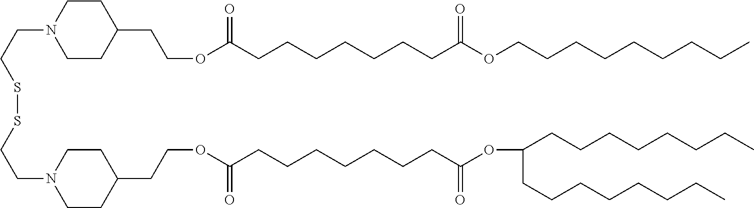 [SMILES: CCCCCCCCCOC(=O)CCCCCCCC(=O)OCCC1CCN(CCSSCCN2CCC(CCOC(=O)CCCCCCCC(=O)OC(CCCCCCCC)CCCCCCCC)CC2)CC1] |
| 75 | 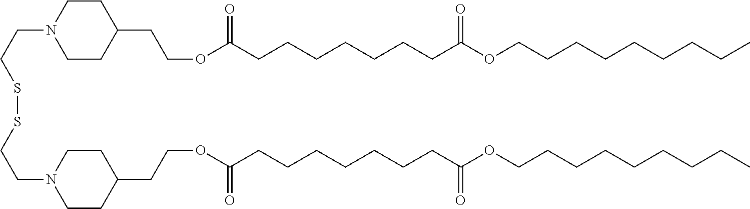 [SMILES: CCCCCCCCCOC(=O)CCCCCCCC(=O)OCCC1CCN(CCSSCCN2CCC(CCOC(=O)CCCCCCCC(=O)OCCCCCCCCC)CC2)CC1] |
| 76 | 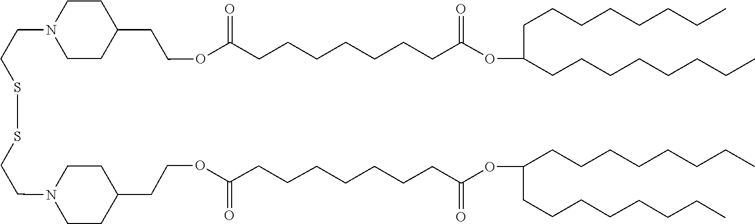 [SMILES: CCCCCCCCC(CCCCCCCC)OC(=O)CCCCCCCC(=O)OCCC1CCN(CCSSCCN2CCC(CCOC(=O)CCCCCCCC(=O)OC(CCCCCCCC)CCCCCCCC)CC2)CC1] |

### Formula (D)

In some embodiments, the ionizable lipid, e.g., cationic lipid, in the LNPs of the present disclosure is represented by Formula (D):

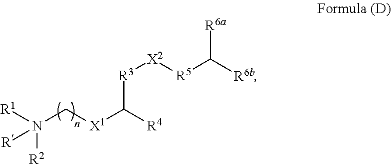

or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all attached is positively charged;
R¹ and R² are each independently hydrogen, C₁-C₆ alkyl, or C₂-C₆ alkenyl;
R³ is C₁-C₁₂ alkylene or C₂-C₁₂ alkenylene;
R⁴ is C₁-C₁₈ unbranched alkyl, C₂-C₁₈ unbranched alkenyl, or

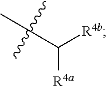

wherein:
   R^{4a} and R^{4b} are each independently C₁-C₁₆ unbranched alkyl or C₂-C₁₆ unbranched alkenyl;
R⁵ is absent, C₁-C₈ alkylene, or C₂-C₈ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6.

In a second embodiment of Formula (D), X¹ and X² are the same; and all other remaining variables are as described for Formula (C).

In a third embodiment of Formula (D), X¹ and X² are each independently —OC(═O)—, —SC(═O)—, —OC(═S)—, —C(═O)O—, —C(═O)S—, or —S—S—; or X¹ and X² are each independently —C(═O)O—, —C(═O)S—, or —S—S—; or X¹ and X² are each independently —C(═O)O— or —S—S—; and all other remaining variables are as described for Formula (D) or the second embodiment of Formula (D).

In a fourth embodiment of Formula (D), the ionizable lipid, e.g., cationic lipid, in the LNPs of the present disclosure, is represented by Formula (D-1):

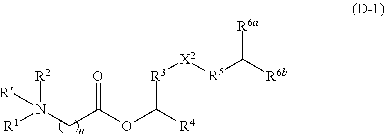

or a pharmaceutically acceptable salt thereof, wherein n is an integer selected from 1, 2, 3, and 4; and all other remaining variables are as described for Formula (D) or the second or third embodiments of Formula (D).

In a fifth embodiment of Formula (D), the ionizable lipid, e.g., cationic lipid, in the LNPs of the present disclosure, is represented by Formula (D-2):

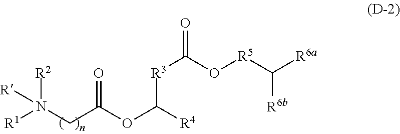

or a pharmaceutically acceptable salt thereof, wherein n is an integer selected from 1, 2, and 3; and all other remaining variables are as described for Formula (D) or the second or third embodiments of Formula (D).

In a sixth embodiment of Formula (D), the ionizable lipid, e.g., cationic lipid, in the LNPs of the present disclosure is represented by Formula (D-3):

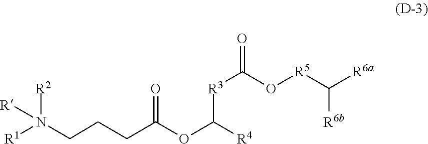

or a pharmaceutically acceptable salt thereof, and all other remaining variables are as described for Formula (D) or the second or third embodiments of Formula (D).

In a seventh embodiment of Formula (D), in the ionizable lipid, e.g., cationic lipid, according to Formula (D), Formula (D-1), Formula (D-2), Formula (D-3), or the second or third embodiments of Formula (D), R¹ and R² are each independently hydrogen, C₁-C₆ alkyl or C₂-C₆ alkenyl, or C₁-C₅ alkyl or C₂-C₅ alkenyl, or C₁-C₄ alkyl or C₂-C₄ alkenyl, or C6 alkyl, or C5 alkyl, or C4 alkyl, or C3 alkyl, or C2 alkyl, or C₁ alkyl, or C6 alkenyl, or C5 alkenyl, or C4 alkenyl, or C3 alkenyl, or C2 alkenyl; and all other remaining variables are as described for Formula (D), Formula (D-1), Formula (D-2), Formula (D-3) or the second or third embodiments of Formula (D).

In an eighth embodiment of Formula (D), the ionizable lipid, e.g., cationic lipid, in the LNPs of the present disclosure is represented by Formula (D-4):

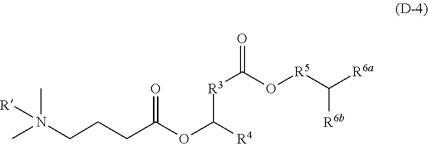

or a pharmaceutically acceptable salt thereof, and all other remaining variables are as described for Formula (D), Formula (D-1), Formula (D-2), Formula (D-3) or the second, third or seventh embodiments of Formula (D).

In a ninth embodiment of Formula (D), in the ionizable lipid, e.g., cationic lipid, according to Formula (D), Formula (D-1), Formula (D-2), Formula (D-3), Formula (D-4) or the second, third or seventh embodiments of Formula (D), R³ is C₁-C₉ alkylene or C₂-C₉ alkenylene, C₁-C₇ alkylene or C₂-C₇ alkenylene, C₁-C₅ alkylene or C₂-C₅ alkenylene, or C₂-C₈ alkylene or C₂-C₈ alkenylene, or C₃-C₇ alkylene or C₃-C₇ alkenylene, or C₅-C₇ alkylene or C₅-C₇ alkenylene; or R³ is C₁₂ alkylene, C₁₁ alkylene, C₁₀ alkylene, C₉ alkylene, or C₈ alkylene, or C₇ alkylene, or C₆ alkylene, or C₅ alkylene, or C₄ alkylene, or C₃ alkylene, or C₂ alkylene, or C₁ alkylene, or C₁₂ alkenylene, C₁₁ alkenylene, C₁₀ alkenylene, C₉ alkenylene, or C₈ alkenylene, or C₇ alkenylene, or C₆ alkenylene, or C₅ alkenylene, or C₄ alkenylene, or C₃ alkenylene, or C₂ alkenylene; and all other remaining variables are as described for Formula (D), Formula (D-1), Formula (D-2), Formula (D-3), Formula (D-4) or the second, third or seventh embodiments of Formula (D).

In a tenth embodiment of Formula (D), in the ionizable lipid, e.g., cationic lipid, according to Formula (D), Formula (D-1), Formula (D-2), Formula (D-3), Formula (D-4) or the second, third or seventh embodiments of Formula (D), R⁵ is absent, C₁-C₆ alkylene, or C₂-C₆ alkenylene; or R⁵ is absent, C₁-C₄ alkylene, or C₂-C₄ alkenylene; or R⁵ is absent; or R⁵ is C₈ alkylene, C₇ alkylene, C₆ alkylene, C₅ alkylene, C₄ alkylene, C₃ alkylene, C₂ alkylene, C₁ alkylene, C₈ alkenylene, C₇ alkenylene, C₆ alkenylene, C₅ alkenylene, C₄ alkenylene, C₃ alkenylene, or C₂ alkenylene; and all other remaining variables are as described for Formula (D), Formula (D-1), Formula (D-2), Formula (D-3), Formula (D-4) or the second, third, seventh, or ninth embodiments of Formula (D).

In an eleventh embodiment of Formula (D), in the ionizable lipid, e.g., cationic lipid, according to Formula (D), Formula (D-1), Formula (D-2), Formula (D-3), Formula (D-4) or the second, third, seventh, ninth or tenth embodiments of Formula (D), R⁴ is C₁-C₁₄ unbranched alkyl, C₂-C₁₄ unbranched alkenyl, or

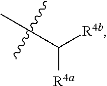

wherein R^{4a} and R^{4b} are each independently C₁-C₁₂ unbranched alkyl or C₂-C₁₂ unbranched alkenyl; or R⁴ is C₂-C₁₂ unbranched alkyl or C₂-C₁₂ unbranched alkenyl; or R⁴ is C₅-C₇ unbranched alkyl or C₅-C₇ unbranched alkenyl; or R⁴ is C₁₆ unbranched alkyl, C₁₅ unbranched alkyl, C₁₄ unbranched alkyl, C₁₃ unbranched alkyl, C₁₂ unbranched alkyl, C₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ unbranched alkyl, C₁ unbranched alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; or R⁴ is

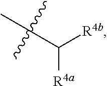

wherein R^{4a} and R^{4b} are each independently C₂-C₁₀ unbranched alkyl or C₂-C₁₀ unbranched alkenyl; or R⁴ is

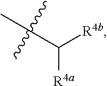

wherein R^{4a} and R^{4b} are each independently C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ alkyl, C₁ alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; and all other remaining variables are as described for Formula (D), Formula (D-1), Formula (D-2), Formula (D-3), Formula (D-4) or the second, third, seventh, ninth or tenth embodiments of Formula (D).

In a twelfth embodiment, in the ionizable lipid, e.g., cationic lipid, according to Formula (D), Formula (D-1), Formula (D-2), Formula (D-3), Formula (D-4), or the second, third, seventh, ninth, tenth or eleventh embodiments of Formula (D), R^{6a} and R^{6b} are each independently C₆-C₁₄ alkyl or C₆-C₁₄ alkenyl; or R^{6a} and R^{6b} are each independently C₈-C₁₂ alkyl or C₈-C₁₂ alkenyl; or R^{6a} and R^{6b} are each independently C₁₆ alkyl, C₁₅ alkyl, C₁₄ alkyl, C₁₃ alkyl, C₁₂ alkyl, C₁₁ alkyl, C₁₀ alkyl, C₉ alkyl, C₈ alkyl, C₇alkyl, C₁₆ alkenyl, C₁₅ alkenyl, C₁₄ alkenyl, C₁₃ alkenyl, C₁₂ alkenyl, C₁₁ alkenyl, C₁₀ alkenyl, C₉ alkenyl, C₈ alkenyl, or C₇ alkenyl; provided that the total number of carbon atoms in R^{6a} and R^{6b} as combined is greater than 15; and all other remaining variables are as described for Formula (D), Formula (D-1), Formula (D-2), Formula (D-3), Formula (D-4), or the second, third, seventh, ninth, tenth or eleventh embodiments of Formula (D).

In a thirteenth embodiment of Formula (D), in the ionizable lipid, e.g., cationic lipid, according to Formula (D), Formula (D-1), Formula (D-2), Formula (D-3), Formula (D-4), or the second, third, seventh, ninth, tenth, eleventh or twelfth embodiments of Formula (D), or a pharmaceutically acceptable salt thereof, R^{6a} and R^{6b} contain an equal number of carbon atoms with each other; or R^{6a} and R^{6b} are the same; or R^{6a} and R^{6b} are both C₁₆ alkyl, C₁₅ alkyl, C₁₄ alkyl, C₁₃ alkyl, C₁₂ alkyl, C₁₁ alkyl, C₁₀ alkyl, C₉ alkyl, C₈ alkyl, C₇ alkyl, C₁₆ alkenyl, Cis alkenyl, C₁₄ alkenyl, C₁₃ alkenyl, C₁₂ alkenyl, C₁₁ alkenyl, C₁₀ alkenyl, C₉ alkenyl, C₈ alkenyl, or C₇ alkenyl; provided that the total number of carbon atoms in R^{6a} and R^{6b} as combined is greater than 15; and all other remaining variables are as described for Formula (D), Formula (D-1), Formula (D-2), Formula (D-3), Formula (D-4) or the second, third, seventh, ninth, tenth, eleventh or twelfth embodiments of Formula (D).

In a fourteenth embodiment of Formula (D), in the ionizable lipid, e.g., cationic lipid, according to Formula (D), Formula (D-1), Formula (D-2), Formula (D-3), Formula (D-4), or the second, third, seventh, ninth, tenth, eleventh, twelfth or thirteenth embodiments of Formula (D), R^{6a} and R^{6b} as defined in any one of the preceding embodiments each contain a different number of carbon atoms with each other; or the number of carbon atoms R^{6a} and R^{6b} differs by one or two carbon atoms; or the number of carbon atoms R^{6a} and R^{6b} differs by one carbon atom; or R^{6a} is C₇ alkyl and R^{6a} is C₈ alkyl, R^{6a} is C₈ alkyl and R^{6a} is C₇ alkyl, R^{6a} is C₈ alkyl and R^{6a} is C₉ alkyl, R^{6a} is C₉ alkyl and R^{6a} is C₈ alkyl, R^{6a} is C₉ alkyl and R^{6a} is C₁₀ alkyl, R^{6a} is C₁₁ alkyl and R^{6a} is C₉ alkyl, R^{6a} is C₁₀ alkyl and R^{6a} is C₁₁ alkyl, R^{6a} is C₁₁ alkyl and R^{6a} is C₁₁ alkyl, R^{6a} is C₁₁ alkyl and R^{6a} is C₁₂ alkyl, R^{6a} is C₁₂ alkyl and R^{6a} is C₁₁ alkyl, R^{6a} is C₇ alkyl and R^{6a} is C₉ alkyl, R^{6a} is C₉ alkyl and R^{6a} is C₇ alkyl, R^{6a} is C₈ alkyl and R^{6a} is C₁₀ alkyl, R^{6a} is C₁₀ alkyl and R^{6a} is C₈ alkyl, R^{6a} is C₉ alkyl and R^{6a} is C₁₁ alkyl, R^{6a} is C₁₁ alkyl and R^{6a} is C₉ alkyl, R^{6a} is C₁₀ alkyl and R^{6a} is C₁₂ alkyl, R^{6a} is C₁₂ alkyl and R^{6a} is C₁₀ alkyl, R^{6a} is C₁₁ alkyl and R^{6a} is C₁₃ alkyl, or R^{6a} is C₁₃ alkyl and R^{6a} is C₁₁ alkyl, etc.; and all other remaining variables are as described for Formula I, Formula II, Formula III, Formula IV, Formula V, or the second, third, seventh, ninth, tenth, eleventh, twelfth or thirteenth embodiments of Formula (D).

In a fifteenth embodiment of Formula (D), R⁴ is C₁-C₁₆ unbranched alkyl, C₂-C₁₆ unbranched alkenyl, or

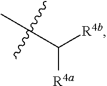

wherein R^{4a} and R^{4b} are as described above for the second, third, fourth, fifth, sixth, seventh, eighth, ninth, tenth, eleventh, twelfth, thirteenth or fourteenth embodiments of Formula (D).

In one embodiment, the ionizable lipid, e.g., cationic lipid, of the present disclosure or the ionizable lipid of Formula (D), Formula (D-1), Formula (D-2), Formula (D-3), or Formula (D-4) is any one lipid selected from the lipids listed in Table 6 below, or a pharmaceutically acceptable salt thereof:

TABLE 6

| Lipid No. | Lipid Structure |
|---|---|
| 77 | 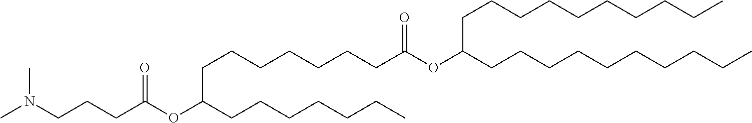 [SMILES: CCCCCCCCCCC(CCCCCCCCCC)OC(=O)CCCCCCCC(CCCCCCC)OC(=O)CCCN(C)C] |
| 78 | 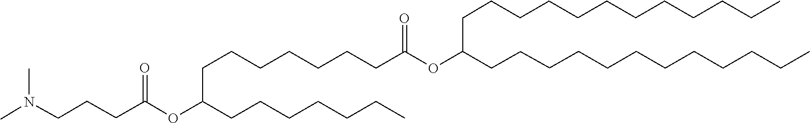 [SMILES: CCCCCCCCCCCCC(CCCCCCCCCCCC)OC(=O)CCCCCCCC(CCCCCCC)OC(=O)CCCN(C)C] |
| 79 | 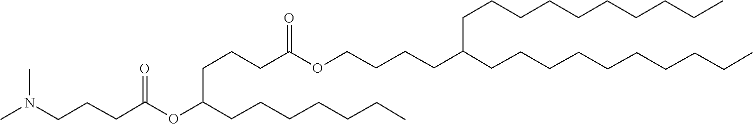 [SMILES: CCCCCCCCCCC(CCCCCCCCCC)CCCCOC(=O)CCCC(CCCCCCC)OC(=O)CCCN(C)C] |
| 80 | 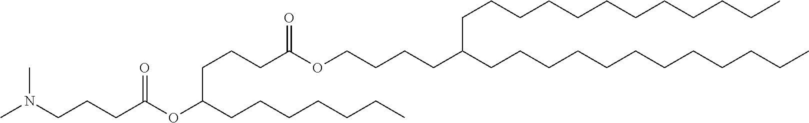 [SMILES: CCCCCCCCCCCCC(CCCCCCCCCCCC)CCCCOC(=O)CCCC(CCCCCCC)OC(=O)CCCN(C)C] |
| 81 | 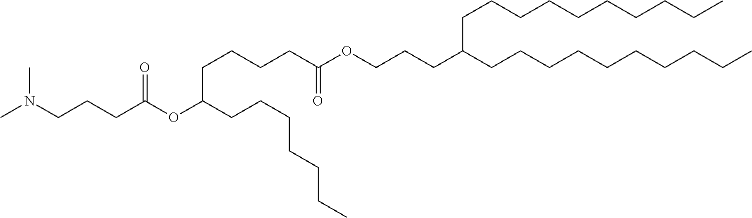 [SMILES: CCCCCCCCCCC(CCCCCCCCCC)CCCOC(=O)CCCCC(CCCCCCC)OC(=O)CCCN(C)C] |
| 82 | 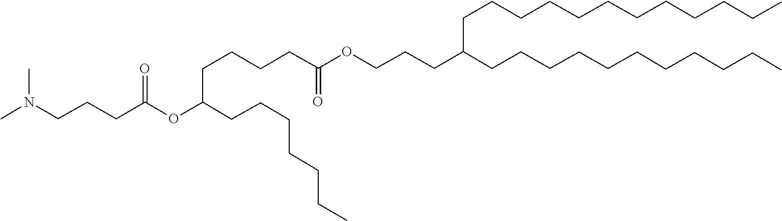 [SMILES: CCCCCCCCCCCCC(CCCCCCCCCCC)CCCOC(=O)CCCCC(CCCCCCC)OC(=O)CCCN(C)C] |
| 83 | 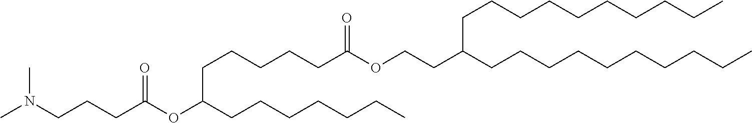 [SMILES: CCCCCCCCCCC(CCCCCCCCCC)CCOC(=O)CCCCCC(CCCCCCC)OC(=O)CCCN(C)C] |
| 84 | 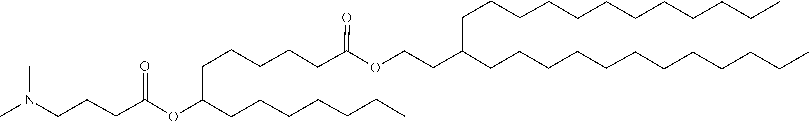 [SMILES: CCCCCCCCCCCCC(CCCCCCCCCCCC)CCOC(=O)CCCCCC(CCCCCCC)OC(=O)CCCN(C)C] |
| 85 | 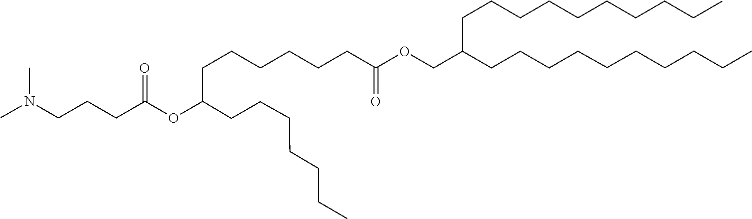 [SMILES: CCCCCCCCCCC(CCCCCCCCCC)COC(=O)CCCCCCC(CCCCCCC)OC(=O)CCCN(C)C] |
| 86 | 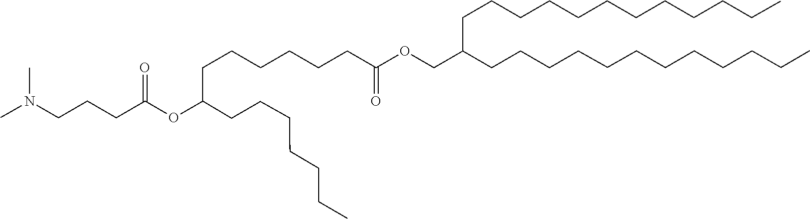 [SMILES: CCCCCCCCCCCCC(CCCCCCCCCCCC)COC(=O)CCCCCCC(CCCCCCC)OC(=O)CCCN(C)C] |
| 87 | 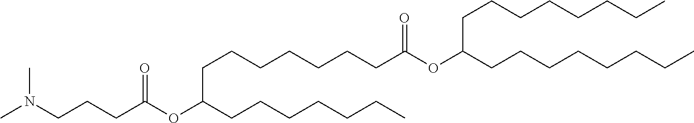 [SMILES: CCCCCCCCC(CCCCCCCC)OC(=O)CCCCCCCC(CCCCCCC)OC(=O)CCCN(C)C] |
| 88 | 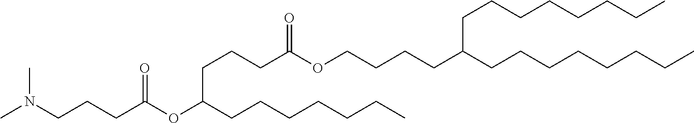 [SMILES: CCCCCCCCC(CCCCCCCC)CCCCOC(=O)CCCC(CCCCCCC)OC(=O)CCCN(C)C] |
| 89 | 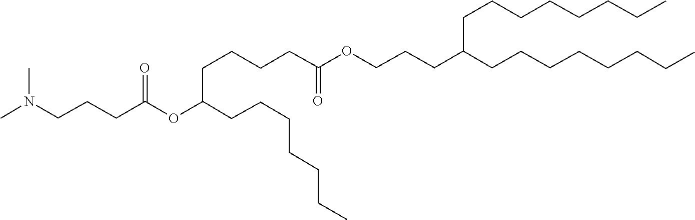 [SMILES: CCCCCCCCC(CCCCCCCC)CCCOC(=O)CCCCC(CCCCCCC)OC(=O)CCCN(C)C] |
| 91 | 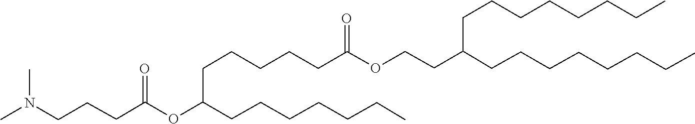 [SMILES: CCCCCCCCC(CCCCCCCC)CCOC(=O)CCCCCC(CCCCCCC)OC(=O)CCCN(C)C] |
| 92 | 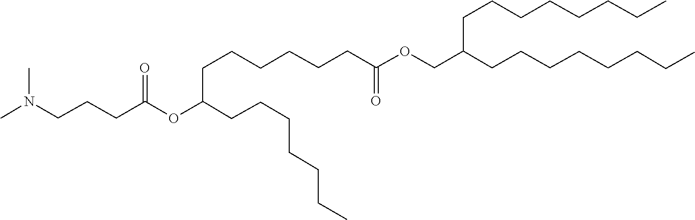 [SMILES: CCCCCCCCC(CCCCCCCC)COC(=O)CCCCCCC(CCCCCCC)OC(=O)CCCN(C)C] |
| 93 | 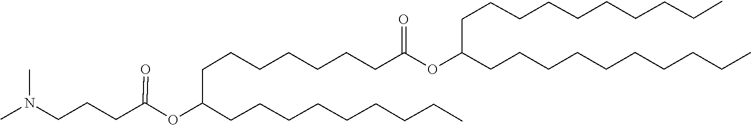 [SMILES: CCCCCCCCCCC(CCCCCCCCCC)OC(=O)CCCCCCCC(CCCCCCCCC)OC(=O)CCCN(C)C] |
| 94 | 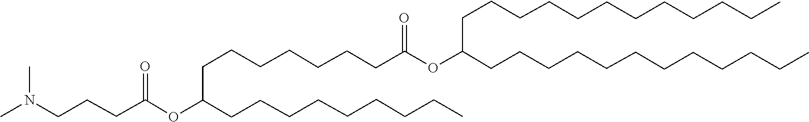 [SMILES: CCCCCCCCCCCCC(CCCCCCCCCCCC)OC(=O)CCCCCCCC(CCCCCCCCC)OC(=O)CCCN(C)C] |
| 95 | 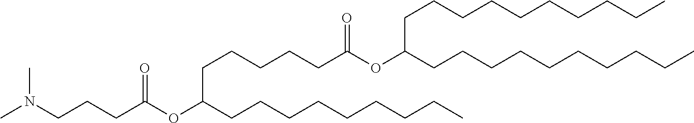 [SMILES: CCCCCCCCCCC(CCCCCCCCCC)OC(=O)CCCCCC(CCCCCCCCC)OC(=O)CCCN(C)C] |
| 96 | 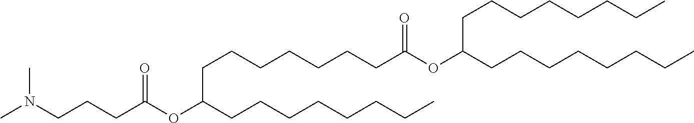 [SMILES: CCCCCCCCC(CCCCCCCC)OC(=O)CCCCCCCC(CCCCCCCC)OC(=O)CCCN(C)C] |
| 97 | 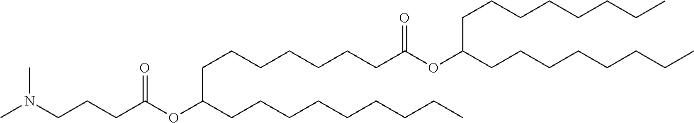 [SMILES: CCCCCCCCCC(CCCCCCCC(=O)OC(CCCCCCCC)CCCCCCCC)OC(=O)CCCN(C)C] |
| 98 | 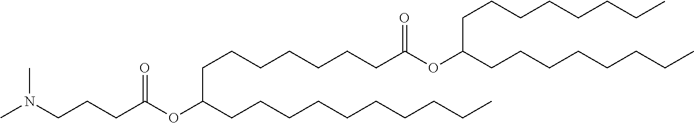 [SMILES: CCCCCCCCCCC(CCCCCCCC(=O)OC(CCCCCCCC)CCCCCCCC)OC(=O)CCCN(C)C] |
| 99 | 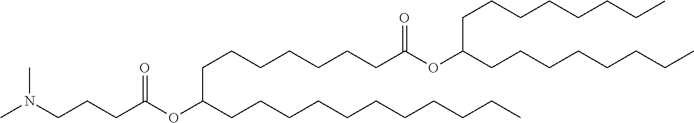 [SMILES: CCCCCCCCCCCC(CCCCCCCC(=O)OC(CCCCCCCC)CCCCCCCC)OC(=O)CCCN(C)C] |
| 100 | 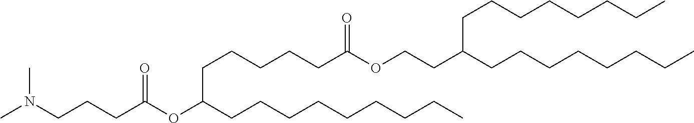 [SMILES: CCCCCCCCCC(CCCCCC(=O)OCCC(CCCCCCCC)CCCCCCCC)OC(=O)CCCN(C)C] |
| 101 | 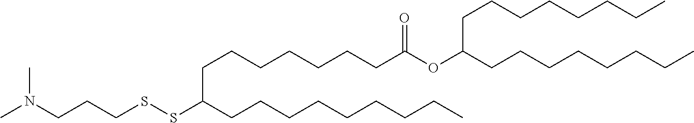 [SMILES: CCCCCCCCCC(CCCCCCCC(=O)OC(CCCCCCCC)CCCCCCCC)SSCCCN(C)C] |
| 113 | 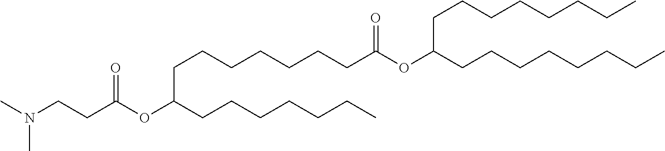 [SMILES: CCCCCCCCC(CCCCCCCC)OC(=O)CCCCCCCC(CCCCCCC)OC(=O)CCN(C)C] |
| 114 | 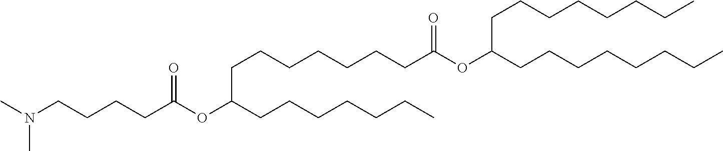 [SMILES: CCCCCCCCC(CCCCCCCC)OC(=O)CCCCCCCC(CCCCCCC)OC(=O)CCCCN(C)C] |
| 115 | 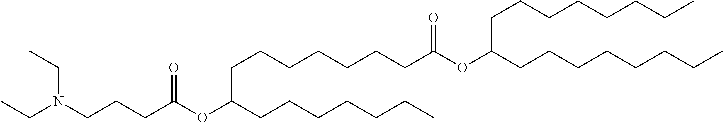 [SMILES: CCCCCCCCC(CCCCCCCC)OC(=O)CCCCCCCC(CCCCCCC)OC(=O)CCCN(CC)CC] |
| 116 | 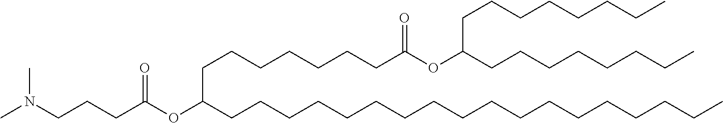 [SMILES: CCCCCCCCCCCCCCCCCCC(CCCCCCCC(=O)OC(CCCCCCCC)CCCCCCCC)OC(=O)CCCN(C)C] |
| 117 | 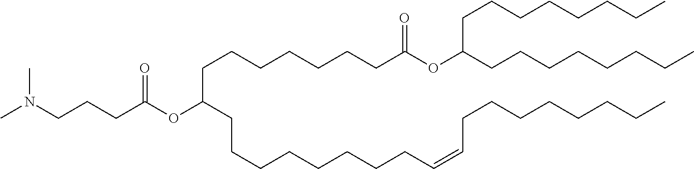 [SMILES: CCCCCCCC/C=C\CCCCCCCCC(CCCCCCCC(=O)OC(CCCCCCCC)CCCCCCCC)OC(=O)CCCN(C)C] |
| 118 | 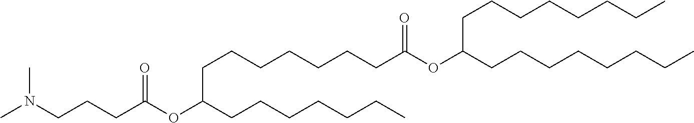 [SMILES: CCCCCCCCC(CCCCCCCC)OC(=O)CCCCCCCC(CCCCCCC)OC(=O)CCCN(C)C] |

In one embodiment, the ionizable lipid in the LNPs of the present disclosure is Ionizable Lipid 87:

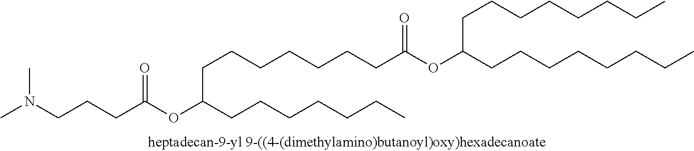 [SMILES: CCCCCCCCC(CCCCCCCC)OC(=O)CCCCCCCC(CCCCCCC)OC(=O)CCCN(C)C]

or a pharmaceutically acceptable salt or ester thereof, or a deuterated analogue thereof.

### Formula (E)

In some embodiments, the ionizable lipid, e.g., cationic lipid, in the LNPs of the present disclosure is represented by Formula (E):

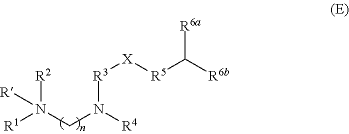

or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is positively charged;
   R¹ and R² are each independently hydrogen or C₁-C₃ alkyl;
   R³ is C₃-C₁₀ alkylene or C₃-C₁₀ alkenylene;
   R⁴ is C₁-C₁₆ unbranched alkyl, C₂-C₁₆ unbranched alkenyl, or

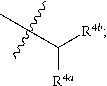

wherein:
   R^{4a} and R^{4b} are each independently C₁-C₁₆ unbranched alkyl or C₂-C₁₆ unbranched alkenyl;
R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6.

In a second embodiment of Formula (E), in the ionizable lipid, e.g., cationic lipid, according to the first embodiment, or a pharmaceutically acceptable salt thereof, X is —OC(═O)—, —SC(═O)—, —OC(═S)—, —C(═O)O—, —C(═O)S—, or —S—S—; and all other remaining variables are as described for Formula I or the first embodiment.

In a third embodiment of Formula (E), the ionizable lipid, e.g., cationic lipid, in the LNPs of the present disclosure is represented by Formula (E-1):

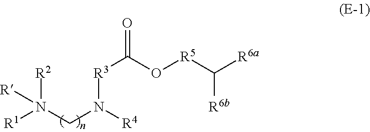

or a pharmaceutically acceptable salt thereof, wherein n is an integer selected from 1, 2, 3, and 4; and all other remaining variables are as described for Formula (E) or the second embodiment of Formula (E). Alternatively, n is an integer selected from 1, 2, and 3; and all other remaining variables are as described for Formula (E) or the second embodiment of Formula (E).

In a fourth embodiment of Formula (E), the ionizable lipid, e.g., cationic lipid, in the LNPs of the present disclosure is represented by Formula (E-2):

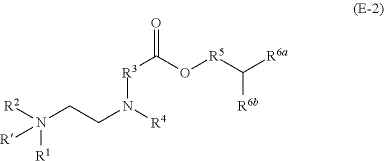

or a pharmaceutically acceptable salt thereof, and all other remaining variables are as described for Formula (E), Formula (E-1) or the second embodiment of Formula (E).

In a fifth embodiment of Formula (E), in the ionizable lipid, e.g., cationic lipid, in the LNPs of the present disclosure, R¹ and R² are each independently hydrogen or C₁-C₂ alkyl, or C₂-C₃ alkenyl; or R′, R¹, and R² are each independently hydrogen, C₁-C₂ alkyl; and all other remaining variables are as described for Formula (E), Formula (E-1) or the second embodiment of Formula (E).

In a sixth embodiment of Formula (E), the ionizable lipid, e.g., cationic lipid, in the LNPs of the present disclosure is represented by Formula (E-3):

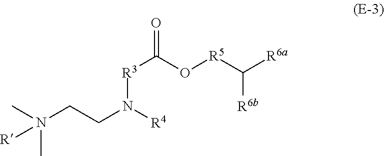

or a pharmaceutically acceptable salt thereof, and all other remaining variables are as described for Formula (E), Formula (E-1), Formula (E-2) or the second or fifth embodiments of Formula (E).

In a seventh embodiment of Formula (E), in the ionizable lipid, e.g., cationic lipid, according to Formula (E), Formula (E-1), Formula (E-2), Formula (E-3) or the second or firth embodiments of Formula (E), R⁵ is absent or C₁-C₈ alkylene; or R⁵ is absent, C₁-C₆ alkylene, or C₂-C₆ alkenylene; or R⁵ is absent, C₁-C₄ alkylene, or C₂-C₄ alkenylene; or R⁵ is absent; or R⁵ is C₈ alkylene, C₇ alkylene, C₆ alkylene, C₅ alkylene, C₄ alkylene, C₃ alkylene, C₂ alkylene, C₁ alkylene, C₈ alkenylene, C₇ alkenylene, C₆ alkenylene, C₅ alkenylene, C₄ alkenylene, C₃ alkenylene, or C₂ alkenylene; and all other remaining variables are as described for Formula (E), Formula (E-1), Formula (E-2), Formula (E-3) or the second or fifth embodiments of Formula (E).

In an eighth embodiment of Formula (E), the ionizable lipid, e.g., cationic lipid, in the LNPs of the present disclosure is represented by Formula (E-4):

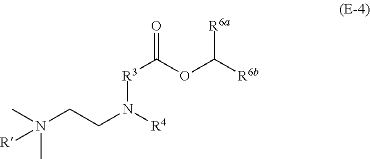

or a pharmaceutically acceptable salt thereof; and all other remaining variables are as described for Formula (E), Formula (E-1), Formula (E-2), Formula (E-3) or the second, fifth or seventh embodiments of Formula (E).

In a ninth embodiment, in the ionizable lipid, e.g., cationic lipid, according to Formula (E), Formula (E-1), Formula (E-2), Formula (E-3), Formula (E-4) or the second, fifth or seventh embodiments of Formula (E), or a pharmaceutically acceptable salt thereof, R⁴ is C₁-C₁₄ unbranched alkyl, C₂-C₁₄ unbranched alkenyl, or

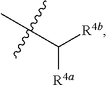

wherein R^{4a} and R^{4b} are each independently C₁-C₁₂ unbranched alkyl or C₂-C₁₂ unbranched alkenyl; or R⁴ is C₂-C₁₂ unbranched alkyl or C₂-C₁₂ unbranched alkenyl; or R⁴ is C₅-C₁₂ unbranched alkyl or C₅-C₁₂ unbranched alkenyl; or R⁴ is C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ unbranched alkyl, C₁ unbranched alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; or R⁴ is

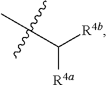

wherein R^{4a} and R^{4b} are each independently C₂-C₁₀ unbranched alkyl or C₂-C₁₀ unbranched alkenyl; or R⁴ is

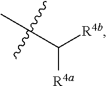

wherein R^{4a} and R^{4b} are each independently C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ alkyl, C₁ alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₁ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; and all other remaining variables are as described for Formula (E), Formula (E-1), Formula (E-2), Formula (E-3), Formula (E-4) or the second, fifth or seventh embodiments of Formula (E).

In a tenth embodiment, in the ionizable lipid, e.g., cationic lipid, according to Formula (E), Formula (E-1), Formula (E-2), Formula (E-3), Formula (E-4) or the second, fifth, seventh or ninth embodiments of Formula (E), R³ is C₃-C₈ alkylene or C₃-C₈ alkenylene, C₃-C₇ alkylene or C₃-C₇ alkenylene, or C₃-C₅ alkylene or C₃-C₅ alkenylene, or R³ is C₈ alkylene, or C₇ alkylene, or C₆ alkylene, or C₅ alkylene, or C₄ alkylene, or C₃ alkylene, or C₁ alkylene, or C₈ alkenylene, or C₇ alkenylene, or C₆ alkenylene, or C₅ alkenylene, or C₄ alkenylene, or C₃ alkenylene; and all other remaining variables are as described for Formula (E), Formula (E-1), Formula (E-2), Formula (E-3), Formula (E-4) or the second, fifth, seventh or ninth embodiments of Formula (E).

In an eleventh embodiment, in the ionizable lipid, e.g., cationic lipid, according to Formula (E), Formula (E-1), Formula (E-2), Formula (E-3), Formula (E-4) or the second, fifth, seventh, ninth or tenth embodiments of Formula (E), R^{6a} and R^{6b} are each independently C₇-C₁₂ alkyl or C₇-C₁₂ alkenyl; or R^{6a} and R^{6b} are each independently C₈-C₁₀ alkyl or C₈-C₁₀ alkenyl; or R^{6a} and R^{6b} are each independently C₁₂ alkyl, C₁ alkyl, C₁₁ alkyl, C₉ alkyl, C₈ alkyl, C₇ alkyl, C₁₂ alkenyl, C₁₁ alkenyl, C₁₀ alkenyl, C₉ alkenyl, C₈ alkenyl, or C₇ alkenyl; and all other remaining variables are as described for Formula (E), Formula (E-1), Formula (E-2), Formula (E-3), Formula (E-4) or the second, fifth, seventh, ninth or tenth embodiments of Formula (E).

In a twelfth embodiment, in the ionizable lipid, e.g., cationic lipid, according to Formula (E), Formula (E-1), Formula (E-2), Formula (E-3), Formula (E-4) or the second, fifth, seventh, ninth, tenth or eleventh embodiments of Formula (E), R^{6a} and R^{6b} contain an equal number of carbon atoms with each other; or R^{6a} and R^{6b} are the same; or R^{6a} and R^{6b} are both C₁₂ alkyl, C₁₁ alkyl, C₁₀ alkyl, C₉ alkyl, C₈ alkyl, C₇ alkyl, C₁₂ alkenyl, C₁₁ alkenyl, C₁₀ alkenyl, C₉ alkenyl, C₈ alkenyl, or C₇ alkenyl; and all other remaining variables are as described for Formula (E), Formula (E-1), Formula (E-2), Formula (E-3), Formula (E-4) or the second, fifth, seventh, ninth, tenth or eleventh embodiments of Formula (E).

In a thirteenth embodiment, in the ionizable lipid, e.g., cationic lipid, according to Formula (E), Formula (E-1), Formula (E-2), Formula (E-3), Formula (E-4), R^{6a} and R^{6b} as defined in any one of the preceding embodiments each contain a different number of carbon atoms with each other; or the number of carbon atoms R^{6a} and R^{6b} differs by one or two carbon atoms; or the number of carbon atoms R^{6a} and R^{6b} differs by one carbon atom; or R^{6a} is C₇ alkyl and R^{6a} is C₈ alkyl, R^{6a} is C₈ alkyl and R^{6a} is C₇ alkyl, R^{6a} is C₈ alkyl and R^{6a} is C₉ alkyl, R^{6a} is C₉ alkyl and R^{6a} is C₈ alkyl, R^{6a} is C₉ alkyl and R^{6a} is C₁₀ alkyl, R^{6a} is C₁₀ alkyl and R^{6a} is C₉ alkyl, R^{6a} is C₁₀ alkyl and R^{6a} is C₁₁ alkyl, R^{6a} is C₁₁ alkyl and R^{6a} is C₁₀ alkyl, R^{6a} is C₁₁ alkyl and R^{6a} is C₁₂ alkyl, R^{6a} is C₁₂alkyl and R^{6a} is C₁₁ alkyl, R^{6a} is C₇ alkyl and R^{6a} is C₉ alkyl, R^{6a} is C₉ alkyl and R^{6a} is C₇ alkyl, R^{6a} is C₈ alkyl and R^{6a} is C₁₀ alkyl, R^{6a} is C₁₀ alkyl and R^{6a} is C₈ alkyl, R^{6a} is C₉ alkyl and R^{6a} is C₁₁ alkyl, R^{6a} is C₁₁ alkyl and R^{6a} is C₉ alkyl, R^{6a} is C₁ alkyl and R^{6a} is C₁₂ alkyl, R^{6a} is C₁₂ alkyl and R^{6a} is C₁₀ alkyl, etc.; and all other remaining variables are as described for Formula (E), Formula (E-1), Formula (E-2), Formula (E-3), Formula (E-4) or the second, fifth, seventh, ninth, tenth, eleventh or twelfth embodiments of Formula (E).

In a fourteenth embodiment, in the ionizable lipid, e.g., cationic lipid, according to Formula (E), Formula (E-1), Formula (E-2), Formula (E-3), Formula (E-4) or the second, fifth, seventh, ninth, tenth, eleventh, twelfth or thirteenth embodiments of Formula (E), R′ is absent; and all other remaining variables are as described for Formula (E), Formula (E-1), Formula (E-2), Formula (E-3), Formula (E-4) or the second, fifth, seventh, ninth, tenth, eleventh, twelfth or thirteenth embodiments of Formula (E).

In one embodiment, the ionizable lipid, e.g., cationic lipid, in the LNPs of the present disclosure or the cationic lipid of Formula (E), Formula (E-1), Formula (E-2), Formula (E-3), Formula (E-4) is any one lipid selected from the lipids in Table 7 or a pharmaceutically acceptable salt thereof.

TABLE 7

| Lipid No. | Lipid Structure |
|---|---|
| 102 | 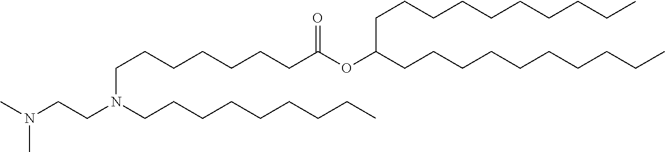 [SMILES: CCCCCCCCCCC(CCCCCCCCCC)OC(=O)CCCCCCCN(CCCCCCCCC)CCN(C)C] |
| 103 | 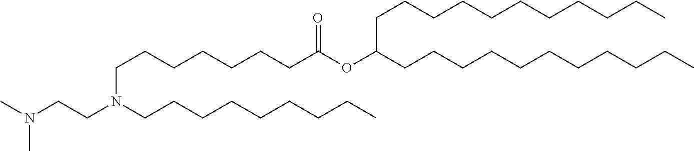 [SMILES: CCCCCCCCCCCC(CCCCCCCCCCC)OC(=O)CCCCCCCN(CCCCCCCCC)CCN(C)C] |
| 104 | 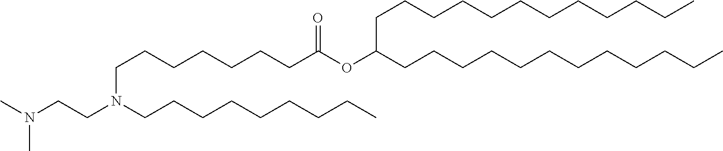 [SMILES: CCCCCCCCCCCCC(CCCCCCCCCCCC)OC(=O)CCCCCCCN(CCCCCCCCC)CCN(C)C] |
| 105 | 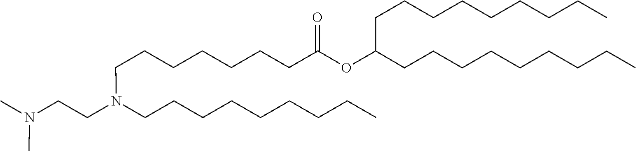 [SMILES: CCCCCCCCCC(CCCCCCCCC)OC(=O)CCCCCCCN(CCCCCCCCC)CCN(C)C] |
| 106 | 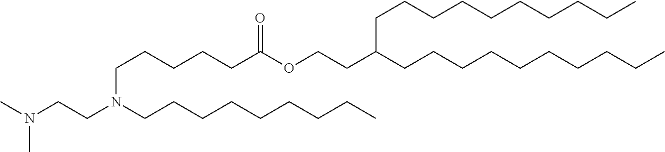 [SMILES: CCCCCCCCCCC(CCCCCCCCCC)CCOC(=O)CCCCCN(CCCCCCCCC)CCN(C)C] |
| 107 | 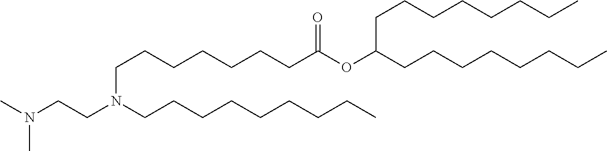 [SMILES: CCCCCCCCCN(CCCCCCCC(=O)OC(CCCCCCCC)CCCCCCCC)CCN(C)C] |
| 108 | 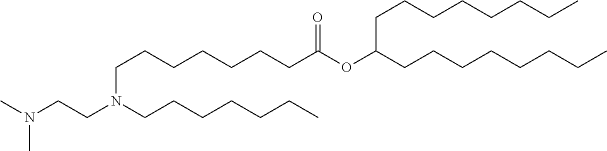 [SMILES: CCCCCCCCC(CCCCCCCC)OC(=O)CCCCCCCN(CCCCCCC)CCN(C)C] |
| 109 | 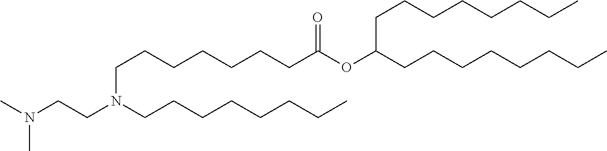 [SMILES: CCCCCCCCC(CCCCCCCC)OC(=O)CCCCCCCN(CCCCCCCC)CCN(C)C] |
| 110 | 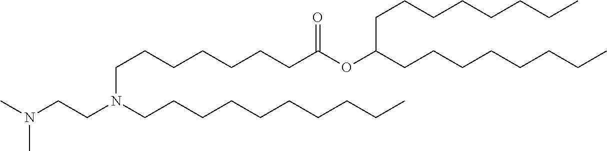 [SMILES: CCCCCCCCCCN(CCCCCCCC(=O)OC(CCCCCCCC)CCCCCCCC)CCN(C)C] |
| 111 | 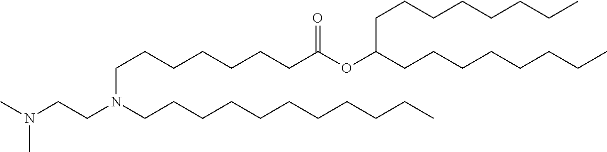 [SMILES: CCCCCCCCCCCN(CCCCCCCC(=O)OC(CCCCCCCC)CCCCCCCC)CCN(C)C] |
| 112 | 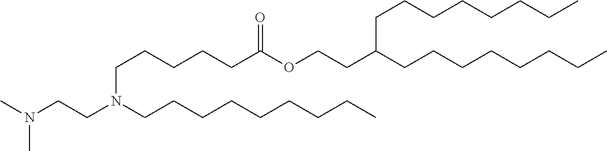 [SMILES: CCCCCCCCCN(CCCCCC(=O)OCCC(CCCCCCCC)CCCCCCCC)CCN(C)C] |

Specific examples are provided in the exemplification section below and are included as part of the cationic or ionizable lipids described herein. Pharmaceutically acceptable salts as well as neutral forms are also included.

### Cleavable Lipids

In some embodiments, the LNPs provided by the present disclosure comprise an ionizable lipid that is also a cleavable lipid. As used herein, the term “cleavable lipid”, which may be used interchangeably with the term “SS-cleavable lipid” refers to an ionizable lipid comprising a disulfide bond (“SS”). The SS in the cleavable lipid is a cleavable unit. In one embodiment, a cleavable lipid comprises an amine, e.g., a tertiary amine, e.g. and a disulfide bond. In this cleavable lipid, an amine can become protonated in an acidic compartment (e.g., in an endosome or a lysosome), leading to LNP destabilization, and the cleavable lipid can become cleaved in a reductive environment (e.g., in the cytoplasm). Cleavable lipids also include pH-activated lipid-like materials, such as ss-OP lipids, ssPalm lipids, ss-M lipids, ss-E lipids, ss-EC lipids, ss-LC lipids and ss-OC lipids, etc.

According to some embodiments, SS-cleavable lipids are described in International Patent Application Publication No. WO2019188867, incorporated by reference in its entirety herein.

In one embodiment, a cleavable lipid may comprise three components: an amine head group, a linker group, and a hydrophobic tail(s). In one embodiment, the cleavable lipid comprises one or more phenyl ester bonds, one of more tertiary amino groups, and a disulfide bond. The tertiary amine groups provide pH responsiveness and induce endosomal escape, the phenyl ester bonds enhance the degradability of the structure (self-degradability) and the disulfide bond becomes cleaved in a reductive environment.

In one embodiment, the cleavable lipid is an ss-OP lipid. In one embodiment, an ss-OP lipid comprises the structure of Lipid A shown below:

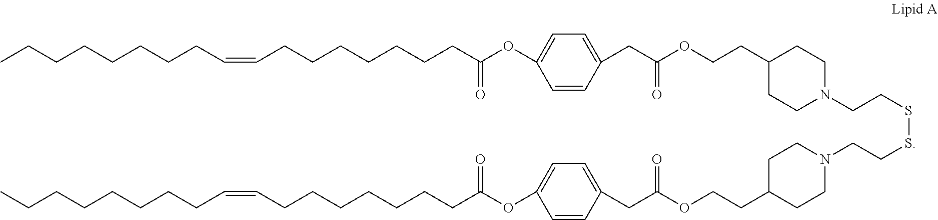 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

In one embodiment, the SS-cleavable lipid is an SS-cleavable and pH-activated lipid-like material (ssPalm). ssPalm lipids are well known in the art. For example, see Togashi et al., Journal of Controlled Release, 279 (2018) 262-270, the entire contents of which are incorporated herein by reference. In one embodiment, the ssPalm is an ssPalmM lipid comprising the structure of Lipid B shown below:

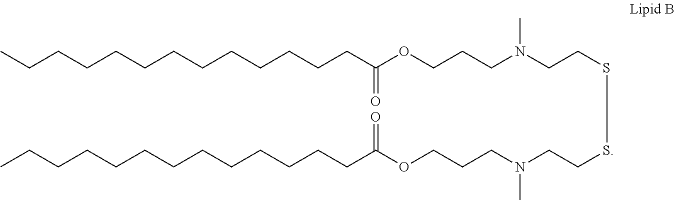 [SMILES: CCCCCCCCCCCCCC(=O)OCCCN(C)CCSCSCCN(C)CCCOC(=O)CCCCCCCCCCCCC]

In one embodiment, the ssPalmE lipid is a ssPalmE-P4-C2 lipid comprising the structure of Lipid C below:

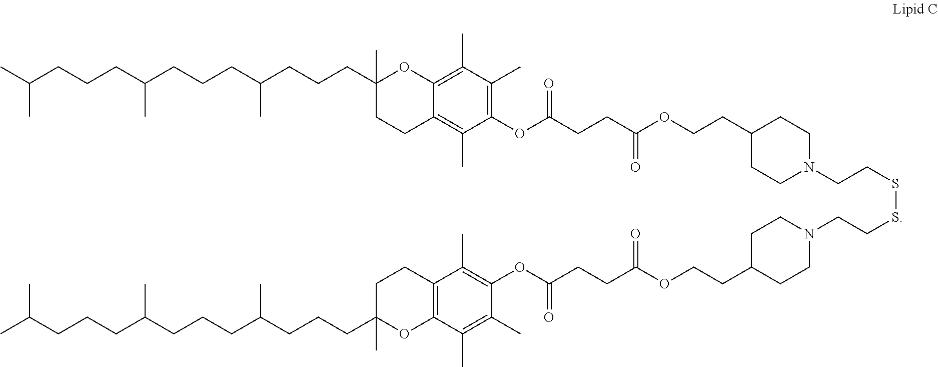 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the ssPalmE lipid is a ssPalmE-Paz4-C2 lipid, comprising the structure of Lipid D below:

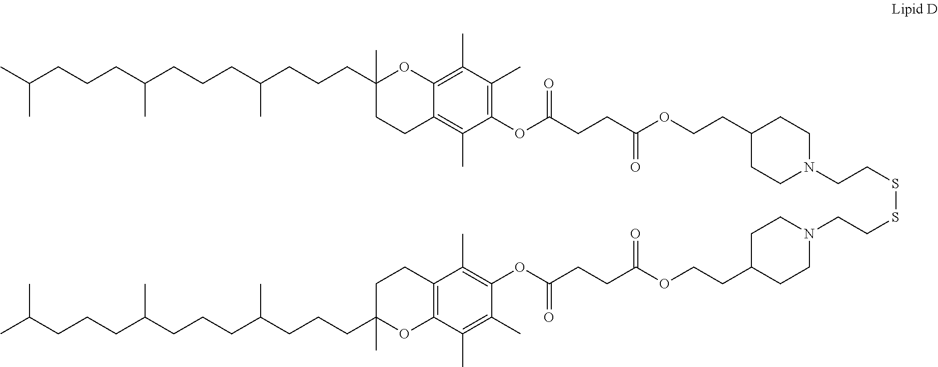 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the cleavable lipid is an ss-M lipid. In one embodiment, an ss-M lipid comprises the structure shown in Lipid E below:

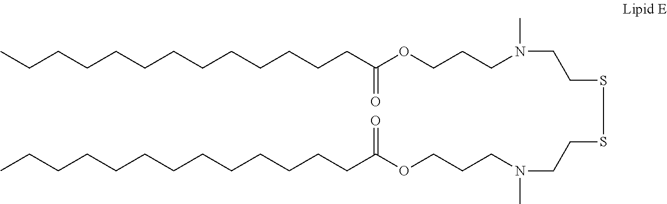 [SMILES: CCCCCCCCCCCCCC(=O)OCCCN(C)CCSSCCN(C)CCCOC(=O)CCCCCCCCCCCCC]

In one embodiment, the cleavable lipid is an ss-E lipid. In one embodiment, an ss-E lipid comprises the structure shown in Lipid F below:

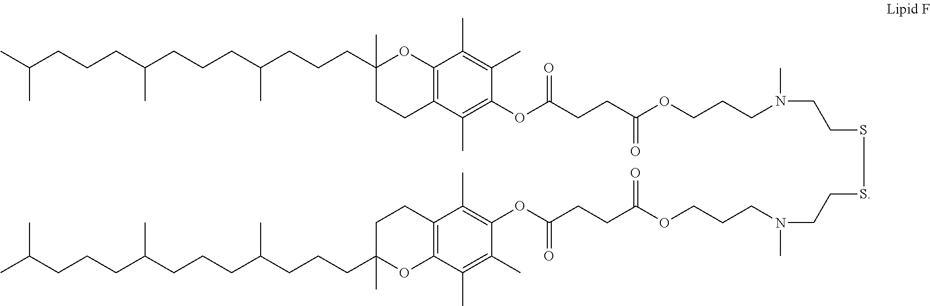 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2]

In one embodiment, the cleavable lipid is an ss-EC lipid. In one embodiment, an ss-EC lipid comprises the structure shown for Lipid G below:

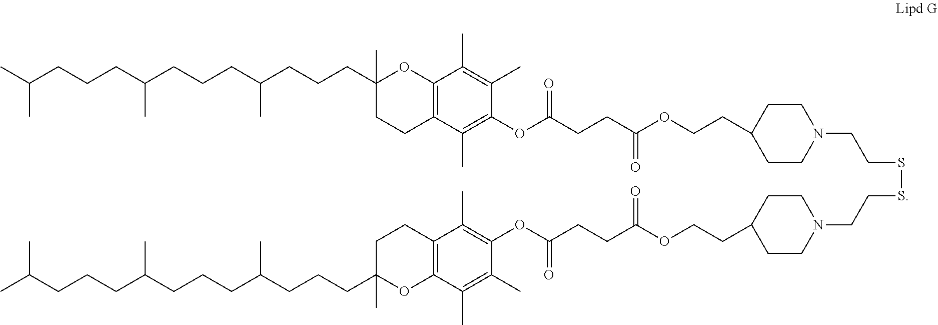 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the cleavable lipid is an ss-LC lipid. In one embodiment, an ss-LC lipid comprises the structure shown for Lipid H below:

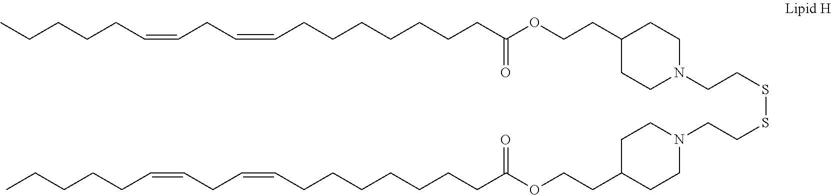 [SMILES: CCCCC/C=C\C/C=C\CCCCCCCC(=O)OCCC1CCN(CCSSCCN2CCC(CCOC(=O)CCCCCCC/C=C\C/C=C\CCCCC)CC2)CC1]

In one embodiment, the cleavable lipid is an ss-OC lipid. In one embodiment, an ss-OC lipid comprises the structure shown for Lipid J below:

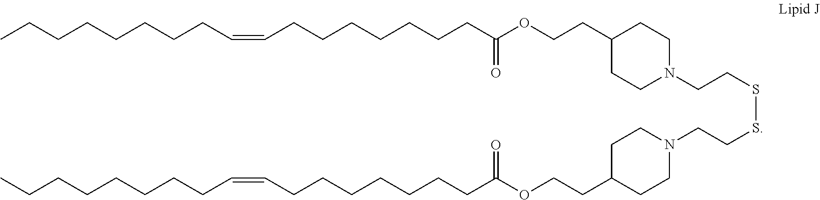 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

### Other Lipids

In some embodiments, the ionizable lipid in the LNPs of the present disclosure is selected from the group consisting of N-[1-(2,3-dioleyloxy)propyll-N,N,N-trimethylammonium chloride (DOTMA); N-[1-(2,3-dioleoyloxy)propyll-N,N,N-trimethylammonium chloride (DOTAP); 1,2-dioleoyl-sn-glycero-3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl-sn-glycero-3-ethylphosphocholine (14:1), N1-[2-((1S)-1-[(3-aminopropyl)amino]-4-[di(3-amino-propyl) aminolbutylc arboxamidoiethy11-3,4-di[oleyloxy]-benzamide (MVL5); Dioctadecylamido-glycylspermine (DOGS); 3b-[N—(N′,N′-dimethylaminoethyl)carbamoyl] cholesterol (DC-Chol); Dioctadecyldimethylammonium Bromide (DDAB); a Saint lipid (e.g., SAINT-2, N-methyl-4-(dioleyl)methylpyridinium); 1,2-dimyristyloxypropyl-3-dimethylhydroxyethylammonium bromide (DMRIE); 1,2-dioleoyl-3-dimethyl-hydroxyethyl ammonium bromide (DORIE); 1,2-dioleoyloxypropyl-3-dimethylhydroxyethyl ammonium chloride (DORI); Di-alkylated Amino Acid (DILA2) (e.g., C18:1-norArg-C16); Dioleyldimethylammonium chloride (DODAC); 1-palmitoyl-2-oleoyl-sn-glycero-3-ethylpho sphocholine (POEPC); and 1,2-dimyristoleoyl-sn-glycero-3-ethylphosphocholine (MOEPC). In some variations, the condensing agent, e.g. a cationic lipid, is a lipid such as, e.g., Dioctadecyldimethylammonium bromide (DDAB), 1,2-dilinoleyloxy-3-dimethylaminopropane (DLinDMA), 2,2-dilinoleyl-4-(2dimethylaminoethyl)-[1,31-dioxolane (DLin-KC2-DMA), heptatriaconta-6,9,28,31-tetraen-19-yl-4-(dimethylamino)butanoate (DLin-MC3-DMA), 1,2-Dioleoyloxy-3-dimethylaminopropane (DODAP), 1,2-Dioleyloxy-3-dimethylaminopropane (DODMA), Morpholinocholesterol (Mo-CHOL), (R)-5-(dimethylamino)pentane-1,2-diy1 dioleate hydrochloride (DODAPen-C1), (R)-5-guanidinopentane-1,2-diy1 dioleate hydrochloride (DOPen-G), and (R)—N,N,N-trimethy1-4,5-bis(oleoyloxy)pentan-1-aminium chloride (DOTAPen).

In some embodiments, the ionizable lipid in the LNP of the present disclosure is represented by the following structure:

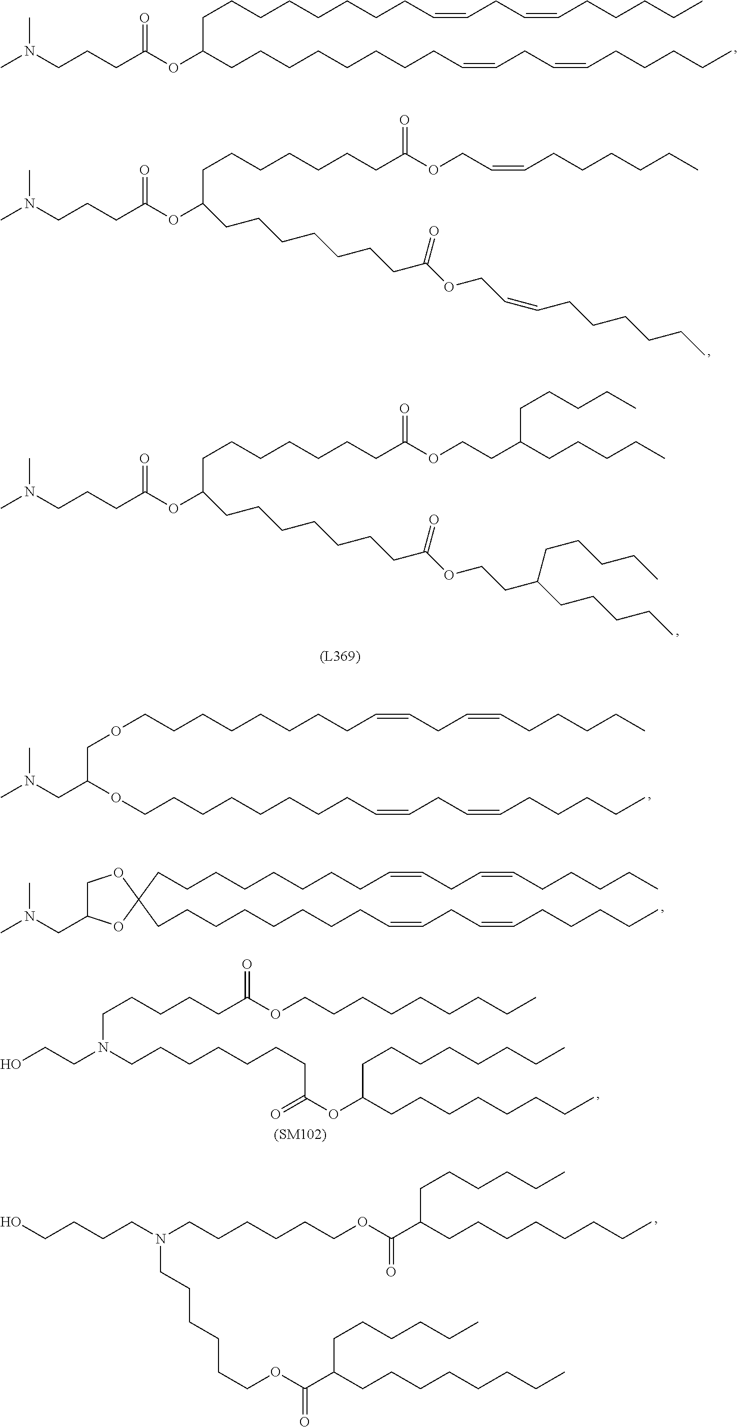

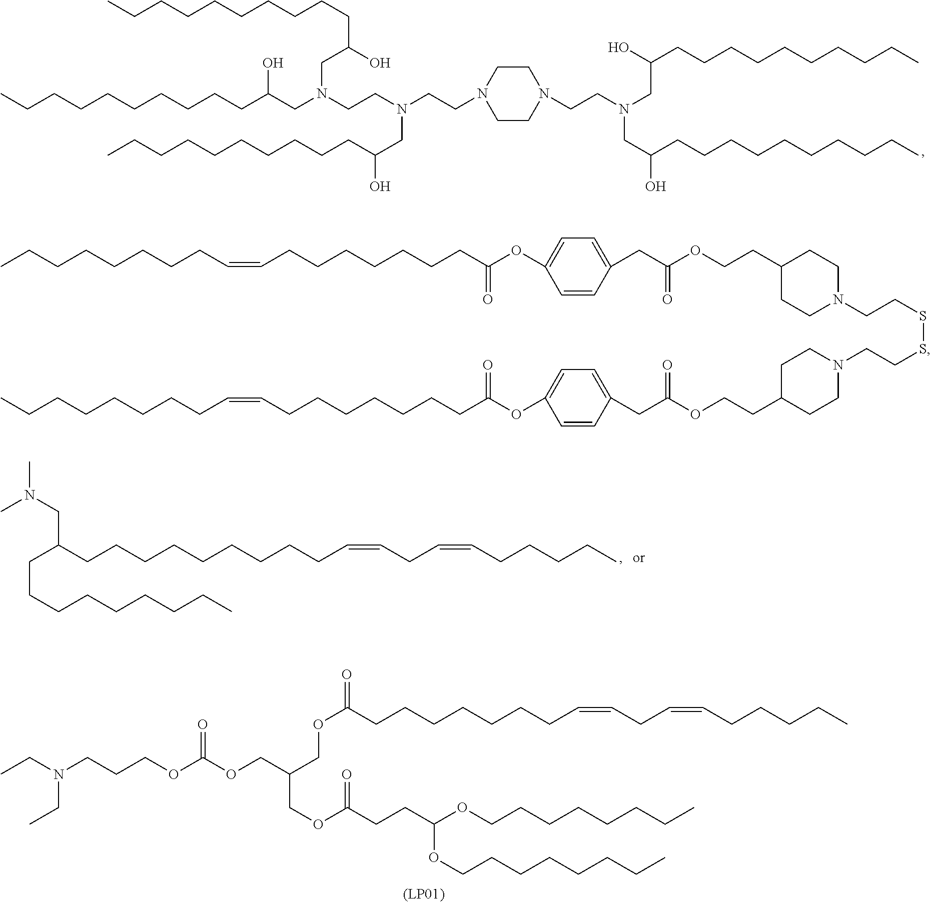

or a pharmaceutically acceptable salt or ester thereof, or a deuterated analogue thereof.

### B. Structural Lipids

In some embodiments, the LNPs provided by the present disclosure comprise a structural lipid. Without wishing to be bound by a specific theory, it is believed that a structural lipid, when present in an LNP, contributes to membrane integrity and stability of the LNP.

In some embodiments, the structural lipid is a sterol, e.g., cholesterol, or a derivative thereof. In one embodiment, the structural lipid is cholesterol. In another embodiment, the structural lipid is a derivative of cholesterol. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In some embodiments, the cholesterol derivative is a polar analogue such as cholesteryl-(4′-hydroxy)-butyl ether. In some embodiments, cholesterol derivative is cholesteryl hemisuccinate (CHEMS).

Exemplary cholesterol derivatives are described in International Patent Application Publication No. WO2009/127060 and U.S. Patent Application Publication No. US2010/0130588, contents of both of which are incorporated herein by reference in their entirety.

In some embodiments, the sterol in the LNPs of the present disclosure is selected from the group consisting of cholesterol, beta-sitosterol, stigmasterol, beta-sitostanol, campesterol, brassicasterol, and derivatives thereof, and any combination thereof. In one embodiment, the sterol is cholesterol. In another embodiment, the sterol is beta-sitosterol.

In some embodiments, the structural lipid, e.g., a sterol, constitutes about 20 mol % to about 50 mol % of the total lipid present in the LNP. In some embodiments, the structural lipid, e.g., a sterol, constitutes about 30 mol % to about 45 mol % of the total lipid present in the LNP. In some embodiments, the structural lipid, e.g., cholesterol, constitutes about 30 mol % of the total lipid present in the LNP.

In some embodiments, the structural lipid is dexamethasone or dexamethasone-palmitate.

### C. Helper Lipids

### Ceramide Helper Lipids

In some embodiments, the LNPs provided by the present disclosure comprise a helper lipid. In some embodiments, the helper lipid is a ceramide. The ceramides in the LNPs of the present disclosure are not conjugated to a polymer, such as polyethylene glycol or PEG. Ceramides are sphingolipids which is a class of cell membrane lipids. Ceramides contain an N-acetylsphingosine (i.e., (E)-N-(1,3-dihydroxyoctadec-4-en-2-yl)acetamide) backbone and a fatty acid linked to the amide group. In some embodiments, the LNPs provided by the present disclosure comprise a ceramide, whereby the fatty acid portion of the ceramide is of a certain length or is a fatty acid having a certain number of carbon atoms as described below. As used herein, the term “helper lipid” refers to an amphiphilic lipid comprising at least one non-polar chain and at least one polar moiety. Without wishing to be bound by a specific theory, it is believed that a helper lipid functions to evade off-targeting of the LNP to the blood compartment, to increase the fusogenicity of the lipid bilayer of the LNP and to facilitate endosomal escape.

In some embodiments, the LNP of the present disclosure comprises ceramide as a helper lipid. In some embodiments, the helper lipid is represented by Formula (II):

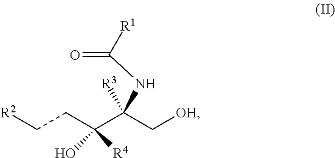

or a salt or an ester thereof, or a deuterated analogue of any of the foregoing, wherein:
[symbol] is a single bond or a double bond;
R¹ is C₁-C₁₇ alkyl or C₂-C₁₇ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments of Formula (II), [symbol] is a single bond. In some embodiments of Formula (II), [symbol] is a double bond.

In some embodiments of Formula (II), R¹ is C₁₀-C₂₀ alkenyl, R² is C₁₀-C₂₀ alkyl and R³ is hydrogen.

In some embodiments of Formula (II), R¹ is C₁-C₁₀ alkyl or C₂-C₁₀ alkenyl.

In some embodiments of Formula (II),
R¹ is C₁-C₁₀ alkyl or C₂-C₁₀ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments of Formula (II), R³ and R⁴ are both hydrogen. In some embodiments of Formula (II), R³ and R⁴ are independently hydrogen or C₁ alkyl.

In some embodiments of Formula (II), R¹ is C₁-C₇ alkyl or C₂-C₇ alkenyl. In one embodiment, R¹ is C₁-C₇ alkyl. In one embodiment, R¹ is C₁ alkyl.

The term “salt” when used to refer to a helper lipid represented by Formula (II) means a pharmaceutically acceptable salt of a helper lipid represented by Formula (II), including both acid and base addition salts. A salt of a helper lipid represented by Formula (II) retains the biological effectiveness and properties of the free acid forms or free base forms of the helper lipid represented by Formula (II), which are not biologically or otherwise undesirable, and which are formed with inorganic acids or organic acids, or inorganic bases or organic bases. Examples of inorganic acids include, but are not limited to, hydrochloric acid, hydrobromic acid, sulfuric acid, nitric acid, phosphoric acid and the like; and examples of organic acids include, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

The term “ester”, when used to refer to a helper lipid represented by Formula (II), means an ester of a helper lipid represented by Formula (II). As a non-limiting example, a hydroxyl group of the helper lipid represented by Formula (II) may be linked to an organic acid such as phosphoric acid or carboxylic acid via the process of esterification to form an ester (e.g., a carboxylate or a phosphate) of a helper lipid represented by Formula (II).

The term “deuterated analogue”, when used to refer to a helper lipid represented by Formula (II), means an analogue of a helper lipid represented by Formula (II), whereby any one or more hydrogen atoms of the helper lipid are substituted with deuterium, which is an isotope of hydrogen.

In one embodiment of Formula (II), or a salt or an ester thereof, or a deuterated analogue of any of the foregoing, [symbol] is a double bond; R¹, R², R³ and R⁴ are as defined above. In an alternative embodiment of Formula (II), or a salt or an ester thereof, or a deuterated analogue of any of the foregoing, [symbol] is a single bond; R¹, R², R³ and R⁴ are as defined above.

In some embodiments of Formula (II), or a salt or an ester thereof, or a deuterated analogue of any of the foregoing, R¹ is C₁-C₁₅ alkyl or C₂-C₁₅ alkenyl.

In some embodiments of Formula (II), or a salt or an ester thereof, or a deuterated analogue of any of the foregoing:
R¹ is C₁-C₁₅ alkyl or C₂-C₁₅ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments of Formula (II), or a salt or an ester thereof, or a deuterated analogue of any of the foregoing, R¹ is C₁-C₁₀ alkyl or C₂-C₁₀ alkenyl.

In some embodiments of Formula (II), or a salt or an ester thereof
R¹ is C₁-C₁₀ alkyl or C₂-C₁₀ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments of Formula (II), or a salt or an ester thereof, or a deuterated analogue of any of the foregoing, R¹ is C₁-C₈ alkyl or C₂-C₈ alkenyl. In one embodiment, R¹ is C₁-C₈ alkyl. In some embodiments of Formula (II), or a salt or an ester thereof, or a deuterated analogue of any of the foregoing, R¹ is C₁-C₇ alkyl or C₂-C₇ alkenyl. In one embodiment, R¹ is C₁-C₇ alkyl. In one embodiment of Formula (II), or a salt or an ester thereof, or a deuterated analogue of any of the foregoing:
R¹ is C₁-C₇ alkyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments of Formula (II), or a salt or an ester thereof, or a deuterated analogue of any of the foregoing, R¹ is C₁ alkyl, C₂ alkyl, C₃ alkyl, C₄ alkyl, C₅ alkyl, C₆ alkyl, or C₇ alkyl. In some embodiments of Formula (II), or a salt or an ester thereof, or a deuterated analogue of any of the foregoing, R¹ is C₁ alkyl, C₂ alkyl, C₃ alkyl, C₄ alkyl, C₅ alkyl, C₆ alkyl, or C₇ alkyl. In some embodiments of Formula (II), or a salt or an ester thereof, or a deuterated analogue of any of the foregoing, R¹ is C₁ alkyl, C₃ alkyl, C₅ alkyl, or C₇ alkyl. In one embodiment of Formula (II), or a salt or an ester thereof, or a deuterated analogue of any of the foregoing, R¹ is C₁ alkyl. In one embodiment of Formula (II), or a salt or an ester thereof, or a deuterated analogue of any of the foregoing, R¹ is C₃ alkyl. In one embodiment of Formula (II), or a salt or an ester thereof, or a deuterated analogue of any of the foregoing, R¹ is C₅ alkyl. In one embodiment of Formula (II), or a salt or an ester thereof, or a deuterated analogue of any of the foregoing, R¹ is C₇ alkyl.

In some embodiments of Formula (II), or a salt or an ester thereof, or a deuterated analogue of any of the foregoing, R² is C₃-C₁₅ alkyl or C₃-C₁₅ alkenyl; and R′, R³ and R⁴ are as defined above. In some embodiments of Formula (II), or a salt or an ester thereof, or a deuterated analogue of any of the foregoing, R² is C₈-C₁₅ alkyl or C₃-C₁₅ alkenyl; and R′, R³ and R⁴ are as defined above. In some embodiments of Formula (II), or a salt or an ester thereof, or a deuterated analogue of any of the foregoing, R² is C₇-C₁₅ alkyl or C₃-C₁₅ alkenyl; and R¹, R³ and R⁴ are as defined above. In some embodiments of Formula (II), or a salt or an ester thereof, or a deuterated analogue of any of the foregoing, R² is C₉-C₁₅ alkyl or C₉-C₁₅ alkenyl; and R′, R³ and R⁴ are as defined above. In some embodiments of Formula (II), or a salt or an ester thereof, or a deuterated analogue of any of the foregoing, R² is C₉ alkyl, C₁₀ alkyl, C₁₁ alkyl, C₁₂ alkyl, C₁₃ alkyl, C₁₄ alkyl, or C₁₅ alkyl; and R¹, R³ and R⁴ are as defined above. In some embodiments of Formula (II), or a salt or an ester thereof, or a deuterated analogue of any of the foregoing, R² is C₉ alkyl; and R′, R³ and R⁴ are as defined above. In some embodiments of Formula (II), or a salt or an ester thereof, or a deuterated analogue of any of the foregoing, R² is C₁₁ alkyl; and R′, R³ and R⁴ are as defined above. In some embodiments of Formula (II), or a salt or an ester thereof, or a deuterated analogue of any of the foregoing, R² is C₁₃ alkyl; and R′, R³ and R⁴ are as defined above.

In some embodiments of Formula (II), or a salt or an ester thereof, or a deuterated analogue thereof, R³ is hydrogen or C₁ alkyl; and R¹, R² and R⁴ are as defined above. In one embodiment of Formula (II), or a salt or an ester thereof, or a deuterated analogue thereof, R³ is hydrogen; and R¹, R² and R⁴ are as defined above. In one embodiment of Formula (II), or a salt or an ester thereof, or a deuterated analogue of any of the foregoing, R³ is C₁ alkyl; and R¹, R² and R⁴ are as defined above.

In some embodiments of Formula (II), or a salt or an ester thereof, or a deuterated analogue of any of the foregoing, R⁴ is hydrogen or C₁ alkyl; and R¹, R² and R³ are as defined above. In one embodiment of Formula (II), or a salt or an ester thereof, or a deuterated analogue of any of the foregoing, R⁴ is hydrogen; and R¹, R² and R³ are as defined above. In one embodiment of Formula (II), or a salt or an ester thereof, or a deuterated analogue of any of the foregoing, R⁴ is C₁ alkyl; and R¹, R² and R³ are as defined above.

In some embodiments of Formula (II), R¹ is C₁-C₇ alkyl or C₂-C₇ alkenyl.

In some embodiments, R¹ is C₁ alkyl, C₃ alkyl, C₅ alkyl, or C₇ alkyl. In some embodiments, R¹ is C₁ alkyl.

In some embodiments of Formula (II), R² is C₃-C₁₅ alkyl or C₃-C₁₅ alkenyl. In some embodiments, R² is C₁₀ alkyl, C₁₁ alkyl, C₁₂ alkyl, C₁₃ alkyl, C₁₄ alkyl, or C₁₅ alkyl. In some embodiments, R² is C₁₂ alkyl, C₁₃ alkyl, or C₁₄ alkyl. In some embodiments, R² is C₁₃ alkyl. In some embodiments, R² is C₁₂ alkyl. In some embodiments, R² is C₁₁ alkyl.

In some embodiments of Formula (II), both R¹ and R² are hydrogen; and [symbol] is a double bond.

In some embodiments of Formula (II), both R¹ and R² are hydrogen and [symbol] is a double bond; and R¹ is C₁ alkyl, C₃ alkyl, C₅ alkyl or C₇ alkyl. In one embodiment, R¹ is C₁ alkyl. In another embodiment, R¹ is C₃ alkyl. In yet another embodiment, R¹ is C₅ alkyl. In yet another embodiment, R¹ is C₇ alkyl.

In some embodiments of Formula (II), both R¹ and R² are hydrogen and [symbol] is a double bond; R¹ is C₁ alkyl, C₃ alkyl, C₅ alkyl or C₇ alkyl and R² is C₉ alkyl, C₁₁, or C₁₃ alkyl. In one embodiment, R² is C₉ alkyl. In one embodiment, R² is C₁₁ alkyl. In another embodiment, R² is C₁₃ alkyl.

In some embodiments of Formula (II), R³ is hydrogen. In some embodiments of Formula (II), R³ is C₁ alkyl.

In some embodiments of Formula (II), R⁴ is hydrogen. In some embodiments of Formula (II), R⁴ is C₁ alkyl.

### Other Helper Lipids

In some embodiments, a helper lipid comprised in an LNP of the present disclosure is a phospholipid, a phosphatidylcholine, or a derivative thereof. In some embodiments, the helper lipid comprised in an LNP of the present disclosure is selected from the group consisting of 1,2-distearoyl-sn-glycero-3-phosphocholine (DSPC), hydrogenated soybean PC (HSPC), phosphatidylserine (PS), 1,2-dioleoyl-sn-glycero-3-phosphocholine (DOPC), 1,2-dipalmitoyl-sn-glycero-3-phosphocholine (DPPC), 1-palmitoyl-2-oleoyl-sn-glycero-3-phosphocholine (POPC), 1,2-dilauroyl-sn-glycero-3-phosphocholine (DLPC), 1-margaroyl-2-oleoyl-sn-glycero-3-phosphocholine (MOPC), 1-palmitoyl-2-linoleoyl-sn-glycero-3-phosphocholine (PLPC), 1-stearoyl-2-myristoyl-sn-glycero-3-phosphocholine (SMPC), 1,2-dimyristoyl-sn-glycero-3-phosphocholine (DMPC), 1,2-dihexanoyl-sn-glycero-3-phosphocholine (DHPC), 1,2-dierucoyl-sn-glycero-3-phosphocholine (DEPC), 1-palmitoyl-2-oleoyl-glycero-3-phosphocholine (POPC), and 1,2-dioleoyl-sn-glycero-3-phosphoethanolamine (DOPE). In one embodiment, the helper lipid comprised in an LNP of the present disclosure is 1,2-distearoyl-sn-glycero-3-phosphocholine (DSPC).

In some embodiments, the helper lipid constitutes about 1 mol % to about 40 mol % of the total lipid present in the LNP, or about 5 mol % to about 15 mol %. In some embodiments, the helper lipid constitutes about 10% mol to about 20 mol % of the total lipid present in the LNP and such LNP having about 10% mol to about 20 mol % of the total lipid present in the LNP demonstrate overall increased tolerability (e.g., as demonstrated in body weight loss profiles in a subject and reduced cytokine response), as compared to the LNP comprising less than 10% of the same helper lipid.

### D. Lipid-Anchored Polymers

In some embodiments, the LNPs provided by the present disclosure comprise at least one type of lipid-anchored polymer, e.g., a first lipid-anchored polymer and/or a second lipid-anchored polymer. As used herein, the term “lipid-anchored polymer” refers to a molecule comprising a lipid moiety covalently attached to a polymer, e.g., via a linker. Without wishing to be bound by a specific theory, it is believed that a lipid-anchored polymer can inhibit aggregation of LNPs and provide steric stabilization. In some embodiments, the LNPs provided by the present disclosure comprise two lipid-anchored polymers, i.e., a first lipid-anchored polymer and a second lipid-anchored polymer.

In some embodiments, the first lipid-anchored polymer comprised in an LNP of the present disclosure is the polymer-conjugated lipid of the present disclosure, e.g., DODA-PG34, DODA-PG45, DODA-PG46, or DODA-PG58.

In some embodiments, an LNP of the present disclosure comprises two types of a lipid-anchored polymer: a) the polymer-conjugated lipid of the present disclosure, e.g., DODA-PG34, DODA-PG45, DODA-PG46, or DODA-PG58, as the first lipid-anchored polymer, and b) a second lipid-anchored polymer.

In some embodiments, a lipid-anchored polymer, e.g., a second lipid-anchored polymer in accordance with the present disclosure comprises:
(i) a lipid moiety comprising at least one hydrophobic tail; and
(ii) a polymer conjugated to the lipid moiety, optionally via a linker.

In some embodiments, a lipid-anchored polymer, e.g., a second lipid-anchored polymer in accordance with the present disclosure comprises:
(i) a lipid moiety comprising at least one hydrophobic tail;
(ii) a polymer;
(iii) a linker, wherein the polymer is conjugated to the lipid moiety via the linker; and
(iv) a targeting moiety conjugated to the polymer.

In some embodiments, a lipid-anchored polymer, e.g., a second lipid-anchored polymer in accordance with the present disclosure comprises:
(i) a lipid moiety comprising at least one hydrophobic tail;
(ii) a polymer;
(iii) a linker, wherein the polymer is conjugated to the lipid moiety via the linker; and
(iv) a reactive species conjugated to the polymer, wherein the reactive species is functionalized to be conjugated to a targeting moiety.

In one embodiment, the at least one (e.g., single or two) hydrophobic tail is a fatty acid. Non-limiting examples of the at least one (e.g., single or two) hydrophobic tail comprising 12 to 22 carbon atoms in a single aliphatic chain backbone include lauric acid, myristic acid, myristoleic acid, octadecylamine, palmitic acid, stearic acid, arachidic acid, palmitoleic acid, sapienic acid, oleic acid, elaidic acid, vaccenic acid, linoleic acid, linoelaidic acid, α-linolenic acid, arachidonic acid, eicosapentaenoic acid, and a derivative thereof.

The term “derivative,” when used herein in reference to hydrophobic tails in a lipid-anchored polymer, refers to a hydrophobic tail that has been modified as compared to the original or native hydrophobic tail. In some embodiments, the derivative contains one or more of the following modifications as compared to the original or native hydrophobic tail: a) carboxylate group has been replaced with an amine group, an amide group, an ether group, or a carbonate group; b) one or more points of saturation, e.g., double bonds, have been introduced into (e.g., via dehydrogenation) the hydrophobic tail; c) one or more points of saturation, e.g., double bonds, have been removed from (e.g., via hydrogenation) the hydrophobic tail; and d) configuration of one or more double bonds, if present, has been changed, e.g., from a cis configuration to a trans configuration, or from a trans configuration to a cis configuration. The derivative contains the same number of carbon atoms as its original or native hydrophobic tail.

As used herein the term “a single aliphatic chain backbone” when referring to a hydrophobic tail in a lipid-anchored polymer refers the main linear aliphatic chain or carbon chain, i.e., the longest continuous linear aliphatic chain or carbon chain. For example, the alkyl chain below that has several branchings contains 18 carbon atoms in a single aliphatic chain backbone, i.e., the longest continuous linear alkyl chain contains 18 carbon atoms. Note that the one or two carbon atoms (all indicated with *) in the several branchings are not included in the carbon atom count in the single aliphatic chain backbone.

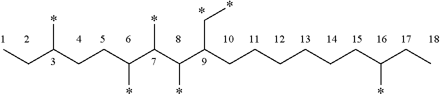 [SMILES: *CC(CCCCCCC(*)CC)C(*)C(*)C(*)CCC(*)CC]

In one embodiment, a second lipid-anchored polymer in accordance with the present disclosure comprises a lipid moiety comprising at least one hydrophobic tail; and a polymer conjugated to the lipid moiety, optionally via a linker, wherein the lipid moiety of the second lipid-anchored polymer comprises a lipid-linker moiety selected from the group consisting of 1,2-dipalmitoyl-sn-glycero-3-phosphocholine (DPPC), 1-palmitoyl-2-oleoyl-glycero-3-phosphocholine (POPC), 1-palmitoyl-2-oleoyl-sn-glycero-3-phosphoethanolamine (POPE), 1-palmitoyl-2-oleoyl-sn-glycero-3-phospho-(1′-rac-glycerol) (POPG), 1,2-dipalmitoyl-sn-glycero-3-phosphoethanolamine (DPPE), 1,2-distearoyl-sn-glycero-3-phosphoethanolamine (DSPE), 1,2-dielaidoyl-sn-phosphatidylethanolamine (DEPE), 1-stearoyl-2-oleoyl-sn-glycero-3-phosphoethanolamine (SOPE), 1,2-dioleoyl-sn-glycero-3-phosphoglycerol (DOPG), 1,2-dipalmitoyl-sn-glycero-3-phosphoglycerol (DPPG), 18-1-trans PE, 1,2-dioleoyl-sn-glycero-3-phospho-L-serine (DOPS), 1,2-diphytanoyl-sn-glycero-3-phosphoethanolamine (DPHyPE); and dioctadecylamine (DODA), dihexadecylamine, distearoyl-rac-glycerol (DSG), 1,2-dipalmitoyl-rac-glycerol (DPG), 1,2-dioleoyl-sn-glycero-3-phosphoethanolamine (DOPE), and a derivative thereof. In some embodiments, the lipid moiety of the second lipid-anchored polymer comprises a lipid-linker moiety selected from the group consisting of DOPE, DSPE, DSG, DODA, DPG and a derivative thereof. In one specific embodiment, the lipid moiety of the second lipid-anchored polymer comprises DSPE. In one embodiment, the second lipid-anchored polymer further comprises a targeting moiety.

A lipid-anchored polymer of the present disclosure may also comprise a reactive species. In some embodiments, the reactive species is conjugated to the polymer in the lipid-anchored polymer. The reactive species present in a lipid-anchored polymer of the present disclosure may be used for conjugation, e.g., to a targeting moiety which has been functionalized with a complementary reactive species, i.e., a reactive species capable of reacting with the reactive species comprised in the lipid-anchored polymer of the present disclosure. In some embodiments, the reactive species conjugated to the lipid-anchored polymer of the present disclosure may be a thiol reagent, a maleimide reagent, or click chemistry reagent, e.g., a reagent selected from the group consisting of an alkyne reagent, such as a dibenzocyclooctyne (DBCO) reagent, a transcyclooctene (TCO) reagent, a tetrazine (TZ) reagent and an azide (AZ) reagent.

### Linkers in Lipid-Anchored Polymers

In some embodiments, in a lipid-anchored polymer of the present disclosure, a lipid moiety is covalently attached to a polymer via a linker. In some embodiments, the linker in the lipid-anchored polymer of the present disclosure is an alkyl linker, a glycerol linker, a phosphate linker, a phosphate ester linker, an ether linker, an ester linker, a diester linker (e.g., a glutary linker, a succinyl linker, etc.), an amide linker, a diamide linker, an amine linker, a peptide linker, a phosphoethanolamine linker, a phosphocholine linker, a carbamate linker, a diamide alkyl linker, a cleavable linker, or any combination thereof. In some embodiments, the linker in the lipid-anchored polymer in the LNPs of the present disclosure is selected from the group consisting of —(CH₂)ₙ—, —C(O)(CH₂)ₙ—, —C(O)O(CH₂)ₙ, —C(O)O(CH₂)ₙ—, —OC(O)(CH₂)ₙC(O)O—, and —NH(CH₂)ₙC(O)O—, wherein n is an number integer ranging from 1 to 20. Accordingly, in some embodiments, the linker is —C(O)(CH₂)ₙ—, and in some embodiments, n is 2, 3, 4, 5, or 6.

In some embodiments, the linker of the second-lipid anchored polymer is a glycerol linker, a phosphate linker, an ether linker, an amide linker, an amine linker, a peptide linker, a phosphoethanolamine linker, a phosphocholine linker, a carbamate linker, or —(CH₂)ₙ—, —C(O)(CH₂)ₙ—, or —C(O)O(CH₂)ₙ, wherein n is an integer ranging from 1 to 20, or any combination thereof.

The term “linker-lipid moiety”, as used herein, refers to a lipid moiety comprising at least one hydrophobic tail that is covalently attached to a linker. In some embodiments, the linker-lipid moiety may be a part of a lipid-anchored polymer.

As used herein, the term “derivative” when used in reference to a linker-lipid moiety means a linker-lipid moiety containing one or more of the following modifications: a) a phosphatidylethanolamine (PE) head group, if present, is modified to convert an amino group into a methylamino group or a dimethylamino group; b) the modified linker-lipid moiety comprises one or more additional functional groups or moieties, such as —OH, —OCH₃, —NH₂, a maleimide, an azide or a cyclooctyne such as dibonzeocyclooctyne (DBCO).

### Polymers in Lipid-Anchored Polymers

In some embodiments, the polymer comprised in the lipid-anchored polymer is selected from the group consisting of polyethylene (PE), polypropylene (PP), polyethylene glycol (PEG), polyglycerol (PG), polyvinyl alcohol (PVOH), polysarcosine (pSar), and a combination thereof. In one embodiment, the polymer is selected from the group consisting of polyethylene glycol (PEG), polyglycerol (PG), polysarcosine (pSar), poly(2-methacryloyloxyethyl phosphorylcholine) (PMPC), and a combination thereof. In one embodiment, the polymer is polyethyelene glycol (PEG).

In some embodiments, the polymer comprised in the lipid-anchored polymer, e.g., the second lipid-anchored polymer, is selected from the group consisting of polyethylene (PE), polypropylene (PP), polyethylene glycol (PEG), polyvinyl alcohol (PVOH), polysarcosine (pSar), polyglycerol (PG), and a derivative of any of the foregoing.

In some embodiments, the polymer comprised in the lipid-anchored polymer of the present disclosure, e.g., the first lipid-anchored polymer and/or the second lipid-anchored polymer, is polyglycerol (PG) or a PG derivative. The PG or the PG derivative may be linear or branched. The PG derivative may be a carboxylated PG, e.g., a glutarylated PG, such as 3-methyl glutarylated PG, or 2-carboxycyclohexane-1-carboxylated PG. In some embodiments, the PG or the PG derivative may comprise an average of 5-100 monomeric units. In some embodiments, the PG or the PG derivative may comprise an average of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60, or about 34, 45, 46, or 58 monomeric units. In one embodiment, the PG or the PG derivative comprises an average of 34, 45, 46 or 58 monomeric units.

In some embodiments, the polymer in the lipid-anchored polymer has a molecular weight of between about 500 Da and about 5000 Da, e.g., between about 1500 Da and about 5000 Da. In some embodiments, the polymer in the lipid-anchored polymer has an average molecular weight of about 2000 Da.

### Targeting Moiety

In some embodiments, an LNP of the present disclosure further comprises one or more targeting moieties. The targeting moiety targets the LNP for delivery to a specific site or a tissue in a subject, e.g., liver. In some embodiments, the targeting moiety is capable of binding to specific liver cells, such as hepatocytes. The targeting moiety may be conjugated to a first lipid-anchored polymer, e.g., a polymer-conjugated lipid of the disclosure, or a second lipid-anchored polymer, as described herein.

In one embodiment, the targeting moiety is capable of binding to the asialoglycoprotein receptor (ASGPR), i.e., hepatocyte-specific ASGPR. In one embodiment, the targeting moiety comprises an N-acetylgalactosamine molecule (GalNAc) or a GalNAc derivative thereof. As used herein, a “GalNAc derivative” refers to a modified GalNAc molecule or a conjugate of one or more GalNAc molecules (modified or unmodified) covalently linked to, for example, a lipid-anchored polymer as defined herein. In one embodiment, the targeting moiety is a tri-antennary or tri-valent GalNAc conjugate (i.e., GalNAc3) which is a ligand conjugate having three GalNAc molecules or three GalNAc derivatives. In one embodiment, the targeting moiety is a tri-antennary GalNAc represented by the following structural formula:

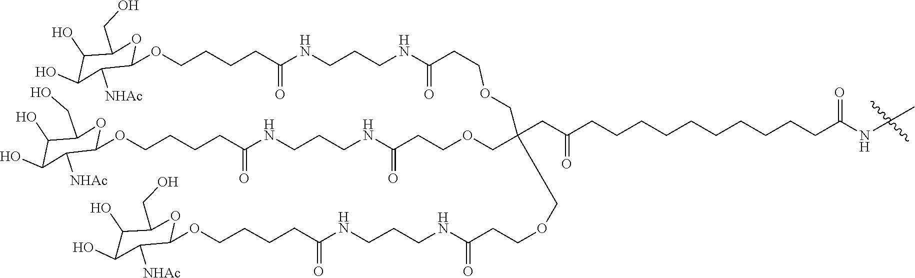 [SMILES: *NC(=O)CCCCCCCCCCC(=O)CC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In one embodiment, the targeting moiety is a tetra-antennary GalNAc conjugate. In one embodiment, the targeting moiety is a tetra-antennary or tetra-valent GalNAc conjugate (i.e., GalNAc4) which is a ligand having four GalNAc molecules or four GalNAc derivatives.

In one embodiment, the targeting moiety is capable of binding to low-density lipoprotein receptors (LDLRs), e.g., hepatocyte-specific LDLRs. In one embodiment, the targeting moiety comprises an apoliprotein E (ApoE) protein, an ApoE polypeptide (or peptide), an apoliprotein B (ApoB) protein, an ApoB polypeptide (or peptide), a fragment of any of the foregoing, or a derivative of any of the foregoing. In one embodiment, the ApoE polypeptide, ApoB polypeptide, or a fragment thereof is a ApoE polypeptide, ApoB polypeptide, or a fragment thereof as disclosed in International Patent Application Publication No. WO2022/261101, which is incorporated herein by reference in its entirety. In one embodiment, the ApoE protein is a modified ApoE protein and the ApoB protein is a modified ApoB protein.

In one embodiment, the ApoE protein has an amino acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98, or at least about 99% sequence identity to the following amino acid sequence: MKVEQAVETEPEPELRQQTEWQSGQRWELALGRFWDYLRWVQTLSEQVQEELLSSQVTQE LRALMDETMKELKAYKSELEEQLTPVAEETRARLSKELQAAQARLGADMEDVCGRLVQYR GEVQAMLGQSTEELRVRLASHLRKLRKRLLRDADDLQKRLAVYQAGAREGAERGLSAIRER LGPLVEQGRVR (SEQ ID NO: 1). In one embodiment, the ApoE protein comprises the amino acid sequence set forth in SEQ ID NO: 1. In one embodiment, the ApoE protein consists essentially of the amino acid sequence set forth in SEQ ID NO: 1. In one embodiment, the ApoE protein consists of the amino acid sequence set forth in SEQ ID NO: 1.

In one embodiment, the ApoE protein has an amino acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98, or at least about 99% sequence identity to the following amino acid sequence: MKVEQAVETEPEPELRQQTEWQSGQRWELALGRFWDYLRWVQTLSEQVQEELLSSQVTQE LRALMDETMKELKAYKSELEEQLTPVAEETRARLSKELQAAQARLGADMEDVCGRLVQYR GEVQAMLGQSTEELRVRLASHLRKLRKRLLRDADDLQKRLAVYQAGAREGAERGLSAIRER LGPLVEQGRVRHHHHHH (SEQ ID NO: 2). In one embodiment, the ApoE protein comprises the amino acid sequence set forth in SEQ ID NO: 2. In one embodiment, the ApoE protein consists essentially of the amino acid sequence set forth in SEQ ID NO: 2. In one embodiment, the ApoE protein consists of the amino acid sequence set forth in SEQ ID NO: 2.

In one embodiment, the ApoE protein has an amino acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98, or at least about 99% sequence identity to the following amino acid sequence: MKVEQAVETEPEPELRQQTEWQSGQRWELALGRFWDYLRWVQTLSEQVQEELLSSQVTQE LRALMDETMKELKAYKSELEEQLTPVAEETRARLSKELQAAQARLGADMEDVSGRLVQYR GEVQAMLGQSTEELRVRLASHLRKLRKRLLRDADDLQKRLAVYQAGAREGAERGLSAIRER LGPLVEQGRVR (SEQ ID NO: 3). In one embodiment, the ApoE protein comprises the amino acid sequence set forth in SEQ ID NO: 3. In one embodiment, the ApoE protein consists essentially of the amino acid sequence set forth in SEQ ID NO: 3. In one embodiment, the ApoE protein consists of the amino acid sequence set forth in SEQ ID NO: 3.

In one embodiment, the ApoE protein has an amino acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98, or at least about 99% sequence identity to the following amino acid sequence: MKVEQAVETEPEPELRQQTEWQSGQRWELALGRFWDYLRWVQTLSEQVQEELLSSQVTQE LRALMDETMKELKAYKSELEEQLTPVAEETRARLSKELQAAQARLGADMEDVSGRLVQYR GEVQAMLGQSTEELRVRLASHLRKLRKRLLRDADDLQKRLAVYQAGAREGAERGLSAIRER LGPLVEQGRVRHHHHHHGGSSGSGC (SEQ ID NO: 4). In one embodiment, the ApoE protein comprises the amino acid sequence set forth in SEQ ID NO: 4. In one embodiment, the ApoE protein consists essentially of the amino acid sequence set forth in SEQ ID NO: 4. In one embodiment, the ApoE protein consists of the amino acid sequence set forth in SEQ ID NO: 4.

As used herein, the term “sequence identity”, when used in reference to a polypeptide or a protein, refers to the ratio of the number of identical amino acids between the 2 aligned sequences over the aligned length, expressed as a percentage. In some embodiments, the 2 aligned sequences are identical in length, i.e., have the same number of amino acids.

In one embodiment, the targeting moiety in an LNP of the present disclosure is an ApoE protein conjugate in an ApoB protein conjugate, which is a conjugate of one or more ApoE and/or ApoB protein molecules (native or modified) or a fragment thereof covalently linked to, for example, a lipid-anchored polymer as defined herein. In one embodiment, the targeting moiety in an LNP of the present disclosure is an ApoE polypeptide conjugate in an ApoB polypeptide conjugate, which is a conjugate of one or more ApoE and/or ApoB polypeptide molecules or a fragment thereof covalently linked to, for example, a lipid-anchored polymer as defined herein.

In one embodiment the targeting moiety is an antibody or an antibody fragment, e.g., an antibody or an antibody fragment that is capable of specifically binding to an antigen present on the surface of a cell. In one embodiment the antibody or an antibody fragment is a monoclonal antibody (mAb), a single chain variable fragment (scFv), a heavy chain antibody (hcAb), a nanobody (Nb), a heavy-chain-only immunoglobulin (HCIg), an immunoglobulin new antigen receptor (IgNAR), variable domain of immunoglobulin new antigen receptor (VNAR), a single-domain antibody, or a variable heavy chain-only antibody (VHH).

In some embodiments, an LNP of the present disclosure comprises a polymer-conjugated lipid of the present disclosure, and the targeting moiety as defined herein (and including GalNAc, ApoE protein, ApoB protein, ApoE polypeptide, ApoB polypeptide, an antibody or an antibody fragment) is conjugated to the polymer-conjugated lipid of the present disclosure.

In some embodiments, an LNP of the present disclosure may comprise a polymer-conjugated lipid of the present disclosure as a first lipid-anchored polymer, and a targeting moiety as described herein conjugated to the polymer-conjugated lipid. In some embodiments the polymer in the polymer-conjugated lipid, e.g., a PG or a PG derivative, is conjugated to a targeting moiety.

In some embodiments, the targeting moiety may be conjugated to the polymer-conjugated lipid via a reactive species. In some embodiments, the reactive species may be a thiol reagent, a maleimide reagent, or click chemistry reagent, e.g., a reagent selected from the group consisting of an alkyne reagent, such as a dibenzocyclooctyne (DBCO) reagent, a transcyclooctene (TCO) reagent, a tetrazine (TZ) reagent and an azide (AZ) reagent. Accordingly, in an exemplary embodiment, the polymer-conjugated lipid of the present disclosure comprising an azide reagent as the reactive species may be reacted with a targeting moiety functionalized with a DBCO reagent as a complementary reactive species to produce a polymer-conjugated lipid conjugated to a targeting moiety via the reactive species. In another exemplary embodiment, the polymer-conjugated lipid of the present disclosure comprising a thiol reagent may be reacted with a targeting moiety functionalized with a maleimide reagent to produce a polymer-conjugated lipid conjugated to a targeting moiety via the reactive species.

In some embodiments, an LNP of the present disclosure may comprise a polymer-conjugated lipid of the present disclosure as a first lipid-anchored polymer, a second lipid-anchored polymer and a targeting moiety as described herein conjugated to the second lipid-anchored polymer.

In some embodiments, the targeting moiety may be conjugated to the second lipid-anchored polymer via a reactive species. In some embodiments, the reactive species may be a thiol reagent, a maleimide reagent, or click chemistry reagent, e.g., a reagent selected from the group consisting of an alkyne reagent, such as a dibenzocyclooctyne (DBCO) reagent, a transcyclooctene (TCO) reagent, a tetrazine (TZ) reagent and an azide (AZ) reagent. Accordingly, in an exemplary embodiment, the second lipid-anchored polymer of the present disclosure comprising an azide reagent as the reactive species may be reacted with a targeting moiety functionalized with a DBCO reagent as a complementary reactive species to produce a second lipid-anchored moiety conjugated to the targeting moiety via a reactive species. In another exemplary embodiment, the polymer-conjugated lipid of the present disclosure comprising a thiol reagent may be reacted with a targeting moiety functionalized with a maleimide reagent to produce a polymer-conjugated lipid comprising a targeting moiety.

Accordingly, in one embodiment of an LNP of the present disclosure, the LNP comprises a second lipid-anchored polymer and the targeting moiety as defined herein (and including GalNAc, ApoE protein, ApoB protein, ApoE polypeptide, ApoB polypeptide, an antibody or an antibody fragment) is conjugated to the second lipid-anchored polymer. The second lipid-anchored polymer contains a lipid moiety conjugated to a polymer, optionally via a linker. In one embodiment, the second lipid-anchored polymer comprises a moiety selected from the group consisting of 1,2-dipalmitoyl-sn-glycero-3-phosphocholine (DPPC), 1-palmitoyl-2-oleoyl-glycero-3-phosphocholine (POPC), 1-palmitoyl-2-oleoyl-sn-glycero-3-phosphoethanolamine (POPE), 1-palmitoyl-2-oleoyl-sn-glycero-3-phospho-(1′-rac-glycerol) (POPG), 1,2-dipalmitoyl-sn-glycero-3-phosphoethanolamine (DPPE), 1,2-distearoyl-sn-glycero-3-phosphoethanolamine (DSPE), 1,2-dielaidoyl-sn-phosphatidylethanolamine (DEPE), 1-stearoyl-2-oleoyl-sn-glycero-3-phosphoethanolamine (SOPE), 1,2-dioleoyl-sn-glycero-3-phosphoglycerol (DOPG), 1,2-dipalmitoyl-sn-glycero-3-phosphoglycerol (DPPG), 18-1-trans PE, 1,2-dioleoyl-sn-glycero-3-phospho-L-serine (DOPS), 1,2-diphytanoyl-sn-glycero-3-phosphoethanolamine (DPHyPE); and dioctadecylamine (DODA), dihexadecylamine, distearoyl-rac-glycerol (DSG), 1,2-dipalmitoyl-rac-glycerol (DPG), 1,2-dioleoyl-sn-glycero-3-phosphoethanolamine (DOPE), and a derivative thereof. In one embodiment, the second lipid-anchored polymer comprises a linker-lipid moiety selected from the group consisting of DOPE, DSPE, DSG, DODA, DPG, and a derivative thereof. In another embodiment the lipid moiety of the second lipid-anchored polymer comprises DSPE.

In one embodiment, the ApoE protein, ApoB protein, ApoE polypeptide, ApoB polypeptide, an antibody, or a fragment thereof, is covalently linked to a lipid-anchored polymer (e.g., first lipid anchored polymer or second lipid-anchored polymer) or to an LNP of the present disclosure via strain promoted alkyne-azide cycloaddition (SPAAC) chemistry, such as via an azide-modified lipid-anchored polymer (e.g., DSG-PEG2000-azide, DSPE-PEG2000-azide, DSG-PEG3400-azide, DSPE-PEG3400-azide, DSG-PEG5000-azide, DSPE-PEG5000-azide) and a dibenzocyclooctyne (DBCO)-functionalized ApoE protein, ApoB protein, ApoE polypeptide, ApoB polypeptide, an antibody or a fragment thereof.

In an exemplary embodiment, the second lipid-anchored polymer conjugated to a targeting moiety is represented by the following structure:

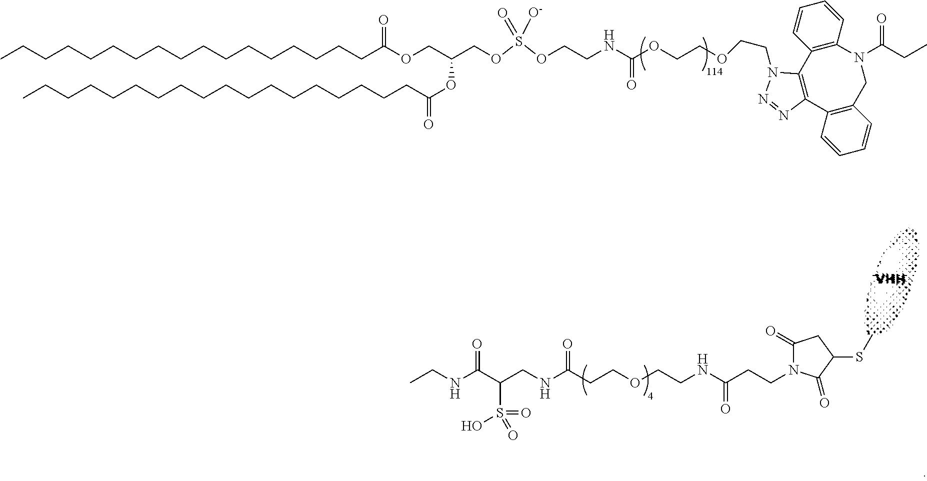

In another exemplary embodiment, the second lipid-anchored polymer conjugated to a targeting moiety is represented by the following structure.

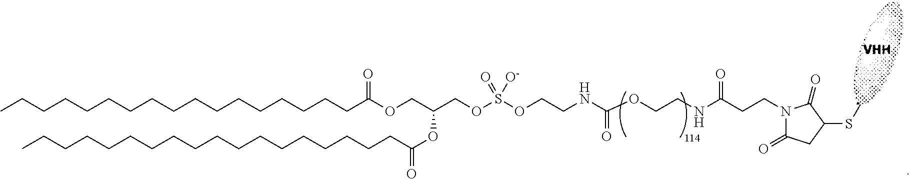

In some embodiments, the LNPs of the present disclosure may comprise a first lipid-anchored polymer and a second lipid-anchored polymer. For example, the LNPs of the present disclosure may comprise a first lipid-anchored polymer that does not comprise a targeting moiety, and a second type of lipid-anchored polymer that comprises a targeting moiety, such as GalNAc. For example, the LNPs of the present disclosure may comprise DSG-PEG2000 modified to comprise an additional OCH₃ group (DSG-PEG2000-OMe) as a first lipid-anchored polymer and DSPE-PEG2000-GalNAc3 as a second lipid-anchored polymer. In another example, the LNPs of the present disclosure may comprise a polymer-conjugated lipid as a first lipid-anchored polymer of the present disclosure and a second-anchored polymer, e.g., a second-anchored polymer conjugated to a targeting moiety. For example, the LNPs of the present disclosure may comprise DODA-PG45 as a first lipid-anchored polymer and DSPE-PEG2000-OH as the second lipid-anchored polymer.

In some embodiments, the LNPs of the present disclosure may comprise a first lipid-anchored polymer and a second lipid-anchored polymer, wherein the second lipid-anchored polymer comprises a targeting moiety. In some embodiments, the second lipid-anchored polymer comprises a lipid moiety selected from the group consisting of DSPE, DSG, DODA, DPG, DOPE, and a derivative of thereof. In some embodiments, the first lipid-anchored polymer is any lipid-anchored polymer as described hereinabove. In one specific embodiment, the first lipid-anchored polymer is the polymer-conjugated lipid of the present disclosure, e.g., DODA-PG34, DODA-PG45, DODA-PG46, or DODA-PG58. For example, the LNPs of the present disclosure may comprise DODA-PG45 as a first lipid-anchored polymer and DSPE-PEG2000-GalNAc3 as the second lipid-anchored polymer.

In some embodiments, the targeting moiety is conjugated to a DSPE-anchored polymer. In some embodiments, the DSPE-anchored polymer is DSPE-PEG or a derivative thereof.

In some embodiments, the targeting moiety is conjugated to a DSG-anchored polymer. In some embodiments, the DSG-anchored polymer is DSG-PEG or a derivative thereof.

In some embodiments, the LNPs provided by the present disclosure comprise a therapeutic nucleic acid (TNA); an ionizable lipid; a sterol; a helper lipid, and a first lipid-anchored polymer, wherein the first lipid-anchored polymer comprises the polymer-conjugated lipid.

In some embodiments, the ionizable lipid constitutes about 20 mol % to about 60 mol % of the total lipid present in the LNP. In some embodiments, the ionizable lipid constitutes about 35 mol % to about 50 mol % of the total lipid present in the LNP. In some embodiments, the sterol constitutes about 20 mol % to about 50 mol % of the total lipid present in the LNP. In some embodiments, the sterol constitutes about 30 mol % to about 45 mol % of the total lipid present in the LNP. In some embodiments, the helper lipid constitutes about 1 mol % to about 40 mol % of the total lipid present in the LNP. In some embodiments, the helper lipid constitutes about 5 mol % to about 15 mol % of the total lipid present in the LNP. In some embodiments, the first lipid-anchored polymer constitutes about 0.5 mol % to about 5 mol % of the total lipid present in the LNP. In some embodiments, the first lipid-anchored polymer constitutes about 1.5 mol % to about 3 mol % of the total lipid present in the LNP. In some embodiments, the second lipid-anchored polymer constitutes about 0.05 mol % to about 5 mol % of the total lipid present in the LNP. In some embodiments, the second lipid-anchored polymer constitutes about 0.1 mol % to about 1 mol % of the total lipid present in the LNP.

Lipid nanoparticles (LNPs) comprising ceDNA are disclosed in International Patent Application No. PCT/US2018/050042, filed on Sep. 7, 2018, which is incorporated herein in its entirety and envisioned for use in the methods and compositions as disclosed therein.

The size of LNPs can be determined by quasi-elastic light scattering using a Malvern Zetasizer Nano ZS (Malvern, UK). In some embodiments, LNPs of the present disclosure have a mean diameter as determined by light scattering of less than about 90 nm, e.g., less than about 80 nm or less than about 75 nm. According to some embodiments, LNPs of the present disclosure have a mean diameter as determined by light scattering of between about 50 nm and about 75 nm or between about 50 nm and about 70 nm.

The pKa of formulated cationic lipids can be correlated with the effectiveness of the LNPs for delivery of nucleic acids (see Jayaraman et al., Angewandte Chemie, International Edition (2012), 51(34), 8529-8533; Semple et al., Nature Biotechnology 28, 172-176 (2010), both of which are incorporated by reference in their entireties). In one embodiment, the pKa of each cationic lipid is determined in lipid nanoparticles using an assay based on fluorescence of 2-(p-toluidino)-6-napthalene sulfonic acid (TNS). LNPs in PBS at a concentration of 0.4 mM total lipid can be prepared using the in-line process as described herein and elsewhere. TNS can be prepared as a 100 mM stock solution in distilled water. Vesicles can be diluted to 24 mM lipid in 2 mL of buffered solutions containing, 10 mM HEPES, 10 mM MES, 10 mM ammonium acetate, 130 mM NaCl, where the pH ranges from 2.5 to 11. An aliquot of the TNS solution can be added to give a final concentration of 1 mM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis can be applied to the fluorescence data and the pKa is measured as the pH giving rise to half-maximal fluorescence intensity.

In one embodiment, relative activity can be determined by measuring luciferase expression in the liver 4 hours following administration via tail vein injection. The activity is compared at a dose of 0.3 and 1.0 mg ceDNA/kg and expressed as ng luciferase/g liver measured 4 hours after administration.

Without limitations, LNP of the present disclosure includes a lipid formulation that can be used to deliver a capsid-free, non-viral DNA vector to a target site of interest (e.g., cell, tissue, organ, and the like). Generally, the LNP comprises capsid-free, non-viral DNA vector and a cationic lipid or a salt thereof.

Further exemplary lipid-anchored polymers (e.g., second lipid-anchored polymer) include, but are not limited to, PEG-lipid conjugates, polyoxazoline (POZ)-lipid conjugates, PG-lipid conjugates, polyamide-lipid conjugates (such as ATTA-lipid conjugates), cationic-polymer lipid (CPL) conjugates, and mixtures thereof. In some embodiments, the second lipid-anchored polymer is a PEGylated lipid, for example, a (methoxy polyethylene glycol)-conjugated lipid. PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, or a mixture thereof. Additional exemplary PEG-lipid conjugates are described, for example, in International Patent Application Publication Nos. WO 1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, U.S. Patent Application Publication Nos. US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and U.S. Pat. Nos. 5,885,613, 6,287,591, 6,320,017, and 6,586,559, the contents of all of which are incorporated herein by reference in their entireties.

Additional examples of PEG-DAA PEGylated lipids include, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In one embodiment, the PEG-lipid can be selected from the group consisting of PEG-DMG, 1,2-dimyristoyl-sn-glycero-3-phosphoethanolamine-N-[methoxy(polyethylene glycol)-2000],

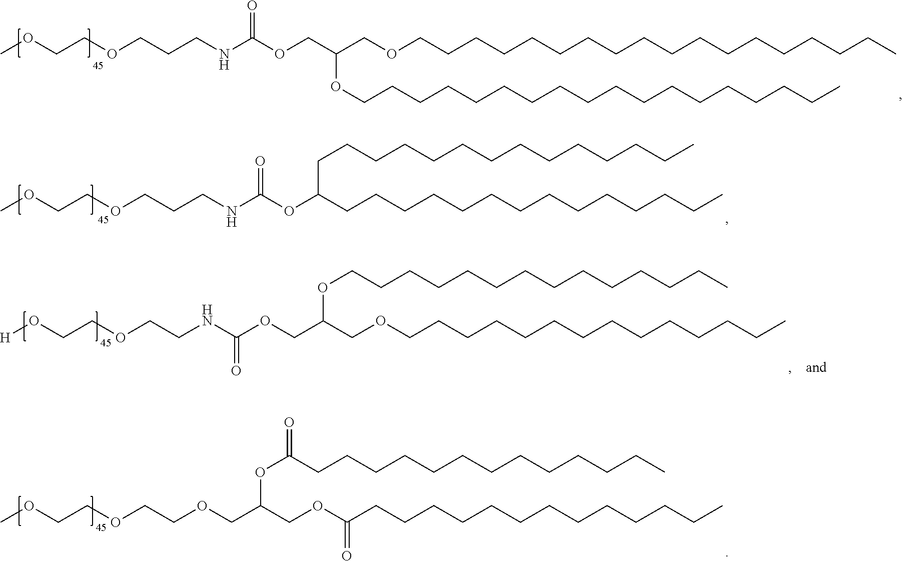

Yet further exemplary lipid-anchored polymers (e.g., second lipid-anchored polymer) include N-(carbonyl-methoxyPEGₙ)-1,2-dimyristoyl-sn-glycero-3-phosphoethanolamine (DMPE-PEGₙ, where n is 350, 500, 750, 1000 or 2000), N-(carbonyl-methoxyPEGₙ)-1,2-distearoyl-sn-glycero-3-phosphoethanolamine (DSPE-PEGₙ, where n is 350, 500, 750, 1000 or 2000), DSPE-polyglycelin-cyclohexyl-carboxylic acid, DSPE-polyglycelin-2-methylglutar-carboxylic acid, 1,2-distearoyl-sn-glycero-3-phosphoethanolamine (DSPE) conjugated polyethylene glycol (DSPE-PEG-OH), polyethylene glycol-dimyristolglycerol (PEG-DMG), or polyethylene glycol-distearoyl glycerol (PEG-DSG). In some examples of DMPE-PEGₙ, where n is 350, 500, 750, 1000 or 2000, the PEG-lipid is N-(carbonyl-methoxypolyethyleneglycol 2000)-1,2-dimyristoyl-sn-glycero-3-phosphoethanolamine (DMPE-PEG 2,000). In some examples of DSPE-PEGₙ. where n is 350, 500, 750, 1000 or 2000, the PEG-lipid is N-(carbonyl-methoxyPEG 2000)-1,2-distearoyl-sn-glycero-3-phosphoethanolamine (DSPE-PEG 2,000). In some embodiments, the PEG-lipid is DSPE-PEG-OH. In some embodiments, the PEG-lipid is PEG-DMG having two C₁₄ hydrophobic tails and PEG2000.

### E. Therapeutic Nucleic Acid

The LNPs provided by the present disclosure also comprise a therapeutic nucleic acid (TNA). According to embodiments, also provided are pharmaceutical compositions comprising the LNPs of the disclosure.

Illustrative therapeutic nucleic acids in the LNPs of the present disclosure can include, but are not limited to, minigenes, plasmids, minicircles, small interfering RNA (siRNA), microRNA (miRNA), antisense oligonucleotides (ASO), ribozymes, deoxyribozymes, closed ended double stranded DNA (e.g., ceDNA, CELiD, linear covalently closed DNA (“ministring”), Doggybone™, protelomere closed ended DNA, or dumbbell linear DNA), dicer-substrate dsRNA, small hairpin RNA (shRNA), asymmetrical interfering RNA (aiRNA), mRNA, tRNA, rRNA, gRNA, and DNA viral vectors, viral RNA vectors, non-viral vectors, and any combination thereof.

In any of the aspects and embodiments provided herein, the therapeutic nucleic acid can be a therapeutic DNA. Said therapeutic DNA can be ceDNA, ssDNA, CELiD, linear covalently closed DNA (“ministring” or otherwise), Doggybone™, protelomere closed ended DNA, dumbbell linear DNA, minigenes, plasmids, or minicircles.

In one embodiment, the therapeutic nucleic acid can be a circular single-stranded polynucleotide comprised of at least three sections, two of which have sufficient complementarity to form a duplex, and an intervening sequence containing the single-stranded nucleic acid to be delivered, as described in described in WO2021/058984, the content of which is incorporated herein by reference in its entirety.

siRNA or miRNA that can downregulate the intracellular levels of specific proteins through a process called RNA interference (RNAi) are also contemplated by the present disclosure to be nucleic acid therapeutics. After siRNA or miRNA is introduced into the cytoplasm of a host cell, these double-stranded RNA constructs can bind to a protein called RISC. The sense strand of the siRNA or miRNA is removed by the RISC complex. The RISC complex, when combined with the complementary mRNA, cleaves the mRNA and release the cut strands. RNAi is by inducing specific destruction of mRNA that results in downregulation of a corresponding protein.

Antisense oligonucleotides (ASO) and ribozymes that inhibit mRNA translation into protein can be nucleic acid therapeutics. For antisense constructs, these single stranded deoxy nucleic acids have a complementary sequence to the sequence of the target protein mRNA, and Watson—capable of binding to the mRNA by Crick base pairing. This binding prevents translation of a target mRNA, and/or triggers RNaseH degradation of the mRNA transcript. As a result, the antisense oligonucleotide has increased specificity of action (i.e., down-regulation of a specific disease-related protein).

In any of the aspects and embodiments provided herein, the therapeutic nucleic acid can be a therapeutic RNA. Said therapeutic RNA can be messenger RNA (mRNA) encoding a protein or peptide, an inhibitor of mRNA translation, agent of RNA interference (RNAi), catalytically active RNA molecule (ribozyme), transfer RNA (tRNA), an RNA that binds an mRNA transcript (ASO), protein or other molecular ligand (aptamer), or a guide RNA (gRNA). In any of the methods provided herein, the agent of RNAi can be a double-stranded RNA, single-stranded RNA, microRNA, short interfering RNA, short hairpin RNA, or a triplex-forming oligonucleotide.

Closed-Ended DNA (ceDNA) Vectors

In some embodiments, LNPs provided by the present disclosure comprise closed-ended DNA (ceDNA).

In some embodiments, the TNA comprises closed-ended linear duplexed (ceDNA) vectors that can express a transgene (e.g., a therapeutic nucleic acid (TNA)). The ceDNA vectors as described herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

ceDNA vectors preferably have a linear and continuous structure rather than a non-continuous structure. The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, it is likely to remain a single molecule. In some embodiments, ceDNA vectors can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects, and also in view of their DNA methylation which is of prokaryotic type for ceDNA-plasmids and of eukaryotic type for the ceDNA vector.

Provided herein are non-viral, capsid-free ceDNA molecules with covalently closed ends (ceDNA). These non-viral capsid free ceDNA molecules can be produced in permissive host cells from an expression construct (e.g., a ceDNA-plasmid, a ceDNA-bacmid, a ceDNA-baculovirus, or an integrated cell-line) containing a heterologous gene (e.g., a transgene, in particular a therapeutic transgene) positioned between two different inverted terminal repeat (ITR) sequences, where the ITRs are different with respect to each other. In some embodiments, one of the ITRs is modified by deletion, insertion, and/or substitution as compared to a wild-type ITR sequence (e.g. AAV ITR); and at least one of the ITRs comprises a functional terminal resolution site (trs) and a Rep binding site. The ceDNA vector is preferably duplex, e.g., self-complementary, over at least a portion of the molecule, such as the expression cassette (e.g., ceDNA is not a double stranded circular molecule). The ceDNA vector has covalently closed ends, and thus is resistant to exonuclease digestion (e.g., exonuclease I or exonuclease III), e.g., for over an hour at 37° C.

In one aspect, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR. In one embodiment, the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. In one embodiment, the first ITR and the second ITR are both modified but are different sequences, or have different modifications, or are not identical modified ITRs, and have different 3D spatial configurations. Stated differently, a ceDNA vector with asymmetric ITRs have ITRs where any changes in one ITR relative to the WT-ITR are not reflected in the other ITR; or alternatively, where the asymmetric ITRs have a the modified asymmetric ITR pair can have a different sequence and different three-dimensional shape with respect to each other.

In one embodiment, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR, where the first ITR (5′ ITR) and the second ITR (3′ ITR) are symmetric, or substantially symmetrical with respect to each other—that is, a ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In one embodiment, a modified ITR pair are substantially symmetrical as defined herein, that is, the modified ITR pair can have a different sequence but have corresponding or the same symmetrical three-dimensional shape. In some embodiments, the symmetrical ITRs, or substantially symmetrical ITRs can be wild type ITRs (WT-ITRs) as described herein. That is, both ITRs have a wild-type sequence from the same AAV serotype. In some other embodiments, the two wild-type ITRs can be from different AAV serotypes. For example, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

The wild-type or mutated or otherwise modified ITR sequences provided herein represent DNA sequences included in the expression construct (e.g., ceDNA-plasmid, ceDNA Bacmid, ceDNA-baculovirus) for production of the ceDNA vector. Thus, ITR sequences actually contained in the ceDNA vector produced from the ceDNA-plasmid or other expression construct may or may not be identical to the ITR sequences provided herein as a result of naturally occurring changes taking place during the production process (e.g., replication error).

In one embodiment, a ceDNA vector in the LNPs of the present disclosure comprising the expression cassette with a transgene which is a therapeutic nucleic acid sequence, can be operatively linked to one or more regulatory sequence(s) that allows or controls expression of the transgene. In one embodiment, the polynucleotide comprises a first ITR sequence and a second ITR sequence, wherein the nucleotide sequence of interest is flanked by the first and second ITR sequences, and the first and second ITR sequences are asymmetrical relative to each other, or symmetrical relative to each other.

In one embodiment, an expression cassette is located between two ITRs in the following order with one or more of: a promoter operably linked to a transgene, a posttranscriptional regulatory element, and a polyadenylation and termination signal. In one embodiment, the promoter is regulatable—inducible or repressible. The promoter can be any sequence that facilitates the transcription of the transgene. In one embodiment the promoter is a CAG promoter, or variation thereof. The posttranscriptional regulatory element is a sequence that modulates expression of the transgene, as a non-limiting example, any sequence that creates a tertiary structure that enhances expression of the transgene which is a therapeutic nucleic acid sequence.

In one embodiment, the posttranscriptional regulatory element comprises WPRE. In one embodiment, the polyadenylation and termination signal comprise BGHpolyA. Any cis regulatory element known in the art, or combination thereof, can be additionally used e.g., SV40 late polyA signal upstream enhancer sequence (USE), or other posttranscriptional processing elements including, but not limited to, the thymidine kinase gene of herpes simplex virus, or hepatitis B virus (HBV). In one embodiment, the expression cassette length in the 5′ to 3′ direction is greater than the maximum length known to be encapsidated in an AAV virion. In one embodiment, the length is greater than 4.6 kb, or greater than 5 kb, or greater than 6 kb, or greater than 7 kb. Various expression cassettes are exemplified herein.

In one embodiment, the expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene which is a therapeutic nucleic acid sequence in the range of 500 to 50,000 nucleotides in length. In one embodiment, the expression cassette can comprise a transgene which is a therapeutic nucleic acid sequence in the range of 500 to 75,000 nucleotides in length. In one embodiment, the expression cassette can comprise a transgene which is a therapeutic nucleic acid sequence in the range of 500 to 10,000 nucleotides in length. In one embodiment, the expression cassette can comprise a transgene which is a therapeutic nucleic acid sequence in the range of 1000 to 10,000 nucleotides in length. In one embodiment, the expression cassette can comprise a transgene which is a therapeutic nucleic acid sequence in the range of 500 to 5,000 nucleotides in length. The ceDNA vectors do not have the size limitations of encapsidated AAV vectors, and thus enable delivery of a large-size expression cassette to the host. In one embodiment, the ceDNA vector is devoid of prokaryote-specific methylation.

In one embodiment, the rigid therapeutic nucleic acid can be a plasmid.

In one embodiment, the ceDNA vectors disclosed herein are used for therapeutic purposes (e.g., for medical, diagnostic, or veterinary uses) or immunogenic polypeptides.

The expression cassette can comprise any transgene which is a therapeutic nucleic acid sequence. In certain embodiments, the ceDNA vector comprises any gene of interest in the subject, which includes protein, enzyme, one or more polypeptides, peptides, ribozymes, peptide nucleic acids, siRNAs, gRNA, mRNA, RNAis, antisense oligonucleotides, antisense polynucleotides, antibodies, antigen binding fragments, or any combination thereof.

In one embodiment, the ceDNA expression cassette can include, for example, an expressible exogenous sequence (e.g., open reading frame) that encodes a protein that is either absent, inactive, or insufficient activity in the recipient subject or a gene that encodes a protein having a desired biological or a therapeutic effect. In one embodiment, the exogenous sequence such as a donor sequence can encode a gene product that can function to correct the expression of a defective gene or transcript. In one embodiment, the expression cassette can also encode corrective DNA strands, encode polypeptides, sense or antisense oligonucleotides, or coding RNAs or non-coding RNAs (e.g., siRNAs, guide RNAs, shRNAs, micro-RNAs, and their antisense counterparts (e.g., antagoMiR)). In one embodiment, expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

Accordingly, the expression cassette can include any gene that encodes a protein, polypeptide or RNA that is either reduced or absent due to a mutation or which conveys a therapeutic benefit when overexpressed is considered to be within the scope of the disclosure. The ceDNA vector may comprise a template or donor nucleotide sequence used as a correcting DNA strand to be inserted after a double-strand break (or nick) provided by a nuclease. The ceDNA vector may include a template nucleotide sequence used as a correcting DNA strand to be inserted after a double-strand break (or nick) provided by a guided RNA nuclease, meganuclease, or zinc finger nuclease.

### Single-Stranded (ss) Nucleic Acid Molecules

In some embodiments, the TNA comprised in an LNP of the present disclosure may be a single-stranded nucleic acid, e.g., a single-stranded DNA or a single-stranded RNA. In one embodiment, the TNA may be a single-stranded RNA, e.g., mRNA. In another embodiment, the TNA may be a single-stranded DNA (ssDNA) molecule, e.g., a synthetic ssDNA molecule.

### 3′ End Stem-Loop Structure

In some aspects, the TNA is a ssDNA molecule comprising at least one nucleic acid sequence of interest flanked by at least one stem-loop structure at the 3′ end. In some embodiments, the ssDNA molecule may further comprise at least one stem-loop structure at the 5′ end. As described herein, the stem-loop structure at the 3′ end may comprise a partial DNA duplex (e.g., with a free 3′-OH group) to prime replication or transcription. The partial DNA duplex functions, in part, to hold the stem-loop structure together.

According to some embodiments, the partial DNA duplex comprises between 4-500 nucleotides, for example between 4-10 nucleotides, between 4-25 nucleotides, between 4-50 nucleotides, between 4-100 nucleotides, between 4-200 nucleotides, between 4-300 nucleotides, between 4-400 nucleotides, between 20-25 nucleotides, between 20-50 nucleotides, between 20-100 nucleotides, between 20-200 nucleotides, between 20-300 nucleotides, between 20-400 nucleotides, between 20-500 nucleotides, between 50-100 nucleotides, between 50-200 nucleotides, between 50-300 nucleotides, between 50-400 nucleotides, between 50-500 nucleotides, 150-200 nucleotides, between 150-300 nucleotides, between 150-400 nucleotides, between 150-500 nucleotides, between 200-300 nucleotides, between 200-400 nucleotides, between 200-500 nucleotides, between 250-300 nucleotides, between 250-400 nucleotides, between 250-500 nucleotides, between 300-400 nucleotides, between 300-500 nucleotides, between 350-400 nucleotides, between 350-500 nucleotides, between 400-500 nucleotides, or between 450-500 nucleotides, and at least one loop on the 3′ end. According to some embodiments, the DNA duplex comprises at least 4, 5, 6, 7, 8, 9, 10, 20, 30, 40, 50, 60, 70, 80, 90, 100, 150, 200, 250, 300, 350, 400, 450 or 500 nucleotides, and at least one loop on the 3′ end.

According to some embodiments, the loop structure at the 3′ end comprises a minimum of between 3-500 unbound nucleotides, for example between 3-450 nucleotides, between 3-400 nucleotides, between 3-350 nucleotides, between 3-300 nucleotides, between 3-250 nucleotides, between 3-200 nucleotides, between 3-150 nucleotides, between 3-100 nucleotides, between 3-90 nucleotides, between 3-80 nucleotides, between 3-70 nucleotides, between 3-60 nucleotides, between 3-50 nucleotides, between 3-40 nucleotides, between 3-30 nucleotides, between 3-20 nucleotides, between 3-10 nucleotides, between 3-5 nucleotides, between 10-450 nucleotides, between 10-400 nucleotides, between 10-350 nucleotides, between 10-300 nucleotides, between 10-250 nucleotides, between 10-200 nucleotides, between 10-150 nucleotides, between 10-100 nucleotides, between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 50-450 nucleotides, between 50-400 nucleotides, between 50-350 nucleotides, between 50-300 nucleotides, between 50-250 nucleotides, between 50-200 nucleotides, between 50-150 nucleotides, between 50-100 nucleotides, between 50-90 nucleotides, between 50-80 nucleotides, between 50-70 nucleotides, between 50-60 nucleotides, between 100-450 nucleotides, between 100-400 nucleotides, between 100-350 nucleotides, between 100-300 nucleotides, between 100-250 nucleotides, between 100-200 nucleotides, between 150-450 nucleotides, between 150-400 nucleotides, between 150-350 nucleotides, between 150-300 nucleotides, between 150-250 nucleotides, between 150-200 nucleotides, between 200-450 nucleotides, between 200-400 nucleotides, between 200-350 nucleotides, between 200-300 nucleotides, between 200-250 nucleotides, between 250-450 nucleotides, between 250-400 nucleotides, between 250-350 nucleotides, between 250-300 nucleotides, between 300-450 nucleotides, between 300-400 nucleotides, between 300-350 nucleotides, between 350-450 nucleotides, between 350-400 nucleotides, or between 400-450 nucleotides.

According to some embodiments, the stem portion of the stem-loop is 4-500 nucleotides in length and the loop portion of the stem-loop is 3-500 nucleotides in length. According to some embodiments, the stem portion of the stem-loop is 4-50 nucleotides in length and the loop portion of the stem-loop is 3-50 nucleotides in length. According to some embodiments, the stem portion of the stem-loop is 4-20 nucleotides in length and the loop portion of the stem-loop is 3-20 nucleotides in length. According to some embodiments, the stem portion of the stem-loop is 4-10 nucleotides in length and the loop portion of the stem-loop is 3-10 nucleotides in length.

According to some embodiments, the loop further comprises one or more nucleic acids or that are used to stabilize the ends. According to other embodiments, the loop further comprises one or more nucleic acids that may be employed in therapeutic methods. According to other embodiments, the loop further comprises one or more nucleic acids that may be employed in diagnostic methods. According to other embodiments, the loop further comprises one or more nucleic acids that that may be employed for research purposes.

According to some embodiments, the minimal nucleic acid structure that is necessary at the 3′ end of the ssDNA is any structure that loops back on itself, i.e., a hairpin structure. However, it is to be understood that a variety of structures are envisioned at the 3′ end, as long as there is at least one stem and one loop. For example, in some embodiments, the ssDNA described herein may comprise at least one stem-loop structure at the 3′ end. In some embodiments, the ssDNA may comprise at least two stem-loop structures at the 3′ end. In some embodiments, the ssDNA may comprise at least three stem-loop structures at the 3′ end. In some embodiments, the ssDNA may comprise at least four stem-loop structures at the 3′ end. In some embodiments, the ssDNA may comprise at least five stem-loop structures at the 3′ end.

According to some embodiments, the nucleotides at the 3′ end form a cruciform DNA structure. A DNA cruciform structure can be formed when both strands form a stem-loop structure at the same location in the molecule, and comprises a four-way junction and two closed hairpin-shaped points.

According to some embodiments, the nucleotides at the 3′ end form a hairpin DNA structure.

Hairpin loop structures in nucleic acids consist of a base-paired stem structure and a loop sequence with unpaired or non-Watson-Crick-paired nucleotides.

According to some embodiments, the nucleotides at the 3′ end form a hammerhead DNA structure, made up of three base paired helices, separated by short linkers of conserved sequence.

According to some embodiments, the nucleotides at the 3′ end form a quadraplex DNA structure. G-quadruplexes are four-stranded DNA secondary structures (G4s) that form from certain guanine-rich sequences.

According to some embodiments, the nucleotides at the 3′ end form a bulged DNA structure.

According to some embodiments, the nucleotides at the 3′ end form a multibranched loop.

According to some embodiments, the nucleotides at the 3′ end do not form a 2 stem-loop structure.

According to some embodiments, the stem structure at the 3′ end comprises one or more nucleotides that are modified to be exonuclease resistant. According to some embodiments, the stem structure at the 3′ end comprises two or more, 3 or more, 4 or more, 5 or more, 6 or more, 7 or more, 8 or more, 9 or more, 10 or more, or 20 or more nucleotides that are modified to be exonuclease resistant.

According to some embodiments, the stem structure at the 3′ end comprises one or more phosphorothioate-modified nucleotides. According to some embodiments, the stem structure at the 3′ end comprises about 2 to about 12 phosphorothioate-modified nucleotides. According to some embodiments, the stem structure at the 3′ end comprises about 4 to about 10 phosphorothioate-modified nucleotides, e.g., about 4 to about 5, about 4 to about 6, about 4 to about 7, about 4 to about 8, about 4 to about 9, about 4 to about 10, about 5 to about 6, about 5 to about 7, about 5 to about 8, about 5 to about 9, about 5 to about 10, about 6 to about 7, about 6 to about 8, about 6 to about 9, about 6 to about 10, about 7 to about 8, about 7 to about 9, about 7 to about 10, about 8 to about 9, about 8 to about 10 or about 9 to about 10. According to some embodiments, the stem structure comprises more than 10 phosphorothioate-modified nucleotides.

According to some embodiments, the phosphorothioate-modified nucleotides are located adjacent to each other.

According to some embodiments, the one or more phosphorothioate-modified nucleotides of the 3′ end are resistant to exonuclease degradation. Boranophosphate modified DNA is also resistant to nuclease degradation, and may be considered as an alternative to phosphorothioate modification.

According to further embodiments, the stem structure may comprise at least one functional moiety. In one embodiment, the at least one functional moiety is an aptamer sequence. In further embodiments, the aptamer sequence has a high binding affinity to a nuclear localized protein.

According to some embodiments, the nucleotides in the loop are chemically modified with functional groups in order to alter their properties.

According to some embodiments, the loop further comprises one or more aptamers.

According to some embodiments, the aptamer is identified from the Apta-index database of aptamers available to the public (aptagen.com/apta-index).

According to some embodiments, the loop further comprises one or more synthetic ribozymes.

According to some embodiments, the loop further comprises one or more antisense oligonucleotides (ASOs).

According to some embodiments, the loop further comprises one or more short-interfering RNAs (siRNAs).

According to some embodiments, the loop further comprises one or more antiviral nucleoside analogues (ANAs).

According to some embodiments, the loop further comprises one or more triplex forming oligonucleotides.

According to some embodiments, the loop further comprises one or more gRNAs or gDNAs.

According to some embodiments, the loop further comprises one or more molecular probes, for example nucleic acid based fluorescent probes.

According to some embodiments, “click” azide-alkyne cycloaddition (Kolb et al., Angew. Chem. Int. Ed. Engl. 2001, 40, 2004-2021) is used to modify the nucleotides in the loop. Click chemistry was developed to join together organic molecules under mild conditions in the presence of a diverse range of functional groups. Most click-mediated modifications are performed on the nitrogenous bases by introducing novel base analogues, attaching fluorophores or isotopic elements for molecular imaging, forming inter-strand linkages between oligonucleotides, and for the bioconjugation of molecules. The best example of click chemistry is the Cu^{I} catalyzed version of Huisgen's [3+2] azide-alkyne cycloaddition reaction (Angew. Chem., Int. Ed. 1963, 2, 633-645), discovered independently by Sharpless and Meldal (the CuAAC reaction) (Angew. Chem., Int. Ed. 2002, 41, 2596-2599).

According to some embodiments, the introduction of active amino or thiol groups into synthesized oligonucleotides provides acceptors for, e.g., subsequent chemical fluorescent labeling.

According to some embodiments, the stem-loop structure may comprise alternative or modified nucleotides, including, but not limited to, ribonucleic acids (RNA), peptide-nucleic acids (PNA), locked nucleic acids (LNA). According to some embodiments, the loop portion of the stem-loop structure may comprise a chemical structure that does not comprise nucleic acids.

### IV. Preparation of Lipid Nanoparticles (LNPs)

Lipid nanoparticles (LNPs) can form spontaneously upon mixing of a therapeutic nucleic acid (e.g., ceDNA, ssDNA, synthetic AAV, etc., as described herein) and a pharmaceutically acceptable excipient that comprises a lipid.

Generally, LNPs can be formed by any method known in the art. For example, the LNPs can be prepared by the methods described, for example, in US2013/0037977, US2010/0015218, US2013/0156845, US2013/0164400, US2012/0225129, and US2010/0130588, content of each of which is incorporated herein by reference in its entirety. In some embodiments, LNPs can be prepared using a continuous mixing method, a direct dilution process, or an in-line dilution process. The processes and apparatuses for preparing lipid nanoparticles using direct dilution and in-line dilution processes are described in US2007/0042031, the content of which is incorporated herein by reference in its entirety. The processes and apparatuses for preparing lipid nanoparticles using stepwise dilution processes are described in US2004/0142025, the content of which is incorporated herein by reference in its entirety.

According to some embodiments, the disclosure provides for an LNP comprising a DNA vector, including a ceDNA vector, ssDNA vector, or synthetic AAV, as described herein and an ionizable lipid. For example, a lipid nanoparticle formulation that is made and loaded with therapeutic nucleic acid like ceDNA obtained by the process as disclosed in International Patent Application No. PCT/US2018/050042, filed on Sep. 7, 2018, which is incorporated by reference in its entirety herein. This can be accomplished by high energy mixing of ethanolic lipids with aqueous synthetic AAV at low pH which protonates the ionizable lipid and provides favorable energetics for synthetic AAV/lipid association and nucleation of particles. The particles can be further stabilized through aqueous dilution and removal of the organic solvent. The particles can be concentrated to the desired level.

Generally, the lipid particles are prepared at a total lipid to synthetic AAV (mass or weight) ratio of from about 10:1 to 30:1. In some embodiments, the lipid to ssDNA molecule or the dsDNA construct ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and synthetic AAV can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo at low pH and to drive membrane association and fusogenicity. Generally, ionizable lipids are lipids comprising at least one amino group that is positively charged or becomes protonated under acidic conditions, for example at pH of 6.5 or lower.

In one embodiment, the LNPs can be prepared by an impinging jet process. Generally, the particles are formed by mixing lipids dissolved in alcohol (e.g., ethanol) with ceDNA dissolved in a buffer, e.g., a citrate buffer, a sodium acetate buffer, a sodium acetate and magnesium chloride buffer, a malic acid buffer, a malic acid and sodium chloride buffer, or a sodium citrate and sodium chloride buffer. The mixing ratio of lipids to ceDNA can be about 45-55% lipid and about 65-45% ceDNA.

The lipid solution can contain an ionizable lipid, a ceramide, a lipid-anchored polymer and a sterol (e.g., cholesterol) at a total lipid concentration of 5-30 mg/mL, more likely 5-15 mg/mL, most likely 9-12 mg/mL in an alcohol, e.g., in ethanol. In the lipid solution, mol ratio of the lipids can range from about 25-98% for the cationic lipid, preferably about 35-65%; about 0-15% for the nonionic lipid, preferably about 0-12%; about 0-15% for the PEG or PEG conjugated lipid molecule, preferably about 1-6%; and about 0-75% for the sterol, preferably about 30-50%.

The ceDNA solution can comprise the ceDNA at a concentration range from 0.3 to 1.0 mg/mL, preferably 0.3-0.9 mg/mL in buffered solution, with pH in the range of 3.5-5.

For forming the LNPs, in one exemplary but nonlimiting embodiment, the two liquids are heated to a temperature in the range of about 15-40° C., preferably about 30-40° C., and then mixed, for example, in an impinging jet mixer, instantly forming the LNP. The mixing flow rate can range from 10-600 mL/min. The tube ID can have a range from 0.25 to 1.0 mm and a total flow rate from 10-600 mL/min. The combination of flow rate and tubing ID can have the effect of controlling the particle size of the LNPs between 30 and 200 nm. The solution can then be mixed with a buffered solution at a higher pH with a mixing ratio in the range of 1:1 to 1:3 vol:vol, preferably about 1:2 vol:vol. If needed this buffered solution can be at a temperature in the range of 15-40° C. or 30-40° C. The mixed LNPs can then undergo an anion exchange filtration step. Prior to the anion exchange, the mixed LNPs can be incubated for a period of time, for example 30 mins to 2 hours. The temperature during incubating can be in the range of 15-40° C. or 30-40° C. After incubating the solution is filtered through a filter, such as a 0.8 μm filter, containing an anion exchange separation step. This process can use tubing IDs ranging from 1 mm ID to 5 mm ID and a flow rate from 10 to 2000 mL/min.

After formation, the LNPs can be concentrated and diafiltered via an ultrafiltration process where the alcohol is removed and the buffer is exchanged for the final buffer solution, for example, phosphate buffered saline (PBS) at about pH 7, e.g., about pH 6.9, about pH 7.0, about pH 7.1, about pH 7.2, about pH 7.3, or about pH 7.4.

The ultrafiltration process can use a tangential flow filtration format (TFF) using a membrane nominal molecular weight cutoff range from 30-500 kD. The membrane format is hollow fiber or flat sheet cassette. The TFF processes with the proper molecular weight cutoff can retain the LNP in the retentate and the filtrate or permeate contains the alcohol; citrate buffer and final buffer wastes. The TFF process is a multiple step process with an initial concentration to a ceDNA concentration of 1-3 mg/mL. Following concentration, the LNPs solution is diafiltered against the final buffer for 10-20 volumes to remove the alcohol and perform buffer exchange. The material can then be concentrated an additional 1-3-fold. The concentrated LNP solution can be sterile filtered.

### V. Pharmaceutical Compositions and Formulations

The present disclosure also provides a pharmaceutical composition comprising the LNPs of the present disclosure and at least one pharmaceutically acceptable excipient.

According to some embodiments, the TNA (e.g., ceDNA) is encapsulated in the LNP. In one embodiment, the LNPs of the disclosure are provided with full encapsulation, partial encapsulation of the therapeutic nucleic acid. In one embodiment, the nucleic acid therapeutics is fully encapsulated in the LNPs to form a nucleic acid containing lipid particle. In one embodiment, the nucleic acid may be encapsulated within the lipid portion of the particle, thereby protecting it from enzymatic degradation.

In one embodiment, encapsulation of TNA (e.g., ceDNA) in the LNPs of the present disclosure can be determined by performing a membrane-impermeable fluorescent dye exclusion assay, which uses a dye that has enhanced fluorescence when associated with nucleic acid, for example, an Oligreen® assay or PicoGreen® assay. Generally, encapsulation is determined by adding the dye to the lipid particle formulation, measuring the resulting fluorescence, and comparing it to the fluorescence observed upon addition of a small amount of nonionic detergent. Detergent-mediated disruption of the lipid bilayer releases the encapsulated TNA (e.g., ceDNA), allowing it to interact with the membrane-impermeable dye. Encapsulation of ceDNA can be calculated as E=(Io−I)/Io, where I and Io refer to the fluorescence intensities before and after the addition of detergent.

Depending on the intended use of the LNPs, the proportions of the components can vary and the delivery efficiency of a particular formulation can be measured using, for example, an endosomal release parameter (ERP) assay.

In one embodiment, the TNA (e.g., ceDNA) can be complexed with the lipid portion of the particle or encapsulated in the lipid position of the lipid particle (e.g., lipid nanoparticle). In one embodiment, the TNA can be fully encapsulated in the lipid position of the lipid particle (e.g., lipid nanoparticle), thereby protecting it from degradation by a nuclease, e.g., in an aqueous solution. In one embodiment, the TNA in the lipid particle (e.g., lipid nanoparticle) is not substantially degraded after exposure of the lipid particle (e.g., lipid nanoparticle) to a nuclease at 37° C. for at least about 20, 30, 45, or 60 minutes. In some embodiments, the TNA in the lipid particle (e.g., lipid nanoparticle) is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In one embodiment, the LNPs are substantially non-toxic to a subject, e.g., to a mammal such as a human.

In one embodiment, the pharmaceutical composition comprising LNPs of the disclosure is an aqueous solution. In one embodiment, the pharmaceutical composition comprising LNPs of the disclosure is a lyophilized powder.

According to some aspects, the at least one pharmaceutically acceptable excipient in the pharmaceutical compositions of the present disclosure is a sucrose, tris, trehalose and/or glycine.

In one embodiment, the pharmaceutical compositions comprising LNPs of the disclosure are suitable for administration to a subject for in vivo delivery to cells, tissues, or organs of the subject. In some embodiments, the pharmaceutical composition is suitable for a desired route of therapeutic administration (e.g., parenteral administration). The pharmaceutical compositions for therapeutic purposes can be formulated as a solution, microemulsion, dispersion, liposomes, or other ordered structure suitable for high TNA (e.g., ceDNA) vector concentration. Sterile injectable solutions can be prepared by incorporating the TNA (e.g., ceDNA) vector compound in the required amount in an appropriate buffer with one or a combination of ingredients enumerated above, as required, followed by filtered sterilization.

Pharmaceutical compositions comprising LNPs of the disclosure are suitable for topical, systemic, intra-amniotic, intrathecal, intracranial, intraarterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

Pharmaceutical compositions for therapeutic purposes typically must be sterile and stable under the conditions of manufacture and storage.

In one embodiment, LNPs are solid core particles that possess at least one lipid bilayer. In one embodiment, the LNPs have a non-bilayer structure, i.e., a non-lamellar (i.e., non-bilayer) morphology. Without limitations, the non-bilayer morphology can include, for example, three dimensional tubes, rods, cubic symmetries, etc. The non-lamellar morphology (i.e., non-bilayer structure) of the LNPs can be determined using analytical techniques known to and used by those of skill in the art. Such techniques include, but are not limited to, Cryo-Transmission Electron Microscopy (“Cryo-TEM”), Differential Scanning calorimetry (“DSC”), X-Ray Diffraction, and the like. For example, the morphology of the lipid particles (lamellar vs. non-lamellar) can readily be assessed and characterized using, e.g., Cryo-TEM analysis as described in US2010/0130588, the content of which is incorporated herein by reference in its entirety.

In one embodiment, the LNPs having a non-lamellar morphology are electron dense.

In one embodiment, the LNPs provided by the present disclosure is either unilamellar or multilamellar in structure. In some aspects, the pharmaceutical composition of the disclosure comprises multi-vesicular particles and/or foam-based particles. By controlling the composition and concentration of the lipid components, one can control the rate at which the lipid conjugate exchanges out of the lipid particle and, in turn, the rate at which the LNP becomes fusogenic. In addition, other variables including, for example, pH, temperature, or ionic strength, can be used to vary and/or control the rate at which the LNP becomes fusogenic. Other methods which can be used to control the rate at which the LNP becomes fusogenic will be apparent to those of ordinary skill in the art based on this disclosure. It will also be apparent that by controlling the composition and concentration of the lipid conjugate, one can control the lipid particle size.

According to some embodiments, for ophthalmic delivery, interfering RNA-ligand conjugates and nanoparticle-ligand conjugates may be combined with ophthalmologically acceptable preservatives, co-solvents, surfactants, viscosity enhancers, penetration enhancers, buffers, sodium chloride, or water to form an aqueous, sterile ophthalmic suspension or solution.

### Unit Dosage

In one embodiment, the pharmaceutical compositions can be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### VI. Methods of Treatment

In some aspects, the present disclosure provides methods of treating a disorder in a subject that comprise administering to the subject an effective amount of an LNP of the disclosure of the pharmaceutical composition comprising the LNP of the disclosure. In some embodiments, the disorder is a genetic disorder.

As used herein, the term “genetic disease” or “genetic disorder” refers to a disease, partially or completely, directly or indirectly, caused by one or more abnormalities in the genome, including and especially a condition that is present from birth. The abnormality may be a mutation, an insertion or a deletion in a gene. The abnormality may affect the coding sequence of the gene or its regulatory sequence.

Provided herein are methods for treating genetic disorders by administering the LNP of the disclosure or the pharmaceutical composition comprising LNPs of the disclosure. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, the LNPs and LNP compositions of the disclosure can be used to deliver transgenes to bring a normal gene into affected tissues for replacement therapy, as well, in some embodiments of any of the aspects and embodiments herein, to create animal models for the disease using antisense mutations. For unbalanced disease states, the LNPs and LNP compositions of the disclosure can be used to create a disease state in a model system, which could then be used in efforts to counteract the disease state. Thus, the LNPs or LNP compositions of the disclosure and methods disclosed herein permit the treatment of genetic diseases. As used herein, a disease state is treated by partially or wholly remedying the deficiency or imbalance that causes the disease or makes it more severe.

In general, the LNPs and LNP compositions of the disclosure can be used to deliver any transgene in accordance with the description above to treat, prevent, or ameliorate the symptoms associated with any disorder related to gene expression. Illustrative disease states include, but are not-limited to: cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Hurler's disease, adenosine deaminase deficiency, metabolic defects, retinal degenerative diseases (and other diseases of the eye), mitochondriopathies (e.g., Leber's hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments of any of the aspects and embodiments herein, the ceDNA vectors as disclosed herein can be advantageously used in the treatment of individuals with metabolic disorders (e.g., ornithine transcarbamylase deficiency).

In one embodiment of any of the aspects or embodiments herein, the LNPs of the disclosure or the pharmaceutical compositions comprising the LNPs of the disclosure can be used to treat, ameliorate, and/or prevent a disease or disorder caused by mutation in a gene or gene product. Exemplary diseases or disorders that can be treated with the LNPs or the LNP compositions of the disclosure include, but are not limited to, metabolic diseases or disorders (e.g., Fabry disease, Gaucher disease, phenylketonuria (PKU), glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., hemophilia A and B, thalassemia, and anemia); cancers and tumors, and genetic diseases or disorders (e.g., cystic fibrosis).

In one embodiment, the LNPs or LNP compositions of the disclosure may be employed to deliver a heterologous nucleotide sequence in situations in which it is desirable to regulate the level of transgene expression (e.g., transgenes encoding hormones or growth factors).

In one embodiment of any of the aspects or embodiments herein, the LNPs or LNP compositions of the disclosure can be used to correct an abnormal level and/or function of a gene product (e.g., an absence of, or a defect in, a protein) that results in the disease or disorder. The LNPs or LNP compositions of the disclosure can produce a functional protein and/or modify levels of the protein to alleviate or reduce symptoms resulting from, or confer benefit to, a particular disease or disorder caused by the absence or a defect in the protein. For example, treatment of OTC deficiency can be achieved by producing functional OTC enzyme; treatment of hemophilia A and B can be achieved by modifying levels of Factor VIII, Factor IX, and Factor X; treatment of PKU can be achieved by modifying levels of phenylalanine hydroxylase enzyme; treatment of Fabry or Gaucher disease can be achieved by producing functional alpha galactosidase or beta glucocerebrosidase, respectively; treatment of MFD or MPSII can be achieved by producing functional arylsulfatase A or iduronate-2-sulfatase, respectively; treatment of cystic fibrosis can be achieved by producing functional cystic fibrosis transmembrane conductance regulator; treatment of glycogen storage disease can be achieved by restoring functional G6Pase enzyme function; and treatment of PFIC can be achieved by producing functional ATP8B1, ABCB11, ABCB4, or TJP2 genes.

In some embodiments, the LNPs or LNP compositions of the disclosure can be used to provide a DNA-based therapeutic to a cell in vitro or in vivo. Examples of DNA-based therapeutics include, but are not limited to, minicircle DNA, minigene, viral DNA (e.g., Lentiviral or AAV genome) or non-viral synthetic DNA vectors, closed-ended linear duplex DNA (ceDNA/CELiD), plasmids, bacmids, Doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

In one embodiment of any of the aspects or embodiments herein, exemplary transgenes encoded by ceDNA in the LNPs or LNP compositions of the disclosure include, but are not limited to: X, lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, β-interferon, interferon-γ, interleukin-2, interleukin-4, interleukin 12, granulocyte-macrophage colony stimulating factor, lymphotoxin, and the like), peptide growth factors and hormones (e.g., somatotropin, insulin, insulin-like growth factors 1 and 2, platelet derived growth factor (PDGF), epidermal growth factor (EGF), fibroblast growth factor (FGF), nerve growth factor (NGF), neurotrophic factor-3 and 4, brain-derived neurotrophic factor (BDNF), glial derived growth factor (GDNF), transforming growth factor-a and -b, and the like), receptors (e.g., tumor necrosis factor receptor). In some exemplary embodiments, the transgene encodes a monoclonal antibody specific for one or more desired targets. In some exemplary embodiments, more than one transgene is encoded by the ceDNA vector. In some exemplary embodiments, the transgene encodes a fusion protein comprising two different polypeptides of interest. In some embodiments of any of the aspects and embodiments herein, the transgene encodes an antibody, including a full-length antibody or antibody fragment, as defined herein. In some embodiments of any of the aspects and embodiments herein, the antibody is an antigen-binding domain or an immunoglobulin variable domain sequence, as that is defined herein. Other illustrative transgene sequences encode suicide gene products (thymidine kinase, cytosine deaminase, diphtheria toxin, cytochrome P450, deoxycytidine kinase, and tumor necrosis factor), proteins conferring resistance to a drug used in cancer therapy, and tumor suppressor gene products.

In one embodiment of any of the aspects or embodiments herein, this disclosure provides a method of providing anti-tumor immunity in a subject, the method comprising administering to the subject an effective amount of any embodiment of an LNP contemplated herein or any embodiment of a pharmaceutical composition comprising an LNP contemplated herein. Furthermore, this disclosure provides a method of treating a subject having a disease, disorder or condition associated with an elevated expression of a tumor antigen, the method comprising administering to the subject an effective amount of any embodiment of an LNP contemplated herein or any embodiment of a pharmaceutical composition comprising an LNP contemplated herein. In some embodiments, the TNA is retained in the spleen for at least about 6 hours, or at least about 9 hours, or at least about 12 hours, or at least about 15 hours, or at least about 18 hours, or at least about 21 hours, or at least about 24 hours, or at least about 27 hours, or at least about 30 hours, or at least about 33 hours, or at least about 36 hours after dosing of an LNP of this disclosure, for example, via intravenous or intratumoral administration. In some embodiments, the amount (i.e., number of copies) of the TNA at the start of a 12, 18, or 24-hour time window post-dosing and the amount of the TNA at the end of the time window are within the same order of magnitude (e.g., 10⁻⁷ copies, 10⁻⁶ copies, 10⁻⁵ copies, 10⁻⁴ copies, 10⁻³ copies, 10⁻² copies, 10⁻¹ copies, 10⁰ copies, 10¹ copies, 10² copies, 10³ copies, etc. or any other suitable therapeutic levels). In other words, there is minimal reduction in concentrations of the TNA in the spleen within a 12, 18, or 24-hour time window post-dosing. In some embodiments, the TNA is a messenger RNA (mRNA).

Examples of solid tumors treatable with an LNP disclosed herein or a pharmaceutical composition comprising the same include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. According to some embodiments, the tumor or cancer is a melanoma, e.g., an advanced stage melanoma. Metastatic lesions of the aforementioned cancers can also be treated or prevented using the methods and compositions of the disclosure. Examples of other solid tumors or cancers that can be treated include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

In further embodiments, the present disclosure provides a method of treating a blood disease, disorder or condition in a subject, the method comprising administering to the subject an effective amount of any embodiment of an LNP contemplated herein or any embodiment of a pharmaceutical composition comprising an LNP contemplated herein. Non-limiting examples of the blood disease, disorder or condition include acute myeloid leukemia (AML), acute lymphoblastic leukemia (ALL), chronic lymphocytic leukemia (CLL), chronic myelogenous leukemia (CML), Hodgkin lymphoma (HL), multiple myeloma, a myelodysplastic syndrome (MDS), non-Hodgkin lymphoma (NHL), adrenoleukodystrophy (ALD), Hurler syndrome, Krabbe disease (Globoid-cell leukodystrophy or GLD), metachromatic leukodystrophy (MLD), severe aplastic anemia (SAA), severe combined immunodeficiency (SCID), sickle cell disease (SCD), thalassemia, Wiskott-Aldrich syndrome, Diamond-Blackfan anemia, essential thrombocytosis, Fanconi anemia, hemophagocytic lymphohistiscytosis (HLH), juvenile myelomonocytic leukemia (JMML), myelofibrosis, polycythemia vera, and a combination thereof. In some embodiments, the TNA is a messenger RNA (mRNA). In some embodiments, the TNA is retained in the bone marrow for at least about 6 hours, or at least about 9 hours, or at least about 12 hours, or at least about 15 hours, or at least about 18 hours, or at least about 21 hours, or at least about 24 hours, or at least about 27 hours, or at least about 30 hours, or at least about 33 hours, or at least about 36 hours after dosing of an LNP of this disclosure, for example, via intravenous or intratumoral administration. In some embodiments, the amount (i.e. number of copies) of the TNA at the start of a 12, 18, or 24-hour time window post-dosing and the number of the TNA at the end of the time window are within the same order of magnitude (e.g., 10⁻⁷ copies, 10⁻⁶ copies, 10⁻⁵ copies, 10⁻⁴ copies, 10⁻³ copies, 10⁻² copies, 10⁻¹ copies, 10⁰ copies, 10¹ copies, 10² copies, 10³ copies, etc. or any other suitable therapeutic levels) or are reduced for less than one order of magnitude. In other words, there is minimal or insignificant reduction in concentrations of the TNA in the bone marrow within a 12, 18, or 24-hour time window post-dosing. In some embodiments, the TNA is a messenger RNA (mRNA).

### Administration

In some embodiments, an LNP or an LNP composition of the disclosure can be administered to an organism for transduction of cells in vivo. In some embodiments, an LNP or an LNP composition of the disclosure can be administered to an organism for transduction of cells ex vivo.

Generally, administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Exemplary modes of administration of an LNP or an LNP composition of the disclosure include oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of the LNP or LNP compositions of the disclosure can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. The most suitable route in any given case will depend on the nature and severity of the condition being treated, ameliorated, and/or prevented and on the nature of the particular ceDNA LNP that is being used. Additionally, ceDNA permits one to administer more than one transgene in a single vector, or multiple ceDNA vectors (e.g., a ceDNA cocktail).

In one embodiment of any of the aspects or embodiments herein, the LNPs or LNP compositions of the disclosure can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In some embodiments, the LNPs of the disclosure or the pharmaceutical compositions comprising the LNPs of the disclosure, when administered to a subject, is characterized by a lower immunogenicity than a reference LNP or a pharmaceutical composition comprising a reference LNP. In some embodiments, the immunogenicity of the LNP of the disclosure or the pharmaceutical composition comprising the LNP of the disclosure may be measured by measuring levels of one or more proinflammatory cytokines. Accordingly, in some embodiments, the LNPs of the disclosure or the pharmaceutical compositions comprising the LNPs of the disclosure, when administered to a subject, elicits a lower pro-inflammatory cytokine response than a reference LNP or a pharmaceutical composition comprising a reference LNP. The term “elicits a lower pro-inflammatory cytokine response than a reference LNP or a pharmaceutical composition comprising a reference LNP”, as used herein, means that the LNP of the disclosure or the pharmaceutical composition comprising the LNP of the disclosure, when administered to a subject, causes a smaller increase in the levels of one or more pro-inflammatory cytokines as compared to a reference LNP or a pharmaceutical composition comprising a reference LNP. Exemplary pro-inflammatory cytokines include, but are not limited to, granulocyte colony stimulating factor (G-CSF), interleukin 1 alpha (IL-1α), interleukin 1 beta (IL-1β), interleukin 6 (IL-6), interleukin 8 (IL-8 or CXCL8), interleukin 11 (IL-11), interleukin 17 (IL-17), interleukin 18 (IL-18), interferon α (IFN-α), interferon β (IFN-β), interferon γ (IFN-γ), C-X-C motif chemokine ligand 10 (CXCL10 or IP-10), monocyte chemoattractant protein 1 (MCP-1), CD40L, CCL2, CCL3, CCL4, CCL5, CCL11, tumor necrosis factor α (TNF-α), and combinations thereof.

### REFERENCES

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited in any manner to the particular methodology, protocols, and reagents, etc., described herein and as such can vary. The terminology used herein is for the purpose of describing particular embodiments only and is not intended to limit the scope of the present disclosure, which is defined solely by the claims.

### EXAMPLES

The following examples are provided by way of illustration, not limitation.

### Example 1. Synthesis of polymer-conjugated lipids

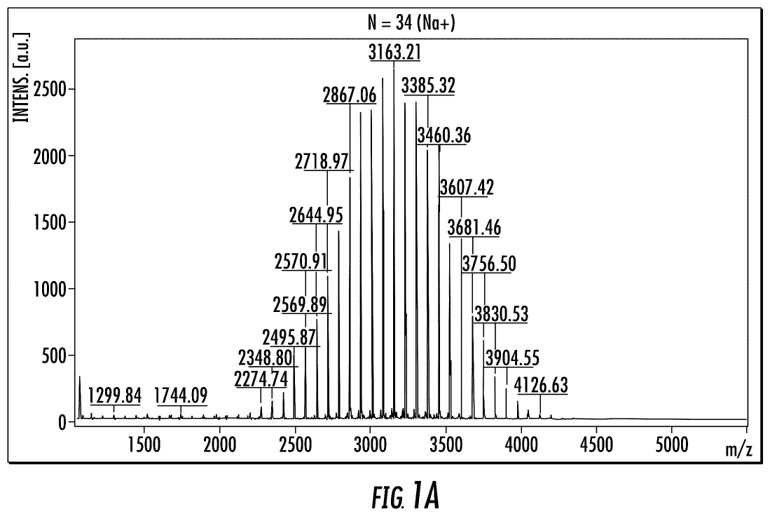

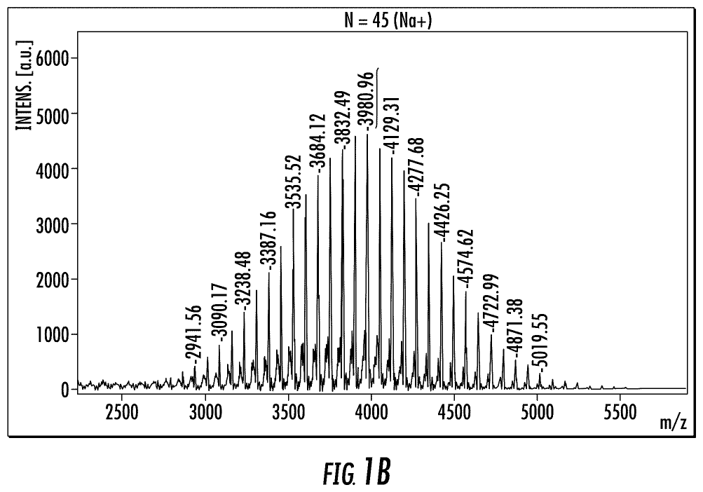

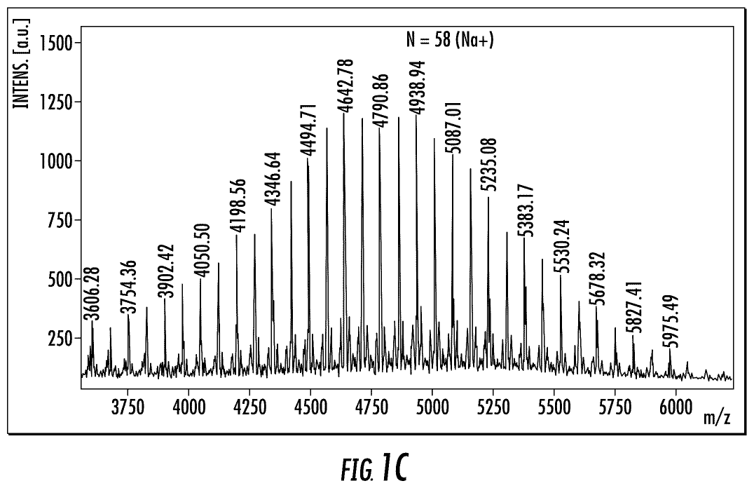

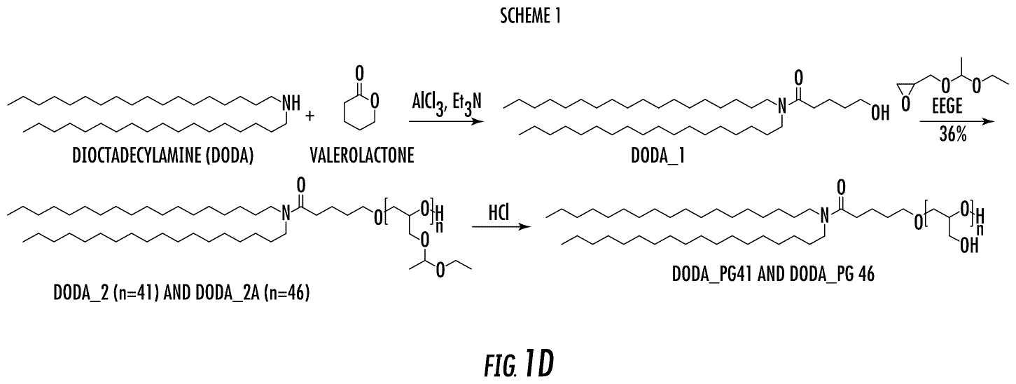

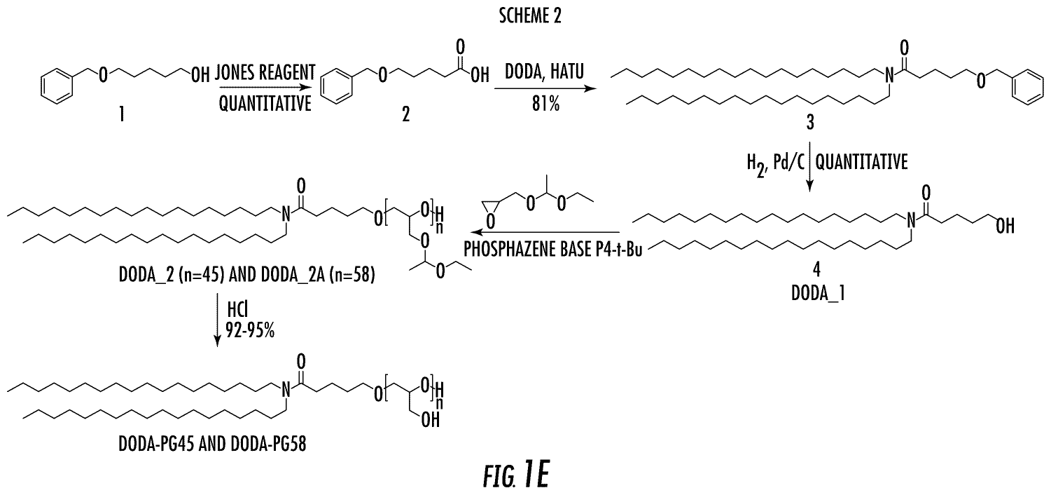

The goal of this experiment was to synthesize exemplary polymer-conjugated lipids for use in LNPs. In this experiment, the polymer-conjugated lipids synthesized were dioctadecylamine (DODA) conjugated to polyglycerol containing 34, 41 and 46 monomeric subunits (DODA-PG34, DODA-PG41 and DODA-PG46, respectively) in accordance with Scheme 1 as shown in FIG. 1D.

### Synthesis of 5-hydroxy-N,N-dioctadecylpentanamide (DODA_1)

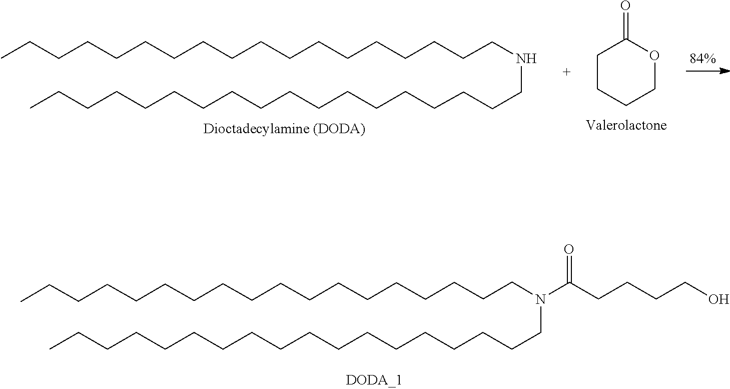 [SMILES: CCCCCCCCCCCCCCCCCCNCCCCCCCCCCCCCCCCCC ; O=C1CCCCO1 ; CCCCCCCCCCCCCCCCCCN(CCCCCCCCCCCCCCCCCC)C(=O)CCCCO]

In an oven dried round bottom flask, freshly prepared suspension of aluminum chloride (1.7 g, 12.75 mmol) and chloroform (10 mL)) was cooled down to 0° C., then triethylamine (2.3 mL in 5.4 mL chloroform) was added to it and allowed to stir under N₂ atmosphere. After addition of trimethylamine, the reaction temperature was raised to the room temperature. In a separate pressure vial, DODA (4.0 g, 7.7 mmol) was dissolved in chloroform (75 mL) and transferred to the oil bath (preset temperature of 55° C.). To the solution of DODA, valerolactone (0.51 g, 5.1 mmol) and AlCl₃ suspension (dropwise) was added consequently. Reaction was allowed to stir for 3 hours at 55-60° C. After completion, the reaction mixture was cooled down to room temperature and quenched with 30 mL of H₂O. The organic layer was washed with H₂O (2×150 mL) and brine (150 mL), dried over anhydrous Na₂SO₄, and evaporated under reduced pressure at rotovap to obtain crude (3.4 g, 84%). The product was used for the next step without purification.

¹H NMR (300 MHz, Chloroform-d₃) δ ppm: 3.60 (m, 2H), 3.14-3.32 (m, 4H), 2.41 (s, 1H), 2.33 (t, J=6.8 Hz, 2H), 1.40-1.77 (m, 8H), 1.09-1.33 (m, 68H), 0.87 (t, J=6.6 Hz, 6H).

### Synthesis of DODA_2 (n=34)

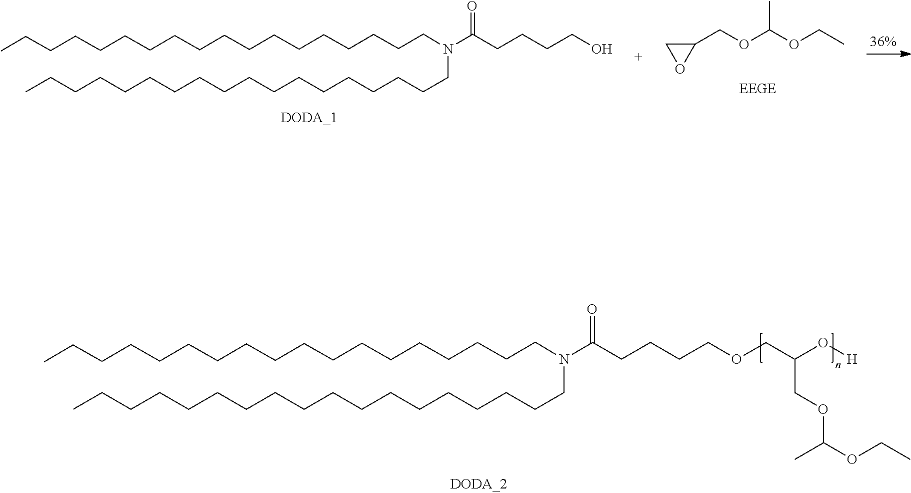

2,3-Epoxy-1-(1-ethoxyethoxy)propane (EEGE) was dried before use by co-evaporation with toluene three times. Compound DODA-1 was also co-evaporated with toluene to azeotrope off any water present and kept over P₂O₅ overnight on high vacuum line. The reaction was carried out under under inert atmosphere and super dry conditions. To a solution of DODA_1 (0.1 g, 0.16 mmol) in toluene in a Schlenk line tube, was added phosphazene base P4-t-Bu solution (0.2 mL, 0.8 M in hexane) under argon atmosphere, and allowed to stir for 15 minutes. Subsequently, 2,3-epoxy-1-(1-ethoxyethoxy)propane) (EEGE) (1.1 g, 7.5 mmol) was added dropwise to the reaction mixture and was allowed to stir overnight under argon atmosphere. The reaction was quenched with 0.1 g benzoic acid and the solvent was evaporated using a rotavapor. The crude was purified using C₄ column using H₂O and MeOH as eluent to afford DODA_2 (0.32 g, 36%).

MALDI-TOF: 3980 (MW(n=34)+Na+) ¹H NMR (300 MHz, d-chloroform) δ ppm: δ 4.69 (q, J=5.4 Hz, 38H), 3.83-3.95 (m, 2H), 3.42-3.65 (m, 272H), 3.24-3.31 (m, 2H), 3.15-3.19 (m, 2H), 1.59-1.63 (m, 27H), 1.17-1.28 (m, 305H), 0.87 (t, J=6.0 Hz, 6H).

### Synthesis of DODA_2 (n 41)

To a solution of DODA_1 (0.17 g, 0.28 mmol) in toluene in a Schlenk line tube under argon atmosphere, was added phosphazene base P4-t-Bu solution (0.2 mL, 0.8 M in hexane) and stirred for 15 minutes. Subsequently, 2,3-epoxy-1-(1-ethoxyethoxy)propane) (EEGE) (1.1 g, 7.5 mmol) was added dropwise to the reaction mixture and stirred overnight under argon atmosphere The reaction was quenched with 0.1 g benzoic acid and concentrated using rotavapor. The crude was purified using C4 column using H₂O and MeOH as eluent to afford DODA_2 (0.32 g, 36%).

¹H NMR (300 MHz, CDCl₃-d₃)) δ ppm: δ 4.69 (q, J=5.4 Hz, 38H), 3.83-3.95 (m, 2H), 3.42-3.65 (m, 272H), 3.24-3.31 (m, 2H), 3.15-3.19 (m, 2H), 1.59-1.63 (m, 27H), 1.17-1.28 (m, 305H), 0.87 (t, J=6.0 Hz, 6H).

EEGE was azeotrope with toluene and desiccated with P₂O₅ before the rection. DODA_1 was also desiccated with P₂O₅.

### Synthesis of DODA_2A (n 46)

To a solution of DODA_1 (0.4 g, 0.06 mmol) in toluene in a Schlenk line tube under argon atmosphere was added phosphazene base P4-t-Bu solution (0.2 mL, 0.8 M in hexane) and stirred for 15 minutes. Then, 2,3-epoxy-1-(1-ethoxyethoxy)propane) (EEGE) (1.1 g, 7.5 mmol) was added dropwise to the reaction mixture and stirred overnight under argon atmosphere. The reaction was quenched with 0.1 g benzoic acid and concentrated using rotovap. The crude was purified using C₄ column using H₂O and MeOH as eluent to afford DODA_2A (0.38 g, 36%).

MALDI-TOF: 7365 (MW(n=46)+Na⁺) ¹H NMR (300 MHz, CDCl₃-d₃) δ ppm: 4.64-4.85 (m, 48H), 3.22-3.35 (m, 392H), 1.10-1.40 (m, 393H), 0.8 (m, 6H).

EEGE was azeotrope with toluene and desiccated with P₂O₅ before the rection. DODA_1 was also desiccated with P₂O₅.

### Synthesis of DODA-PG34

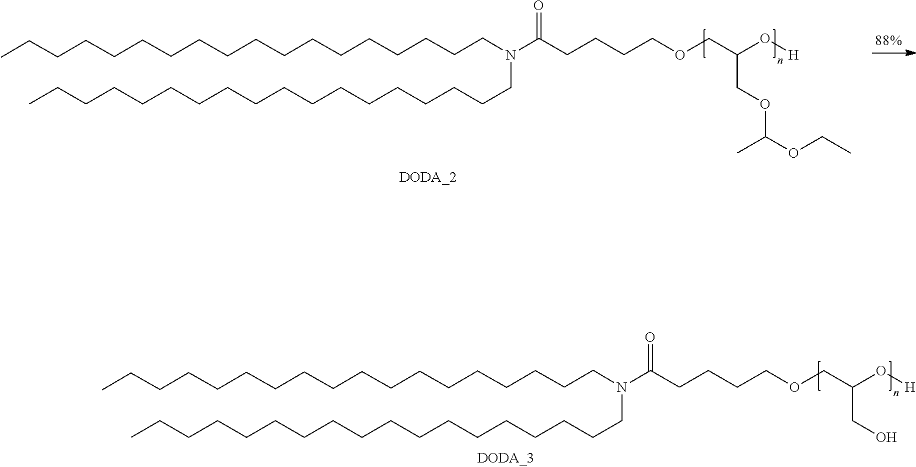

To a solution of DODA_2 in MeOH was added HCl (0.1 mL, 1M in ethyl acetate) dropwise and stirred for 4 hours at room temperature. Subsequently, the reaction mixture was evaporated at rotavapor to obtain white solid. Further, trituration was done by dissolving the white solid in a minimum amount of methanol and adding Et₂O (chilled). After addition of Et₂O, solid crashed out, and then the mixture was centrifuged (4.4 RPM for 10 min) to separate the product DODA_3 (0.16 g, 88%).

HPLC/ELSD: >98.6%. MALDI-TOF: 3533 (MW+23(Na)). ¹H NMR (300 MHz, DMSO-d₆)) δ ppm: δ 3.72-4.59 (m, 42H), 3.27-3.64 (m, 233H), 3.13-3.22 (m, 7H), 1.38-1.58 (m, 10H), 1.23 (s, 61H), 0.85 (t, J=5.9 Hz, 6H). FIG. 1A is a MALDI-TOF spectrum of DODA-PG34.

### Synthesis of DODA-PG41

To a solution of DODA_2 in MeOH was added HCl (0.1 mL, 1M in ethyl acetate) dropwise and stirred for 4 hours at room temperature. Subsequently, the reaction mixture was concentrated via rotavapor. The white solid formed and was dissolved in a minimum amount of MeOH, and ice cold Et₂O was added to precipitate out the product. The mixture was centrifuged (4.4 RPM for 10 min) to separate the product DODA-PG41.

HPLC/ELSD: >99%. MALDI-TOF: 3682.36 (MW+23(Na)). ¹H NMR (300 MHz, DMSO-d₆)) δ ppm: δ 3.72-4.59 (m, 42H), 3.37-3.53 (m, 240H), 3.22-3.13 (m, 7H), 2.49-2.51 (m, 50H), 2.12-2.23 (m, 2H), 1.58-1.38 (m, 9H), 1.23 (s, 61H), 0.85 (t, J=5.9 Hz, 6H).

### Synthesis of DODA-PG46

To a solution of DODA_2A in MeOH was added HCl (0.1 mL, 1M in ethyl acetate) dropwise and stirred for 4 hours at room temperature. Subsequently, the reaction mixture was concentrated via rotovapor. The white solid was formed and was dissolved in a minimum amount of MeOH, and cooled Et₂O was added to precipitate out the product. The mixture was centrifuged (4.4 RPM for 10 min) to separate the product (DODA-PG46). (0.22 g, 88%). HPLC/ELSD: >98%. MALDI-TOF: 4051.93 (MW+23(Na)).

¹H NMR (300 MHz, DMSO-d₆)) δ ppm: δ 3.37-3.54 (m, 598H), 2.20-2.33 (m, 6H), 1.38-1.58 (m, 6H), 1.23 (s, 56H), 1.07-1.11 (m, 56H), 0.85 (t, J=5.9 Hz, 6H).

### Example 2. Alternative Synthesis of Polymer-Conjugated Lipids

The goal of this experiment was to synthesize exemplary polymer-conjugated lipids for use in LNPs using a synthesis method that is different from the synthesis method described in Example 1. In this experiment, the polymer-conjugated lipids synthesized were dioctadecylamine (DODA) conjugated to polyglycerol containing 45 and 58 monomeric subunits (DODA-PG45 and DODA-PG58, respectively) in accordance with Scheme 2 as shown in FIG. 1E.

### Scheme 2

### Synthesis of 5-(benzyloxy)pentanoic acid (2)

Compound 1 (5.02 g, 25.8 mmol) was dissolved in 70 mL of acetone, cooled to 0° C. and treated with 42 mL (85.1 mmol) of Jones reagent (2.0 M CrO₃ in H₂SO₄). The reaction mixture was stirred for 2 hours at ambient temperature and quenched by addition of 15 mL of i-PrOH at 0° C. The mixture was diluted with 200 mL of EtOAc and washed twice with water, brine and dried over Na₂SO₄. The solvents were distilled off, providing 5.2 grams of compound 2, which was used in the next step without further purification.

¹H NMR (300 MHz, d-chloroform) δ ppm: 7.25-7.40 (m, 5H), 4.50 (2H), 3.49 (t, J=6.3 Hz, 2H), 2.31-2.44 (m, 2H), 1.60-1.80 (m, 4H).

### Synthesis of 5-hydroxy-N,N-dioctadecylpentanamide (3)

Compound 2 (1.37 g, 6.58 mmol) was dissolved in 50 mL of chloroform, and DIPEA (3.35 mL, 24.8 mmol) was added, followed by DMAP (0.19 g 1.5 mmol), DODA (3.26 g, 6.21 mmol) and HATU (2.9 g, 7.45 mmol). The coupling reaction was run at 48-50° C. overnight. The reaction mixture was cooled to ambient temperature, diluted with dichloromethane and washed with NaHCO₃ (sat), water and brine. The organic layer was dried over Na₂SO₄, filtered and concentrated. The crude material was purified by normal phase column chromatography (Hexanes-EtOAc), providing 3.6 g (81% yield) of amide 3.

¹H NMR (300 MHz, d-chloroform) δ ppm: 7.25-7.40 (m, 5H), 4.50 (2H), 3.49 (t, J=6.3 Hz, 2H), 3.22-3.35 (m, 2H), 3.12-3.25 (m, 2H), 2.25-2.35 (m, 2H), 1.60-1.80 (m, 4H), 1.40-1.55 (m, 4H), 1.10-1.33 (m, 65H), 0.85-0.95 (m, 6H).

### Synthesis of 5-hydroxy-N,N-dioctadecylpentanamide (DODA-1) (4)

Compound 3 (3.6 g, 5.1 mmol) was dissolved in MeOH/EtOAC (70 mL/100 mL) mixture and underwent deprotection reaction using 0.5 g of Pd/C in a Parr reactor under 40 psi. The conversion was quantitative, providing 3.1 grams of DODA-1 which was used for the next step without further purification.

¹H NMR (300 MHz, d-chloroform) δ ppm: 3.61 (t, J=6.0 Hz, 2H), 3.22-3.35 (m, 2H), 3.12-3.25 (m, 2H), 2.30-2.40 (m, 2H), 2.15 (br s, 1H), 1.70-1.83 (m, 2H), 1.40-1.75 (m, 6H), 1.1-1.4 (m, 63H), 0.85-0.95 (m, 6H).

### Synthesis of DODA_2 (n 45)

2,3-Epoxy-1-(1-ethoxyethoxypropane) was dried before use by co-evaporation with toluene three times. Compound DODA-1 was also co-evaporated with toluene to azeotrope off any water present and kept over P₂O₅ overnight on high vacuum line. The reaction was carried under inert atmosphere and very dry conditions.

Compound DODA-1 (0.2 g, 0.32 mmol, 1 eq.) was dissolved in 2 mL of dry toluene and a catalytic amount of P4-tBu (0.4 mL, 0.8 M in hexane) was added. The reaction mixture was stirred for 20-30 minutes at ambient temperature, followed by addition of dry 2,3-Epoxy-1-(1-ethoxyethoxypropane) (4.49 g, 30.7 mmol, 96 eq.) in 1 mL of toluene. The stirring continued for 16 hours and then polymerization reaction was stopped by quenching with solid benzoic acid (˜300 mg), stirred for 20 min, concentrated and kept on a vacuum line for 1 hour. The crude was purified by RP chromatography (C₄—40 g, H₂O-i-PrOH) following ELSD signal.

MALDI-TOF revealed formation of 0.5 g oligomer with n=9. This material (0.5 g, 0.26 mmol) was mixed with 1.5 mL of toluene and subjected to further polymerization under conditions described above: P4-tBu (0.36 mL, 0.8 M in hexane), dry 2,3-Epoxy-1-(1-ethoxyethoxypropane) (2.7 g, 18.5 mmol, 71 eq) in 1 mL of toluene. After reaction was run overnight it was quenched with 0.26 grams of benzoic acid, stirred for 15 minutes, concentrated and purified by RP chromatography (C₄—40 g, H₂O-i-PrOH), providing 1.7 g (73%) of polymer with n=45.

MALDI-TOF: 7302 (MW(n=45)+Na⁺) ¹H NMR (300 MHz, d-chloroform) δ ppm: 4.64-4.85 (m, 47H), 3.22-3.35 (m, 378H), 1.10-1.40 (m, 393H), 0.8 (m, 6H).

Synthesis of DODA_2A (n=58)

2,3-Epoxy-1-(1-ethoxyethoxypropane) and compound DODA-1 was dried the way as described above.

The synthesis of DODA_2A (n=58) was done stepwise. Initially DODA with n=23 was synthesized using 60 eq excess of 2,3-epoxy-1-(1-ethoxyethoxypropane) according to procedure written above. DODA with 23 units (0.72 grams, 1.6 mmol) was dissolved in 2 mL of dry toluene and treated with 0.3 mL of P4-tBu (0.8M/hexanes) stirring for 20 minutes before 2,3-epoxy-1-(1-ethoxyethoxypropane) was added (2.3 grams, 144 mmol) in 0.5 mL of toluene. The reaction was stirred overnight and then quenched with 160 mg of benzoic acid and the crude was purified by RP chromatography (C4—40 g, H₂O-i-PrOH) following ELSD signal. 1.7 g of DODA_2A (n=58) was isolated.

MALDI-TOF: 9126 (MW+23(Na)). ¹H NMR (300 MHz, d-chloroform) δ ppm: 4.64-4.85 (m, 65H), 3.22-3.35 (m, 490H), 1.10-1.40 (m, 483H), 0.8 (m, 6H).

### Synthesis of DODA-PG45

DODA_2 (1.7 g, 0.24 mmol) was dissolved in MeOH (44 mL) and treated with of 1N HCl/EtOAc (0.45 mL, 0.45 mmol) and stirred for 4 hours at ambient temperature. The reaction mixture was concentrated dissolved in 4 mL of MeOH and treated with 35 mL of ice-cold Et₂O.

The cloudy-oily mixture was centrifuged at 4.4×10³×g for 10 min, the solvents were decanted and sonication procedure was repeated two times using 35 ml of Et₂O. After decanting the last supernatant a light brown oil residue was obtained which was filtered under N₂-blanket, washed with ice cold ether and kept under vacuum over P₂O₅, providing 910 mg (95%) of off-white solid-DODA-PG45.

HPLC/ELSD: >98.6%. MALDI-TOF: 3980 (MW+23(Na)). ¹H NMR (300 MHz, d-chloroform) δ ppm: 3.30-4.50 (m, 390H), 2.20-2.33 (m, 2H), 1.40-1.50 (m, 8H), 1.15-1.30 (m, 63H), 0.8 (m, 6H). FIG. 1B is a MALDI-TOF spectrum of DODA-PG45.

### Synthesis of DODA-PG58

DODA_2A (1.5 g, 0.19 mmol) was dissolved in MeOH (40 mL) and treated with 1N HCl/EtOAc (0.4 mL, 0.4 mmol) and stirred for 4 h at ambient temperature. The reaction mixture was concentrated, dissolved in 3 mL of MeOH and treated with 30 mL of ice-cold Et₂O.

The cloudy-oily mixture was centrifuged at 4.4×103×g for 10 minutes, the solvents were decanted, and the sonication procedure was repeated the same way as described for the analog above providing 770 mg (93%) of DODA-PG58.

HPLC/ELSD: >98%. MALDI-TOF: 4938 (MW+23(Na)). ¹H NMR (300 MHz, d-chloroform) δ ppm: 3.80-4.75 (br s, 88H), 3.20-4.60 (m, 383H), 2.20-2.30 (m, 2H), 1.40-1.50 (m, 8H), 1.15-1.30 (m, 63H), 0.8 (m, 6H). FIG. 1C is a MALDI-TOF spectrum of DODA-PG58.

### Example 3. Preparation of LNPs Comprising Polymers PEG and PG Conjugated Lipids

The goal of this experiment was to prepare LNP formulations using different anchored polymers. LNP formulations were prepared using polymer-conjugated lipids such as DSPE-PEG2K-OH, DODA-PG45, and DSPE-PMPC50, in the same way. The specific formulations that were prepared in this experiment are shown and described in Table 8.

Generally, LNPs were prepared as follows: a lipid composition described in Table 8 dissolved in ethanol was mixed with an aqueous solution of DNA at pH 4. The resulting mixture was exhaustively dialyzed against a phosphate buffered saline (PBS) solution and then concentrated using spin filtration. The LNP was characterized using dynamic light scattering to measure size and polydispersity index (PDI), and Picogreen fluorescent method to quantify encapsulation efficiency (EE). For dosing of animals, the solutions were diluted to the desired concentration using phosphate buffered saline solution.

TABLE 8

| | Diameter | | EE | |
|---|---|---|---|---|
| Formulation | (nm) | PDI | (%) | Lipid composition |
| 179 | 73.5 | 0.055 | 98.0 | Lipid 87:DSPC:Chol:DSPE- |
| | | | | PEG2K-OH |
| | | | | (47.5:10.0:39.5:3.0) |
| 180 | 72.5 | 0.048 | 96.6 | Lipid 87:DSPC:Chol:DSPE- |
| | | | | PEG2K-OH:DSPE-PEG2K- |
| | | | | GalNAc3 |
| | | | | (47.5:10.0:39.5:2.5:0.5) |
| 181 | 83.2 | 0.038 | 98.4 | Lipid 87:DSPC:Chol:DODA- |
| | | | | PG45:DSPE-PEG2K-OH |
| | | | | (47.5:10.0:40.5:1.5:0.5) |
| 182 | 81.8 | 0.056 | 97.7 | Lipid 87:DSPC:Chol:DODA- |
| | | | | PG45:DSPE-PEG2K-GalNAc3 |
| | | | | (47.5:10.0:40.5:1.5:0.5) |
| 183 | 75.2 | 0.047 | 98.0 | Lipid 87:DSPC:Chol:DODA- |
| | | | | PG45:DSPE-PEG2K-OH |
| | | | | (47.5:10.0:39.5:2.5:0.5) |
| 184 | 76.0 | 0.046 | 96.1 | Lipid 87:DSPC:Chol:DODA- |
| | | | | PG45:DSPE-PEG2K-GalNAc3 |
| | | | | (47.5:10.0:39.5:2.5:0.5) |

### Example 4. In Vivo Expression of Nucleic Acids in LNP Formulations Containing Different Anchored Polymers

The goal of this study was to evaluate the in vivo expression of nucleic acids that are formulated and comprise of LNPs with various anchored polymers, such as DSPE-PEG2K-OH or DODA-PG45. To this end, CD-1 mice (males) were intravenously (IV) injected with ceDNA nucleic acid carrying a firefly luciferase reporter construct that was formulated in LNPs comprising DSPE-PEG2K-OH or DODA-PG45 (composition in Table 8) at a dose of 0.5 mg/kg (0 day).

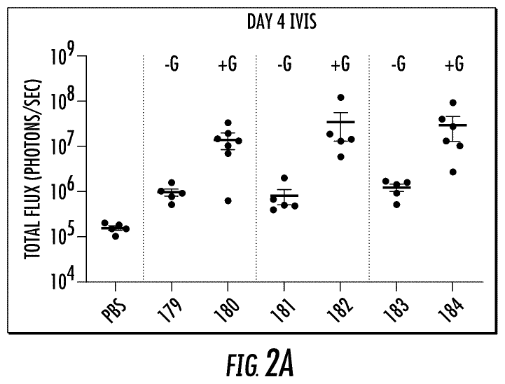

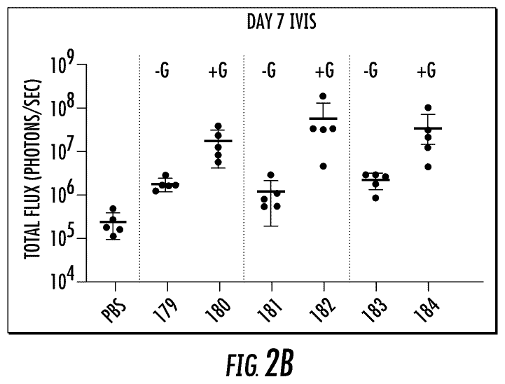

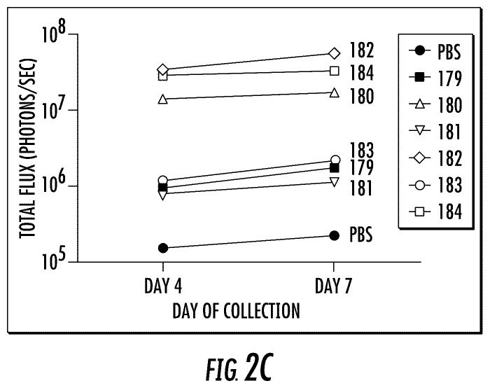

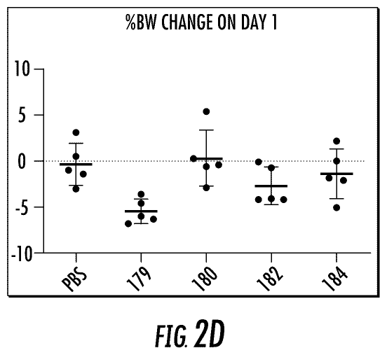

Whole-body luciferase bioluminescence was measured by In Vivo Imaging System (IVIS) at Day 4 and Day 7. FIG. 2A shows the total flux measured by the total photon counts per the region of interest, i.e., the liver, measured by IVIS at Day 4 post-dosing for tested LNPs and for a negative control (PBS) injected with saline instead of formulated ceDNA. FIG. 2B shows the total flux measured for tested LNPs and negative control at Day 7 post-dosing. FIG. 2C shows the total flux measured for tested LNPs and negative control across two collection days (Day 4 and Day 7). The results shown in FIG. 2C indicate that administration of formulated LNPs with different anchored polymers in combination with a targeting ligand, i.e., GalNAc3 (Formulations 180, 182, and 184) results in higher expression of luciferase as compared to untagged LNPs (Formulations 179, 181, and 183) at both Day 4 and Day 7. FIG. 2D shows the percentage change in body weight (BW) of mice at Day 1 post-dosing. The results indicate that the tested LNPs with targeting ligand GalNAc3 (Formulations 180, 182, and 184), caused a smaller change in body weight in mice as compared to untagged LNPs.

The results presented in Example 4 demonstrate that a GalNAc3 targeted LNP of the disclosure comprising anchored polymers (DSPE-PEG2K-OH or DODA-PG45) when delivered in vivo supports the expression of nucleic acids without triggering any major tolerability issues and other adverse events in

Unexpectedly, the data presented in Example 4 demonstrate that only half the amount (1.5 mol %) of DODA-PG45 in an untargeted LNP formulated with PG-containing anchored polymer (Formulation 181), resulted in about the same level of transgene expression as compared to double the amount (3.0 mol %) of DSPE-PEG2K-OH in an untargeted LNP formulated with PEG-containing anchored polymer (Formulation 179). The significance of this unexpected discovery on achieving advantageous stealth/endosomal tradeoff for PG-containing LNP formulations relative to PEG-containing LNP formulations is further explored in the subsequent Examples.

### Example 5. Analysis of Luciferase mRNA Expression in mRNA Formulated LNP

The goal of this study was to quantify the expression of a luciferase mRNA formulated with LNPs containing different anchored polymers. LNP formulations used in this study are shown in Table 9. Freshly isolated mouse hepatocytes were seeded on collagen-coated plates at a cell density of 25,000 per well. The assay plate was then incubated for 4 hours at 37° C., 5% CO₂ in a humidified incubator to allow for cell attachment. Following the attachment period, each well was treated for 1 hour with 100 ng of mRNA formulated LNPs containing no mouse serum. Following treatment, the plate was washed twice in 1×dPBS and maintenance media was added to each well. The following morning, viability readouts were collected using CelltiterFluor and expression readout was collected using “One-Step” Luciferase Assay on a SpectraMax spectrometer. The final analytics were plotted as RLU values of Luciferase Activity which represent Luciferase expression normalized by viability. The negative control in this experiment was a high expressing LNP (Formulation 070) which was either uninhibited or competitively inhibited with 55 μM of Free TetraGalNAc.

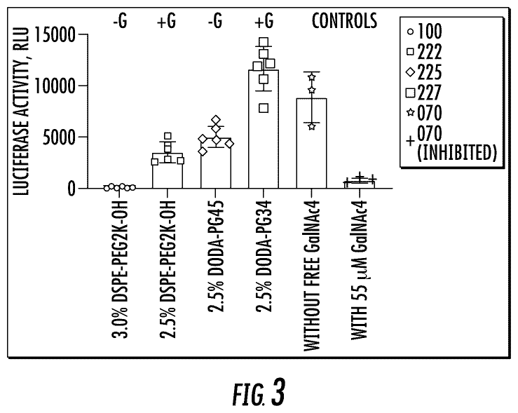

FIG. 3 is a bar graph showing luciferase activity for the tested LNP formulations containing different lipid-anchored polymers. The results are shown in FIG. 3, and indicate that an LNP formulated with helper lipid DSPC, and anchored polymer DODA-PG34 and DSPE-PEG2K-GalNAc3 (Formulation 227) showed higher luciferase activity than uninhibited control.

TABLE 9

| | Diameter | | EF | |
|---|---|---|---|---|
| Formulation | (nm) | PDI | (%) | Lipid composition |
| 100 | 89.8 | 0.128 | 92.7 | Lipid 87:DSPC:Chol:DSPE- |
| | | | | PEG2K-OH:DiD |
| | | | | (47.5:10.0:39.0:3.0:0.5) |
| 222 | 77.9 | 0.047 | 96.1 | Lipid 87:DSPC:Chol:DSPE- |
| | | | | PEG2K-OH:DSPE-PEG2K- |
| | | | | GalNAc3:DiD |
| | | | | (47.5:10.0:39.0:2.5:0.5:0.5) |
| 225 | 80.0 | 0.038 | 96.0 | Lipid 87:DSPC:Chol:DODA- |
| | | | | PG45:DSPE-PEG2K- |
| | | | | GalNAc3:DiD |
| | | | | (47.5:10.0:39.0:2.5:0.5:0.5) |
| 227 | 81.5 | 0.046 | 96.4 | Lipid 87:DSPC:Chol:DODA- |
| | | | | PG34:DSPE-PEG2K- |
| | | | | GalNAc3:DiD |
| | | | | (47.5:10.0:39.0:2.5:0.5:0.5) |
| 070 | 73.5 | 0.085 | 94.5 | Lipid Z:DSPC:Chol:DSPE- |
| | | | | PEG2K-OH:DSPE-PEG2K- |
| | | | | GalNAc3:DiD |
| | | | | (47.5:10.0:39.0:2.5:0.5:0.5) |

Ionizable Lipid Z (structure not shown) belongs to a different class of ionizable lipids compared to Ionizable Lipid 87, where both the headgroup and lipid tail moieties are structurally different from those of Ionizable Lipid 87.

### Example 6. Evaluation of Opsonization-Driven Uptake of LNPs in Primary Mouse Hepatocytes

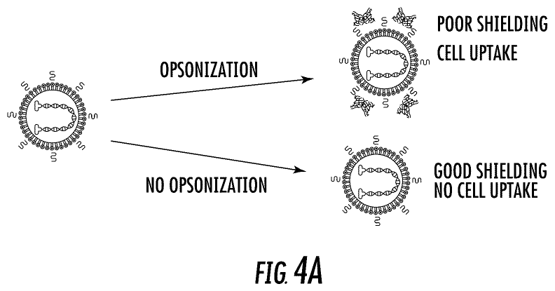

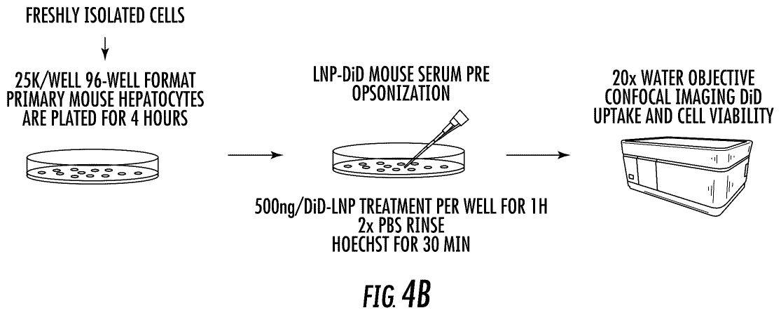

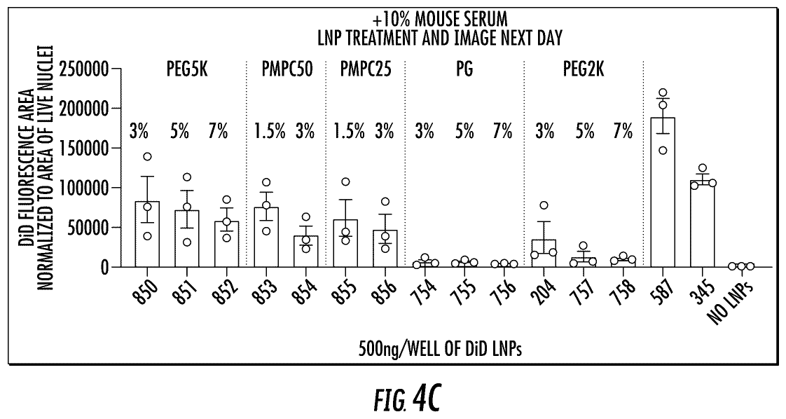

The goal of this assay was to screen for “stealthy” LNPs via differential uptake of the fluorophore DiD in primary mouse hepatocytes via mouse serum opsonization. Without being bound by a specific theory, it is hypothesized that stealthy untargeted LNPs bind to minimal proteins from the serum, thus leading to minimal primary hepatocyte uptake, whereas non-stealthy LNPs bind to serum proteins, leading to a high cell uptake. This hypothesis is depicted as a schematic in FIG. 4A.

A schematic of the assay is shown in FIG. 4B. Briefly, freshly isolated hepatocytes were seeded on collagen-coated plates at a cell density of 25,000 per well, after which the assay plate was incubated for 4 hours at 37° C., 5% CO₂, in a humidified incubator. After the attachment period, each well was treated with 500 ng of DiD-labeled LNP containing 10% mouse serum for 1 hour. LNP formulations evaluated in this study are shown in Table 10. Following treatment, the plate was washed twice in 1×dPBS and maintenance media was added to each well. The following morning, an image of the plate was obtained on a Phenix confocal microscope using a 20× water objective. Image analysis was performed and the data were plotted as DiD fluorescence area normalized to area of live nuclei.

FIG. 4C is a bar graph showing DiD fluorescence normalized to area of live nuclei measured for the various LNP formulations containing different lipid-anchored polymers. The results shown in FIG. 4C indicate that LNP formulations comprising the DODA-PG45 anchored polymer showed minimal primary hepatocyte uptake. These results suggest that among all anchored polymers tested, PG anchored polymers performed the best in inhibiting opsonization-driven uptake.

TABLE 10

| | Diameter | | EE | |
|---|---|---|---|---|
| Formulation | (nm) | PDI | (%) | Lipid composition |
| 850 | 101.2 | 0.10 | 98 | Lipid Z:DSPC:Chol:DSG-PEG5K-OMe:DiD |
| | | | | (47.5:10.0:39.0:3.0:0.5) |
| 851 | 106.4 | 0.06 | 98 | Lipid Z:DSPC:Chol:DSG-PEG5K-OMe:DiD |
| | | | | (47.5:10.0:37.0:5.0:0.5) |
| 852 | 112.2 | 0.07 | 98 | Lipid Z:DSPC:Chol:DSG-PEG5K-OMe:DiD |
| | | | | (47.5:10.0:35.0:7.0:0.5) |
| 853 | 84.5 | 0.07 | 95 | Lipid Z:DSPC:Chol:DSPE-PMPC50:DiD |
| | | | | (47.5:10.0:40.5:1.5:0.5) |
| 854 | 63.1 | 0.17 | 95 | Lipid Z:DSPC:Chol:DSPE-PMPC50:DiD |
| | | | | (47.5:10.0:39.0:3.0:0.5) |
| 855 | 91.1 | 0.07 | 99 | Lipid Z:DSPC:Chol:DSPE-PMPC25:DiD |
| | | | | (47.5:10.0:40.5:1.5:0.5) |
| 856 | 74.8 | 0.10 | 97 | Lipid Z:DSPC:Chol:DSPE-PMPC25:DiD |
| | | | | (47.5:10.0:39.0:3.0:0.5) |
| 754 | 78.58 | 0.05 | 96.88 | Lipid Z:DSPC:Chol:DODA-PG45:DiD |
| | | | | (47.5:10.0:39.0:3.0:0.5) |
| 755 | 80.99 | 0.20 | 93.13 | Lipid Z:DSPC:Chol:DODA-PG45:DiD |
| | | | | (47.5:10.0:37.0:5.0:0.5) |
| 756 | 72.40 | 0.25 | 82.75 | Lipid Z:DSPC:Chol:DODA-PG45:DiD |
| | | | | (47.5:10.0:35.0:7.0:0.5) |
| 204 | 77.31 | 0.03 | 93.92 | Lipid Z:DSPC:Chol:DSG-PEG2K-OMe:DiD |
| | | | | (47.5:10.0:39.0:3.0:0.5) |
| 757 | 73.94 | 0.09 | 91.31 | Lipid Z:DSPC:Chol:DSG-PEG2K-OMe:DiD |
| | | | | (47.5:10.0:37.0:5.0:0.5) |
| 758 | 70.68 | 0.16 | 86.56 | Lipid Z:DSPC:Chol:DSG-PEG2K-OMe:DiD |
| | | | | (47.5:10.0:35.0:7.0:0.5) |
| 587 | 76.21 | 0.11 | 92.97 | Lipid Z:DSPC:Chol:DMG-PEG2K:DiD |
| | | | | (47.5:10.0:39.0:3.0:0.5) |
| 345 | 95.77 | 0.12 | 98.04 | Lipid Z:C2 ceramide:Chol:DSG-PEG2K-OMe:DiD |
| | | | | (47.5:10.0:39.0:2.5:0.5) |

### Example 7. Evaluation of the Effect of Anchored Polymer Composition on Opsonization-Driven LNP Uptake in Primary Mouse Hepatocytes

The aim of this study was to screen for “stealthy” LNP formulations comprising polymer-conjugated lipids of the present disclosure that varied in their identity and percentage composition, wherein a stealthy LNP is defined as one that has minimal uptake into cells in the absence of a targeting ligand. The benchmark LNP was prepared using 47.5% Lipid Z, 10% DSPC, 39% cholesterol, 3% DSG-PEG2K, and 0.5% DiD. As the % of polymer is decreased the amount of cholesterol is increased to compensate. Freshly isolated hepatocytes were seeded on collagen-coated plates at a cell density of 25,000 per well, after which the assay plate was incubated for 4 hours at 37° C., 5% CO₂, in a humidified incubator. After the attachment period, each well was treated with 500 ng of DiD-labeled LNP containing 10% Mouse Serum for 1 hour. Following treatment, the plate was washed twice in 1×dPBS and maintenance media was added to each well. The following morning, an image of the plate was obtained on a Phenix confocal microscope using a 20× water objective.

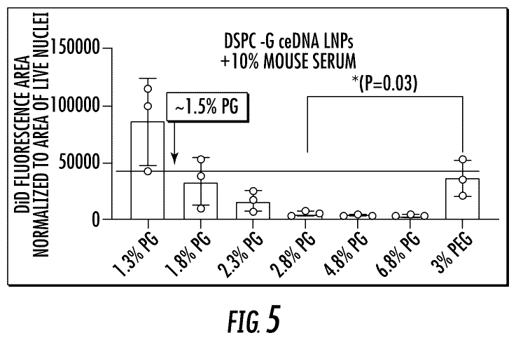

FIG. 5 is a bar graph showing DiD fluorescence area normalized to area of live nuclei for the tested LNP formulations containing different amounts of polyglycerol-conjugated lipids, wherein it is apparent that the use of 1.8% DODA-PG within the LNP provides a comparable level of stealth protection compared to 3% PEG. Additionally, as the amount of DODA-PG is increased, the level of non-targeted uptake is decreased compared to the 3% PEG benchmark, indicating that one could use a lower amount of PG and maintain a similar level of stealthiness. The results shown in FIG. 5 indicate that significantly lower opsonization-driven uptake is observed for LNPs containing 2.8-6.8 percent (%) of PG as compared to LNPs containing 3% PEG, thereby suggesting that PG may provide better shielding to the LNP base composition than 3% PEG.

### Example 8. Evaluation of Endosomal Escape of LNP Formulations

The aim of this study was to evaluate the efficiency of endosomal escape of LNP formulations comprised of anchored polymers that varied in their identity and percentage composition. The benchmark LNP was prepared using 47.5% Lipid Z, 10% DSPC, 39% cholesterol, 2.95% DSG-PEG2K, 0.05% DSPE-PEG77-GalNAc3, and 0.5% DiD. As the % of polymer is decreased the amount of cholesterol is increased to compensate. All compositions used 0.05% GalNAc3 as a targeting ligand for active targeting to primary hepatocytes through the ASGPR uptake pathway, regardless of polymer %. Freshly isolated mouse hepatocytes were seeded on collagen-coated plates at a cell density of 25,000 per well, after which the assay plate was incubated for 4 hours at 37° C., 5% CO₂, in a humidified incubator. After the attachment period, each well was treated with 500 ng of DiD-labeled LNP containing 10% mouse serum for 1 hour. Following treatment, the plate was washed twice in 1×dPBS and maintenance media was added to each well. The following morning, viability readouts were collected using CelltiterFluor and expression readout was collected using “One-Step” Luciferase Assay on a SpectraMax spectrometer. DiD uptake was quantified from images obtained on a Phenix confocal microscope using a 20× water objective. The results were plotted as Luciferase (mLuc) expression normalized to DiD uptake, which represents expression normalized by viability and DiD uptake. When the uptake and mLuc expression are compared as a ratio, one can indirectly surmise the ability of each composition to escape the endosome.

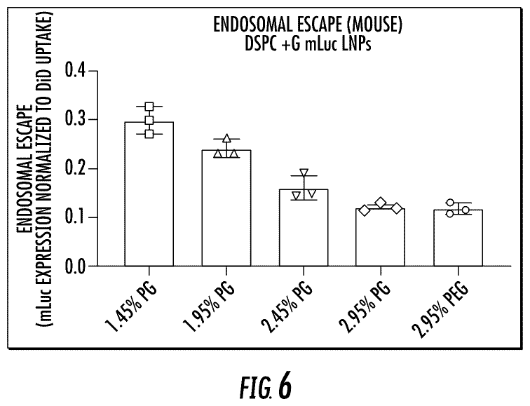

FIG. 6 is a bar graph showing the amount of endosomal escape measured as the amount of luciferase expression normalized to DiD uptake in mouse hepatocytes treated with LNP formulations containing different amounts of polyglycerol-conjugated lipids and a control. It is demonstrated in FIG. 6 that by increasing the amount of DODA-PG, the ability for the LNP to escape the endosome is reduced. This is important when the data from FIG. 5 is considered where less PG can result in a higher level of stealthiness. Thus, an LNP containing less DODA-PG can be used to achieve a similar level of stealth while also enhancing the endosomal escape potential of the LNP.

Taken together, the results described in Examples 6, 7 and 8, along with a comparison of FIGS. 4, 5 and 6 suggest that PG-containing LNPs display a more advantageous stealth/endosomal escape trade off compared to PEG-containing LNPs. In particular, FIG. 5 shows that LNPs formulated with 2.8% of PG was significantly more stealthy compared to LNPs formulated with about the same amount (3%) of PEG. FIG. 5 also shows that LNPs formulated with ˜1.5% PG, as indicated by the arrow, would have about the same level of stealthiness of LNPs formulated with 3% PEG, as indicated by the horizontal line. In contrast, FIG. 6 shows the inverse relationship between the amount of an polymer-conjugated lipid in an LNP formulation and the level of endosomal escape.

Specifically, FIG. 6 shows that LNPs formulated with a relatively low amount (1.45%) of PG maintained a relatively high level of endosomal escape compared to LNPs formulated with significantly higher amount (2.95%) of PG or PEG. The ability of LNPs formulated with PG-containing anchored polymer to achieve this advantageous stealth/endosomal tradeoff as compared to LNPs formulated with PEG-containing anchored polymer is further supported by FIG. 4 wherein the stealthiness of LNPs formulated with PEG suffers as the amount of PEG decreases, in contrast to LNPs formulated with PG for which the stealthiness does not suffer as the amount of PG decreases.

### Example 9. Analysis of Whole Blood Clearance of LNPs Formulated with Ionizable Lipid: Lipid Z, and Different Polymer-Conjugated Lipids

The goal of this study was to measure and compare the pharmacokinetic (PK) properties of novel LNPs formulations containing the ionizable lipid Lipid Z, along with DSPC, cholesterol and different polymer-conjugated lipids as described in Table 11 with a control LNP formulated with ionizable Lipid 87, cholesterol, and DSG-PEG2K-OMe (Formulation 829). Formulations of control LNP and Lipid Z carrying LNPs were injected via IV bolus in the tail vein of CD-1 mice. Whole blood samples were collected for qPCR at 2 min, 1 hour, 3 hour and 6-hour time-points, and K₂EDTA was added as an anticoagulant at 50 L/aliquot. Body weight, mortality, and clinical observations were recorded.

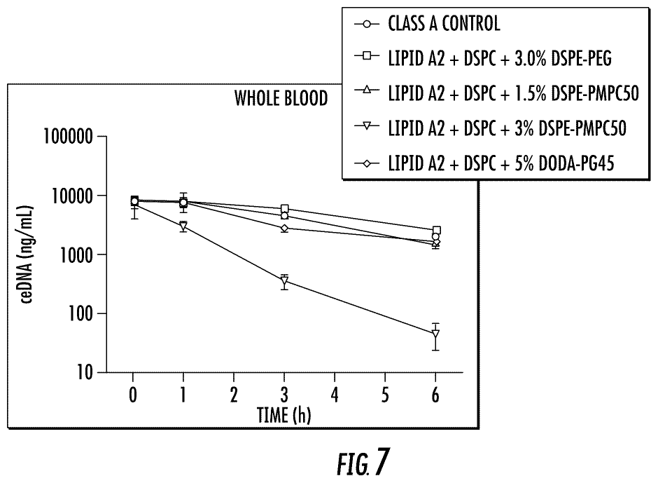

The whole blood clearance of the Control LNP, and the different Lipid Z carrying LNPs are shown in FIG. 7, while individual pharmacokinetic parameters are reported in Table 11. Whole blood concentrations of ceDNA in mice treated with the control LNP and LNPs carrying Lipid Z, DSPC, cholesterol and either 3% DSPE-PEG2K-OMe, or 1.5% DSPE-PMPC50, or 5% DODA-PG45, as measured by the AUCₗₐₛₜ, and t_{1/2} values, were observed to show no significant differences. These results indicate that the higher retention of ceDNA in the bloodstream, and hence the less rapid clearance of the ceDNA-luciferase cargo from the bloodstream as delivered by 3.0% DSPE-PEG, 1.5% DSPE-PMPC50, and 5% DODA-PG45-containing LNPs (Formulations 023, 777, and 678) could be beneficial in the reduction of off-target delivery to non-target cells, including but not limited to blood cells, such as leukocytes, neutrophils, eosinophils, basophils, macrophages, and monocytes, or to immune cells such as T-cells, B-cells, and macrophages.

TABLE 11

| Formulation; | | | | | | | |
|---|---|---|---|---|---|---|---|
| Dose: 1 | Cₘₐₓ | AUCₗₐₛₜ | t_{1/2} | Diameter | | EE | |
| mg/kg | (ng/ml) | (hr*ng/ml) | (hr) | (nm) | PDI | (%) | Lipid Composition |
| 829 | 8130 | 29700 | 2.58 | 93.90 | 0.092 | 96.6 | Lipid 87:Chol:DSG-PEG2K-OMe |
| | | | | | | | (47.5:49.5:3.0) |
| 023 | 7970 | 33400 | 3.17 | 69.38 | 0.062 | 95.6 | Lipid Z:DSPC:Chol/DSPE-PEG2K-OMe |
| | | | | | | | (47.5:10.0:39.5:3.0) |
| 777 | 8560 | 29600 | 2.09 | 82.49 | 0.076 | 96.3 | Lipid Z:DSPC:Chol:DSPE-PMPC50 |
| | | | | | | | (47.5:10.0:41.0:1.5) |
| 740 | 6870 | 7710 | 0.82 | 62.81 | 0.114 | 96.4 | Lipid Z:DSPC:Chol:DSPE-PMPC50 |
| | | | | | | | (47.5:10.0:39.5:3.0) |
| 678 | 8040 | 24500 | 2.34 | 69.49 | 0.046 | 95.3 | Lipid Z:DSPC:Chol:DODA-PG45 |
| | | | | | | | (47.5:10.0:37.5:5.0) |

### Example 10. In Vivo Expression of Nucleic Acids in LNP Formulations Containing Ceramides and Anchored Polymers

The goal of this study was to evaluate the in vivo expression of nucleic acids formulated as LNPs with ceramides as the helper lipid, in combination with various anchored polymers such as DSPE-PEG2K-OH or DODA-PG45. To this end, CD-1 mice (males) were intravenously (IV) injected with ceDNA nucleic acid carrying firefly luciferase reporter construct formulated as in the disclosure with LNPs comprising various helper lipids (ceramide and DSPC) in combination with different anchored polymers (DSPE-PEG2K-OH or DODA-PG45) at 2 different doses of either 1 mg/kg or 2.0 mg/kg (0 day). The LNPs used in the experiment are shown in Table 12.

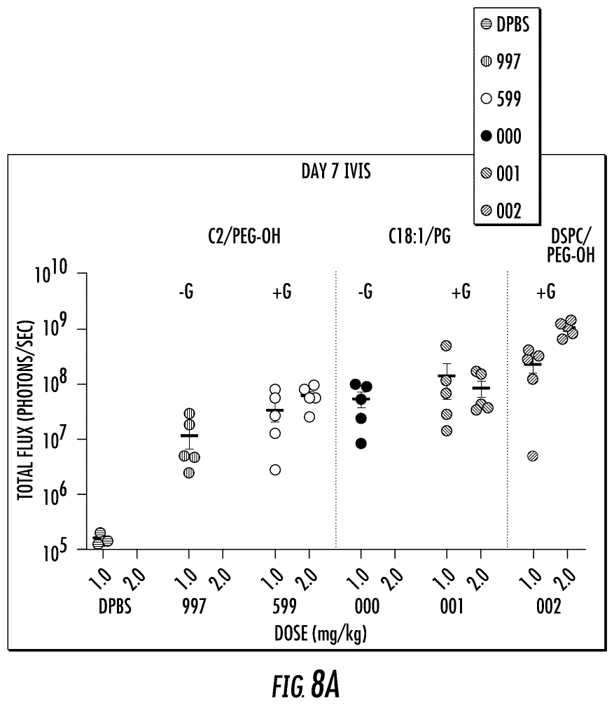

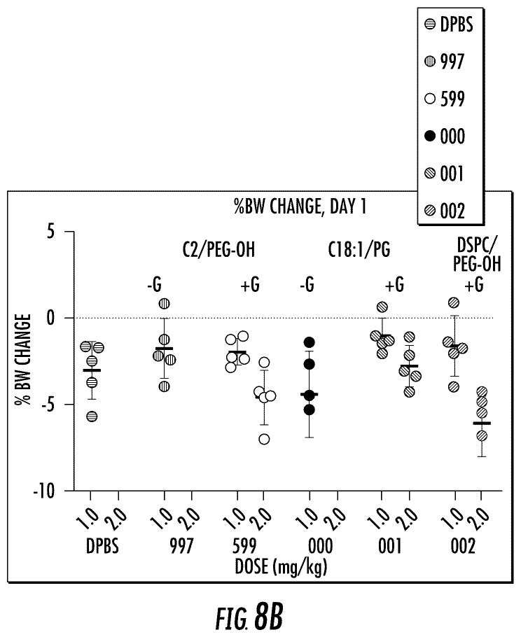

Whole-body luciferase bioluminescence was measured by In Vivo Imaging System (IVIS) at Day 7. FIG. 8A shows the total flux quantified by total photon counts per the region of interest, i.e., the liver, was measured by IVIS at Day 7 post-dosing for tested LNPs and for a negative control (DPBS) injected with saline instead of formulated ceDNA. The results shown in FIG. 8A indicate that administration of formulated LNPs with helper lipid DSPC and polymer conjugated lipid DSPE-PEG2K-OH (formulation no: 002) resulted in a dose-dependent expression of nucleic acid at Day 7, while other LNPs formulated with either C2 ceramide and DSPE-PEG2K-OH or C18:1 ceramide and DODA-PG45 did not show a dose-dependent increase. The percentage change in body weight of mice at Day 1 is shown in FIG. 8B. These results indicate that the tested GalNAc3 targeted LNPs with either DSPC or ceramide helper lipids and different anchored polymers, caused a milder body weight change in mice as compared to an untagged LNP with ceramide helper lipid and DODA-PG45.

Overall, the results presented in this example demonstrate that a GalNAc3 targeted LNP of the disclosure comprising different helper lipids and anchored polymers when delivered in vivo could support expression of nucleic acids without triggering any major tolerability issues and other adverse events in mice that could be clinically observed (e.g., rough hair coat, facial swelling).

TABLE 12

| | Diameter | | EE | |
|---|---|---|---|---|
| Formulation | (nm) | PDI | (%) | Lipid composition |
| 997 | 72.3 | 0.13 | 95.6 | Lipid Z:C2 Ceramide:Cho l:DSG-PEG2K-OH:DSPE-PEG3400-OMe |
| | | | | (47.5:10:39.5:2.5:0.5) |
| 599 | 72.7 | 0.09 | 95.6 | Lipid Z:C2 Ceramide:Chol:DSG-PEG2K-OH:DSPE-PEG3400-GalNAc3A |
| | | | | (47.5:10:39.5:2.5:0.5) |
| 000 | 82.7 | 0.05 | 95.9 | Lipid Z:C18:1 Ceramide:Chol:DODA-PG45:DSPE-PEG3400-OMe |
| | | | | (47.5:10:39.5:2.5:0.5) |
| 001 | 81.6 | 0.08 | 95.5 | Lipid Z:C18:1 Ceramide:Chol:DODA-PG45:DSPE-PEG3400-GalNAc3A |
| | | | | (47.5:10:39.5:2.5:0.5) |
| 002 | 81.1 | 0.09 | 95.4 | Lipid Z:DSPC:Chol:DSG-PEG2K-OH:DSPE-PEG3400-GalNAc3A |
| | | | | (47.5:10:39.5:2.5:0.5) |

### Example 11. Evaluating Immunogenicity of Exemplary LNPs of the Disclosure

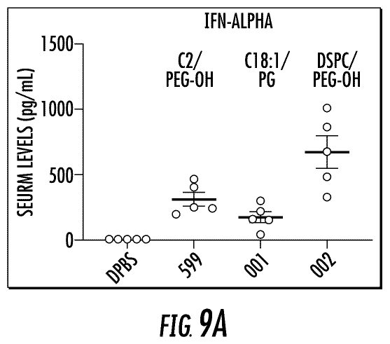

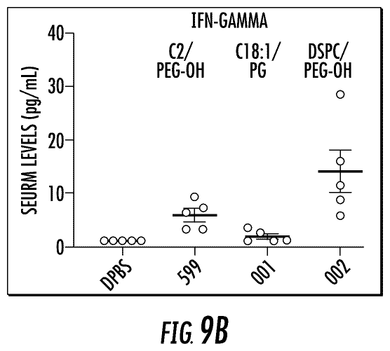

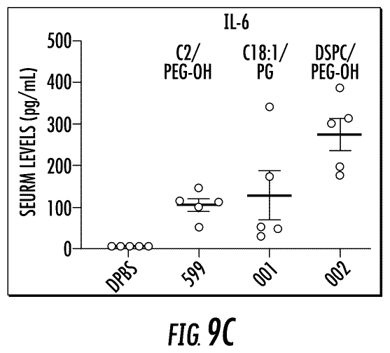

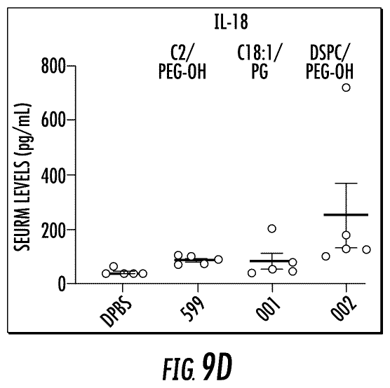

The goal of this study was to evaluate the immunogenicity of exemplary formulated LNPS comprised of DSPC or ceramide helper lipids, and DSG-PEG2K-OH or DODA-PG45 polymer conjugated lipids. The immunogenicity profiles of LNPs containing helper lipids DSPC, C2 ceramide or C18:1 ceramide were compared. The formulations of the LNPs evaluated in this study are given in Table 13. Blood serum was collected at 6 hours post-dosing, and the levels of cytokines that are implicated in the regulation of innate immune response, i.e., IFN-alpha, IL-6, IFN-gamma, TNF-alpha, IL-18, and IP-10 were measured for each animal. The results are shown in FIGS. 9A-9D, and indicate that at a dosage of 2.0 mg/kg, the blood serum levels of IFN-alpha, IL-6, IFN-gamma, TNF-alpha, and IL-18 were lower for the C₂ and C_{18:1} ceramide-containing LNPs as compared to DSPC-containing LNPs. These results also show that some cytokine levels trend lower for PG-containing LNPs, and higher for PEG-containing LNPs, especially in case with IFN-alpha.

These results suggest that the identity of the helper lipid, as well as the identity of the polymer in the anchor lipid-conjugated polymer, directly affects the immunogenicity of LNPs formulated as in the disclosure.

TABLE 13

| | Diameter | | EE | |
|---|---|---|---|---|
| Formulation | (nm) | PDI | (%) | Lipid composition |
| 997 | 72.3 | 0.13 | 95.6 | Lipid Z:C2 Ceramide:Cho l:DSG-PEG2K-OH:DSPE-PEG3400-OMe |
| | | | | (47.5:10:39.5:2.5:0.5) |
| 599 | 72.7 | 0.09 | 95.6 | Lipid Z:C2 Ceramide:Chol:DSG-PEG2K-OH:DSPE-PEG3400-GalNAc3A |
| | | | | (47.5:10:39.5:2.5:0.5) |
| 000 | 82.7 | 0.05 | 95.9 | Lipid Z:C18:1 Ceramide:Chol:DODA-PG45:DSPE-PEG3400-OMe |
| | | | | (47.5:10:39.5:2.5:0.5) |
| 001 | 81.6 | 0.08 | 95.5 | Lipid Z:C18:1 Ceramide:Chol:DODA-PG45:DSPE-PEG3400-GalNAc3A |
| | | | | (47.5:10:39.5:2.5:0.5) |
| 002 | 81.1 | 0.09 | 95.4 | Lipid Z:DSPC:Chol:DSG-PEG2K-OH:DSPE-PEG3400-GalNAc3A |
| | | | | (47.5:10:39.5:2.5:0.5) |

### Example 12. Preparation of DSPE-PEG5k-Mal-Protein

This example describes a method for the preparation of an LNP conjugated to a protein ligand of interest, which requires the inclusion of an additional cysteine residue not present in the native protein sequence. The protein ligand of interest is initially reduced with 10 molar equivalents of TCEP for 30 minutes at 23° C. After reduction, TCEP is removed using a Zeba spin column. The reduced ligand is then incubated for 3 hours at 23° C. with LNPs formulated with DSPE-PEG5k-Maleimide using a mole percentage of 0.5%. The ratio of ligand to DSPE-PEG5k-maleimide is vaned from 0.3 down to 0.02. SDS-PAGE is used to confirm whether the conjugation occurred and to what extent.

### Example 13. Preparation of DSPE-PEG5k-DBCO-Protein

This example describes a method for the preparation of an LNP-conjugated to a protein ligand of interest, which requires the inclusion of an additional cysteine residue not present in the native protein sequence. The protein ligand of interest is initially reduced with 10 molar equivalents of TCEP for 30 minutes at 23° C. After reduction, TCEP is removed using a Zeba spin column. The reduced ligand is then incubated with 10 molar equivalents of Sulfo DBCO-PEG4-maleimide for 3 hours at 23° C. The excess DBCO reagent is then removed using a Zeba spin column. The extent of labelling and overall protein purity is confirmed using a UPLC-QTOF.

The DBCO labelled protein is then incubated for 16 hours at 23° C. with LNPs formulated with DSPE-PEG5K-N₃ using a mole percentage of 0.5%. The ratio of ligand to DSPE-PEG5K-N₃ is vaned from 0.3 down to 0.02. SDS-PAGE is used to confirm whether the conjugation occurred and to what extent.

### Example 14

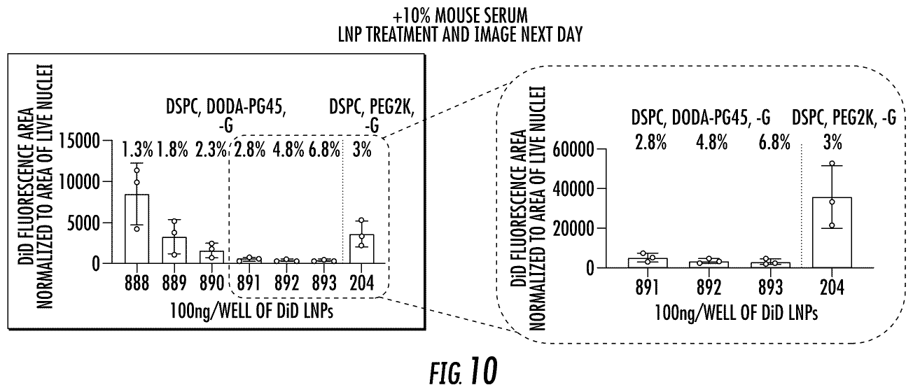

FIG. 10 is a bar graph showing DiD fluorescence area normalized to area of live nuclei for the tested LNP formulations containing different amounts of polyglycerol-conjugated lipids, and formulated with DSPE-PEG5K-N₃ using a mole percentage of 0.5%. The formulations of the LNPs evaluated in this study are given in Table 14. As the amount of DODA-PG is increased, the level of non-targeted uptake was decreased compared to the 3% PEG benchmark, indicating that one could use a lower amount of PG and maintain a similar level of stealthiness, even where the LNPs were formulated with DSPE-PEG5K-N₃, whereby the LNPs can be readily conjugated with a protein-based targeting ligand as described above. The results shown in FIG. 10 indicate that significantly lower opsonization-driven uptake was observed for LNPs containing 2.8-6.8 percent (%) of PG as compared to LNPs containing 3% PEG, thereby suggesting that PG may provide better shielding to the LNP base composition than 3% PEG.

TABLE 14

| | Diameter | | EE | |
|---|---|---|---|---|
| Formulation | (nm) | PDI | (%) | Lipid composition |
| 888 | 83.2 | 0.03 | 97.2 | Lipid Z:DSPC:Chol:DODA- |
| | | | | PG45:DSPE-PEG5000N₃:DiD |
| | | | | (47.5:10.0:40.5:1.3:0.2:0.5) |
| 889 | 77.5 | 0.02 | 97.9 | Lipid Z:DSPC:Chol:DODA- |
| | | | | PG45:DSPE-PEG5000N₃:DiD |
| | | | | (47.5:10.0:40.0:1.8:0.2:0.5) |
| 890 | 74.1 | 0.05 | 97.1 | Lipid Z:DSPC:Chol:DODA- |
| | | | | PG45:DSPE-PEG5000N₃:DiD |
| | | | | (47.5:10.0:39.5:2.3:0.2:0.5) |
| 891 | 70.9 | 0.06 | 95.6 | Lipid Z:DSPC:Chol:DODA- |
| | | | | PG45:DSPE-PEG5000N₃:DiD |
| | | | | (47.5:10.0:39.0:2.8:0.2:0.5) |
| 892 | 62.9 | 0.09 | 98.1 | Lipid Z:DSPC:Chol:DODA- |
| | | | | PG45:DSPE-PEG5000N₃:DiD |
| | | | | (47.5:10.0:37.0:4.8:0.2:0.5) |
| 893 | 65.6 | 0.19 | 98.4 | Lipid Z:DSPC:Chol:DODA- |
| | | | | PG45:DSPE-PEG5000N₃:DiD |
| | | | | (47.5:10.0:35.0:6.8:0.2:0.5) |

1. A polymer-conjugated lipid, comprising:
(i) a polyglycerol (PG) or a PG derivative;
(ii) a lipid moiety represented by Formula (I) 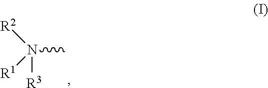
or a pharmaceutically acceptable salt thereof, wherein:
R¹ is absent, hydrogen, C₁-C₆ alkyl, or a hydrophobic tail comprising 10-30 carbon atoms;
R² is absent, hydrogen, C₁-C₆ alkyl, or a hydrophobic tail comprising 10-30 carbon atoms;
wherein, when R¹ and R² are each hydrogen, C₁-C₆ alkyl, or a hydrophobic tail comprising 10-30 carbon atoms, N is positively charged; and
R³ is a hydrophobic tail comprising 10-30 carbon atoms; and
(iii) a linker conjugating the PG or the PG derivative to the lipid moiety,
wherein [symbol] in Formula (I) is a bond conjugating the lipid moiety and the linker.

2. The polymer-conjugated lipid of claim 1, wherein the PG derivative is a carboxylated PG.

3. The polymer-conjugated lipid of claim 2, wherein the carboxylated PG is a glutarylated PG or a 2-carboxycyclohexane-1-carboxylated PG.

4.-5. (canceled)

6. The polymer-conjugated lipid of claim 1, wherein the PG or the PG derivative is linear or branched.

7. The polymer-conjugated lipid of claim 1, wherein R¹ is absent, and wherein R² and R³ are each independently a hydrophobic tail comprising 14, 15, 16, 17, 18, 19, 20, 21, or 22 carbon atoms.

8. The polymer-conjugated lipid of claim 7, wherein R² and R³ are each independently a hydrophobic tail comprising 16, 17, 18, 19, 20, 21, or 22 carbon atoms.

9. The polymer-conjugated lipid of claim 8, wherein R² and R³ are each independently a hydrophobic tail comprising 18 carbon atoms, and wherein the lipid moiety is dioctadecylamine (DODA).

10. The polymer-conjugated lipid of claim 9, wherein the lipid moiety conjugated to a linker is represented by the following structure:

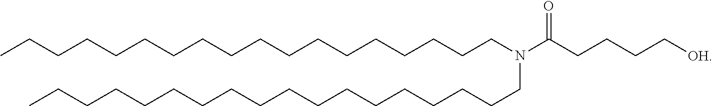 [SMILES: CCCCCCCCCCCCCCCCCCN(CCCCCCCCCCCCCCCCCC)C(=O)CCCCO]

11. The polymer-conjugated lipid of claim 1, wherein: the PG or the PG derivative comprises about 5 to 100 monomeric units.

12. The polymer-conjugated lipid of claim 11, wherein the PG or the PG derivative comprises about 5, 6, 7, 8, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 monomeric units.

13. The polymer-conjugated lipid of claim 12, wherein the PG or the PG derivative comprises about 8, 34, 39, 45, 46, 58, or 68 monomeric units.

14. (canceled)

15. The polymer-conjugated lipid of claim 13, wherein the PG or the PG derivative comprises about 39 monomeric units.

16. The polymer-conjugated lipid of claim 13, wherein the PG or the PG derivative comprises about 68 monomeric units.

17. The polymer-conjugated lipid of claim 13, wherein the PG or the PG derivative comprises about 46 monomeric units.

18. The polymer-conjugated lipid of claim 13, wherein the PG or the PG derivative comprises about 58 monomeric units.

19. The polymer-conjugated lipid of claim 1, wherein the linker is selected from the group consisting of an alkyl linker, a glycerol linker, a phosphate linker, a phosphate ester linker, an ether linker, an ester linker, a diester linker, an amide linker, a diamide linker, an amine linker, a peptide linker, a phosphoethanolamine linker, a phosphocholine linker, a carbamate linker, a diamide alkyl linker, a cleavable linker, and any combination thereof.

20. The polymer-conjugated lipid of claim 1, wherein;
the linker is selected from the group consisting of —(CH₂)ₙ—, —C(O)(CH₂)ₙ—, —C(O)O(CH₂)ₙ, —OC(O)(CH₂)ₙC(O)O—, and —NH(CH₂)ₙC(O)O—, wherein n is an integer ranging from 1 to 20;
wherein the linker is a glutaryl linker or a succinyl linker; or
wherein the linker is —C(O)(CH₂)ₙ—, and wherein n is 2, 3, 4, 5, or 6.

21.-28. (canceled)

29. The polymer-conjugated lipid of claim 1, further comprising a reactive species conjugated to the PG or the PG derivative, wherein the reactive species is functionalized to be conjugated to a targeting moiety.

30. The polymer-conjugated lipid of claim 29, wherein the reactive species is a click chemistry reagent, maleimide, or thiol.

31. The polymer-conjugated lipid of claim 30, wherein the click chemistry reagent is selected from the group consisting of a dibenzocyclooctyne (DBCO) reagent, a transcylooctene (TCO) reagent, a tetrazine (Tz) reagent, an alkyne reagent, and an azide reagent.

32. The polymer-conjugated lipid of claim 29, further comprising a targeting moiety conjugated to the PG or the PG derivative via the reactive species.

33. The polymer-conjugated lipid of claim 32, wherein the targeting moiety is conjugated to the PG or the PG derivative via a dibenzocyclooctyne (DBCO)-azide conjugation, an azide-alkyne conjugation, a TCO-Tz conjugation, or a thiol-maleimide conjugation.

34. The polymer-conjugated lipid of claim 32, wherein the targeting moiety is capable of binding to a liver cell or a T cell.

35.-38. (canceled)

39. The polymer-conjugated lipid of claim 32, wherein the targeting moiety is an antibody or an antibody fragment, wherein the antibody or the antibody fragment is capable of specifically binding an antigen present on the surface of a cell.

40. The polymer-conjugated lipid of claim 39, wherein the antibody or the antibody fragment is selected from the group consisting of a monoclonal antibody (mAb), a single chain variable fragment (scFᵥ), a heavy chain antibody (hcAb), a nanobody (Nb), a heavy-chain-only immunoglobulin (HCIg), an immunoglobulin new antigen receptor (IgNAR), variable domain of immunoglobulin new antigen receptor (V_{NAR}), a single-domain antibody, and a variable heavy chain-only antibody (V_{HH}).

41.-193. (canceled)

194. The polymer-conjugated lipid of claim 40, wherein the antibody or the antibody fragment is a single chain variable fragment (scFᵥ) or a variable heavy chain-only antibody (V_{HH}).

195. The polymer-conjugated lipid of claim 194, wherein the antibody or the antibody fragment is a variable heavy chain-only antibody (V_{HH}).

196. The polymer-conjugated lipid of claim 39, wherein the cell is a T cell.

197. A polymer-conjugated lipid represented by the following structure: 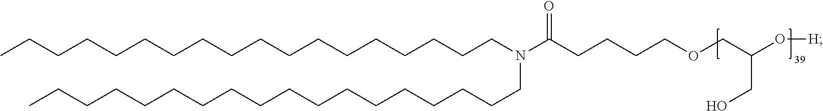
or a pharmaceutically acceptable salt or ester thereof, or a deuterated analogue thereof.

198. A polymer-conjugated lipid represented by the following structure: 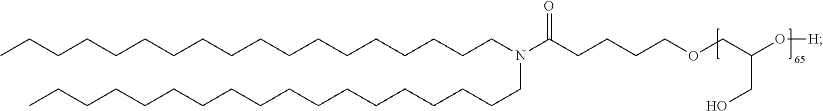
or a pharmaceutically acceptable salt or ester thereof, or a deuterated analogue thereof.
